SEQUENCE LISTING (6 sequences)

SEQ ID NO: 1 - DNA, 20 bp, synthetic construct
  source 1..20: mol_type = other DNA
agacagatgctccaattgga

SEQ ID NO: 2 - DNA, 20 bp, synthetic construct
  source 1..20: mol_type = other DNA
aaaaagcttccgcctgatgg

SEQ ID NO: 3 - DNA, 20 bp, synthetic construct
  source 1..20: mol_type = other DNA
atgaaaagccagatggccag

SEQ ID NO: 4 - protein, 384 aa, Homo sapiens
MAEQTYSWAYSLVDSSQVSTFLISILLIVYGSFRSLNMDFENQDKEKDSNSSSGSFNGNSTNNSIQTIDSTQALFLPIGASVSLLVMFFFFDSVQVVFTICTAVLATIAFAFLLLPMCQYLTRPCSPQNKISFGCCGRFTAAELLSFSLSVMLVLIWVLTGHWLLMDALAMGLCVAMIAFVRLPSLKVSCLLLSGLLIYDVFWVFFSAYIFNSNVMVKVATQPADNPLDVLSRKLHLGPNVGRDVPRLSLPGKLVFPSSTGSHFSMLGIGDIVMPGLLLCFVLRYDNYKKQASGDSCGAPGPANISGRMQKVSYFHCTLIGYFVGLLTATVASRIHRAAQPALLYLVPFTLLPLLTMAYLKGDLRRMWSEPFHSKSSSSRFLEV

SEQ ID NO: 5 - DNA, 20 bp, synthetic construct
  source 1..20: mol_type = other DNA
gtcaccaatcctgtccctag

SEQ ID NO: 6 - DNA, 20 bp, synthetic construct
  source 1..20: mol_type = other DNA
gagtagcgcgagcacagcta

(19) United States
(12) Patent Application Publication
(10) Pub. No.: US 2026/0048120 A1
(43) Pub. Date: Feb. 19, 2026

(54) MODIFIED IMMUNE CELLS AND USES THEREOF

(71) Applicant: Peking University, Beijing (CN)

(72) Inventors: Wensheng WEI, Beijing (CN); Zeguang WU, Beijing (CN); Qiezhong LAMAO, Beijing (CN); Jinxin YANG, Beijing (CN); Yuanyuan QIU, Beijing (CN)

(21) Appl. No.: 19/120,629

(22) PCT Filed: Oct. 12, 2023
(86) PCT No.: PCT/CN2023/124214

§ 371 Date: Apr. 11, 2025

(30) Foreign Application Priority Data
Oct. 12, 2022 (WO) PCT/CN2022/124803
Aug. 2, 2023 (WO) PCT/CN2023/110694

(51) Int. Cl. A61K 40/11; A61K 35/17; A61K 40/13; A61K 40/15; A61K 40/31; A61K 40/32; A61K 40/50; A61P 37/06; C07K 14/725; C12N 5/0781; C12N 5/0783; C12N 9/22; C12N 9/78; C12N 15/10; C12N 15/113; G01N 33/68

(52) U.S. Cl. CPC A61K 40/11; A61K 35/17; A61K 40/13; A61K 40/15; A61K 40/31; A61K 40/32; A61K 40/50; A61P 37/06; C07K 14/7051; C12N 5/0635; C12N 5/0636; C12N 5/0646; C12N 9/222; C12N 9/78; C12N 15/10; C12N 15/1137; C12Y 305/04002; C12Y 305/04005; G01N 33/68; C12N 2310/20; C12N 2510/00

(57) ABSTRACT

It relates to immune cells (e.g., T cells such as CAR-T cells, NK cells such as CAR-NK cells) modified to have no or reduced expression and/or function of one or more target proteins selected from the group consisting of: Signal Peptide Peptidase Like 3 (SPPL3), FADD, FAS, CASP8, ARID1A, BAK1, BID, ETS1, IKZF2, and HIST1H1B (such as SPPL3), uses thereof, and methods for generating thereof. Also provided are uses of the one or more target proteins (e.g., SPPL3) as a biomarker.

### CROSS REFERENCE TO RELATED APPLICATIONS

This patent application is a national stage application under 35 U.S.C. § 371 of International Application No. PCT/CN2023/124214, filed internationally on Oct. 12, 2023, which claims priority benefit of International Application No. PCT/CN2022/124803 filed on Oct. 12, 2022, and International Application No. PCT/CN2023/110694 filed on Aug. 2, 2023, the contents of each of which are incorporated herein by reference in their entirety.

### REFERENCE TO AN ELECTRONIC SEQUENCE LISTING

The contents of the electronic sequence listing (165392001442seqlist.xml; Size: 6,407 bytes; and Date of Creation: Oct. 10, 2023) are herein incorporated by reference in their entirety.

### FIELD OF THE PRESENT APPLICATION

The present application relates to immune cells (e.g., T cells such as CAR-T cells, NK cells such as CAR-NK cells) modified to have no or reduced expression and/or function of one or more target proteins selected from the group consisting of: Signal Peptide Peptidase Like 3 (SPPL3), FADD, FAS, CASP8, ARID1A, BAK1, BID, ETS1, IKZF2, and HIST1H1B (such as immune cells modified to have no or reduced expression and/or function of SPPL3 protein), uses thereof, and methods for generating thereof. Also provided are uses of the one or more target proteins (e.g., SPPL3) as a biomarker.

### BACKGROUND OF THE PRESENT APPLICATION

Immunotherapeutic approaches, including adoptive immune cell therapy (e.g., CAR-T therapy, CAR-NK therapy), are playing an increasingly important role in the treatment of cancer, viral infections, and other pathophysiological autoimmune conditions. Compared to autologous immune cell therapy which often requires a long and expensive customized manufacturing process and is not suitable for all patients, allogeneic immune cell therapy (e.g., universal CAR-T or CAR-NK therapy) has become a more appealing approach where immune cells are derived from healthy donors and can provide off-the-shelf products suitable for many patients instead of only a single person.

Universal CAR-T or CAR-NK therapy has following advantages: i) can be pre-made and supplied directly from stock, rather than preparation as needed which takes weeks; ii) the raw material T cells can be obtained from healthy donors, screened with strict procedures such as with strong activity and no contamination of diseased cells (e.g., tumor cells); iii) has wide range of adaptations, patients will not lose treatment opportunities due to failure of preparation or non-compliance with preparation standards; iv) product quality is controllable, such as in accordance with good manufacturing practice (GMP) standards; and v) suitable for industrial manufacture, which can effectively reduce production and treatment costs.

Poor persistence of modified immune cells (e.g., allogeneic CAR-T or CAR-NK cells) can limit their therapeutic effectiveness (L. Jafarzadeh et al., Front Immunol. 2020; 11:702), and remains one of the biggest challenges for their applications. First, patient's immune system (e.g., host T cells, NK cells) will recognize infused non-HLA matched allogeneic immune cells as foreign and reject them, namely, host-versus-graft (HvG) responses. To overcome this issue, researchers have knocked out beta-2 microglobulin (B2M) required for human leucocyte antigen (HLA) class I expression in CAR-T cells using the clustered regularly interspaced short palindromic repeats (CRISPR)/Cas9 (CRISPR-associated protein 9) (CRISPR/Cas9) system, to prevents host TCRαβ cells recognizing donor CAR-T cells as foreign via HLA class I (Ren et al., Clin. Cancer Res. 2017; 23:2255-2266). However, T cells with reduced HLA class I expression are also be targeted by host natural killer (NK) cells, representing a hurdle to prevent allogeneic T cell rejection (Liu et al. Curr. Res. Transl. Med. 2018; 66:39-42; Ludigs et al., Nat Commun. 2016; 7:10554). Second, in addition to targeting T cells with reduced HLA class I expression, host NK cells can kill activated T cells (e.g., allogeneic CAR-T cells) due to the expression of ligands on the activated T cells, such as those that can be recognized by NKG2D or NKp46 expressed on NK cells (Waggoner et al., Nature. 2011; 481(7381):394-398; Cerboni et al., Blood. 2007; 110(2):606-15). Third, allogeneic immune cells (e.g., CAR-T or CAR-NK cells) can experience activation-induced cell death (AICD), which is a programmed cell death process due to the interaction of Fas receptor (Fas, CD95) and Fas ligands (FasL, CD95 ligand). Normally, FasL is a killer effector molecule expressed and/or secreted by immune cells (e.g., T cells, and NK cells), Fas is an apoptotic receptor expressed on the surface of tumor cells, and the interaction of Fas and Fas ligands is a mechanism for immune cells to kill tumor cells. However, activated immune cells (e.g., T cells, B cells, and NK cells) also express Fas in addition to FasL, thus will be killed by themselves or each other. See, e.g., Huan et al., Hum Cell. 2022; 35(2):441-447. All above mechanisms contribute to the poor persistence of allogeneic immune cells in vivo.

Adoptive immunotherapy can also have limited application due to graft-versus-host-disease (GvHD) in patients. For example, CAR-T cells expressing endogenous T cell receptors (TCRs) may recognize major and minor histocompatibility antigens following administration to an allogeneic patient, leading to non-specific effects and development of GvHD in patients. To overcome this issue, researchers have knocked out endogenous TCR in allogeneic T cells (e.g., CAR-T cells) by gene editing such as CRISPR/Cas9.

The activity of NK cells is regulated by a complex interplay of various cell surface inhibitory and activating receptors. Inhibitory receptors include killer immunoglobulin-like receptors (KIRs) and CD94/NKG2A, recognize major histocompatibility complex (MHC) or HLA class I molecules, allow NK cells to recognize autologous cells and prevent them from attacking the host tissue. When no matching MHC class I molecule is present, the inhibition of NK cytotoxicity is released and the balance is shifted towards NK cell activation via activating receptor engagement. During viral infections or malignant transformation, the transformed cells decrease MHC class I antigen expression on cell surface to avoid recognition by T cells. However, NK cells can recognize such transformed cells as “altered self” whose abnormal level of MHC class I expression results in decreased engagement of inhibitory KIRs to provide effector response and cytotoxic killing of the transformed cells (Nayyar et al. Front Oncol. 2019; 9:51).

Activated NK cells can kill target cells through the following pathways: i) perforin and granzyme, released from cytoplasmic granules of NK cells to dissolve or activate apoptosis of target cells; ii) TNFα and IFNγ, released by activated NK cells to induce tumor cell apoptosis, including changing the stability of target cell lysosomes, affecting target cell membrane phospholipid metabolism, changing target cell pH, and activating target cell endonucleases to degrade genomic DNA; and iii) FasL and TNF-related apoptosis-inducing ligand (TRAIL), secreted/expressed by activated NK cells to interact with death receptors (Fas, DR4/TRAIL-R1, and DR5/TRAIL-R2) on target cells, inducing death receptors' conformational changes that promote binding of adaptor protein Fas-associated death domain protein (FADD), which then binds to caspase-8 precursor protein to initiate the cascade reaction of caspase, activates caspase-3, leading to degradation of intracellular structural and functional proteins and eventually target cell apoptosis.

The CRISPR/Cas9 system enables editing at targeted genomic sites with high efficiency and specificity. One of its extensive applications is to identify functions of coding genes, non-coding RNAs and regulatory elements through high-throughput pooled screening in combination with next generation sequencing (“NGS”) analysis. By introducing a pooled single-guide RNA (“sgRNA”) or paired-guide RNA (“pgRNA”) library into cells expressing Cas9 or catalytically inactive Cas9 (dCas9) fused with effector domains, investigators can perform multifarious genetic screens by generating diverse mutations, large genomic deletions, transcriptional activation or transcriptional repression.

To generate a high-quality cell library of gRNAs for any given pooled CRISPR screen, one must use a low multiplicity of infection (“MOI”) during cell library construction to ensure that each cell on average harbors less than one sgRNA or pgRNA to minimize the false discovery rate (FDR) of the screen. To further reduce the FDR and increase data reproducibility, in-depth coverage of gRNAs and multiple biological replicates are often necessary to obtain hit genes with high statistical significance, resulting in increased workload. Additional difficulties may arise when one performs a large number of genome-wide screens, when cell materials for library construction are limited, or when one conducts more challenging screens (i.e., in vivo screens) for which it is difficult to obtain experimental replicates or control the MOI. The “internal barcodes (“iBAR”) methods previously developed by the Applicant (see WO2020125762, the content of which is incorporated herein by reference in its entirety) provide a reliable and highly efficient screening strategy for large-scale target identification in eukaryotic cells, with much lower false-positive and false-negative rates, and allow cell library generation using a high MOI. For example, compared to a conventional CRISPR/Cas screen with a low MOI of 0.3, the iBAR methods can reduce the starting cell numbers for more than 20-fold (e.g., at an MOI of 3) to more than 70-fold (e.g., at an MOI of 10), while maintaining high efficiency and accuracy. The iBAR system is particularly useful for cell-based screens in which the cells are available in limited quantities, or for in vivo screens in which viral infection to specific cells or tissues is difficult to control at low MOI.

The disclosures of all publications, patents, patent applications and published patent applications referred to herein are hereby incorporated herein by reference in their entirety.

### BRIEF SUMMARY OF THE PRESENT APPLICATION

The present invention in one aspect provides an immune cell, wherein the immune cell is modified to have no or reduced expression and/or function of one or more target proteins selected from the group consisting of: SPPL3, FADD, FAS, CASP8, ARID1A, BAK1, BID, ETS1, IKZF2, and HIST1H1B. In some embodiments, the immune cell is modified to have no or reduced expression and/or function of SPPL3 protein. In some embodiments, the immune cell has at least about 10% less activation-induced cell death (AICD) compared to a reference immune cell not having the modification to reduce or eliminate expression and/or function of the SPPL3 protein. In some embodiments, the expression of the SPPL3 protein is reduced or inhibited by an antisense RNA, an siRNA, or an shRNA specifically recognizing an RNA encoding the SPPL3 protein. In some embodiments, the immune cell is modified to express a dominant negative SPPL3 protein variant or dominant negative fragment thereof. In some embodiments, the immune cell is genetically modified at the SPPL3 locus or at SPPL3 RNA. In some embodiments, the SPPL3 locus is modified by gene editing, or the SPPL3 RNA is modified by RNA editing. In some embodiments, the gene editing or RNA editing is mediated by non-homologous end-joining (NHEJ), homology directed repair (HDR), zinc-finger nuclease (ZFN), transcription activator-like effector nuclease (TALEN), or CRISPR/Cas. In some embodiments, the gene editing comprises gene knockout (KO). In some embodiments, the gene editing or RNA editing comprises base editing. In some embodiments, the gene editing or RNA editing is mediated by CRISPR/Cas. In some embodiments, the gene editing or RNA editing comprises contacting a precursor immune cell with i) a guide RNA (gRNA) construct, wherein the gRNA construct comprises or encodes a gRNA comprising a guide sequence that is complementary to a target site in the SPPL3 locus or SPPL3 RNA; and optionally ii) a Cas component comprising a Cas protein or a nucleic acid encoding the Cas protein, under a condition that allows introduction of the gRNA construct and the optional Cas component into the precursor immune cell. In some embodiments, the precursor immune cell expresses a Cas protein. In some embodiments, the Cas protein has endonuclease activity. In some embodiments, the Cas protein is fusion protein comprising i) a dead Cas protein (dCas) and ii) an adenine deaminase (ADA) or a cytidine deaminase (CDA) or functional fragment thereof. In some embodiments, the Cas protein is Cas9. In some embodiments, the guide sequence is encoded by a nucleic acid sequence comprising the sequence of SEQ ID NO: 1.

In some embodiments according to any one of the immune cells described above, the immune cell has or is further modified to have no or reduced expression and/or function of one or more other proteins selected from the group consisting of TCRα, TCRβ, TCRγ, TCRδ, HLA-A, HLA-B, HLA-C, HLA-E, HLA-F, HLA-G, B2M, PD-1, TIM-3, LAG-3, CTLA-4, CISH, Fas, FADD, CASP8, ARID1A, BAK1, BID, ETS1, IKZF2, HIST1H1B, B7-H6, MICA, MICB, ULBP1, ULBP2, ULBP3, ULBP4, ULBP5, ULBP6, and ligands of NKp46.

In some embodiments according to any one of the immune cells described above, the immune cell expresses or is further modified to express an engineered receptor, such as a chimeric antigen receptor (CAR), an engineered TCR, or a T cell antigen coupler (TAC). In some embodiments, the engineered receptor is a CAR comprising: i) an extracellular antigen binding domain specifically recognizing a target antigen; ii) a transmembrane domain; and iii) an intracellular signaling domain. In some embodiments, the modification to reduce or eliminate expression and/or function of the one or more target proteins: i) does not down-regulate or eliminate expression and/or function of the engineered receptor; or ii) down-regulates expression and/or function of the engineered receptor by at most about 30%.

In some embodiments according to any one of the immune cells described above, the immune cell is a T cell, a B cell, or a natural killer (NK) cell. In some embodiments, the immune cell is a T cell, such as selected from the group consisting of a helper CD4+ T cell, a cytotoxic CD8+ T cell, a memory T cell, a regulatory CD4+ T cell, a natural killer T (NKT) cell, a mucosal associated invariant T (MAIT) cell, a double negative T (DNT) cell, and a γδ T cell. In some embodiments, the modification to reduce or eliminate expression and/or function of the one or more target proteins: i) reduces at least about 10% cell surface expression of one or more of Fas, HLA-A, HLA-B, HLA-C, HLA-E, B7-H6, MICA, MICB, ULBP1, ULBP2, ULBP3, ULBP4, ULBP5, ULBP6, and ligands of NKp46; ii) reduces at least about 10% killing by an allogeneic T cell; and/or iii) reduces at least about 10% killing by an autologous or allogeneic NK cell.

In some embodiments according to any one of the immune cells described above, the immune cell has at least about 10% longer in vivo persistence compared to a reference immune cell not having the modification to reduce or eliminate expression and/or function of the one or more target proteins.

In some embodiments according to any one of the immune cells described above, the immune cell is autologous or allogeneic.

The present invention in another aspect provides a method of identifying an individual as a suitable donor of an immune cell with prolonged in vivo persistence, comprising examining the expression and/or function of one or more target proteins selected from the group consisting of SPPL3, FADD, FAS, CASP8, ARID1A, BAK1, BID, ETS1, IKZF2, and HIST1H1B in the individual, wherein the identification of reduced or abolished expression and/or function of the one or more target proteins compared to a reference identifies the individual as the suitable donor. In some embodiments, the method comprises examining the expression and/or function of SPPL3 protein in the individual, wherein the identification of reduced or abolished expression and/or function of the SPPL3 protein compared to a reference identifies the individual as the suitable donor. In some embodiments, the method further comprises examining the expression and/or function of one or more other proteins selected from the group consisting of TCRα, TCRβ, TCRγ, TCRδ, HLA-A, HLA-B, HLA-C, HLA-E, HLA-F, HLA-G, B2M, PD-1, TIM-3, LAG-3, CTLA-4, CISH, Fas, FADD, CASP8, ARID1A, BAK1, BID, ETS1, IKZF2, HIST1H1B, B7-H6, MICA, MICB, ULBP1, ULBP2, ULBP3, ULBP4, ULBP5, ULBP6, and ligands of NKp46, wherein the identification of reduced or abolished expression and/or function of one or more of the other proteins further identifies the individual as the suitable donor. In some embodiments, the reference is the average expression and/or function of the one or more target proteins in a population of individuals. In some embodiments, the reference is the average expression and/or function of the SPPL3 protein in a population of individuals. In some embodiments, examining the expression and/or function of the one or more target proteins and/or the one or more other proteins comprises examining the sequence of the nucleic acid encoding the one or more target proteins and/or the one or more other proteins, wherein the identification of a mutation in the nucleic acid that reduces expression and/or function of the one or more target proteins and/or the one or more other proteins identifies the individual as the suitable donor. In some embodiments, examining the expression and/or function of the SPPL3 protein and/or the one or more other proteins comprises examining the sequence of the nucleic acid encoding the SPPL3 protein and/or the one or more other proteins, wherein the identification of a mutation in the nucleic acid that reduces expression and/or function of the SPPL3 protein and/or the one or more other proteins identifies the individual as the suitable donor.

The present invention in another aspect provides a method of excluding an individual as a suitable donor of an immune cell with prolonged in vivo persistence, comprising examining the expression and/or function of one or more target proteins selected from the group consisting of SPPL3, FADD, FAS, CASP8, ARID1A, BAK1, BID, ETS1, IKZF2, and HIST1H1B in the individual, wherein the individual is excluded as the suitable donor if no reduced or abolished expression and/or function of one or more target proteins compared to a reference is identified. In some embodiments, the method comprises examining the expression and/or function of SPPL3 protein in the individual, wherein the individual is excluded as the suitable donor if no reduced or abolished expression and/or function of the SPPL3 protein compared to a reference is identified. In some embodiments, the reference is the average expression and/or function of the one or more target proteins in a population of individuals. In some embodiments, the reference is the average expression and/or function of the SPPL3 protein in a population of individuals.

The present invention in another aspect provides a method of i) prolonging in vivo persistence, ii) reducing AICD, and/or iii) reducing host-versus-graft (HvG) response of an immune cell, comprising modifying the immune cell to reduce or eliminate expression and/or function of one or more target proteins selected from the group consisting of: SPPL3, FADD, FAS, CASP8, ARID1A, BAK1, BID, ETS1, IKZF2, and HIST1H1B. In some embodiments, the method comprises modifying the immune cell to reduce or eliminate expression and/or function of SPPL3 protein. In some embodiments, the expression of the SPPL3 protein is reduced or inhibited by an antisense RNA, an siRNA, or an shRNA specifically recognizing an RNA encoding the SPPL3 protein. In some embodiments, the immune cell is modified to express a dominant negative SPPL3 protein variant or dominant negative fragment thereof. In some embodiments, the immune cell is genetically modified at the SPPL3 locus or at SPPL3 RNA. In some embodiments, the SPPL3 locus is modified by gene editing, or wherein the SPPL3 RNA is modified by RNA editing. In some embodiments, the gene editing or RNA editing is mediated by NHEJ, HDR, ZFN, TALEN, or CRISPR/Cas. In some embodiments, the gene editing comprises gene KO. In some embodiments, the gene editing or RNA editing comprises base editing. In some embodiments, wherein the gene editing or RNA editing is mediated by CRISPR/Cas. In some embodiments, the method comprises contacting a precursor immune cell with i) a gRNA construct, wherein the gRNA construct comprises or encodes a gRNA comprising a guide sequence that is complementary to a target site in the SPPL3 locus or SPPL3 RNA; and optionally ii) a Cas component comprising a Cas protein or a nucleic acid encoding the Cas protein, under a condition that allows introduction of the gRNA construct and the optional Cas component into the precursor immune cell. In some embodiments, the precursor immune cell expresses a Cas protein. In some embodiments, the Cas protein has endonuclease activity. In some embodiments, the Cas protein is fusion protein comprising i) a dCas and ii) an ADA or a CDA or functional fragment thereof. In some embodiments, the Cas protein is Cas9. In some embodiments, the guide sequence is encoded by a nucleic acid sequence comprising the sequence of SEQ ID NO: 1.

In some embodiments according to any one of the methods of i) prolonging in vivo persistence, ii) reducing AICD, and/or iii) reducing HvG response of an immune cell described above, the method further comprises modifying the immune cell to reduce or eliminate expression and/or function of one or more other proteins selected from the group consisting of TCRα, TCRβ, TCRγ, TCRδ, HLA-A, HLA-B, HLA-C, HLA-E, HLA-F, HLA-G, B2M, PD-1, TIM-3, LAG-3, CTLA-4, CISH, Fas, FADD, CASP8, ARID1A, BAK1, BID, ETS1, IKZF2, HIST1H1B, B7-H6, MICA, MICB, ULBP1, ULBP2, ULBP3, ULBP4, ULBP5, ULBP6, and ligands of NKp46. In some embodiments, the immune cell is genetically modified at one or more loci or RNAs encoding the one or more other proteins.

In some embodiments according to any one of the methods of i) prolonging in vivo persistence, ii) reducing AICD, and/or iii) reducing HvG response of an immune cell described above, the immune cell expresses an engineered receptor. In some embodiments, the method further comprises introducing into the immune cell a nucleic acid encoding an engineered receptor. In some embodiments, the nucleic acid encoding the engineered receptor, the nucleic acid encoding the gRNA against SPPL3, and/or the nucleic acid encoding the Cas protein are on different vectors. In some embodiments, the engineered receptor is a CAR, an engineered TCR, or a TAC. In some embodiments, the engineered receptor is a CAR comprising: i) an extracellular antigen binding domain specifically recognizing a target antigen; ii) a transmembrane domain; and iii) an intracellular signaling domain. In some embodiments, the modification to reduce or eliminate expression and/or function of the one or more target proteins: i) does not down-regulate or eliminate expression and/or function of the engineered receptor; or ii) down-regulates expression and/or function of the engineered receptor by at most about 30%.

In some embodiments according to any one of the methods of i) prolonging in vivo persistence, ii) reducing AICD, and/or iii) reducing HvG response of an immune cell described above, the immune cell is a T cell, a B cell, or an NK cell. In some embodiments, the immune cell is a T cell, such as selected from the group consisting of a helper CD4+ T cell, a cytotoxic CD8+ T cell, a memory T cell, a regulatory CD4+ T cell, an NKT cell, an MAIT cell, a DNT cell, and a γδ T cell. In some embodiments, the modification to reduce or eliminate expression and/or function of the one or more target proteins: i) reduces at least about 10% cell surface expression of one or more of Fas, HLA-A, HLA-B, HLA-C, HLA-E, B7-H6, MICA, MICB, ULBP1, ULBP2, ULBP3, ULBP4, ULBP5, ULBP6, and ligands of NKp46; ii) reduces at least about 10% killing by an allogeneic T cell; and/or iii) reduces at least about 10% killing by an autologous or allogeneic NK cell.

In some embodiments according to any one of the methods of i) prolonging in vivo persistence, ii) reducing AICD, and/or iii) reducing HvG response of an immune cell described above, the modification to reduce or eliminate expression and/or function of the one or more target proteins: i) prolongs at least about 10% in vivo persistence of the immune cell; and/or ii) reduces at least about 10% AICD of the immune cell, compared to a same immune cell not modified to reduce or eliminate expression and/or function of the one or more target proteins.

The present invention in another aspect provides a method of identifying a target gene in an immune cell whose mutation increases resistance to AICD, comprising: a) providing an immune cell library comprising a plurality of immune cells, wherein each of the plurality of immune cells has a mutation at a hit gene (hit gene mutation), wherein the hit gene in at least two of the plurality of immune cells are different from each other; wherein the immune cell library is generated by contacting an initial population of immune cells with i) a guide RNA (sgRNA) library comprising a plurality of sgRNA constructs, wherein each sgRNA construct comprises or encodes an sgRNA, and wherein each sgRNA comprises a guide sequence that is complementary to a target site in a corresponding hit gene; and ii) a Cas component comprising a Cas protein or a nucleic acid encoding the Cas protein, under a condition that allows introduction of the sgRNA constructs and the Cas component into the initial population of immune cells and generation of the mutations at the hit genes; b) contacting the immune cell library with a Fas ligand (FasL); c) obtaining from the immune cell library an AICD-resistant immune cell population; and d) identifying the target gene based on the difference between the profiles of sgRNAs or hit gene mutations in the AICD-resistant immune cell population and a control immune cell population.

In some embodiments according to any one of the methods of identifying a target gene described above, the control immune cell population is i) a subpopulation of the immune cell library before step b); or ii) a same immune cell library cultured under the same condition and not contacted with the FasL. In some embodiments, the method further comprises i) obtaining the control immune cell population before step b); or ii) culturing a same immune cell library under the same condition and not contacting with the FasL.

In some embodiments according to any one of the methods of identifying a target gene described above, the identification of the target gene is based on the difference between the profiles of sgRNAs in the AICD-resistant immune cell population and the control immune cell population.

In some embodiments according to any one of the methods of identifying a target gene described above, the profiles of sgRNAs in the AICD-resistant immune cell population and the control immune cell population are identified by next generation sequencing (NGS). In some embodiments, the method comprises comparing sgRNA sequence counts obtained from the AICD-resistant immune cell population with sgRNA sequence counts obtained from the control immune cell population, wherein the hit genes whose corresponding sgRNA guide sequences are identified as enriched in the AICD-resistant immune cell population compared to the control immune cell population with an false discovery rate (FDR)≤0.2 are identified as target genes whose mutations increase resistance to AICD (AICD resistant genes).

In some embodiments according to any one of the methods of identifying a target gene described above, the sgRNA library and the Cas component are introduced into the initial population of immune cells sequentially. In some embodiments, the initial population of immune cells express the Cas protein before introducing the sgRNA library. In some embodiments, the Cas protein is Cas9. In some embodiments, each sgRNA comprises the guide sequence fused to a second sequence, and wherein the second sequence comprises a repeat-anti-repeat stem loop that interacts with Cas9. In some embodiments, the second sequence of each sgRNA further comprises a stem loop 1, a stem loop 2, and/or a stem loop 3.

In some embodiments according to any one of the methods of identifying a target gene described above, each sgRNA further comprises an internal barcode (iBAR) sequence (sgRNA^{iBAR}), wherein each sgRNA^{iBAR} is operable with the Cas protein to modify the hit gene. In some embodiments, each sgRNA^{iBAR} comprises in the 5′-to-3′ direction a first stem sequence and a second stem sequence, wherein the first stem sequence hybridizes with the second stem sequence to form a double-stranded RNA (dsRNA) region that interacts with the Cas protein, and wherein the iBAR sequence is disposed between the 3′ end of the first stem sequence and the 5′ end of the second stem sequence. In some embodiments, the Cas protein is Cas9, and wherein the iBAR sequence of each sgRNA^{iBAR} is inserted in the loop region of the repeat-anti-repeat stem loop. In some embodiments, each guide sequence comprises about 17 to about 23 nucleotides. In some embodiments, the sgRNA library is an sgRNA^{iBAR} library, wherein the sgRNA^{iBAR} library comprises a plurality of sets of sgRNA^{iBAR} constructs, wherein each set of sgRNA^{iBAR} constructs comprise four sgRNA^{iBAR} constructs each comprising or encoding an sgRNA^{iBAR}, wherein the guide sequences for the four sgRNA^{iBAR} constructs are the same, wherein the iBAR sequence for each of the four sgRNA^{iBAR} constructs is different from each other, and wherein the guide sequence of each set of sgRNA^{iBAR} constructs is complementary to a different target site in the hit gene. In some embodiments, the immune cell library has at least about 500-fold coverage for each sgRNA^{iBAR}.

In some embodiments according to any one of the methods of identifying a target gene described above, at least about 95% of the sgRNA constructs in the sgRNA library are introduced into the initial population of immune cells.

In some embodiments according to any one of the methods of identifying a target gene described above, the immune cell library has at least about 2000-fold coverage for each sgRNA.

In some embodiments according to any one of the methods of identifying a target gene described above, the sgRNA library comprises at least about 2000 sgRNA constructs.

In some embodiments according to any one of the methods of identifying a target gene described above, each sgRNA construct in the sgRNA library is a plasmid.

In some embodiments according to any one of the methods of identifying a target gene described above, each sgRNA construct in the sgRNA library is a viral vector, such as a lentiviral vector. In some embodiments, the sgRNA library is contacted with the initial population of immune cells at a multiplicity of infection (MOI) of at least about 2.

In some embodiments according to any one of the methods of identifying a target gene described above, step c) comprise using fluorescence-activated cell sorting (FACS) or centrifugation to obtain the AICD-resistant immune cell population. In some embodiments, step c) further comprises contacting the immune cell library with a viability indicator, such as one or more of propidium iodide (PI), DAPI, 7-AAD, and Annexin V. In some embodiments, the AICD-resistant immune cell population is Annexin V⁻ negative and DAPI⁻ negative.

In some embodiments according to any one of the methods of identifying a target gene described above, step b) comprises culturing the immune cell library for about 16 hours in the presence of FasL.

In some embodiments according to any one of the methods of identifying a target gene described above, the sgRNA sequence counts are subject to median ratio normalization followed by mean-variance modeling. In some embodiments, the sgRNA library is an sgRNA^{iBAR} library, and the variance of each guide sequence is adjusted based on data consistency among the iBAR sequences in the sgRNA^{iBAR} sequences corresponding to the guide sequence. In some embodiments, the data consistency among the iBAR sequences in the sgRNA^{iBAR} sequences corresponding to each guide sequence is determined based on the direction of the fold change of each iBAR sequence, wherein the variance of the guide sequence is increased if the fold changes of the iBAR sequences are in different directions with respect to each other.

In some embodiments according to any one of the methods of identifying a target gene described above, the method comprises: a) providing the immune cell library; b1) contacting the immune cell library with a first FasL (first FasL treatment step); c1-i) obtaining from the immune cell library a first AICD-resistant immune cell population (first obtaining step); c1-ii) optionally culturing the first AICD-resistant immune cell population for about 6 days (optional first recovery step); b2) optionally contacting the first AICD-resistant immune cell population with a second FasL (optional second FasL treatment step); c2-i) optionally obtaining from step b2) a second AICD-resistant immune cell population (optional second obtaining step); c2-ii) optionally culturing the second AICD-resistant immune cell population for about 6 days (optional second recovery step); b3) optionally contacting the second AICD-resistant immune cell population with a third FasL (optional third FasL treatment step); c3-i) optionally obtaining from step b3) a final AICD-resistant immune cell population (optional third obtaining step); c3-ii) optionally culturing the final AICD-resistant immune cell population for about 6 days (optional third recovery step); and d) identifying the target gene based on the difference between the profiles of sgRNAs or hit gene mutations in the final AICD-resistant immune cell population and the control immune cell population. In some embodiments, the first, second, and third FasL have the same concentration. In some embodiments, the first, second, and third FasL have different concentrations.

In some embodiments according to any one of the methods of identifying a target gene described above, the method comprises: a) providing the immune cell library; b1) contacting the immune cell library with a FasL (first FasL treatment step); c1-i) centrifuging the immune cell library after step b1) to obtain a first AICD-resistant immune cell population (first obtaining step); c1-ii) culturing the first AICD-resistant immune cell population for about 6 days (first recovery step); b2) contacting the first AICD-resistant immune cell population with the FasL (second FasL treatment step); c2-i) centrifuging the first AICD-resistant immune cell population after step b2) to obtain a second AICD-resistant immune cell population (second obtaining step); c2-ii) culturing the second AICD-resistant immune cell population for about 6 days (second recovery step); b3) contacting the second AICD-resistant immune cell population with the FasL (third FasL treatment step); c3-i) centrifuging the second AICD-resistant immune cell population after step b3) to obtain a final AICD-resistant immune cell population (third obtaining step); c3-ii) culturing the final AICD-resistant immune cell population for about 6 days (third recovery step); and d) comparing sgRNA sequence counts obtained from the final AICD-resistant immune cell population with sgRNA sequence counts obtained from the control immune cell population, wherein the hit genes whose corresponding sgRNA guide sequences are identified as enriched in the final AICD-resistant immune cell population compared to the control immune cell population with an FDR≤0.2 are identified as target genes whose mutations increase resistance to AICD (AICD resistant genes).

In some embodiments according to any one of the methods of identifying a target gene described above, further comprising ranking the identified target genes, wherein the target gene ranking is based on the degree of enrichment of the sgRNA guide sequences in the AICD-resistant immune cell population compared to the control immune cell population. In some embodiments, the sgRNA library is an sgRNA^{iBAR} library, and wherein the target gene ranking is further adjusted based on data consistency among the iBAR sequences in the sgRNA^{iBAR} sequences corresponding to the guide sequence of the target gene. In some embodiments, further comprising assigning an AICD resistant score to the identified target gene, wherein target genes whose mutations increase resistance to AICD are ranked from high to low based on the fold of enrichment of the sgRNA guide sequences in the AICD-resistant immune cell population compared to the control immune cell population, and each target gene is assigned an AICD resistant score from high to low accordingly.

Also provided are methods of generating an immune cell with increased resistance to AICD, comprising inactivating an AICD resistant gene identified by any of the methods of identifying a target gene described above in a precursor immune cell. In some embodiments, the immune cell is a T cell, a B cell, or an NK cell. In some embodiments, the immune cell is a T cell, such as selected from the group consisting of a helper CD4+ T cell, a cytotoxic CD8+ T cell, a memory T cell, a regulatory CD4+ T cell, an NKT cell, an MAIT cell, a DNT cell, and a γδ T cell. In some embodiments, the precursor immune cell expresses an engineered receptor. In some embodiments, the method further comprises introducing into the precursor immune cell a nucleic acid encoding an engineered receptor. In some embodiments, the engineered receptor is a CAR.

Also provided are immune cells obtained by any of the methods described above, such as methods of generating an immune cell with increased resistance to AICD, or methods of i) prolonging in vivo persistence, ii) reducing AICD, and/or iii) reducing HvG response of an immune cell. In some embodiments, the immune cell is autologous or allogeneic.

Also provided are pharmaceutical compositions comprising any of the immune cells described above, and optionally a pharmaceutically acceptable excipient.

The present invention in another aspect provides a method of treating a disease in an individual, comprising administering to the individual an effective amount of any of the immune cells described above, or any of the pharmaceutical compositions described above. In some embodiments, the disease is associated with the expression of a target antigen, and wherein the immune cell expresses an engineered receptor specifically recognizing the target antigen, such as CAR. In some embodiments, the disease is a cancer, an infection, an inflammation, an autoimmune disease, or an immune-related disease characterized by effector cell exhaustion.

Kits and articles of manufacture that are useful for the methods described herein are also provided, such as kits for generating a modified immune cell (e.g., modified T cell such as modified CAR-T cell) with increased resistance to AICD.

### BRIEF DESCRIPTION OF THE DRAWINGS

FIG. 1 shows exemplary procedure for screening AICD/FasL resistant genes in immune cells (e.g., T cells).

FIG. 2 shows exemplary procedure for obtaining control or treatment sample for screening AICD/FasL resistant genes in immune cells (e.g., T cells).

FIG. 3 shows exemplary procedure for testing NK cell killing frequency in the mixture of SPPL3-KO T cells and control (NT-KO) T cells.

FIG. 4 shows an exemplary target gene identification workflow for Cas9⁺ sgRNA^{iBAR} immune cell (e.g., T cell) library.

FIG. 5 shows testing of appropriate FasL concentration for identifying AICD/FasL resistant genes in Cas9⁺ sgRNA^{iBAR} Jurkat cell library. Annexin V and DAPI staining indicates dead cells.

FIG. 6 shows robust rank aggregation (RRA) score vs. gene rank of the AICD/FasL resistance screening in Cas9⁺ sgRNA^{iBAR} Jurkat cell library. 10 exemplary AICD/FasL resistant genes with high scores were identified with names.

FIGS. 7A and 7B show that 100 ng/mL FasL treatment led to 25.7% cell survival rate (Annexin V⁻ & DAPI⁻) in control (NT-KO) Jurkat T cells, which was statistically significantly lower than 63.1% cell survival rate in SPPL3-KO Jurkat T cells. FIG. 7C shows that SPPL3-KO Jurkat T cells were much more resistant to FasL-mediated AICD under various FasL concentrations compared to control (NT-KO) Jurkat T cells.

FIG. 8A shows ratio of CellTrace™ Violet (CTV) labeled SPPL3-KO primary T cells vs. CellTrace™ carboxyfluorescein succinimidyl ester (CFSE) labeled control (NT-KO) primary T cells treated with various concentration of FasL. The higher the FasL concentration, the higher percentage of CTV labeled SPPL3-KO primary T cells. FIG. 8B shows accumulated data demonstrating the results obtained using three T-cell donors. FIG. 8C shows the comparison of anti-Fas monoclonal antibody binding on SPPL3^{KO} and AAVS1^{KO} T cells. Staining controls are indicated as black lines. The data presented in panels A and C are representative of three independent experiments. The data in panels A and B are reported as means±SDs (n=8). The horizontal lines indicate the mean. Statistical analysis was performed using the Mann-Whitney U test, and ***p<0.001.

FIG. 9 shows compared to control (NT-KO) Jurkat T cells, SPPL3-KO Jurkat T cells had lower expression of NK cell ligands (B7-H6, NKG2D ligand, NKp46 ligand), as well as lower expression of HLA-A,B,C.

FIG. 10 shows compared to control (NT-KO) primary T cells, SPPL3-KO primary T cells had lower expression of Fas, NKG2D ligands, and HLA-A,B,C.

FIG. 11 shows when treated with NK cells, the frequency of CellTrace™ Violet (CTV)-labeled SPPL3-KO Jurkat T cells increased relative to the control (NT-KO) Jurkat T cells as the E:T ratio increased.

FIG. 12A shows when treated with NK cells, the frequency of CTV-labeled SPPL3-KO primary T cells increased relative to CFSE-labeled control (NT-KO) primary T cells as the E:T ratio increased. FIG. 12B shows accumulated results of T cells from 3 donors treated with NK cells separately: the percentage of CTV-labeled SPPL3-KO primary T cells significantly increased in the mixture of SPPL3-KO and control primary T cells as the E:T ratio increased. *** indicates statistical significance.

FIG. 13 shows SPPL3-KO anti-CD19 CAR Jurkat T cells and control (NT-KO) anti-CD19 CAR Jurkat T cells had similar killing efficiency against CD19⁺ RAJI cells, and that higher killing efficiency (lower CD19⁺ RAJI cell percentage) was consistently associated with higher E:T ratios compared to the group with no Jurkat T cells (E:T=0).

FIG. 14A shows CFSE-labeled PBMCs cultured alone (negative control) had no proliferated CD8+ T cells (upper panel; the percentage of CFSE-low cells was 0.48), while CFSE-labeled PBMCs cultured together with irradiated allogeneic primary T cells stimulated CD8+T proliferation (lower panel; the percentage of CFSE-low cells was 33.6). FIG. 14B shows irradiated SPPL3-KO primary T cells induced significantly less allogeneic T cell proliferation (CFSE-low CD8+) compared to irradiated control primary T cells. FIG. 14B shows data from 2 different T cell donors. * indicates statistical significance. FIG. 14C shows a comparison of anti-HLA-ABC monoclonal binding on SPPL3^{KO} and AAVS1^{KO} T cells. Staining controls (IgG2a) are indicated as black lines. The plot shown is representative of three independent experiments.

FIG. 15A and FIG. 15B show SPPL3 ablation in primary T cells mitigated the allogeneic antigen specific T-cell killing. FIG. 15A shows schematic of NY-ESO-1 TCR-T cell killing assay. FIG. 15B shows that SPPL3^{KO} T cells were much more resistant to New York esophageal squamous cell carcinoma 1 (NY-ESO-1) specific TCR-T cells killing. FIG. 15B also shows data from 2 different T cell donors. *** indicates statistical significance.

FIG. 16A shows an evaluation of allogeneic NK cells killing against activated T cells. SPPL3^{KO} and AAVS1^{KO} T cells were incubated with NaP overnight. Subsequently, SPPL3^{KO} T cells were cultured with control AAVS1^{KO} T cells in equal numbers and either treated with purified NK cells or left untreated for 6 hours. Flow cytometry was used to measure the percentages of SPPL3^{KO} T cells. FIG. 16B shows accumulated data obtained using different sgRNAs targeting SPPL3. The data presented represent three donors. The bars with an E:T ratio greater than 0 include NK cells as effector (E) cells. FIG. 16C shows a schematic representation of the experiment setup to evaluate NK cell killing against T cells in vivo. FIG. 16D shows an evaluation of NK cell killing against T cells in the spleen and liver. The bar marked by + include NK cells. FIG. 16E shows a comparison of binding of NKG2D-Fc on SPPL3^{KO} and AAVS1^{KO} T cells. Staining controls are indicated as black lines. The plot shown is representative of three independent experiments. The data in FIG. 16B (n=6) and FIG. 16D (n=4) are reported as means±SDs. Horizontal lines indicate the mean. Statistical analysis was performed using the Mann-Whitney U test, *p<0.05; **p<0.01; ***p<0.001. FIG. 16F shows binding of anti-HLA-ABC monoclonal antibody on SPPL3^{KO} and AAVS1^{KO} Jurkat T cells, with staining controls indicated as black lines. FIG. 16G shows an analysis strategy of human T cells from NSG mice. FIG. 16H shows the binding of anti-B7-H6 antibody, NKG2D-Fc, and NKp46-Fc on SPPL3^{KO} and AAVS1^{KO} Jurkat T cells, with staining controls indicated as black lines. The data presented in FIGS. 16F and 16H are representative of three independent experiments.

FIG. 17A shows SPPL3^{KO} primary T cells and control (AAVS1^{KO}) CAR-T cells had similar proliferation capacity. FIG. 17B shows SPPL3^{KO}/TCR^{KO}/anti-CD19 CAR-T cells (top line) and control AAVS1^{KO}/TCR^{KO}/anti-CD19 CAR-T cells (bottom line) had similar killing efficiency against CD19⁺ NALM6 cells. FIG. 17C shows SPPL3^{KO}/TCR^{KO}/anti-CD19 CAR-T cells (top line in both graphs) exhibited a growth advantage over control AAVS1^{KO}/TCR^{KO}/anti-CD19 CAR-T cells (bottom line in both graphs) during repetitive CD19⁺ NALM6 cell stimulations. FIG. 17D shows SPPL3^{KO}/TCR^{KO}/anti-CD19 CAR-T cells eliminated tumors in vivo. FIG. 17E shows a Kaplan-Meier plot of the overall survival of all groups of mice in FIG. 17D. The dashed line represents the no T-cell group and an overlapping solid light grey line represents the mock T-cell group (T cells without CAR). Both groups had 0% survival by day 15. The lines representing the groups that received anti-CD19 CAR-T cells (without other gene editing), SPPL3^{KO}/TCR^{KO}/anti-CD19 CAR-T cells (SPPL3^{KO}), or B2M^{KO}/TCR^{KO}/anti-CD19 CAR-T cells (B2M^{KO}) are labeled in FIG. 17E. FIG. 17F shows graphs of bioluminescence imaging (BLI) signals for animals in the indicated groups of FIG. 17D. The dashed line represents the no T-cell group and an overlapping solid line represents the mock T-cell group (T cells without CAR). The lines representing the groups that received anti-CD19 CAR-T cells (without other gene editing), SPPL3^{KO}/TCR^{KO}/anti-CD19 CAR-T cells (SPPL3^{KO}), or B2M^{KO}/TCR^{KO}/anti-CD19 CAR-T cells (B2M^{KO}) are labeled in FIG. 17F. FIG. 17E and FIG. 17F show SPPL3^{KO}/TCR^{KO}/anti-CD19 CAR-T cells inhibited tumor growth and improved animal survival for up to 120 days. FIG. 17G shows SPPL3^{KO}/TCR^{KO}/anti-CD19 CAR-T cells displayed improved survival over control CAR-T cells (AAVS1^{KO}/TCR^{KO}/anti-CD19 CAR-T and B2M^{KO}/TCR^{KO}/anti-CD19 CAR-T) under the allogeneic immune environment. FIG. 17G shows data from 2 different T cell donors and 5 different PBMC donors. ** and *** indicate statistical significance. “n.s.” means no statistical significance.

FIG. 18A shows the experimental design for determining whether SPPL3^{KO}/TCR^{KO}/anti-CD19 CAR-T cells exhibit enhanced expansion upon repetitive CD19⁺ NALM6 cell stimulations in vivo. Mice engrafted with CD19⁺ NALM6 cells were treated with either SPPL3-deficient or control anti-CD19 CAR-T cells and subsequently re-challenged with CD19⁺ NALM6 cells at the indicated time points. FIG. 18B shows a Kaplan-Meier plot of the overall survival of all groups. The dashed line represents the no T-cell group and an overlapping solid line represents the mock T-cell group (T cells without CAR). All mice in both the no T-cell group and the mock T-cell group died around day 18. The lines representing the groups that received anti-CD19 CAR-T cells (without other gene editing) or SPPL3^{KO}/TCR^{KO}/anti-CD19 CAR-T cells (SPPL3^{KO}) are labeled in FIG. 18B. FIG. 18C shows a quantification of tumor burden from BLI images. The dashed line represents the no T-cell group and an overlapping solid grey line represents the mock T-cell group (T cells without CAR). The lines representing the groups that received anti-CD19 CAR-T cells (without other gene editing) or SPPL3^{KO}/TCR^{KO}/anti-CD19 CAR-T cells (SPPL3^{KO}) are labeled in FIG. 18C. FIG. 18D shows the percentage of anti-CD19 CAR-T positive cells in the blood of mice given SPPL3^{KO}/TCR^{KO}/anti-CD19 CAR-T cells or anti-CD19 CAR-T cells (without other gene editing). Dash vertical lines in FIGS. 18C-18D indicate the days mice were given tumor cell rechallenge as shown in FIG. 18A (n=6 per group). *** indicates statistical significance. “n.s.” means no statistical significance.

FIG. 19A shows the experimental design for assessing the cytotoxic function of SPPL3^{KO}/TCR^{KO}/anti-CD19 CAR-T cells. FIG. 19B shows BLI images of the tumor burden in mice (n=6 per group). FIG. 19C shows a Kaplan-Meier plot of the overall survival of all groups of mice in FIG. 19B. The dashed line represents the no T-cell group and the solid light grey line (next to nearly overlapped with the dashed line) represents the mock T-cell group (T cells without CAR). The groups that received anti-CD19 CAR-T cells (without other gene editing), SPPL3^{KO}/TCR^{KO}/anti-CD19 CAR-T cells, or B2M^{KO}/TCR^{KO}/anti-CD19 CAR-T cells are indicated in FIG. 19C. FIG. 19D shows a quantification of the tumor burden from BLI images of FIG. 19B. The dashed line represents the no T-cell group and the solid grey line nearly overlapped with the dashed line represents the mock T-cell group (T cells without CAR). The groups that received anti-CD19 CAR-T cells (without other gene editing), SPPL3^{KO}/TCR^{KO}/anti-CD19 CAR-T cells, or B2M^{KO}/TCR^{KO}/anti-CD19 CAR-T cells are indicated in FIG. 19D.

### DETAILED DESCRIPTION OF THE PRESENT APPLICATION

Poor persistence of modified immune cells (e.g., CAR-T or CAR-NK cells) is one of the biggest challenges for their effective applications in immunotherapy (e.g., cancer immunotherapy). The present invention provides immune cells (e.g., allogeneic CAR-T or CAR-NK cells) with improved persistence (e.g., in vivo), such as reducing (e.g., reducing at least about any of 10%, 20%, 30%, 40%, 50%, 60%, 70%, 80%, 90%, 95%, or more) or eliminating one or more of activation-induced cell death (AICD), and host-versus-graft (HvG) responses, such as being attacked by host T cells and/or NK cells. Also provided are methods of identifying target genes that modulate immune cell persistence (e.g., via high-throughput screening by NGS), methods of preparing such immune cells, and uses thereof, such as serving as biomarkers for immune cell donor selection or exclusion, or markers of quality control of modified immune cells with improved persistence.

Inventors of the present application discovered that immune cells (e.g., T cells such as CAR-T cells, NK cells such as CAR-NK cells) modified to have no or reduced (e.g., reducing at least about any of 10%, 20%, 30%, 40%, 50%, 60%, 70%, 80%, 90%, 95%, or more) expression (RNA and/or protein expression) and/or function of one or more target proteins selected from the group consisting of: SPPL3, FADD, FAS, CASP8, ARID1A, BAK1, BID, ETS1, IKZF2, and HIST1H1B (e.g., immune cells modified to have no or reduced expression and/or function of SPPL3 protein) have one or more following characteristics: i) have at least about 10% (e.g., at least about any of 20%, 30%, 40%, 50%, 60%, 70%, 80%, 90%, 95%, or 100%) less AICD; ii) have at least about 10% (e.g., at least about any of 20%, 30%, 40%, 50%, 60%, 70%, 80%, 90%, 95%, or 100%) less cell surface expression of one or more ligands or receptors involved in induction of killing by immune cells (e.g., T cells, NK cells), and/or AICD, e.g., Fas, HLA-A, HLA-B, HLA-C, HLA-E, B7-H6, MICA, MICB, ULBP1, ULBP2, ULBP3, ULBP4, ULBP5, ULBP6, or ligands of NKp46, iii) are subjected to at least about 10% (e.g., at least about any of 20%, 30%, 40%, 50%, 60%, 70%, 80%, 90%, 95%, or 100%) less killing by an allogeneic T cell; iv) are subjected to at least about 10% (e.g., at least about any of 20%, 30%, 40%, 50%, 60%, 70%, 80%, 90%, 95%, or 100%) less killing by an autologous or allogeneic NK cell; v) do not have down-regulated (e.g., down-regulated at least about any of 10%, 20%, 30%, 40%, 50%, 60%, 70%, 80%, 90%, 95%, or more) of eliminated expression and/or function (e.g., mediating cytotoxicity) of engineered receptors (e.g., CAR) of the immune cells, or have at most about 30% (e.g., at most about any of 25%, 20%, 10%, 5%, 3% 1%, or less) down-regulated expression and/or function of the engineered receptors, compared to a reference immune cell not having the modification to reduce or eliminate expression and/or function of the one or more target proteins; vi) confers stronger anti-tumor activity through reducing tumor size in an animal provided a modified immune cell by at least about 10% (e.g., at least about any of 20%, 30%, 40%, 50%, 60%, 70%, 80%, 90%, 95%, or 100%), relative to an animal provided a reference immune cell not having the modification to reduce or eliminate expression and/or function of the one or more target proteins; vii) confers stronger anti-tumor activity through increasing animal survival of those provided with a modified immune cell by at least about 10% (e.g., by at least about any of 20%, 30%, 40%, 50%, 60%, 70%, 80%, 90%, 95%, 100%, 110%, 120%, 130%, 140%, or 150%), relative to an animal provided a reference immune cell not having the modification to reduce or eliminate expression and/or function of the one or more target proteins; viii) confers improved survival of the modified immune cell in an allogeneic immune environment by at least about 10% (e.g., by at least about any of 20%, 30%, 40%, 50%, 60%, 70%, 80%, 90%, 95%, 100%), relative to a reference immune cell not having the modification to reduce or eliminate expression and/or function of the one or more target proteins; and ix) confers enhanced expansion of the immune cells having the modification to reduce or eliminate expression and/or function of the one or more target proteins upon repetitive stimulations of target cells of the immune cells by at least about 10% (e.g., by at least about any of 20%, 30%, 40%, 50%, 60%, 70%, 80%, or 90%), relative to a reference immune cell not having the modification to reduce or eliminate expression and/or function of the one or more target proteins. These characteristics contribute to the persistence of the modified immune cells while retaining their therapeutic functions, e.g., mediated by engineered receptors (e.g., CAR) expressed on the modified immune cells.

In one aspect, the present invention provides immune cells (e.g., CAR-T or CAR-NK cells) modified to have no or reduced (e.g., reducing at least about any of 10%, 20%, 30%, 40%, 50%, 60%, 70%, 80%, 90%, 95%, or more) expression (RNA and/or protein expression) and/or function of one or more target proteins selected from the group consisting of: SPPL3, FADD, FAS, CASP8, ARID1A, BAK1, BID, ETS1, IKZF2, and HIST1H1B. In some embodiments, there is provided immune cells (e.g., CAR-T or CAR-NK cells) modified to have no or reduced (e.g., reducing at least about any of 10%, 20%, 30%, 40%, 50%, 60%, 70%, 80%, 90%, 95%, or more) expression (RNA and/or protein expression) and/or function of SPPL3 protein, such as knock-out (KO) SPPL3, e.g., mediated by CRISPR/Cas. Also provided are pharmaceutical compositions comprising the modified immune cells described herein, and optionally a pharmaceutically acceptable excipient.

In another aspect, the present invention provides methods of treating a disease (e.g., cancer) in an individual, comprising administering to the individual an effective amount of any of the modified immune cells described herein (e.g., target protein-modified CAR-T or CAR-NK cells), or pharmaceutical compositions thereof.

In another aspect, the present invention provides methods of identifying an individual (e.g., human) as a suitable donor of an immune cell (e.g., T cells, NK cells) with prolonged in vivo persistence, comprising examining the expression (RNA and/or protein expression) and/or function of one or more target proteins selected from the group consisting of SPPL3, FADD, FAS, CASP8, ARID1A, BAK1, BID, ETS1, IKZF2, and HIST1H1B (e.g., examining the expression and/or function of SPPL3 protein) in the individual, wherein the identification of reduced (e.g., reducing at least about any of 10%, 20%, 30%, 40%, 50%, 60%, 70%, 80%, 90%, 95%, or 100%) or abolished expression and/or function of the one or more target proteins compared to a reference (e.g., the average expression and/or function of the one or more target proteins (e.g., SPPL3) in a population of individuals) identifies the individual as the suitable donor.

In another aspect, the present invention provides methods of excluding an individual (e.g., human) as a suitable donor of an immune cell (e.g., T cells, NK cells) with prolonged in vivo persistence, comprising examining the expression (RNA and/or protein expression) and/or function of one or more target proteins selected from the group consisting of SPPL3, FADD, FAS, CASP8, ARID1A, BAK1, BID, ETS1, IKZF2, and HIST1H1B (e.g., examining the expression and/or function of SPPL3 protein) in the individual, wherein the individual is excluded as the suitable donor if no reduced (e.g., reducing at least about any of 10%, 20%, 30%, 40%, 50%, 60%, 70%, 80%, 90%, 95%, or 100%) or abolished expression and/or function of the one or more target proteins compared to a reference (e.g., the average expression and/or function of the one or more target proteins (e.g., SPPL3) in a population of individuals) is identified.

In another aspect, the present invention provides methods of: i) prolonging in vivo persistence, ii) reducing AICD, and/or iii) reducing HvG response of an immune cell (e.g., T cells such as CAR-T cells, NK cells such as CAR-NK cells), comprising modifying the immune cell to reduce (e.g., reduce at least about any of 10%, 20%, 30%, 40%, 50%, 60%, 70%, 80%, 90%, 95%, or more) or eliminate expression (RNA and/or protein expression) and/or function of one or more target proteins selected from the group consisting of: SPPL3, FADD, FAS, CASP8, ARID1A, BAK1, BID, ETS1, IKZF2, and HIST1H1B, e.g., modifying the immune cell to reduce or eliminate expression and/or function of SPPL3 protein, such as by KO SPPL3 mediated by CRISPR/Cas.

In another aspect, the present invention provides methods of identifying a target gene in an immune cell whose mutation increases resistance to AICD, comprising: a) providing an immune cell library comprising a plurality of immune cells (e.g., T cells, B cells, NK cells), wherein each of the plurality of immune cells has a mutation at a hit gene (hit gene mutation), wherein the hit gene in at least two of the plurality of immune cells are different from each other; wherein the immune cell library is generated by contacting an initial population of immune cells with i) a guide RNA (sgRNA) library comprising a plurality of sgRNA constructs, wherein each sgRNA construct comprises or encodes an sgRNA, and wherein each sgRNA comprises a guide sequence that is complementary to a target site in a corresponding hit gene; and ii) a Cas component comprising a Cas protein or a nucleic acid encoding the Cas protein (e.g., Cas9), under a condition that allows introduction of the sgRNA constructs and the Cas component into the initial population of immune cells and generation of the mutations at the hit genes; b) contacting the immune cell library with a Fas ligand (FasL); c) obtaining from the immune cell library an AICD/FasL-resistant immune cell population; and d) identifying the target gene based on the difference between the profiles of sgRNAs or hit gene mutations in the AICD-resistant immune cell population and a control immune cell population. In some embodiments, the control immune cell population is i) a subpopulation of the immune cell library before step b); or ii) a same immune cell library cultured under the same condition and not contacted with the FasL.

Also provided are methods of generating any of the modified immune cells described herein, comprising reducing (e.g., reducing at least about any of 10%, 20%, 30%, 40%, 50%, 60%, 70%, 80%, 90%, 95%, or more) or eliminating the expression (RNA and/or protein expression) and/or function of one or more target proteins selected from the group consisting of: SPPL3, FADD, FAS, CASP8, ARID1A, BAK1, BID, ETS1, IKZF2, and HIST1H1B (e.g., reducing or eliminating the expression and/or function of SPPL3 protein), or one or more target proteins identified using any of the screening methods described herein, such as genetically modifying the one or more DNA loci encoding the one or more target proteins, e.g., by NHEJ, HDR, zinc-finger nuclease (ZFN), transcription activator-like effector nuclease (TALEN), or CRISPR/Cas, or modifying the one or more RNAs encoding the one or more target proteins, e.g., by using antisense RNA, siRNA, shRNA, leveraging endogenous ADAR for programmable editing of RNA (“LEAPER”; see, e.g., WO2020074001 and Qu et al. (Nat Biotechnol. 2019; 37(9):1059-1069), or RNA Editing for Programmable A to I Replacement (“REPAIR”; see, e.g., Cox et al., “RNA editing with CRISPR-Cas13,” Science. 2017; 358(6366):1019-1027), etc., the contents of each of which are incorporated herein by reference in their entirety.

Also provided by modified immune cells obtained by any of the methods described herein.

The present invention in one aspect provides a method of identifying a target gene in an immune cell (e.g., T cell, B cell, or NK cell, such as CAR-T cell) whose mutation increases resistance to AICD, comprising: a) providing an immune cell library comprising a plurality of immune cells, wherein each of the plurality of immune cells has a mutation (e.g., inactivating mutation) at a hit gene (“hit gene mutation”), wherein the hit gene in at least two of the plurality of immune cells are different from each other; b) contacting the immune cell library with a FasL; c) obtaining from the immune cell library an AICD-resistant immune cell population; and d) identifying the target gene based on the difference between the profiles of hit gene mutations in the AICD-resistant immune cell population and a control immune cell population. In some embodiments, the control immune cell population is i) a subpopulation of the immune cell library before step b); or ii) a same immune cell library cultured under the same condition and not contacted with the FasL.

Also provided are gRNA or sgRNA or sgRNA^{iBAR} molecules, constructs, sets, or libraries, which are useful for conducting the screening methods described herein. Modified immune cells (e.g., CAR-T or CAR-NK cells) comprising the gRNA or sgRNA or sgRNA^{iBAR} molecules, constructs, sets, or libraries, and methods of generating thereof, are also provided. Further provided are target genes whose aberration (e.g., inactivation such as knock-out, or reduced or abolished expression) increases resistance to AICD of immune cells, such as SPPL3, FADD, FAS, CASP8, ARID1A, BAK1, BID, ETS1, IKZF2, and HIST1H1B (e.g., SPPL3). gRNA or sgRNA or sgRNA^{iBAR} molecules, constructs, sets, or libraries against AICD resistant genes identified herein, modified immune cells (e.g., CAR-T or CAR-NK cells) comprising thereof, pharmaceutical compositions thereof, and kits, are also provided. In some embodiments, there is provided a nucleic acid encoding a gRNA, an sgRNA, or an sgRNA^{iBAR} that targets SPPL3, wherein the nucleic acid comprises the sequence of SEQ ID NO: 1. In some embodiments, there is provided a gRNA, an sgRNA, or an sgRNA^{iBAR} that targets SPPL3, wherein the gRNA, sgRNA, or sgRNA^{iBAR} comprises a guide sequence encoded by a nucleic acid comprising the sequence of SEQ ID NO: 1.

### I. DEFINITIONS

The present invention will be described with respect to particular embodiments and with reference to certain drawings but the invention is not limited thereto. Any reference signs in the claims shall not be construed as limiting the scope. In the drawings, the size of some of the elements may be exaggerated and not drawn on scale for illustrative purposes. Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

As used herein, “internal barcode” or “iBAR” refers to an index inserted into or appended to a molecule, which is useful for tracing the identity and performance of the molecule. The iBAR can be, for example, a short nucleotide sequence inserted in or appended to a guide RNA for a CRISPR/Cas system, as exemplified by the present invention. Multiple iBARs can be used to trace the performance of a single guide RNA sequence within one experiment, thereby providing replicate data for statistical analysis without having to repeat the experiment.

“CRISPR system” or “CRISPR/Cas system” refers collectively to transcripts and other elements involved in the expression and/or directing the activity of CRISPR-associated (“Cas”) genes. For example, a CRISPR/Cas system may include sequences encoding a Cas gene, a tracr (trans-activating CRISPR) sequence (e.g., tracrRNA or an active partial tracrRNA), a tracr-mate sequence (e.g., encompassing a “direct repeat” and a tracrRNA-processed partial direct repeat in an endogenous CRISPR system), a guide sequence (also referred to as a “spacer” in an endogenous CRISPR system), and other sequences and transcripts derived from a CRISPR locus.

In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. A CRISPR complex may comprise a guide sequence hybridized to a target sequence and complexed with one or more Cas proteins.

The term “guide sequence” refers to a contiguous sequence of nucleotides in a guide RNA which has partial or complete complementarity to a target sequence in a target polynucleotide and can hybridize to the target sequence by base pairing facilitated by a Cas protein. In a CRISPR/Cas9 system, a target sequence is adjacent to a PAM site. The PAM sequence, and its complementary sequence on the other strand, together constitutes a PAM site.

The term “guide RNA” is used herein interchangeably with gRNA, and refers to nucleic acid-based molecules, including but not limited to, capable of forming protein-RNA complexes with Cas proteins and comprising sequences (e.g., guide sequence or spacer) that are sufficiently complementary to the target sequence to hybridize to the target sequence and direct the specific binding of the Cas protein-RNA complexes to the target sequence. In some embodiments, the gRNA comprises or is an crRNA. In some embodiments, the gRNA comprises two RNA strands, wherein the spacer sequence and the direct repeat (DR) sequence are in different RNA strands, e.g., an crRNA strand and a tracrRNA strand. In some embodiments, the gRNA is one RNA strand, e.g., an sgRNA.

The terms “single guide RNA,” “synthetic guide RNA” and “sgRNA” are used interchangeably and refer to a polynucleotide sequence comprising a guide sequence and any other sequence necessary for the function of the sgRNA and/or interaction of the sgRNA with one or more Cas proteins to form a CRISPR complex. In some embodiments, an sgRNA comprises a guide sequence fused to a second sequence comprising a tracr sequence derived from a tracr RNA and a tracr mate sequence derived from a crRNA. A tracr sequence may contain all or part of the sequence from the tracrRNA of a naturally-occurring CRISPR/Cas system. The term “guide sequence” refers to the nucleotide sequence within the guide RNA that specifies the target site and may be used interchangeably with the terms “guide” or “spacer.” The term “tracr mate sequence” may also be used interchangeably with the term “direct repeat(s).” “sgRNA^{iBAR}” as used herein refers to a single-guide RNA having an iBAR sequence.

The term “operable with a Cas protein” means that a guide RNA can interact with the Cas protein to form a CRISPR complex.

As used herein the term “wild type” is a term of the art understood by skilled persons and means the typical form of an organism, strain, gene or characteristic as it occurs in nature as distinguished from mutant or variant forms.

As used herein the term “variant” should be taken to mean the exhibition of qualities that have a pattern that deviates from what occurs in nature.

“Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

“Doubling time” or “population doubling time” (PDT) herein refers to the time it takes for a cell population to double in size. Cell doubling time=ln(2)/(growth rate). Growth rate (gr) refers to the amount of doubling in one unit of time.

$$gr = \frac{{log}_{2} ( N ( t ) / N ( 0 ) )}{t} ,$$

in which N(t) is the number of cells at time t, N(0) is the number of cells at time 0, t is time (usually in hours). When a cell population is an exponentially growing population, i.e., every individual cell doubles with every cell cycle, the growth rate only depends on the length of the cell cycle,

$$gr = \frac{{log}_{2} ( N ( t ) / N ( 0 ) )}{t} .$$

“Construct” as used herein refers to a nucleic acid molecule (e.g., DNA or RNA), or a vehicle capable of delivering such nucleic acid molecule. For example, when used in the context of a gRNA or an sgRNA, a construct refers to the gRNA or sgRNA molecule, a nucleic acid molecule (e.g., isolated DNA, or viral vector) encoding the gRNA or sgRNA, or a vehicle capable of delivering a nucleic acid molecule encoding the gRNA or sgRNA, such as a lentivirus carrying a nucleic acid molecule encoding the gRNA or sgRNA. When used in the context of a protein, a construct refers to a nucleic acid molecule comprising a nucleotide sequence that can be transcribed to an RNA or expressed as a protein. A construct may contain necessary regulatory elements operably linked to the nucleotide sequence that allow transcription or expression of the nucleotide sequence when the construct is present in a host cell.

“Operably linked” as used herein means that expression of a gene is under the control of a regulatory element (e.g., a promoter) with which it is spatially connected. A regulatory element may be positioned 5′ (upstream) or 3′ (downstream) to a gene under its control. The distance between the regulatory element (e.g., promoter) and a gene may be approximately the same as the distance between that regulatory element (e.g., promoter) and a gene it naturally controls and from which the regulatory element is derived. As it is known in the art, variation in this distance may be accommodated without loss of function in the regulatory element (e.g., promoter).

The term “vector” is used to describe a nucleic acid molecule that may be engineered to contain a cloned polynucleotide or polynucleotides that may be propagated in a host cell. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double-stranded DNA loop into which additional DNA segments can be inserted, such as by standard molecular cloning techniques. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operably linked. Such vectors are referred to herein as “expression vectors.” Recombinant expression vectors can comprise a nucleic acid of the invention in a form suitable for expression of the nucleic acid in a host cell, which means that the recombinant expression vectors include one or more regulatory elements, which may be selected on basis of the host cells to be used for expression, that is operably linked to the nucleic acid sequence to be expressed.

A “host cell” refers to a cell that may be or has been a recipient of a vector or isolated polynucleotide. Host cells may be prokaryotic cells or eukaryotic cells. In some embodiments, the host cell is a eukaryotic cell that can be cultured in vitro and modified using the methods described herein. The term “cell” includes the primary subject cell and its progeny.

As used herein, the term “autologous” is meant to refer to any material derived from the same individual to whom it is later to be re-introduced into the individual.

“Allogeneic” refers to a graft derived from a different individual of the same species. “Allogeneic T cell” refers to a T cell from a donor having a tissue human leukocyte antigen (HLA) type that matches the recipient. Typically, matching is performed on the basis of variability at three or more loci of the HLA gene, and a perfect match at these loci is preferred.

In some instances allogeneic transplant donors may be related (usually a closely HLA matched sibling), syngeneic (a monozygotic “identical” twin of the patient) or unrelated (donor who is not related and found to have very close degree of HLA matching). The HLA genes fall in two categories (Type I and Type II). In general, mismatches of the Type-I genes (i.e., HLA-A, HLA-B, or HLA-C) increase the risk of graft rejection. A mismatch of an HLA Type II gene (i.e., HLA-DR, or HLA-DQB1) increases the risk of graft-versus-host disease (GvHD).

The term “donor subject” or “donor” refers to a subject whose cells are being obtained f or further in vitro engineering. The donor subject can be a patient that is to be treated with a population of cells generated by the methods described herein (i.e., an autologous donor), or can be an individual who donates a blood sample (e.g., lymphocyte sample) that, upon generation of the population of cells generated by the methods described herein, will be used to treat a different individual or patient (i.e., an allogeneic donor). Those subjects who receive the modified cells (e.g., modified immune cells described herein or prepared by the present methods) can be referred to as “recipient” or “recipient subject.”

“Multiplicity of infection” or “MOI” are used interchangeably herein to refer to a ratio of agents (e.g., phage, virus, or bacteria) to their infection targets (e.g., cell or organism). For example, when referring to a group of cells inoculated with viral particles, the multiplicity of infection or MOI is the ratio between the number of viral particles (e.g., viral particles comprising an sgRNA library) and the number of target cells present in a mixture during viral transduction.

A “phenotype” of a cell as used herein refers to an observable characteristic or trait of a cell, such as its morphology, development (e.g., growth, proliferation, differentiation, or death), homeostasis, biochemical or physiological property, phenology, or behavior. A phenotype may result from expression of genes in a cell, influence from environmental factors, or interactions between the two. In some embodiments, the phenotype is growth, differentiation, and/or maturation. In some embodiments, the phenotype is inhibition of growth or proliferation. In some embodiments, the phenotype is persistence in vivo. In some embodiments, the phenotype is death. In some embodiments, the phenotype is effector function (e.g., cytokine release and/or cytotoxic killing) of an immune cell, or reduced or absent effector function. In some embodiments, the phenotype is immune cell (e.g., T cell) exhaustion.

“Immune cell exhaustion” refers to decrease of immune cell function, e.g., reduced proliferative capacity, reduced effector function, and upregulation of immunosuppressive molecules. “T cell exhaustion” refers to decrease of T cell function, “NK cell exhaustion” refers to decrease of NK cell function, which may occur as a result of an infection (e.g., a chronic infection) or a disease (e.g., cancer). T cell exhaustion is associated with increased expression of PD-1, TIM-3, TIGIT, and/or LAG-3, apoptosis, reduced cytotoxicity, and/or reduced cytokine secretion. Accordingly, the terms “ameliorate T cell exhaustion,” “inhibit T cell exhaustion,” “reduce T cell exhaustion” and the like refer to a condition of restored functionality of T cells characterized by one or more of the following: decreased expression and/or level of one or more of PD-1, TIM-3, TIGIT, and/or LAG-3; increased memory cell formation and/or maintenance of memory markers (e.g., CD62L); prevention of apoptosis; increased antigen-induced cytokine (e.g., IFN-γ, IL-2, and/or TNF-α) production and/or secretion; enhanced cytotoxicity/killing capacity; increased recognition of tumor targets with low surface antigen; enhanced proliferation in response to antigen. NK cell exhaustion is associated with increased expression of PD-1, TIGIT, TIM-3, LAG-3, and/or NKG2A, downregulated expression of activating receptors (e.g., NKG2D, CD16, NCRs (NKp30, NKp44, and NKp46), CD226, 2B4), apoptosis, reduced cytotoxicity, and/or reduced cytokine (e.g., IFN-γ, and/or TNF-α) secretion. Accordingly, the terms “ameliorate NK cell exhaustion,” “inhibit NK cell exhaustion,” “reduce NK cell exhaustion” and the like refer to a condition of restored functionality of NK cells characterized by one or more of the following: decreased expression and/or level of one or more of PD-1, TIM-3, TIGIT, LAG-3, and/or NKG2A; prevention of apoptosis; increased cytokine (e.g., IFN-γ, and/or TNF-α) production and/or secretion; enhanced cytotoxicity/killing capacity; increased recognition of tumor targets with low surface antigen; enhanced proliferation in response to antigen (e.g., MHC class I molecule); enhanced expression of one or more activating receptors (e.g., NKG2D, CD16, NCRs (NKp30, NKp44, and NKp46), CD226, 2B4).

The term “stimulation,” as used herein, refers to a primary response induced by ligation of a cell surface moiety. For example, in the context of receptors, such stimulation entails the ligation of a receptor and a subsequent signal transduction event. With respect to stimulation of a T cell, such stimulation refers to the ligation of a T cell surface moiety that in one embodiment subsequently induces a signal transduction event, such as binding the TCR/CD3 complex. Further, the stimulation event may activate a cell and upregulate or downregulate expression or secretion of a molecule, such as downregulation of TGF-β. Thus, ligation of cell surface moieties, even in the absence of a direct signal transduction event, may result in the reorganization of cytoskeletal structures, or in the coalescing of cell surface moieties, each of which could serve to enhance, modify, or alter subsequent cellular responses.

The term “activation,” as used herein, refers to the state of a cell following sufficient cell surface moiety ligation to induce a noticeable biochemical or morphological change. Within the context of T cells, such activation refers to the state of a T cell that has been sufficiently stimulated to induce cellular proliferation. Activation of a T cell may also induce cytokine production and performance of regulatory or cytolytic effector functions. Within the context of other cells, this term infers either up or down regulation of a particular physico-chemical process. The term “activated T cells” indicates T cells that are currently undergoing cell division, cytokine production, performance of reg. or cytol. Effector functions, and/or has recently undergone the process of “activation.”

A “dominant negative” gene product or protein is one that interferes with the function of another gene product or protein. The other gene product affected can be the same or different from the dominant negative protein. Dominant negative gene products can be of many forms, including truncations, full length proteins with point mutations or fragments thereof, or fusions of full-length wild type or mutant proteins or fragments thereof with other proteins.

An “isolated” nucleic acid molecule described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides herein is in a form other than in the form or setting in which it is found in nature. Isolated nucleic acid molecules therefore are distinguished from nucleic acid encoding the polypeptides herein existing naturally in cells.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s).

The term “transfected” or “transformed” or “transduced” as used herein refers to a process by which exogenous nucleic acid is transferred or introduced into the host cell (e.g., immune cell). A “transfected” or “transformed” or “transduced” cell is one which has been transfected, transformed or transduced with exogenous nucleic acid. The cell includes the primary subject cell and its progeny.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer or immune diseases.

The term “effective amount” used herein refers to an amount of an agent, such as modified immune cells described herein, or a pharmaceutical composition thereof, sufficient to treat a specified disorder, condition or disease, such as ameliorate, palliate, lessen, and/or delay one or more of its symptoms (e.g., cancer, infectious disease, GvHD, transplantation rejection, autoimmune disorders, or radiation sickness). In reference to cancer, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay recurrence. An effective amount can be administered in one or more administrations. The effective amount of the agent (e.g., modified immune cells) or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. In the case of infectious disease, such as viral infection, the therapeutically effective amount of modified immune cells described herein or compositions thereof can reduce the number of cells infected by the pathogen; reduce the production or release of pathogen-derived antigens; inhibit (i.e., slow to some extent and preferably stop) spread of the pathogen to uninfected cells; and/or relieve to some extent one or more symptoms associated with the infection. In some embodiments, the therapeutically effective amount is an amount that extends the survival of a patient.

As used herein, an “individual” or a “subject” refers to a mammal, including, but not limited to, human, bovine, horse, feline, canine, rodent, or primate. In some embodiments, the individual is a human.

A “patient” as used herein includes any human who is afflicted with a disease (e.g., cancer). The terms “subject,” “individual,” and “patient” are used interchangeably herein.

Where the term “comprising” is used in the present description and claims, it does not exclude other elements or steps.

It is understood that embodiments of the present application described herein include “consisting of” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

For the recitation of numeric ranges of nucleotides herein, each intervening number therebetween, is explicitly contemplated. For example, for the range of 19-21 nt, the number 20 nt is contemplated in addition to 19 nt and 21 nt, and for the range of MOI, each intervening number therebetween, whether it is integral or decimal, is explicitly contemplated.

As used herein and in the appended claims, the singular forms “a,” “or,” and “the” include plural referents unless the context clearly dictates otherwise.

It will be understood by one of ordinary skill in the art that uracil and thymine can both be represented by ‘t’, instead of ‘u’ for uracil and ‘t’ for thymine; in the context of a ribonucleic acid, it will be understood that ‘t’ is used to represent uracil unless otherwise indicated.

### II. MODIFIED IMMUNE CELLS

In some embodiments, there is provided immune cells (e.g., T cells such as CAR-T cells, or NK cells such as CAR-NK cells) modified to have no or reduced (e.g., reducing at least about any of 10%, 20%, 30%, 40%, 50%, 60%, 70%, 80%, 90%, 95%, or more) expression (RNA and/or protein expression) and/or function of one or more target proteins selected from the group consisting of: SPPL3, FADD, FAS, CASP8, ARID1A, BAK1, BID, ETS1, IKZF2, and HIST1H1B. In some embodiments, the target protein is selected from one or more of: ARID1A, BAK1, BID, ETS1, IKZF2, HIST1H1B, and SPPL3. In some embodiments, the target protein is SPPL3 protein. Thus in some embodiments, there is provided immune cells (e.g., T cells such as CAR-T cells, or NK cells such as CAR-NK cells) modified to have no or reduced (e.g., reducing at least about any of 10%, 20%, 30%, 40%, 50%, 60%, 70%, 80%, 90%, 95%, or more) expression (RNA and/or protein expression) and/or function of SPPL3 protein. In some embodiments, the immune cells are further modified to have no or reduced (e.g., reducing at least about any of 10%, 20%, 30%, 40%, 50%, 60%, 70%, 80%, 90%, 95%, or more) expression (RNA and/or protein expression) and/or function of a TCR protein (e.g., one or more of TCRα, TCRβ, TCRγ, TCRδ). In some embodiments, the modification is mediated by gene editing (e.g., CRISPR/Cas). In some embodiments, there is provided immune cells (e.g., T cells such as CAR-T cells, or NK cells such as CAR-NK cells) with SPPL3 gene knock-out (KO) (“SPPL3^{KO}”). In some embodiments, there is provided immune cells (e.g., T cells such as CAR-T cells, or NK cells such as CAR-NK cells) with SPPL3 gene KO and TCR gene KO (“SPPL3^{KO}/TCR^{KO}”). In some embodiments, the immune cell is a T cell, such as a CAR-T cell (e.g., anti-CD19 CAR-T cell).

In some embodiments, the modified immune cell has at least about 10% (e.g., at least about any of 20%, 30%, 40%, 50%, 60%, 70%, 80%, 90%, 95%, or 100%) less, such as at least about 20% less, activation-induced cell death (AICD) compared to a reference immune cell not having the modification to reduce or eliminate expression (RNA and/or protein expression) and/or function of the one or more target proteins, e.g., SPPL3 protein. In some embodiments, the modified immune cell has at least about 10% (e.g., at least about any of 20%, 30%, 40%, 50%, 60%, 70%, 80%, 90%, 95%, or 100%) less, such as at least about 20% less, AICD compared to a reference immune cell not having the modification to reduce or eliminate expression (RNA and/or protein expression) and/or function of the SPPL3 protein.

In some embodiments, the expression of the one or more target proteins (e.g., SPPL3 protein) is reduced (e.g., reducing at least about any of 10%, 20%, 30%, 40%, 50%, 60%, 70%, 80%, 90%, 95%, or more) or inhibited by an antisense RNA, an siRNA, or an shRNA specifically recognizing an RNA encoding the one or more target proteins (e.g., SPPL3 protein).

In some embodiments, the function of the one or more target proteins (e.g., SPPL3 protein) is reduced (e.g., reducing at least about any of 10%, 20%, 30%, 40%, 50%, 60%, 70%, 80%, 90%, 95%, or more) or inhibited by a chemically modified mRNA, such as a chemically modified mRNA encoding a dominant negative inhibitor (e.g., dominant negative variant or fragment thereof, or dominant negative binding partner) of the one or more target proteins (e.g., SPPL3 protein). In some embodiments, the immune cell is modified to express a dominant negative SPPL3 protein variant or dominant negative fragment thereof. In some embodiments, the immune cell is modified to express a dominant negative binding partner of SPPL3 protein.

In some embodiments, the expression (RNA and/or protein expression) and/or function of the one or more target proteins (e.g., SPPL3 protein) is reduced (e.g., reducing at least about any of 10%, 20%, 30%, 40%, 50%, 60%, 70%, 80%, 90%, 95%, or more) or inhibited by a small molecule compound, a nucleic acid (or vector comprising thereof), a lipid, and/or a protein molecule.

In some embodiments, the immune cell is genetically modified at the DNA locus encoding the one or more target proteins. In some embodiments, the immune cell is genetically modified at the SPPL3 locus. In some embodiments, the DNA locus (e.g., SPPL3 locus) is modified by a mutagenic agent. Mutagenic agents can be classified into three categories: physical (e.g., gamma rays, ultraviolet (UV) radiations), chemical (e.g., ethyl methane sulphonate (EMS)) and transposable elements (such as transposons, retrotransposons, T-DNA, retroviruses). In some embodiments, the mutagenic agent or condition is any of ionizing radiation (IR), UV radiation, an alkylating agent (e.g., nitrogen mustard gas, methyl methanesulfonate (MMS), EMS, N-ethyl-N-nitrosourea (ENU)), an aromatic amine (e.g., 2-aminofluorene), an polycyclic aromatic hydrocarbon (PAH; e.g., dibenzo[a,l]pyrene, naphthalene, anthracene, pyrene), crosslinking, insertional mutagenesis (e.g., mediated by transposons or viruses), or other toxins (e.g., aflatoxin, N-nitrosamines). In some embodiments, the DNA locus (e.g., SPPL3 locus) is modified by gene editing. In some embodiments, the gene editing is mediated by site-directed mutagenesis (SDM). In some embodiments, the gene editing is mediated by random-and-extensive mutagenesis (REM). In some embodiments, the gene editing is accomplished by PCR method(s). In some embodiments, the gene editing is accomplished by non-PCR method(s). Any known gene editing methods can be used herein, including but not limited to, non-homologous end-joining (NHEJ)-mediated, homology directed repair (HDR)-mediated, zinc-finger nuclease (ZFN)-mediated, transcription activator-like effector nuclease (TALEN)-mediated, or CRISPR/Cas mediated gene editing. HDR can occur either non-conservatively or conservatively. In some embodiments, HDR is mediated through single-strand annealing (SSA) pathway. In some embodiments, HDR is mediated through classical double-strand break repair (DSBR) pathway, synthesis-dependent strand-annealing (SDSA) pathway, or break-induced repair (BIR) pathway. In some embodiments, the cell modification method described herein further comprises introducing a nucleic acid template (e.g., contains a desired mutation), such as to be inserted at a double-strand break (DSB) to modify a target genomic sequence (e.g., via HDR). Gene editing can introduce one or more mutations in the DNA locus encoding the one or more target proteins (e.g., SPPL3 protein), including but not limited to, insertion, deletion, substitution (e.g., nonsynonymous substitution), truncation, translocation, point mutation, etc. In some embodiments, the mutation is a frameshift mutation, a loss-of-function (LOF) mutation, a dominant negative mutation, a missense mutation, or a nonsense mutation. In some embodiments, the gene editing comprises gene knockout (KO). In some embodiments, the gene editing comprises base editing (e.g., introducing nonsynonymous substitution). In some embodiments, base editing introduces a stop codon which may reduce the expression of functional RNA and/or protein. In some embodiments, base editing introduces a mutation that affects the function of the RNA and/or protein. In some embodiments, the gene editing is mediated by CRISPR/Cas. In some embodiments, the Cas protein has endonuclease activity. In some embodiments, the Cas protein is fusion protein comprising i) a dead Cas protein (dCas) and ii) an adenine base editor (ABE) or adenine deaminase (ADA), or a cytidine base editor (CBE) or cytidine deaminase (CDA), or functional fragment thereof. Cytidine base editors can convert target C:G base pairs to T:A base pairs, and adenine base editors can convert A:T base pairs to G:C base pairs, collectively, these two classes of base editors enable the targeted installation of all possible transition mutations (C-to-T, G-to-A, A-to-G, T-to-C, C-to-U, and A-to-U).

In some embodiments, the immune cell is genetically modified at the RNA encoding the one or more target proteins. In some embodiments, the immune cell is genetically modified at SPPL3 RNA. In some embodiments, the RNA encoding the one or more target proteins (e.g., SPPL3 protein) is modified by RNA editing. RNA editing can introduce one or more mutations in the RNA encoding the one or more target proteins (e.g., SPPL3 protein), including but not limited to, insertion, deletion, substitution (e.g., nonsynonymous substitution), truncation, point mutation, etc. In some embodiments, the mutation is a frameshift mutation, an LOF mutation, a dominant negative mutation, a missense mutation, or a nonsense mutation. In some embodiments, the RNA editing comprises base editing (e.g., introducing nonsynonymous substitution, such as C to U, A to I). Any known RNA editing methods can be used herein (see, e.g., Guillermo Aquino-Jarquin, “Novel Engineered Programmable Systems for ADAR-Mediated RNA Editing,” Mol Ther Nucleic Acids. 2020; 19:1065-1072; the content of which is incorporated herein by reference in its entirety), including but not limited to, leveraging endogenous ADAR for programmable editing of RNA (“LEAPER”; see, e.g., WO2020074001 and Qu et al. (Nat Biotechnol. 2019; 37(9):1059-1069), or RNA Editing for Programmable A to I Replacement (“REPAIR”; see, e.g., Cox et al., “RNA editing with CRISPR-Cas13,” Science. 2017; 358(6366):1019-1027), recruiting endogenous ADAR to specific transcripts for oligonucleotide-mediated RNA editing (“RESTORE”; see, e.g., Merkle et al., “Precise RNA editing by recruiting endogenous ADARs with antisense oligonucleotides,” Methods Mol Biol. 2021; 2181:331-349), CRISPR-Cas-Inspired RNA Targeting System (“CIRTS”; see, e.g., Rauch et al., “Programmable RNA-Guided RNA Effector Proteins Built from Human Parts,” Cell. 2019; 178(1):122-134.e12), RNA Editing for Specific C to U Exchange (“RESCUE”; see, e.g., Abudayyeh et al., “A cytosine deaminase for programmable single-base RNA editing,” Science. 2019; 365(6451):382-386), or CLUSTER (see, e.g., P. Reautschnig et al., “CLUSTER guide RNAs enable precise and efficient RNA editing with endogenous ADAR enzymes in vivo,” Nat Biotechnol. 2022 May; 40(5):759-768), the contents of each of which are incorporated herein by reference in their entirety. In some embodiments, the RNA editing is mediated by LEAPER. In some embodiments, the RNA editing is mediated by CRISPR/Cas, such as by fusing an adenine base editor (ABE) or adenine deaminase (ADA), or a cytidine base editor (CBE) or cytidine deaminase (CDA), or functional fragment thereof to a dead Cas (dCas, e.g., dCas13) protein.

Methods for making CRISPRs that recognize pre-determined DNA or RNA sites are known in the art. Any known CRISPR/Cas systems suitable for gene editing or RNA editing can be used herein. To date, two classes (class 1 and class 2), six types (I-VI) of CRISPR-Cas systems have been characterized based on the outstanding functional and evolutionary modularity of the system. See, e.g., Nidhi et el., “Novel CRISPR-Cas Systems: An Updated Review of the Current Achievements, Applications, and Future Research Perspectives,” Int J Mol Sci. 2021; 22(7): 3327, the content of which is incorporated herein by reference in its entirety. Among class 2 CRISPR-Cas systems, the type II Cas9 system and the type V-A/B/E/J Cas12a/Cas12b/Cas12e/Cas12j systems have been utilized for genome editing and provided broad prospects for biomedical research. Cas13a (C2c2) is a type VI-A RNA-guided RNA-targeting CRISPR effector, which can be used in RNA editing described herein. The CRISPR/Cas system used herein can create double-strand break (DSB) or single-strand break at the pre-determined nucleic acid site. In some embodiments, CRISPR/Cas system used herein is a CRISPR/Cas9 system.

In some embodiments, the gene editing or RNA editing comprises contacting a precursor immune cell (e.g., precursor T cell or precursor NK cell) with i) a guide RNA (gRNA) construct, wherein the gRNA construct comprises or encodes a gRNA comprising a guide sequence that is complementary to a target site in the DNA locus or RNA encoding the one or more target proteins (e.g., SPPL3 protein); and optionally ii) a Cas component comprising a Cas protein or a nucleic acid encoding the Cas protein (e.g., Cas9), under a condition that allows introduction of the gRNA construct and the optional Cas component into the precursor immune cell. In some embodiments, the precursor immune cell already expresses a Cas protein before introducing the gRNA construct. In some embodiments, the precursor immune cell does not express a Cas protein before introducing the gRNA construct and the Cas component. In some embodiments, the precursor immune cell expresses an engineered receptor (e.g., CAR, engineered TCR, or TAC) before introducing the gRNA construct and/or the Cas component. In some embodiments, the Cas protein has endonuclease activity. In some embodiments, the Cas protein is fusion protein, such as a fusion protein comprising i) a dCas (e.g., dCas13a, dCas9) and ii) an ADA (e.g., TadA, such as TadA8e) or a CDA or functional fragment thereof. In some embodiments, the Cas protein is Cas9, such as dCas9. In some embodiments, the gRNA is an sgRNA. In some embodiments, the gRNA comprises (or consists essentially of, or consists of) a crRNA. In some embodiments, the gRNA comprises (or consists essentially of, or consists of) a crRNA and a tracrRNA.

In some embodiments, the immune cell (e.g., T cell such as CAR-T cell) modified to have no or reduced (e.g., reducing at least about any of 10%, 20%, 30%, 40%, 50%, 60%, 70%, 80%, 90%, 95%, or more) expression (RNA and/or protein expression) and/or function of one or more target proteins selected from the group consisting of SPPL3, FADD, FAS, CASP8, ARID1A, BAK1, BID, ETS1, IKZF2, and HIST1H1B has or is further modified to have no or reduced (e.g., reducing at least about any of 10%, 20%, 30%, 40%, 50%, 60%, 70%, 80%, 90%, 95%, or more) expression (RNA and/or protein expression) and/or function of one or more other proteins selected from the group consisting of TCRα, TCRβ, TCRγ, TCRδ, HLA-A, HLA-B, HLA-C, HLA-E, HLA-F, HLA-G, B2M, PD-1, TIM-3, LAG-3, CTLA-4, CISH, SPPL3, Fas, FADD, CASP8, ARID1A, BAK1, BID, ETS1, IKZF2, HIST1H1B, B7-H6, MICA, MICB, ULBP1, ULBP2, ULBP3, ULBP4, ULBP5, ULBP6, and ligands of NKp46. In some embodiments, the immune cell modified to have no or reduced (e.g., reducing at least about any of 10%, 20%, 30%, 40%, 50%, 60%, 70%, 80%, 90%, 95%, or more) expression (RNA and/or protein expression) and/or function of SPPL3 protein has or is further modified to have no or reduced (e.g., reducing at least about any of 10%, 20%, 30%, 40%, 50%, 60%, 70%, 80%, 90%, 95%, or more) expression (RNA and/or protein expression) and/or function of one or more other proteins selected from the group consisting of TCRα, TCRβ, TCRγ, TCRδ, HLA-A, HLA-B, HLA-C, HLA-E, HLA-F, HLA-G, B2M, PD-1, TIM-3, LAG-3, CTLA-4, CISH, Fas, FADD, CASP8, ARID1A, BAK1, BID, ETS1, IKZF2, HIST1H1B, B7-H6, MICA, MICB, ULBP1, ULBP2, ULBP3, ULBP4, ULBP5, ULBP6, and ligands of NKp46. In some embodiments, the immune cell (e.g., T cell such as CAR-T cell) modified to have no or reduced (e.g., reducing at least about any of 10%, 20%, 30%, 40%, 50%, 60%, 70%, 80%, 90%, 95%, or more) expression (RNA and/or protein expression) and/or function of SPPL3 protein has or is further modified to have no or reduced (e.g., reducing at least about any of 10%, 20%, 30%, 40%, 50%, 60%, 70%, 80%, 90%, 95%, or more) expression (RNA and/or protein expression) and/or function of a TCR protein (e.g., one or more of TCRα, TCRβ, TCRγ, TCRδ). In some embodiments, the immune cell modified to have no or reduced (e.g., reducing at least about any of 10%, 20%, 30%, 40%, 50%, 60%, 70%, 80%, 90%, 95%, or more) expression (RNA and/or protein expression) and/or function of SPPL3 protein has or is further modified to have no or reduced (e.g., reducing at least about any of 10%, 20%, 30%, 40%, 50%, 60%, 70%, 80%, 90%, 95%, or more) expression (RNA and/or protein expression) and/or function of one or more other proteins selected from the group consisting of Fas, FADD, CASP8, ARID1A, BAK1, BID, ETS1, IKZF2, and HIST1H1B.

In some embodiments, the immune cell (e.g., T cell) expresses or is further modified to express an engineered receptor. In some embodiments, the immune cell already expresses an engineered receptor before modified to reduce or eliminate expression and/or function of the one or more target proteins (e.g., SPPL3 protein). In some embodiments, the immune cell is modified to reduce or eliminate expression and/or function of the one or more target proteins (e.g., SPPL3 protein), and further modified to express an engineered receptor. In some embodiments, the modifications to reduce or eliminate expression and/or function of the one or more target proteins (e.g., SPPL3 protein) and to express an engineered receptor occur simultaneously. In some embodiments, the modifications to reduce or eliminate expression and/or function of the one or more target proteins (e.g., SPPL3 protein) and to express an engineered receptor occur sequentially. Any engineered receptor that can transduce signal to the immune cell (e.g., inducing cellular proliferation, cytokine production, and/or performance of regulatory or cytolytic effector functions) and/or recognize a target antigen can be used herein. In some embodiments, the engineered receptor is a chimeric antigen receptor (CAR), an engineered TCR, or a T cell antigen coupler (TAC). In some embodiments, the engineered receptor is monovalent.

In some embodiments, the engineered receptor is multivalent. In some embodiments, the engineered receptor is monospecific, e.g., monovalent and monospecific, or multivalent and monospecific. In some embodiments, the engineered receptor is multispecific (e.g., bispecific). Any CAR, engineered TCR, or TAC can be used herein.

In some embodiments, the CAR comprises: i) an extracellular antigen binding domain specifically recognizing one or more target antigens (e.g., tumor antigens) or target epitopes (e.g., tumor epitopes); ii) a transmembrane domain; and iii) an intracellular signaling domain. In some embodiments, the extracellular antigen binding domain is selected from the group consisting of an extracellular domain of a ligand, a single-domain antibody (sdAb), a single chain Fv (scFv), and a Fab. In some embodiments, the transmembrane domain is derived from a molecule selected from the group consisting of TCRα, TCRβ, TCRγ, TCRδ, CD3ζ, CD3ε, CD3γ, CD3δ, CD4, CD5, CD6, CD7, CD8α, CD9, CD16, CD22, CD27, CD28, CD33, CD37, CD45, CD47, CD52, CD64, CD80, CD86, CD134, 4-1BB, CD152, CD154, CISH, and PD-1. In some embodiments, the transmembrane domain is derived from CD8α. In some embodiments, the intracellular signaling domain comprises a primary intracellular signaling domain derived from a molecule selected from the group consisting of CD3ζ, CD3γ, CD3ε, CD3δ, FcRγ, FcRβ, CD5, CD22, CD79a, CD79b, CD66d, FcγRIIa, DAP10, and DAP12. In some embodiments, the primary intracellular signaling domain is derived from CD3ζ. In some embodiments, the intracellular signaling domain further comprises a co-stimulatory signaling domain derived from a co-stimulatory molecule selected from the group consisting of CARD11, CD2, CD4, CD7, CD19, CD27, CD28, CD30, CD40, CD160, ICAM-1, OX40, 4-1BB, SELPLG, LIGHT, HVEM, B7-H3, ICOS, PD-1, SLAMF7, LFA-1, NKG2C, CDS, GITR, BAFFR, NKp80, IPO-3, SLAMF8, LTBR, LAT, GADS, SLP-76, PAG/Cbp, NKp44, NKp30, NKp46, NKG2D, CD83, SLAMF1, CTLA-4, LAG-3, PD-L2, PD-L1, DAP10, TRIM, ZAP70, a ligand that specifically binds with CD83, and any combination thereof. In some embodiments, the co-stimulatory signaling domain is derived from 4-1BB. In some embodiments, the CAR further comprises a hinge domain located between the C-terminus of the extracellular antigen binding domain and the N-terminus of the transmembrane domain. In some embodiments, the hinge domain is derived from CD8a or CD28. In some embodiments, the CAR comprises from N′ to C′: extracellular antigen binding domain (e.g., scFv)-optional hinge domain-transmembrane domain-primary intracellular signaling domain. In some embodiments, the CAR comprises from N′ to C′: extracellular antigen binding domain (e.g., scFv)-optional hinge domain-transmembrane domain-co-stimulatory signaling domain-primary intracellular signaling domain.

In some embodiments, the CAR is anti-CD19 CAR. In some embodiments, the anti-CD19 CAR is the anti-CD19 CAR from tisagenlecleucel CAR-T cell (CTL019, e.g., Kymriah®). See, e.g., U.S. Pat. No. 9,499,629, the content of which is incorporated herein by reference in its entirety. In some embodiments, the anti-CD19 CAR comprises i) an extracellular antigen binding domain that is an scFv specifically recognizing CD19; ii) a transmembrane domain derived from CD8α; and iii) an intracellular signaling domain comprising a primary intracellular signaling domain derived from CD3ζ. In some embodiments, the anti-CD19 CAR comprises from N′ to C′: anti-CD19 scFv-CD8α hinge domain-CD8α transmembrane domain-4-1BB co-stimulatory signaling domain-CD3ζ primary intracellular signaling domain.

In some embodiments, there is provided a T cell modified to have no or reduced (e.g., reducing at least about any of 10%, 20%, 30%, 40%, 50%, 60%, 70%, 80%, 90%, 95%, or more) expression (RNA and/or protein expression) and/or function of SPPL3 protein, wherein the T cell expresses a CAR or is further modified to express a CAR. In some embodiments, there is provided a CAR-T cell modified to have no or reduced (e.g., reducing at least about any of 10%, 20%, 30%, 40%, 50%, 60%, 70%, 80%, 90%, 95%, or more) expression (RNA and/or protein expression) and/or function of SPPL3 protein. In some embodiments, there is provided a T cell modified to have no or reduced (e.g., reducing at least about any of 10%, 20%, 30%, 40%, 50%, 60%, 70%, 80%, 90%, 95%, or more) expression (RNA and/or protein expression) and/or function of SPPL3 protein and TCR protein (e.g., one or more of TCRα, TCRβ, TCRγ, TCRδ), wherein the T cell expresses a CAR or is further modified to express a CAR. In some embodiments, there is provided a CAR-T cell modified to have no or reduced (e.g., reducing at least about any of 10%, 20%, 30%, 40%, 50%, 60%, 70%, 80%, 90%, 95%, or more) expression (RNA and/or protein expression) and/or function of SPPL3 protein and TCR protein (e.g., one or more of TCRα, TCRβ, TCRγ, TCRδ). In some embodiments, the T cell (e.g., CAR-T cell) is genetically modified at the SPPL3 locus by gene editing (e.g., by CRISPR/Cas). In some embodiments, the T cell (e.g., CAR-T cell) is genetically modified at the TCR locus by gene editing (e.g., by CRISPR/Cas). In some embodiments, the T cell (e.g., CAR-T cell) is genetically modified at the SPPL3 locus and at the TCR locus both by gene editing (e.g., by CRISPR/Cas). In some embodiments, the gene editing of the T cell (e.g., CAR-T cell) comprises contacting a precursor T cell with i) a gRNA construct, wherein the gRNA construct comprises or encodes a gRNA comprising a guide sequence that is complementary to a target site in the SPPL3 locus; and optionally ii) a Cas component comprising a Cas protein (e.g., Cas9) or a nucleic acid encoding the Cas protein, under a condition that allows introduction of the gRNA construct and the optional Cas component into the precursor T cell. In some embodiments, the guide sequence is encoded by a nucleic acid sequence comprising the sequence of SEQ ID NO: 1. In some embodiments, the precursor T cell expresses a Cas protein. In some embodiments, the CAR comprises: i) an extracellular antigen binding domain specifically recognizing one or more target antigens (e.g., tumor antigens) or target epitopes (e.g., tumor epitopes) (e.g., anti-CD19 scFv or sdAb); ii) a transmembrane domain (e.g., CD8α transmembrane domain); and iii) an intracellular signaling domain (e.g., CD3ζ primary intracellular signaling domain). In some embodiments, the CAR is an anti-CD19 CAR, e.g., derived from tisagenlecleucel (CTL019, e.g., Kymriah®). In some embodiments, there is provided an anti-CD19 CAR-T cell with SPPL3^{KO} (“SPPL3^{KO}/anti-CD19 CAR-T cell”) In some embodiments, there is provided an anti-CD19 CAR-T cell with SPPL3^{KO} and TCR^{KO} (“SPPL3^{KO}/TCR^{KO}/anti-CD19 CAR-T cell”).

In some embodiments, the engineered TCR comprises: (a) an extracellular ligand binding domain comprising an antigen-binding fragment (e.g., sdAb, scFv, Fab, DARPin) that specifically recognizes one or more target antigens (e.g., tumor antigens) or target epitopes (e.g., tumor epitopes); (b) an optional first linker; (c) an optional extracellular domain of a first TCR subunit (e.g., Cα, Cβ, Cδ, Cγ, CD3ε) or a portion thereof; (d) a transmembrane domain of a second TCR subunit (e.g., TCRα, TCRβ); and (e) an intracellular signaling domain comprising an intracellular signaling domain of a third TCR subunit (e.g., TCRα, TCRβ); wherein the first, the second, and the third TCR subunits are independently selected from the group consisting of TCRα, TCRβ, TCRγ, TCRδ, CD3E, CD3γ, CD3δ, and CD3ζ. In some embodiments, the first, the second, and the third TCR subunits are the same (e.g., all CD3ε, all TCRα, or all TCRβ). In some embodiments, the first, the second, and the third TCR subunits are different. In some embodiments, the engineered TCR further comprises a hinge domain located between the C-terminus of the extracellular ligand binding domain and the N-terminus of the transmembrane domain. In some embodiments, the hinge domain is derived from CD8α.

Typically, a TAC comprises (i) an antigen-binding domain, (ii) a TCR binding domain (e.g., scFv), and (iii) a co-receptor domain (e.g., hinge, transmembrane, and/or cytosolic region). See, e.g., Helsen et al. Nat Commun. 2018; 9(1):3049. In some embodiments, the TAC comprises: (a) an extracellular ligand binding domain comprising an antigen-binding fragment (e.g., sdAb, scFv, Fab, DARPin) that specifically recognizes one or more target antigens (e.g., tumor antigens) or target epitopes (e.g., tumor epitopes); (b) an optional first linker; (c) an extracellular TCR binding domain (e.g., sdAb, scFv, Fab, DARPin) that specifically recognizes the extracellular domain of a TCR subunit (e.g., CD3ε); (d) an optional second linker; (e) an optional extracellular domain of a first TCR co-receptor (e.g., CD4, CD8) or portion thereof; (f) a transmembrane domain comprising a transmembrane domain of a second TCR co-receptor (e.g., CD4, CD8); and (g) an optional intracellular signaling domain comprising an intracellular signaling domain of a third TCR co-receptor (e.g., CD4, CD8); wherein the TCR subunit is selected from the group consisting of CD3ε, CD3δ, CD3γ, TCRα, TCRβ, TCRγ, and TCRδ; and wherein the first, the second, and the third TCR co-receptors are each independently selected from the group consisting of CD4, CD8, and CD28. In some embodiments, the first, the second, and the third TCR co-receptors are the same. In some embodiments, the first, the second, and the third TCR co-receptors are different. In some embodiments, the TAC further comprises a hinge domain (e.g., derived from CD8α) located between the C-terminus of the extracellular ligand binding domain and the N-terminus of the transmembrane domain.

In some embodiments, the modification to reduce or eliminate expression (RNA and/or protein expression) and/or function of the one or more target proteins (e.g., SPPL3 protein) does not down-regulate or eliminate expression (RNA and/or protein expression) and/or function of the engineered receptor (e.g., CAR). In some embodiments, the modification to reduce or eliminate expression and/or function of the one or more target proteins (e.g., SPPL3 protein) down-regulates expression (RNA and/or protein expression) and/or function of the engineered receptor by at most about 30% (e.g., at most about any of 25%, 20%, 15%, 10%, 5%, 1%, or less).

In some embodiments, the immune cell is a T cell, such as selected from the group consisting of a helper CD4+ T cell, a cytotoxic CD8+ T cell, a memory T cell, a regulatory CD4+ T cell, an NKT cell, an MAIT cell, a DNT cell, and a γδ T cell. In some embodiments, the modification to reduce or eliminate expression (RNA and/or protein expression) and/or function of the one or more target proteins (e.g., SPPL3 protein): i) reduces at least about 10% (e.g., reducing at least about any of 20%, 30%, 40%, 50%, 60%, 70%, 80%, 90%, 95%, or 100%) cell surface expression of one or more of Fas, HLA-A, HLA-B, HLA-C, HLA-E, B7-H6, MICA, MICB, ULBP1, ULBP2, ULBP3, ULBP4, ULBP5, ULBP6, and ligands of NKp46; ii) reduces at least about 10% (e.g., reducing at least about any of 20%, 30%, 40%, 50%, 60%, 70%, 80%, 90%, 95%, or 100%) killing by an allogeneic T cell; and/or iii) reduces at least about 10% (e.g., reducing at least about any of 20%, 30%, 40%, 50%, 60%, 70%, 80%, 90%, 95%, or 100%) killing by an autologous or allogeneic NK cell.

In some embodiments, the modified immune cell has at least about 10% (e.g., at least about any of 20%, 30%, 40%, 50%, 60%, 70%, 80%, 90%, 1-fold, 1.5-fold, 2-fold, 5-fold, 10-fold, 20-fold, 50-fold, or more) longer in vivo persistence compared to a reference immune cell not having the modification to reduce or eliminate expression (RNA and/or protein expression) and/or function of the one or more target proteins (e.g., SPPL3 protein).

The immune cells modified to reduce or eliminate expression (RNA and/or protein expression) and/or function of the one or more target proteins (e.g., SPPL3 protein) and/or other proteins (e.g., TCR protein) and/or modified to express an engineered receptor (e.g., CAR) can be autologous or allogeneic.

### Immune Cells

The term “immune cell” as referred to herein includes cells that are of hematopoietic origin and that play a role in the immune response. “Immune cells” used herein include unmodified immune cells (e.g., parental immune cells such as primary immune cells, immune cells from cell lines), reference immune cells, and modified immune cells, such as immune cells modified to reduce or eliminate expression (RNA and/or protein expression) and/or function of the one or more target proteins (e.g., SPPL3 protein) and/or other proteins (e.g., TCR) and/or modified to express an engineered receptor (e.g., CAR). Immune cells include lymphocytes, such as B cells and T cells; natural killer (NK) cells; myeloid cells, such as monocytes, macrophages, dendritic cells, eosinophils, mast cells, basophils, and granulocytes. In one embodiment, the immune cell is an immune effector cell. The term “immune effector cell,” as that term is used herein, refers to a cell that is involved in an immune response, e.g., in the promotion of an immune effector response. Examples of immune effector cells include T cells, B cells, NK cells, natural killer T (NKT) cells, mast cells, and myeloid-derived phagocytes, etc. Immune cells described herein can be autologous or allogeneic.

“Immune effector function” or “immune effector response,” as that term is used herein, refers to function or response, e.g., of an immune effector cell, that enhances or promotes an immune attack of a target cell. For example, an immune effector function or response refers a property of a T or NK cell that promotes killing or the inhibition of growth or proliferation of a target cell (e.g., tumor cell). In the case of a T cell, primary stimulation and co-stimulation are examples of immune effector function or response.

The term “stimulation,” refers to a primary response induced by binding of a stimulatory molecule (e.g., a TCR/CD3 complex or CAR) with its cognate ligand (or tumor antigen in the case of a CAR) thereby mediating a signal transduction event, such as, but not limited to, signal transduction via the TCR/CD3 complex or signal transduction via the appropriate NK receptor or signaling domains of the CAR. Stimulation can mediate altered expression of certain molecules.

In some embodiments, the immune cell is a recombinant or modified immune cell. The term “recombinant” includes reference to a cell that has been modified by the introduction of a heterologous nucleic acid, or a cell derived from a cell that has been modified in such a manner, but does not encompass the alteration of the cell by naturally occurring events (e.g., spontaneous mutation, natural transformation, natural transduction, natural transposition) such as those occurring without deliberate human intervention. The recombinant immune cell may be a non-naturally occurring cell. The recombinant immune cell may also be an engineered cell. In one embodiment, the recombinant immune cell is an engineered immune cell such as an engineered T-cell (e.g., CAR-T cell) or engineered NK cell (e.g., CAR-NK cell). In some embodiments, the recombinant immune cell is an isolated immune cell.

In some embodiments, the immune cell is a hematopoietic stem cell or a pluripotent stem cell. In some embodiments, the immune cell is a T cell, a B cell, or an NK cell. In some embodiments, the immune cell is a modified T cell (e.g., SPPL3-KO T cell, CAR-T, or SPPL3-KO CAR-T), a modified B cell (e.g., SPPL3-KO B cell), or a modified NK cell (e.g., SPPL3-KO NK cell, CAR-NK, or SPPL3-KO CAR-NK).

In some embodiments, the immune cell is a T cell, such as a modified T cell, e.g., CAR-T cell. In some embodiments, the T cell is selected from the group consisting of a helper CD4+ T cell, a cytotoxic CD8+ T cell, a memory T cell, a regulatory CD4+ T cell, an NKT cell, a mucosal associated invariant T (MAIT) cell, a double negative T (DNT) cell, and a γδ T cell. In some embodiments, the T cell is a naive T cell, a memory stem T cell, a central memory T cell, an effector memory T cell, an effector T cell, a Th1 cell, a Tc1 cell, a Th2 cell, a Tc2 cell, a Th3 cell, a Th9 cell, a Th17 cell, a Th22 cell, or a T (regulatory) Treg cell.

DNT cells, also known as CD3⁺CD4⁻CD8⁻ T cells, or TCRαβ⁺CD4⁻CD8⁻ T cells, which also lack iNKT cell markers (e.g., CD56), are a subset of mature T lymphocytes. DNT cells in tumor tissues were found to have lower expression of Fas compared to CD4⁺ or CD8⁺ T cells; while FasL expression in DNT cells was found to be higher compared to other T cell populations (K. Okamura et al. “The potential target of double negative T cells in cancer immunotherapy,” Meeting Abstract e15180|2020 ASCO Annual Meeting I).

γδ T cells are usually double negative (CD4⁻CD8⁻). γδ T cells do not require antigen processing or major-histocompatibility-complex (MHC) molecules for epitope presentation. Some γδ T cells recognize MHC class Ib molecules or butyrophilin molecules. Because of non-MHC-restricted antigen recognition and high cytokine (e.g., IL-17, IFN-γ) secretion, γδ T cells are suitable for effective cancer treatment.

Also see “isolation and culture of immune cells” subsection below for examples and sources of immune cells.

### Pharmaceutical Compositions

Also provided are pharmaceutical compositions comprising any of the immune cells described herein (e.g., SPPL3 KO immune cells), and optionally a pharmaceutically acceptable excipient. Pharmaceutical compositions can be prepared by mixing any modified immune cells described herein with suitable pharmaceutically acceptable carriers, excipients or stabilizers (Remington's Pharmaceutical Sciences 16th edition, Osol, A. Ed. (1980)), in the form of lyophilized formulations or aqueous solutions.

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers are used to control the pH in a range which optimizes the therapeutic effectiveness, especially if stability is pH dependent. Suitable buffering agents for use with the present invention include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives are added to retard microbial growth, and are typically present in a range from 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. Tonicity agents can be present in any amount between 0.1% to 25% by weight, preferably 1 to 5%, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they must be sterile. The pharmaceutical compositions herein generally are placed into a container having a sterile access port, for example, an intravenous solution bag or vial having a stopper pierceable by a hypodermic injection needle.

### Activation-Induced Cell Death (AICD)

AICD, programmed cell death that results from the interaction of Fas receptors (e.g., Fas, CD95) with Fas ligands (e.g., FasL, CD95 ligand), helps to maintain peripheral immune tolerance and/or kill tumor cells. The AICD effector cell (e.g., T cells, and NK cells) expresses FasL, and apoptosis is induced in the cell (e.g., tumor cells, immune cells such as T cells, B cells, and NK cells) expressing the Fas receptor. AICD is a negative regulator of activated T lymphocytes resulting from repeated stimulation of their T cell receptors (TCRs). Alteration of this process may lead to autoimmune diseases (Zhang J, et al. (2004) Cell Mol Immunol. 1(3):186-92).

Mechanistically, the binding of a Fas ligand to a Fas receptor triggers trimerization of the Fas receptor, whose cytoplasmic domain is then able to bind the death domain of the adaptor protein FADD (Fas-associated protein with death domain). Procaspase 8 binds to FADD's death effector domain and proteolytically self-activates caspase 8; Fas, FADD, and procaspase 8 together form a death-inducing signaling complex. Activated caspase 8 is released into the cytosol, where it activates the caspase cascade that initiates apoptosis (Nagata S. (1997) Cell. 88(3):355-65s.

The balance of activation-induced proliferation and death of effector cells is a key point in the homeostatic expansion of immune cells. AICD is extremely important to lymphocyte development, acting to remove self-reactive immature lymphocytes. Immature lymphocytes whose antigen receptors bind self-antigen strongly and give a strong activation signal are eliminated by apoptosis (D. R. Green et al., “Activation-Induced Apoptosis in Lymphoid Systems,” Sem. Immunol. 4:379-388 (1992)). When lymphocytes mature, productive activation requires not only the primary stimulation of the cells' antigen receptor, but also costimulatory signals. In T cells, the binding of CD28 by its ligands of the B7 family serves this costimulatory role, whereas in B cells, co-stimulation occurs via binding of CD40 to its ligand gp39 (E. A. Clark & J. A. Ledbetter, “How B and T Cells Talk to Each Other,” Nature 367:425-428 (1994)). In the absence of co-stimulation, lymphocytes that receive only antigen receptor stimulation become non-responsive or anergic and their growth is inhibited (E. A. Clark & J. A. Ledbetter (1994), supra).

Because activated immune cells (e.g., T cells, B cells, and NK cells) express Fas in addition to FasL, they can be killed by themselves or each other that express FasL. See, e.g., Huan et al., Hum Cell. 2022; 35(2):441-447. Hence, AICD contributes to the poor persistence of therapeutic immune cells (e.g., CAR-T, CAR-NK) in vivo, and their limitations in clinical uses.

### SPPL3

Signal peptide peptidase like 3 (SPPL3; also known as IMP2, PSH1, or PSL4) is a multi-transluminal protein located in Golgi-associated vesicle membrane, plasma membrane, and rough endoplasmic reticulum. SPPL3 enables aspartic endopeptidase activity, intramembrane cleaving and protein homodimerization activity. Many of its substrates locate at the Golgi, and are involved in N- and O-linked glycan modification and glycosaminoglycan biosynthesis. SPPL3 is crucial for cleaving and extracellular release of luminal domain of glycosyltransferases and glycosidases. Shedding of glycan-modifying enzymes can impair their activities in the Golgi. Increased SPPL3 expression is associated with hypoglycosylation of many secretory and membrane proteins; decreased SPPL3 expression is associated with hyperglycosylated proteins. Also see Mentrup et al., “Latest emerging functions of SPP/SPPL intramembrane proteases,” Eur J Cell Biol. 2017; 96(5):372-382, the content of which is incorporated herein by reference in its entirety. In the absence of SPPL3, researchers noticed upregulated cell surface neolacto-series glycosphingolipids (GSLs), which in turn sterically impeded antibody and receptor interactions with HLA class I (HLA-I) glycoproteins and reduced CD8+ T cell activation (Jongsma et al., Immunity. 2021; 54(1):132-150.e9). SPPL3 also has non-proteolytic function, including interacting with stromal interaction molecule 1 (STIM1) and Orai1, enhancing TCR signal to greatly induce calcium influx and NFAT activation crucial for lymphocyte signaling (Mentrup et al., supra).

In some embodiments, the SPPL3 protein is a human SPPL3 protein. In some embodiments, the human SPPL3 protein comprises the sequence of SEQ ID NO: 4.

SEQ ID NO: 4
MAEQTYSWAYSLVDSSQVSTFLISILLIVYGSFRSLNMDFENQDKEKDSNSSSGSFNGNSTNNSIQTIDS
TQALFLPIGASVSLLVMFFFFDSVQVVFTICTAVLATIAFAFLLLPMCQYLTRPCSPQNKISFGCCGRFT
AAELLSFSLSVMLVLIWVLTGHWLLMDALAMGLCVAMIAFVRLPSLKVSCLLLSGLLIYDVFWVFFS
AYIFNSNVMVKVATQPADNPLDVLSRKLHLGPNVGRDVPRLSLPGKLVFPSSTGSHFSMLGIGDIVMP
GLLLCFVLRYDNYKKQASGDSCGAPGPANISGRMQKVSYFHCTLIGYFVGLLTATVASRIHRAAQPA
LLYLVPFTLLPLLTMAYLKGDLRRMWSEPFHSKSSSSRFLEV

### III. METHODS OF GENERATING MODIFIED IMMUNE CELLS

One aspect of the present invention provides methods of generating any of the immune cells described herein, such as immune cells (e.g., CAR-T or CAR-NK cells) modified to reduce or eliminate expression (RNA and/or protein expression) and/or function of the one or more target proteins (e.g., SPPL3 protein) and/or other proteins (e.g., TCR protein). In some embodiments, the method of generating a modified immune cell comprises inactivating or reducing (e.g., reducing at least about any of 10%, 20%, 30%, 40%, 50%, 60%, 70%, 80%, 90%, 95%, or more) expression (RNA and/or protein expression) of one or more target genes identified by any of the target gene identification methods described herein in an immune cell, such as one or more target genes (e.g., AICD/FasL resistant genes) selected from the group consisting of SPPL3, FADD, FAS, CASP8, ARID1A, BAK1, BID, ETS1, IKZF2, and HIST1H1B (e.g., SPPL3). In some embodiments, the method of generating a modified immune cell comprises expressing an engineered receptor (e.g., CAR) in an immune cell (e.g., primary immune cell, or immune cell modified to reduce or eliminate expression and/or function of the one or more target proteins (e.g., SPPL3 protein) and/or other proteins (e.g., TCR)).

In some embodiments, there is provided a method of i) prolonging (e.g., prolonging at least about any of 10%, 20%, 30%, 40%, 50%, 60%, 70%, 80%, 90%, 95%, 1-fold, 2-fold, 5-fold, 10-fold, 20-fold, 50-fold, 100-fold, or more) in vivo persistence, ii) reducing (e.g., reducing at least about any of 10%, 20%, 30%, 40%, 50%, 60%, 70%, 80%, 90%, 95%, or 100%) AICD (or increasing (e.g., increasing at least about any of 10%, 20%, 30%, 40%, 50%, 60%, 70%, 80%, 90%, 95%, 1-fold, 2-fold, 5-fold, 10-fold, 20-fold, 50-fold, 100-fold, or more) resistance to AICD), and/or iii) reducing (e.g., reducing at least about any of 10%, 20%, 30%, 40%, 50%, 60%, 70%, 80%, 90%, 95%, or 100%) host-versus-graft (HvG) response of an immune cell (e.g., CAR-T or CAR-NK cell), comprising modifying the immune cell to reduce (e.g., reducing at least about any of 10%, 20%, 30%, 40%, 50%, 60%, 70%, 80%, 90%, 95%, or more) or eliminate expression (RNA and/or protein expression) and/or function of one or more target proteins identified herein (e.g., AICD/FasL resistant gene-encoded proteins), such as selected from the group consisting of: SPPL3, FADD, FAS, CASP8, ARID1A, BAK1, BID, ETS1, IKZF2, and HIST1H1B. In some embodiments, the method comprises modifying the immune cell to reduce or eliminate expression and/or function of SPPL3 protein. Thus in some embodiments, there is provided a method of i) prolonging (e.g., prolonging at least about any of 10%, 20%, 30%, 40%, 50%, 60%, 70%, 80%, 90%, 95%, 1-fold, 2-fold, 5-fold, 10-fold, 20-fold, 50-fold, 100-fold, or more) in vivo persistence, ii) reducing (e.g., reducing at least about any of 10%, 20%, 30%, 40%, 50%, 60%, 70%, 80%, 90%, 95%, or 100%) AICD (or increasing (e.g., increasing at least about any of 10%, 20%, 30%, 40%, 50%, 60%, 70%, 80%, 90%, 95%, 1-fold, 2-fold, 5-fold, 10-fold, 20-fold, 50-fold, 100-fold, or more) resistance to AICD), and/or iii) reducing (e.g., reducing at least about any of 10%, 20%, 30%, 40%, 50%, 60%, 70%, 80%, 90%, 95%, or 100%) host-versus-graft (HvG) response of an immune cell (e.g., CAR-T or CAR-NK cell), comprising modifying the immune cell to reduce (e.g., reducing at least about any of 10%, 20%, 30%, 40%, 50%, 60%, 70%, 80%, 90%, 95%, or more) or eliminate expression (RNA and/or protein expression) and/or function of SPPL3 protein. In some embodiments, the immune cell is a T cell. In some embodiments, the immune cell (e.g., T cell) expresses a CAR. In some embodiments, the immune cell (e.g., T cell) is further modified to express a CAR. In some embodiments, the immune cell (e.g., T cell such as CAR-T) is further modified to reduce (e.g., reducing at least about any of 10%, 20%, 30%, 40%, 50%, 60%, 70%, 80%, 90%, 95%, or more) or eliminate expression (RNA and/or protein expression) and/or function of a TCR protein.

In some embodiments, there is provided a method of generating an immune cell (e.g., CAR-T or CAR-NK) with increased (e.g., increasing at least about any of 10%, 20%, 30%, 40%, 50%, 60%, 70%, 80%, 90%, 95%, 1-fold, 2-fold, 5-fold, 10-fold, 20-fold, 50-fold, 100-fold, or more) resistance to AICD, comprising inactivating an AICD/FasL resistant gene identified using any of the target gene screening methods described herein, such as a gene selected from the group consisting of SPPL3, FADD, FAS, CASP8, ARID1A, BAK1, BID, ETS1, IKZF2, and HIST1H1B, in a precursor immune cell. In some embodiments, there is provided a method of generating an immune cell (e.g., CAR-T or CAR-NK) with increased (e.g., increasing at least about any of 10%, 20%, 30%, 40%, 50%, 60%, 70%, 80%, 90%, 95%, 1-fold, 2-fold, 5-fold, 10-fold, 20-fold, 50-fold, 100-fold, or more) resistance to AICD, comprising inactivating SPPL3 gene in a precursor immune cell. In some embodiments, the immune cell is a T cell, a B cell, or an NK cell. In some embodiments, the immune cell is a T cell, such as selected from the group consisting of a helper CD4+ T cell, a cytotoxic CD8+ T cell, a memory T cell, a regulatory CD4+ T cell, an NKT cell, an MAIT cell, a DNT cell, and a γδ T cell. In some embodiments, the precursor immune cell expresses an engineered receptor (e.g., CAR, TAC, engineered TCR). In some embodiments, the method further comprises introducing into the precursor immune cell a nucleic acid encoding an engineered receptor. In some embodiments, the engineered receptor is a CAR. In some embodiments, there is provided a method of generating an immune cell (e.g., T cell) with increased (e.g., increasing at least about any of 10%, 20%, 30%, 40%, 50%, 60%, 70%, 80%, 90%, 95%, 1-fold, 2-fold, 5-fold, 10-fold, 20-fold, 50-fold, 100-fold, or more) resistance to AICD, comprising inactivating SPPL3 gene in a precursor immune cell that expresses a CAR (e.g., anti-CD19 CAR). In some embodiments, the method further comprises inactivating a TCR gene (e.g., one or more of TCRα, TCRβ, TCRγ, TCRδ) in the precursor immune cell.

In some embodiments, the method of generating a modified immune cell comprises creating one or more mutations (e.g., inactivating mutations) at one or more target genes identified by any of the target gene identification methods described herein, such as one or more target genes (e.g., AICD/FasL resistant genes) encoding the one or more target proteins (e.g., SPPL3 protein) described herein. In some embodiments, the method comprises contacting an initial population of immune cells (e.g., CAR-T cells or CAR-NK cells) with a mutagenic agent, and selecting modified immune cells comprising one or more mutations (e.g., inactivating mutations) at one or more target genes identified herein, such as one or more target genes encoding the one or more target proteins (e.g., SPPL3 protein) described herein. In some embodiments, the method comprises creating one or more mutations (e.g., inactivating mutations) at one or more target genes identified herein, such as one or more target genes encoding the one or more target proteins (e.g., SPPL3 protein) described herein, in a precursor immune cell (e.g., CAR-T or CAR-NK cell) by gene editing, such as any gene editing methods known in the art or described herein. For example, non-homologous end joining (NHEJ)- or HDR (e.g., homologous recombination)-mediated gene modification or disruption, or ZFN-, TALEN-, or CRISPR/Cas-mediated gene modification or disruption. Methods of detecting such mutations are well known in the art, such as by PCR. In some embodiments, the method comprises creating one or more mutations (e.g., inactivating mutations) at one or more target gene products identified herein, such as one or more RNAs encoding the one or more target proteins (e.g., SPPL3 protein) described herein, or the one or more target proteins described herein, in a precursor immune cell by RNA editing (e.g., LEAPER, REPAIR) or protein editing.

In some embodiments, the method of generating a modified immune cell further comprises modifying the immune cell (e.g., target protein-modified immune cell) to reduce (e.g., reducing at least about any of 10%, 20%, 30%, 40%, 50%, 60%, 70%, 80%, 90%, 95%, or more) or eliminate expression (RNA and/or protein expression) and/or function of one or more other proteins selected from the group consisting of TCRα, TCRβ, TCRγ, TCRδ, HLA-A, HLA-B, HLA-C, HLA-E, HLA-F, HLA-G, B2M, PD-1, TIM-3, LAG-3, CTLA-4, CISH, SLLP3, Fas, FADD, CASP8, ARID1A, BAK1, BID, ETS1, IKZF2, HIST1H1B, B7-H6, MICA, MICB, ULBP1, ULBP2, ULBP3, ULBP4, ULBP5, ULBP6, and ligands of NKp46. In some embodiments, the method further comprises modifying the immune cell to reduce or eliminate expression and/or function of one or more other proteins selected from the group consisting of Fas, FADD, CASP8, ARID1A, BAK1, BID, ETS1, IKZF2, HIST1H1B. In some embodiments, the method further comprises modifying the immune cell to reduce or eliminate expression and/or function of one or more other proteins selected from the group consisting of TCRα, TCRβ, TCRγ, TCRδ, HLA-A, HLA-B, HLA-C, HLA-E, HLA-F, HLA-G, B2M, PD-1, TIM-3, LAG-3, CTLA-4, CISH, B7-H6, MICA, MICB, ULBP1, ULBP2, ULBP3, ULBP4, ULBP5, ULBP6, and ligands of NKp46. In some embodiments, the method further comprises modifying the immune cell to reduce or eliminate expression and/or function of a TCR protein (e.g., one or more of TCRα, TCRβ, TCRγ, TCRδ).

In some embodiments, the expression of the one or more target proteins (e.g., SPPL3 protein) and/or other proteins (e.g., TCR) is reduced or inhibited by an antisense RNA, an siRNA, or an shRNA specifically recognizing an RNA encoding the one or more target proteins (e.g., SPPL3 protein) and/or other proteins (e.g., TCR). In some embodiments, the method of generating a modified immune cell comprises introducing into a precursor immune cell (e.g., CAR-T or CAR-NK cell) a nucleic acid (e.g., vector) encoding an antisense RNA, an siRNA, or an shRNA specifically recognizing an RNA encoding the one or more target proteins (e.g., SPPL3 protein) and/or other proteins (e.g., TCR). In some embodiments, the method comprises introducing into the immune cell (e.g., CAR-T or CAR-NK cell) an antisense RNA, an siRNA, or an shRNA specifically recognizing an RNA encoding the one or more target proteins (e.g., SPPL3 protein) and/or other proteins (e.g., TCR).

In some embodiments, the expression (RNA and/or protein expression) and/or function of the one or more target proteins (e.g., SPPL3 protein) and/or other proteins (e.g., TCR) is reduced or inhibited by a small molecule compound, a nucleic acid (or vector comprising thereof), a lipid, and/or a protein molecule (e.g., a dominant negative binding partner of the target protein or other protein). In some embodiments, the method of generating a modified immune cell comprises introducing into, or contacting, a precursor immune cell (e.g., CAR-T or CAR-NK cell) the nucleic acid (or vector comprising thereof), the lipid, and/or the protein molecule (or a nucleic acid encoding the protein molecule). In some embodiments, the method comprises contacting a precursor immune cell (e.g., CAR-T or CAR-NK cell) with the small molecule compound. In some embodiments, the contacting is in vivo, in vitro, or ex vivo. In some embodiments, the in vivo contacting of the immune cell is by administration of the small molecule compound, nucleic acid (or vector comprising thereof), lipid, and/or protein molecule to an individual (e.g., human) having the immune cell. Any suitable administration route can be used herein, including but not limited to, intravenous, subcutaneous, intratumoral, intramuscular, or oral administration.

In some embodiments, the function of the one or more target proteins (e.g., SPPL3 protein) and/or other proteins (e.g., TCR) is reduced or inhibited by a chemically modified mRNA, such as a chemically modified mRNA encoding a dominant negative inhibitor (e.g., dominant negative variant or fragment thereof, or dominant negative binding partner thereof) of the one or more target proteins (e.g., SPPL3 protein) and/or other proteins (e.g., TCR). In some embodiments, the method of generating a modified immune cell comprises introducing into a precursor immune cell (e.g., CAR-T or CAR-NK cell) the chemically modified mRNA.

In some embodiments, the immune cell is modified to express a dominant negative variant (e.g., catalytically inactive, binding inactive, activation deficient or inactive) or dominant negative fragment thereof of the one or more target proteins (e.g., SPPL3 protein) and/or other proteins (e.g., TCR). In some embodiments, the immune cell is modified to express a dominant negative binding partner or fragment thereof of the one or more target proteins (e.g., SPPL3 protein) and/or other proteins (e.g., TCR). In some embodiments, the method of generating a modified immune cell comprises introducing into a precursor immune cell (e.g., CAR-T or CAR-NK cell) a nucleic acid (e.g., vector, or RNA) encoding the dominant negative variant or fragment thereof, or the dominant negative binding partner or fragment thereof, of the one or more target proteins (e.g., SPPL3 protein) and/or other proteins (e.g., TCR). In some embodiments, the DNA locus or RNA encoding the one or more target proteins (e.g., SPPL3 protein) and/or other proteins (e.g., TCR) in the immune cell is modified (e.g., via gene editing or RNA editing, or mutagenic agent) to express a dominant negative variant or dominant negative fragment thereof.

In some embodiments, the immune cell is genetically modified at the DNA locus encoding the one or more target proteins (e.g., SPPL3 protein) and/or other proteins (e.g., TCR). In some embodiments, the immune cell is genetically modified at the SPPL3 locus. In some embodiments, the DNA locus (e.g., SPPL3 locus) is modified by a mutagenic agent. Mutagenic agents can be classified into three categories: physical (e.g., gamma rays, ultraviolet radiations), chemical (e.g., ethyl methane sulphonate or EMS) and transposable elements (such as transposons, retrotransposons, T-DNA, retroviruses). In some embodiments, the method of generating a modified immune cell comprises contacting a precursor immune cell (e.g., CAR-T or CAR-NK cell) with a mutagenic agent to reduce or eliminate the expression (RNA and/or protein expression) and/or function of the one or more target proteins (e.g., SPPL3 protein) and/or other proteins (e.g., TCR). In some embodiments, the DNA locus encoding the one or more target proteins (e.g., SPPL3 protein) and/or other proteins (e.g., TCR) is modified by gene editing, including but not limited to, NHEJ, HDR, ZFN, TALEN, or CRISPR/Cas mediated gene editing. In some embodiments, the gene editing comprises gene KO. In some embodiments, the gene editing comprises base editing (e.g., introducing nonsynonymous substitution). In some embodiments, the base editing is mediated by CRISPR/Cas, such as by fusing an ADE or adenine deaminase (ADA), or a CBE or cytidine deaminase (CDA), or functional fragment thereof, to a dead Cas (e.g., dCas9) protein. In some embodiments, the method comprises introducing into a precursor immune cell (e.g., CAR-T or CAR-NK cell) one or more nucleic acids (e.g., vector or RNA) encoding the ZFN, TALEN, or CRISPR/Cas system specifically recognizing a target sequence of a gene encoding one or more target proteins (e.g., SPPL3) and/or other proteins (e.g., TCR). In some embodiments, two or more nucleic acids encoding different components of the ZFN, TALEN, or CRISPR/Cas system are introduced into the immune cell simultaneously. In some embodiments, two or more nucleic acids encoding different components of the ZFN, TALEN, or CRISPR/Cas system are introduced into the immune cell sequentially. In some embodiments, a nucleic acid template (e.g., with a desired modification and homologous arms for HDR) is further introduced into the immune cell, either sequentially or simultaneously with the ZFN, TALEN, or CRISPR/Cas system. In some embodiments, the method of generating a modified immune cell further comprises introducing into the precursor immune cell or the modified immune cell (e.g., SPPL3 modified) one or more nucleic acids encoding a CAR.

In some embodiments, the immune cell is genetically modified at the RNA encoding the one or more target proteins (e.g., SPPL3 protein) and/or other proteins (e.g., TCR), such as by RNA editing (e.g., LEAPER, REPAIR, RESTORE, CIRTS, RESCUE, CLUSTER). In some embodiments, the RNA editing comprises base editing (e.g., introducing nonsynonymous substitution). In some embodiments, the RNA editing is mediated by CRISPR/Cas, such as by fusing an adenine deaminase (ADA) or a cytidine deaminase (CDA) or functional fragment thereof to a dead Cas (e.g., dCas13a) protein. In some embodiments, the method of generating a modified immune cell comprises introducing into a precursor immune cell (e.g., CAR-T or CAR-NK cell) one or more nucleic acids (e.g., vector or RNA) encoding the CRISPR/Cas or LEAPER system specifically recognizing a target sequence of an RNA encoding the one or more target proteins (e.g., SPPL3) and/or other proteins (e.g., TCR). In some embodiments, two or more nucleic acids encoding different components of the CRISPR/Cas system are introduced into the immune cell simultaneously. In some embodiments, two or more nucleic acids encoding different components of the CRISPR/Cas system are introduced into the immune cell sequentially. In some embodiments, the method of generating a modified immune cell comprises introducing into a precursor immune cell (e.g., CAR-T or CAR-NK cell) an adenosine deaminase-recruiting RNA (arRNA) construct comprising or encoding an arRNA for editing a target sequence of an RNA encoding the one or more target proteins (e.g., SPPL3) and/or other proteins (e.g., TCR), wherein the target sequence comprises a target adenosine (A), wherein the arRNA comprises a complementary RNA sequence (e.g., gRNA) that hybridizes to the target sequence of the RNA, and wherein the arRNA is capable of recruiting an adenosine deaminase acting on RNA (ADAR) (e.g., endogenous ADAR1) to deaminate the target A in the target sequence. In some embodiments, the method of generating a modified immune cell further comprises introducing into the precursor immune cell or the modified immune cell (e.g., SPPL3 modified) one or more nucleic acids encoding a CAR.

In some embodiments, there is provided a method of generating an immune cell (e.g., CAR-T or CAR-NK cell) modified to reduce (e.g., reducing at least about any of 10%, 20%, 30%, 40%, 50%, 60%, 70%, 80%, 90%, 95%, or more) or eliminate expression (RNA and/or protein expression) and/or function of the one or more target proteins identified herein (e.g., AICD/FasL resistant gene-encoded proteins) or selected from the group consisting of: SPPL3, FADD, FAS, CASP8, ARID1A, BAK1, BID, ETS1, IKZF2, and HIST1H1B (e.g., SPPL3 protein) and/or other proteins (e.g., TCR), comprising contacting a precursor immune cell (e.g., precursor T cell or precursor NK cell) with i) a gRNA construct (e.g., a gRNA, or a vector (e.g., viral vector such as lentiviral vector) carrying a nucleic acid encoding the gRNA), wherein the gRNA construct comprises or encodes a gRNA comprising a guide sequence that is complementary (e.g., at least about any of 60%, 70%, 80%, 90%, 95%, 96%, 97%, 98%, 99%, or 100% complementary) to a target site in the DNA locus or RNA encoding the one or more target proteins (e.g., SPPL3 protein) and/or other proteins (e.g., TCR); and optionally ii) a Cas component comprising a Cas protein or a nucleic acid encoding the Cas protein (e.g., Cas9), under a condition that allows introduction of the gRNA construct and the optional Cas component into the precursor immune cell. In some embodiments, there is provided a method of i) prolonging (e.g., prolonging at least about any of 10%, 20%, 30%, 40%, 50%, 60%, 70%, 80%, 90%, 95%, 1-fold, 2-fold, 5-fold, 10-fold, 20-fold, 50-fold, 100-fold, or more) in vivo persistence, ii) reducing (e.g., reducing at least about any of 10%, 20%, 30%, 40%, 50%, 60%, 70%, 80%, 90%, 95%, or 100%) AICD (or increasing (e.g., increasing at least about any of 10%, 20%, 30%, 40%, 50%, 60%, 70%, 80%, 90%, 95%, 1-fold, 2-fold, 5-fold, 10-fold, 20-fold, 50-fold, 100-fold, or more) resistance to AICD), and/or iii) reducing (e.g., reducing at least about any of 10%, 20%, 30%, 40%, 50%, 60%, 70%, 80%, 90%, 95%, or 100%) HvG response of an immune cell (e.g., CAR-T or CAR-NK cell), comprising contacting a precursor immune cell (e.g., precursor T cell or precursor NK cell) with i) a gRNA construct (e.g., a gRNA, or a vector (e.g., viral vector such as lentiviral vector) carrying a nucleic acid encoding the gRNA), wherein the gRNA construct comprises or encodes a gRNA comprising a guide sequence that is complementary (e.g., at least about any of 60%, 70%, 80%, 90%, 95%, 96%, 97%, 98%, 99%, or 100% complementary) to a target site in the DNA locus or RNA encoding one or more target proteins identified herein (e.g., AICD/FasL resistant gene-encoded proteins) or selected from the group consisting of: SPPL3, FADD, FAS, CASP8, ARID1A, BAK1, BID, ETS1, IKZF2, and HIST1H1B (e.g., SPPL3 protein); and optionally ii) a Cas component comprising a Cas protein or a nucleic acid encoding the Cas protein (e.g., Cas9), under a condition that allows introduction of the gRNA construct and the optional Cas component into the precursor immune cell, thereby reducing or eliminating expression (RNA and/or protein expression) and/or function of the one or more target proteins (e.g., SPPL3 protein). In some embodiments, the precursor immune cell already expresses a Cas protein before introducing the gRNA construct. In some embodiments, the precursor immune cell does not express a Cas protein before introducing the gRNA construct and the Cas component. In some embodiments, the method further comprises introducing a vector (e.g., viral vector such as lentiviral vector) carrying a nucleic acid encoding a Cas protein (e.g., Cas9), or a Cas (e.g., Cas9) mRNA, into the precursor immune cell or the precursor immune cell comprising said gRNA construct. In some embodiments, the method further comprises introducing into the immune cell a nucleic acid template (e.g., comprising a mutation) for modifying the target site of the DNA locus encoding the one or more target proteins (e.g., SPPL3 protein) and/or other proteins (e.g., TCR) via HDR. In some embodiments, the Cas protein has endonuclease activity. In some embodiments, the Cas protein is fusion protein, such as a fusion protein comprising i) a dCas (e.g., dCas13, dCas9) and ii) an adenine base editor (ABE) or ADA (e.g., TadA, such as TadA8e), or a cytidine base editor (CBE) or CDA, or functional fragment thereof. In some embodiments, the Cas protein is a dCas13-ADAR fusion protein, such as dCas13a-ADAR1, dCas13a-ADAR2, dCas13b-ADAR1, dCas13b-ADAR2, and functional variants thereof that are capable of performing targeted RNA editing (e.g., C to U, A to I). In some embodiments, the Cas protein is Cas9, such as dCas9. In some embodiments, the Cas protein is a dCas9-ADA (e.g., TadA) or dCas9-CDA fusion protein. In some embodiments, the precursor immune cell (e.g., before target protein modification) has reduced (e.g., reducing at least about any of 10%, 20%, 30%, 40%, 50%, 60%, 70%, 80%, 90%, 95%, or more) or no expression (RNA and/or protein expression) and/or function of one or more other proteins selected from the group consisting of TCRα, TCRβ, TCRγ, TCRδ, HLA-A, HLA-B, HLA-C, HLA-E, HLA-F, HLA-G, B2M, PD-1, TIM-3, LAG-3, CTLA-4, CISH, SPPL3, Fas, FADD, CASP8, ARID1A, BAK1, BID, ETS1, IKZF2, HIST1H1B, B7-H6, MICA, MICB, ULBP1, ULBP2, ULBP3, ULBP4, ULBP5, ULBP6, and ligands of NKp46. In some embodiments, the precursor immune cell (e.g., target protein-modified immune cell) is further modified to reduce or eliminate expression and/or function of the one or more other proteins (e.g., TCR), such as genetically modified at one or more loci or RNAs encoding the one or more other proteins. In some embodiments, the method further comprises contacting the precursor immune cell (e.g., target protein-modified immune cell) with i) a gRNA construct, wherein the gRNA construct comprises or encodes a gRNA comprising a guide sequence that is complementary to a target site in the DNA locus or RNA encoding the one or more other proteins (e.g., one or more of Fas, FADD, CASP8, ARID1A, BAK1, BID, ETS1, IKZF2, HIST1H1B); and optionally ii) a Cas component comprising a Cas protein or a nucleic acid encoding the Cas protein (e.g., Cas9), under a condition that allows introduction of the gRNA construct and the optional Cas component into the precursor immune cell, thereby reducing or eliminating expression (RNA and/or protein expression) and/or function of the one or more other proteins. In some embodiments, the precursor immune cell (e.g., target protein-modified immune cell) already has the Cas component, only the gRNA construct targeting the one or more other proteins is further introduced. In some embodiments, the RNA encoding the one or more target proteins (e.g., SPPL3 protein) and/or other proteins is modified by LEAPER. Thus in some embodiments, there is provided a method of generating an immune cell (e.g., CAR-T or CAR-NK cell) modified to reduce (e.g., reducing at least about any of 10%, 20%, 30%, 40%, 50%, 60%, 70%, 80%, 90%, 95%, or more) or eliminate expression (e.g., functional protein expression) and/or function of the one or more target proteins identified herein or selected from the group consisting of: SPPL3, FADD, FAS, CASP8, ARID1A, BAK1, BID, ETS1, IKZF2, and HIST1H1B (e.g., SPPL3 protein) and/or other proteins (e.g., TCR), comprising contacting a precursor immune cell (e.g., precursor T cell or precursor NK cell) with an arRNA construct (e.g., vector, RNA) comprising or encoding an arRNA for editing a target sequence of an RNA encoding the one or more target proteins (e.g., SPPL3) and/or other proteins (e.g., TCR), under a condition that allows introduction of the arRNA construct into the precursor immune cell, wherein the target sequence comprises a target adenosine (A), wherein the arRNA comprises a complementary (e.g., at least about any of 60%, 70%, 80%, 90%, 95%, 96%, 97%, 98%, 99%, or 100% complementary) RNA sequence (e.g., gRNA) that hybridizes to the target sequence of the RNA, and wherein the arRNA is capable of recruiting an ADAR (e.g., endogenous ADAR1) to deaminate the target A in the target sequence, wherein the deamination of the target A in the target sequence reduces or eliminates expression and/or function of the one or more target proteins and/or other proteins. In some embodiments, there is provided a method of i) prolonging (e.g., prolonging at least about any of 10%, 20%, 30%, 40%, 50%, 60%, 70%, 80%, 90%, 95%, 1-fold, 2-fold, 5-fold, 10-fold, 20-fold, 50-fold, 100-fold, or more) in vivo persistence, ii) reducing (e.g., reducing at least about any of 10%, 20%, 30%, 40%, 50%, 60%, 70%, 80%, 90%, 95%, or 100%) AICD (or increasing (e.g., increasing at least about any of 10%, 20%, 30%, 40%, 50%, 60%, 70%, 80%, 90%, 95%, 1-fold, 2-fold, 5-fold, 10-fold, 20-fold, 50-fold, 100-fold, or more) resistance to AICD), and/or iii) reducing (e.g., reducing at least about any of 10%, 20%, 30%, 40%, 50%, 60%, 70%, 80%, 90%, 95%, or 100%) HvG response of an immune cell (e.g., CAR-T or CAR-NK cell), comprising contacting a precursor immune cell (e.g., precursor T cell or precursor NK cell) with an arRNA construct (e.g., vector, RNA) comprising or encoding an arRNA for editing a target sequence of an RNA encoding one or more target proteins identified herein or selected from the group consisting of: SPPL3, FADD, FAS, CASP8, ARID1A, BAK1, BID, ETS1, IKZF2, and HIST1H1B (e.g., SPPL3 protein), under a condition that allows introduction of the arRNA construct into the precursor immune cell, wherein the target sequence comprises a target adenosine (A), wherein the arRNA comprises a complementary (e.g., at least about any of 60%, 70%, 80%, 90%, 95%, 96%, 97%, 98%, 99%, or 100% complementary) RNA sequence (e.g., gRNA) that hybridizes to the target sequence of the RNA, and wherein the arRNA is capable of recruiting an ADAR (e.g., endogenous ADAR1) to deaminate the target A in the target sequence, wherein the deamination of the target A in the target sequence reduces or eliminates expression and/or function of the one or more target proteins. In some embodiments, the precursor immune cell expresses an engineered receptor (e.g., CAR, engineered TCR, or TAC) before introducing the gRNA construct and/or the Cas component, or the arRNA construct. In some embodiments, the method further comprises introducing into the precursor immune cell (e.g., target protein-modified immune cell) a nucleic acid encoding an engineered receptor. In some embodiments, the engineered receptor is a CAR comprising: i) an extracellular antigen binding domain (e.g., sdAb, scFv, Fab) specifically recognizing one or more target antigens (e.g., tumor antigen) or target epitopes, ii) a transmembrane domain; and iii) an intracellular signaling domain. In some embodiments, the methods of modifying the one or more target proteins and/or other proteins are the same, e.g., both via CRISPR-Cas mediated gene editing. In some embodiments, the methods of modifying the one or more target proteins and/or other proteins are different, e.g., some via CRISPR-Cas mediated gene editing, some via LEAPER mediated RNA editing. In some embodiments, the gRNA construct or arRNA construct targeting the one or more target proteins (e.g., SPPL3 protein), the gRNA construct or arRNA construct targeting the one or more other proteins (e.g., one or more of Fas, FADD, CASP8, ARID1A, BAK1, BID, ETS1, IKZF2, HIST1H1B), the Cas component, and/or the nucleic acid encoding the engineered receptor (e.g., CAR) are introduced into the precursor immune cell simultaneously. In some embodiments, the gRNA construct or arRNA construct targeting the one or more target proteins (e.g., SPPL3 protein), the gRNA construct or arRNA construct targeting the one or more other proteins (e.g., one or more of Fas, FADD, CASP8, ARID1A, BAK1, BID, ETS1, IKZF2, HIST1H1B), the Cas component, and/or the nucleic acid encoding the engineered receptor (e.g., CAR) are introduced into the precursor immune cell sequentially, for example, i) the gRNA construct targeting the one or more target proteins and/or other proteins+the Cas component first, then the nucleic acid encoding the engineered receptor; ii) the Cas component first, then the gRNA construct targeting the one or more target proteins and/or other proteins, then the nucleic acid encoding the engineered receptor; or iii) the nucleic acid encoding the engineered receptor first, then the gRNA construct targeting the one or more target proteins and/or other proteins+the Cas component, etc. In some embodiments, the nucleic acid encoding the engineered receptor, the nucleic acid encoding the gRNA or arRNA against the one or more target proteins (e.g., SPPL3 protein), the nucleic acid encoding the gRNA or arRNA against the one or more other proteins, and/or the nucleic acid encoding the Cas protein are on different vectors. In some embodiments, at least two of the nucleic acid encoding the engineered receptor, the nucleic acid encoding the gRNA or arRNA against the one or more target proteins (e.g., SPPL3 protein), the nucleic acid encoding the gRNA or arRNA against the one or more other proteins, and/or the nucleic acid encoding the Cas protein are on the same vector, either under the same promoter control, or under separate promoter controls. In some embodiments, the nucleic acid encoding the engineered receptor, the nucleic acid encoding the gRNA or arRNA against the one or more target proteins (e.g., SPPL3 protein), the nucleic acid encoding the gRNA or arRNA against the one or more other proteins, and/or the nucleic acid encoding the Cas protein are connected by one or more IRES linking sequences and under the same promoter control. In some embodiments, the modification to reduce or eliminate expression (RNA and/or protein expression) and/or function of the one or more target proteins (e.g., SPPL3 protein) and/or other proteins does not down-regulate or eliminate expression (RNA and/or protein expression) and/or function of the engineered receptor (e.g., CAR). In some embodiments, the modification to reduce or eliminate expression and/or function of the one or more target proteins (e.g., SPPL3 protein) and/or other proteins (e.g., TCR) down-regulates expression (RNA and/or protein expression) and/or function of the engineered receptor by at most about 30% (e.g., at most about any of 25%, 20%, 15%, 10%, 5%, 1%, or less). In some embodiments, the immune cell is a T cell, a B cell, or an NK cell. In some embodiments, the immune cell is a T cell, such as selected from the group consisting of a helper CD4+ T cell, a cytotoxic CD8+ T cell, a memory T cell, a regulatory CD4+ T cell, an NKT cell, an MAIT cell, a DNT cell, and a γδ T cell. In some embodiments, the modification to reduce or eliminate expression (RNA and/or protein expression) and/or function of the one or more target proteins (e.g., SPPL3 protein) and/or other proteins (e.g., TCR): i) reduces at least about 10% (e.g., reducing at least about any of 20%, 30%, 40%, 50%, 60%, 70%, 80%, 90%, 95%, or 100%) cell surface expression of one or more of Fas, HLA-A, HLA-B, HLA-C, HLA-E, B7-H6, MICA, MICB, ULBP1, ULBP2, ULBP3, ULBP4, ULBP5, ULBP6, and ligands of NKp46; ii) reduces at least about 10% (e.g., reducing at least about any of 20%, 30%, 40%, 50%, 60%, 70%, 80%, 90%, 95%, or 100%) killing by an allogeneic T cell; and/or iii) reduces at least about 10% (e.g., reducing at least about any of 20%, 30%, 40%, 50%, 60%, 70%, 80%, 90%, 95%, or 100%) killing by an autologous or allogeneic NK cell. In some embodiments, the modification to reduce or eliminate expression (RNA and/or protein expression) and/or function of the one or more target proteins (e.g., SPPL3 protein) and/or other proteins (e.g., TCR) i) prolongs at least about 10% (e.g., at least about any of 20%, 30%, 40%, 50%, 60%, 70%, 80%, 90%, 1-fold, 1.5-fold, 2-fold, 5-fold, 10-fold, 20-fold, 50-fold, or more) in vivo persistence of the modified immune cell; and/or ii) reduces at least about 10% (e.g., at least about any of 20%, 30%, 40%, 50%, 60%, 70%, 80%, 90%, 95%, or 100%) AICD of the modified immune cell, compared to a reference immune cell (e.g., precursor immune cell, such as CAR-T or CAR-NK cell) not having the modification to reduce or eliminate expression (RNA and/or protein expression) and/or function of the one or more target proteins (e.g., SPPL3 protein) and/or other proteins (e.g., TCR). In some embodiments, the guide sequence is encoded by a nucleic acid sequence comprising the sequence of SEQ ID NO: 1.

In some embodiments, there is provided a method of generating a T cell (e.g., CAR-T cell, such as anti-CD19 CAR-T cell) modified to reduce (e.g., reducing at least about any of 10%, 20%, 30%, 40%, 50%, 60%, 70%, 80%, 90%, 95%, or more) or eliminate expression (RNA and/or protein expression) and/or function of SPPL3 protein, comprising contacting a precursor T cell (e.g., precursor CAR-T cell) with a) a gRNA construct (e.g., a gRNA, or a vector (e.g., viral vector such as lentiviral vector) carrying a nucleic acid encoding the gRNA), wherein the gRNA construct comprises or encodes a gRNA comprising a guide sequence that is complementary (e.g., at least about any of 60%, 70%, 80%, 90%, 95%, 96%, 97%, 98%, 99%, or 100% complementary) to a target site in the SPPL3 DNA locus; and optionally b) a Cas component comprising a Cas protein or a nucleic acid encoding the Cas protein (e.g., Cas9), under a condition that allows introduction of the gRNA construct and the optional Cas component into the precursor T cell. In some embodiments, there is provided a method of generating a T cell (e.g., CAR-T cell, such as anti-CD19 CAR-T cell) modified to reduce (e.g., reducing at least about any of 10%, 20%, 30%, 40%, 50%, 60%, 70%, 80%, 90%, 95%, or more) or eliminate expression (RNA and/or protein expression) and/or function of SPPL3 protein and TCR protein, comprising contacting a precursor T cell (e.g., precursor CAR-T cell) with a) a first gRNA construct (e.g., a gRNA, or a vector (e.g., viral vector such as lentiviral vector) carrying a nucleic acid encoding the gRNA), wherein the first gRNA construct comprises or encodes a gRNA comprising a guide sequence that is complementary (e.g., at least about any of 60%, 70%, 80%, 90%, 95%, 96%, 97%, 98%, 99%, or 100% complementary) to a target site in the SPPL3 DNA locus; b) a second gRNA construct (e.g., a gRNA, or a vector (e.g., viral vector such as lentiviral vector) carrying a nucleic acid encoding the gRNA), wherein the second gRNA construct comprises or encodes a gRNA comprising a guide sequence that is complementary (e.g., at least about any of 60%, 70%, 80%, 90%, 95%, 96%, 97%, 98%, 99%, or 100% complementary) to a target site in the TCR DNA locus; and optionally c) a Cas component comprising a Cas protein or a nucleic acid encoding the Cas protein (e.g., Cas9), under a condition that allows introduction of the gRNA construct and the optional Cas component into the precursor T cell. In some embodiments, there is provided a method of i) prolonging in vivo persistence, ii) reducing AICD (or increasing resistance to AICD), and/or iii) reducing HvG response of a T cell (e.g., CAR-T cell, such as anti-CD19 CAR-T cell), comprising contacting a precursor T cell (e.g., precursor CAR-T cell) with a) a gRNA construct (e.g., a gRNA, or a vector (e.g., viral vector such as lentiviral vector) carrying a nucleic acid encoding the gRNA), wherein the gRNA construct comprises or encodes a gRNA comprising a guide sequence that is complementary (e.g., at least about any of 60%, 70%, 80%, 90%, 95%, 96%, 97%, 98%, 99%, or 100% complementary) to a target site in the SPPL3 DNA locus; and optionally b) a Cas component comprising a Cas protein or a nucleic acid encoding the Cas protein (e.g., Cas9), under a condition that allows introduction of the gRNA construct and the optional Cas component into the precursor T cell. In some embodiments, there is provided a method of i) prolonging in vivo persistence, ii) reducing AICD (or increasing resistance to AICD), and/or iii) reducing HvG response of a T cell (e.g., CAR-T cell, such as anti-CD19 CAR-T cell), comprising contacting a precursor T cell (e.g., precursor CAR-T cell) with a) a first gRNA construct (e.g., a gRNA, or a vector (e.g., viral vector such as lentiviral vector) carrying a nucleic acid encoding the gRNA), wherein the first gRNA construct comprises or encodes a gRNA comprising a guide sequence that is complementary (e.g., at least about any of 60%, 70%, 80%, 90%, 95%, 96%, 97%, 98%, 99%, or 100% complementary) to a target site in the SPPL3 DNA locus; b) a second gRNA construct (e.g., a gRNA, or a vector (e.g., viral vector such as lentiviral vector) carrying a nucleic acid encoding the gRNA), wherein the second gRNA construct comprises or encodes a gRNA comprising a guide sequence that is complementary (e.g., at least about any of 60%, 70%, 80%, 90%, 95%, 96%, 97%, 98%, 99%, or 100% complementary) to a target site in the TCR DNA locus; and optionally c) a Cas component comprising a Cas protein or a nucleic acid encoding the Cas protein (e.g., Cas9), under a condition that allows introduction of the gRNA construct and the optional Cas component into the precursor T cell. In some embodiments, the first gRNA construct (e.g., DNA) encoding the first gRNA and the second gRNA construct (e.g., DNA) encoding the second gRNA are on 2 vectors. In some embodiments, the first gRNA construct (e.g., DNA) encoding the first gRNA and the second gRNA construct (e.g., DNA) encoding the second gRNA are on the same vector. In some embodiments, the precursor CAR-T cell already expresses a Cas protein before introducing the gRNA construct(s). In some embodiments, the precursor CAR-T cell does not express a Cas protein before introducing the gRNA construct(s) and the Cas component. In some embodiments, the method further comprises introducing a vector (e.g., viral vector such as lentiviral vector) carrying a nucleic acid encoding a Cas protein (e.g., Cas9), or a Cas (e.g., Cas9) mRNA, into the precursor T cell or the precursor T cell comprising said gRNA construct(s). In some embodiments, the nucleic acid encoding the Cas protein and the gRNA construct(s) encoding the gRNA are on different vectors. In some embodiments, the nucleic acid encoding the Cas protein and the gRNA construct(s) encoding the gRNA are on the same vector. In some embodiments, the Cas component and the gRNA construct(s) are introduced simultaneously. In some embodiments, the Cas component and the gRNA construct(s) are introduced sequentially. In some embodiments, the guide sequence targeting SPPL3 is encoded by a nucleic acid sequence comprising the sequence of SEQ ID NO: 1. In some embodiments, the precursor T cell is a CAR-T cell, i.e., already expresses a CAR before introducing the gRNA construct(s) and/or the Cas component. In some embodiments, the precursor T cell does not express a CAR before introducing the gRNA construct(s) and/or the Cas component. In some embodiments, the method further comprises introducing a vector (e.g., viral vector such as lentiviral vector) carrying a nucleic acid encoding a CAR, or an mRNA encoding a CAR, into the precursor T cell or the precursor T cell comprising said gRNA construct(s) and/or said Cas component. In some embodiments, the nucleic acid(s) encoding CAR, and the Cas component and/or the gRNA construct(s) are introduced simultaneously (e.g., can be on the same vector or different vectors). In some embodiments, the nucleic acid(s) encoding CAR, and the Cas component and/or the gRNA construct(s) are introduced sequentially.

In some embodiments, when a population of precursor immune cells (e.g., CAR-T or CAR-NK cell) are used for the production of modified immune cells described herein, the method also includes one or more isolation and/or enrichment steps, for example, isolating and/or enriching immune cells that comprise one or more mutations (e.g., inactivating mutations) in the target gene or target RNA, the gRNA construct or arRNA construct, the Cas component, and/or the engineered component (e.g., CAR), or immune cells that have reduced or no expression (RNA and/or protein expression) and/or function of the one or more target proteins (e.g., SPPL3 protein) and/or other proteins (e.g., TCR), from the population of immune cells contacted with any of the modifying agents described herein. Such isolation and/or enrichment steps can be performed using any known techniques in the art and described herein, such as FACS or magnetic-activated cell sorting (MACS), or based on target gene reporter (e.g., by expressing or not expressing a reporter of the target gene). Also see methods described in “optional enrichment step” subsection below.

In some embodiments, the method further comprises selecting from the population of modified immune cells those i) with reduced (e.g., reducing at least about any of 10%, 20%, 30%, 40%, 50%, 60%, 70%, 80%, 90%, 95%, or more) or no expression and/or function of the one or more target proteins (e.g., SPPL3 protein) and/or other proteins (e.g., TCR) described or identified herein; and/or ii) with no down-regulation or at most about 30% (e.g., at most about any of 25%, 20%, 10%, 5%, 1%, or less) down-regulation of an engineered receptor expressed in the modified immune cells.

In some embodiments, the engineered receptor (e.g., CAR), the gRNA construct or arRNA construct, and/or the Cas component, are introduced into the precursor immune cells by transducing/transfecting the nucleic acid (DNA or RNA) or vector encoding thereof (e.g., non-viral vector, or viral vector such as lentiviral vector), or a virus (e.g., lentivirus) comprising a nucleic acid encoding thereof. In some embodiments, the Cas component (e.g., Cas9 protein) is introduced into the precursor immune cells by inserting proteins into the cell membrane while passing cells through a microfluidic system, such as CELL SQUEEZE® (see, for example, U.S. Patent Application Publication No. 20140287509).

Methods of introducing vectors (e.g., viral vectors) or isolated nucleic acids into a mammalian cell are known in the art. The nucleic acids or vectors described herein can be transferred into an immune cell by physical, chemical, or biological methods.

Physical methods for introducing a vector (e.g., viral vector) into a cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001) Molecular Cloning: A Laboratory Manual, Cold Spring Harbor Laboratory, New York. In some embodiments, the vector (e.g., viral vector) is introduced into the cell by electroporation.

Biological methods for introducing a vector into a cell include the use of DNA and RNA vectors. Viral vectors have become the most widely used method for inserting genes into mammalian, e.g., human cells.

Chemical means for introducing a vector (e.g., viral vector) into a cell include colloidal dispersion systems, such as macromolecule complexes, nanocapsules, microspheres, beads, and lipid-based systems including oil-in-water emulsions, micelles, mixed micelles, and liposomes.

An exemplary colloidal system for use as a delivery vehicle in vitro is a liposome (e.g., an artificial membrane vesicle).

In some embodiments, RNA molecules (e.g., gRNA, arRNA, or mRNA encoding Cas) may be prepared by a conventional method (e.g., in vitro transcription) and then introduced into the immune cell via known methods such as mRNA electroporation. See, e.g., Rabinovich et al., Human Gene Therapy 17:1027-1035.

In some embodiments, viral vectors (lentiviral vector) or viruses (e.g., lentiviruses) comprising the nucleic acid encoding any of the engineered receptor (e.g., CAR), the gRNAs or arRNAs, and/or Cas protein described herein are contacted with the precursor immune cell at an MOI of at least about 1, such as at least about any of 1, 1.5, 2, 2.5, 3, 3.5, 4, 4.5, 5, 5.5, 6, 6.5, 7, 8, 9, or 10, such as at an MOI of about 3.

In some embodiments, the transduced/transfected immune cell is propagated ex vivo after introduction of the vector or isolated nucleic acid. In some embodiments, the transduced/transfected immune cell is cultured to propagate for at least about any of 1 day, 2 days, 3 days, 4 days, 5 days, 6 days, 7 days, 10 days, 12 days, or 14 days, such as 7 days. In some embodiments, the transduced/transfected immune cell is further evaluated or screened to select desired modified immune cells described herein.

Reporter genes may be used for identifying potentially transfected/transduced cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA/RNA has been introduced into the recipient cells. Suitable reporter genes may include genes encoding luciferase, beta-galactosidase, chloramphenicol acetyl transferase, secreted alkaline phosphatase, or the green fluorescent protein (GFP) gene (e.g., Ui-Tei et al. FEBS Letters 479: 79-82 (2000)). Suitable expression systems are well known and may be prepared using known techniques or obtained commercially. Antibiotic selection markers can also be used to identifying potentially transfected/transduced cells.

Other methods to confirm the presence of any of the nucleic acids described herein (e.g., gRNA construct or arRNA construct) or the presence of a mutation (e.g., inactivating mutation) in a target gene in a modified immune cell, include, for example, molecular biological assays well known to those of skill in the art, such as Southern and Northern blotting, RT-PCR, PCR, DNA-seq, or RNA-seq; biochemical assays, such as detecting the presence or absence of a particular peptide, e.g., by immunological methods (such as ELISAs and Western blots), Fluorescence-activated cell sorting (FACS), or Magnetic-activated cell sorting (MACS).

Also provided are modified immune cells generated by any of the immune cell generation/modification methods described herein. In some embodiments, the modified immune cell is autologous. In some embodiments, the modified immune cell is allogeneic.

### Source and Culture of Immune Cells

Immune cells described herein (e.g., T cells, NK cells, B cells) can be obtained from a number of sources, including peripheral blood mononuclear cells (PBMCs), bone marrow, lymph node tissue, cord blood, thymus tissue, tissue from a site of infection, ascites, pleural effusion, spleen tissue, and tumors. In some embodiments, any number of immune cell lines available in the art, may be used. In some embodiments, immune cells can be obtained from a unit of blood collected from a subject using any number of techniques known to the skilled artisan, such as FICOLL™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed, and the cells directly resuspended in culture media.

In some embodiments, the immune cell is provided from an umbilical cord blood bank, a peripheral blood bank, or derived from an induced pluripotent stem cell (iPSC), multipotent and pluripotent stem cell, or a human embryonic stem cell. In some embodiments, the immune cells are derived from cell lines. The immune cells in some embodiments are obtained from a xenogeneic source, for example, from mouse, rat, non-human primate, and pig. In some embodiments, the immune cells are human cells. In some aspects, the immune cells are primary cells, such as those isolated directly from a subject and/or isolated from a subject and frozen. In some embodiments, after blood collection, PBMCs are separated from the donor blood samples, then T cells, B cells, or NK cells are isolated from the PBMCs, e.g., using the immunomagnetic bead method. In some embodiments, the cells include one or more subsets of immune cells. The skilled artisan would recognize that multiple rounds of selection (e.g., positive or negative) can also be used. For example, for T cells, can be whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some cases, the immune cell is allogeneic in reference to one or more intended recipients. In some cases, the immune cell is suitable for transplantation, such as without inducing GvHD in the recipient. In some embodiments, the immune cell is an allogeneic CAR-T or CAR-NK cells. In some embodiments, the immune cell (e.g., allogeneic T cell) is modified to express an engineered receptor, such as CAR or engineered TCR. In some embodiments, the T cell (e.g., allogeneic T cell) is modified to knock-out endogenous TCR.

Among the sub-types and subpopulations of T cells and/or of CD4+ and/or of CD8+ T cells are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells (CTLs), mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

Immune cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. Many freezing solutions and parameters are known in the art and will be useful in this context. Any methods of controlled freezing may be used as well as uncontrolled freezing immediately at −20° C. or in liquid nitrogen.

In some embodiments, cryopreserved cells are thawed and washed as described herein and allowed to rest for one hour at room temperature prior to activation.

Also contemplated in the present application is the collection of blood samples or apheresis product from a subject at a time period prior to when the expanded cells as described herein might be needed. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the immune cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities.

In some embodiments, immune cells are obtained from a patient directly following treatment. In this regard, it has been observed that following certain cancer treatments, in particular treatments with drugs that damage the immune system, shortly after treatment during the period when patients would normally be recovering from the treatment, the quality of immune cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including immune cells, during this recovery phase. Further, in certain embodiments, mobilization, and conditioning regimens can be used to create a condition in a subject wherein repopulation, recirculation, regeneration, and/or expansion of particular cell types is favored, especially during a defined window of time following therapy.

### Activation and Expansion of Immune Cells

In some embodiments, the cells are incubated and/or cultured prior to or in connection with genetic engineering. The incubation steps can include culture, cultivation, stimulation, activation, and/or propagation or expansion. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

Whether prior to or after modification of the immune cells, the immune cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; 9,938,498; US20060121005; and Magee et al. (“Chapter Nine—Isolation, culture and propagation of natural killer cells,” Natural Killer Cells, Basic Science and Clinical Application, 2010, Pages 125-135).

Generally, T cells can be expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999). In some embodiments, paramagnetic beads can be used for expansion/activation, e.g., DYNABEADS® M-450 CD3/CD28 T paramagnetic beads.

NK cells express characteristic NK cell surface receptors, and lack both TCR rearrangement and T cell, B cell, monocyte and/or macrophage cell surface markers. Human NK cells are characterized by the presence of the cell-surface markers CD16 and CD56, and the absence of the T cell receptor (CD3). Human bone marrow-derived NK cells are further characterized by the CD2+CD16+CD56+CD3− phenotype, further containing the T-cell receptor zeta-chain [zeta(Q-TCR], and often characterized by NKp46, NKp30 or NKp44. Inhibitory NK cell receptors include HLA-E (CD94/NKG2A); HLA-C (group 1 or 2), KIR2DL; KIR3DL (HLA-B Bw4) and HLA-A3 or A4+ peptide. Activating NK cell receptors include HLA-E (CD94/NKG2C); KIR2DS (HLA-C) and KIR3DS (HLA-Bw4). Other receptors include the NK cell receptor protein-1 (termed NK1.1 in mice) and the low affinity receptor for the Fc portion of IgG (FcγRIII; CD16).

In some embodiments, the immune cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each immune cell (e.g., T cell) in the initial population to be expanded); and incubating the culture (e.g. for a time sufficient to expand the numbers of immune cells (e.g., T cell)). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of immune cells (e.g., T cell).

In some embodiments, the immune cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

Conditions appropriate for immune cell (e.g., T cell) culture include an appropriate media (e.g., Minimal Essential Media or RPMI Media 1640 or, X-vivo 15 (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan.

Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In some embodiments, the methods include assessing expression of one or more markers on the surface of the modified cells or cells to be engineered. In one embodiment, the methods include assessing surface expression of TCR or CD3ε, for example, by affinity-based detection methods such as by flow cytometry. In some aspects, where the method reveals surface expression of the antigen or other marker, the gene encoding the antigen or other marker is disrupted or expression otherwise repressed for example, using the methods described herein.

### Isolation and Enrichment of Immune Cells (e.g., Modified Immune Cells)

In some embodiments, the methods described herein further comprise isolating or enriching immune cells comprising a mutation (e.g., inactivating mutation) in a target gene or target RNA, and/or certain other traits (e.g., expressing CAR). In some embodiments, the methods described herein further comprise isolating or enriching immune cells comprising the Cas component, the gRNA construct or arRNA construct, and/or the engineered receptor (e.g., CAR) described herein, or has reduced or no expression (RNA and/or protein expression) and/or function of the one or more target proteins (e.g., SPPL3 protein) and/or other proteins described herein.

In some embodiments, the isolation methods include the separation of different cell types based on the absence or presence in the cell of one or more specific molecules, such as surface markers, e.g., surface proteins (e.g., CAR), intracellular markers, or nucleic acid (e.g., sgRNA, arRNA, and/or nucleic acid encoding Cas). In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. In some embodiments, the isolation comprises separation of cells and cell populations based on the cells' expression of selectable marker genes (e.g., antibiotic resistance genes such as puromycin, or fluorescent protein-encoding genes). Such separation steps can be based on positive selection, in which the cells having bound the reagents, resistant to antibiotics, or expressing fluorescent proteins are retained for further use, and/or negative selection, in which the cells having not bound to the antibody or binding partner or not expressing fluorescent proteins are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques. In some embodiments, T cells not expressing certain markers, e.g., exhaustion markers, are isolated. In some embodiments, T cells not expressing or expressing reduced markers, e.g., one or more of SPPL3, Fas, HLA-A, HLA-B, HLA-C, HLA-E, B7-H6, MICA, MICB, ULBP1, ULBP2, ULBP3, ULBP4, ULBP5, ULBP6, and ligands of NKp46, are isolated.

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some aspects, the sample or composition of cells to be separated is incubated with small, magnetizable or magnetically responsive material, such as magnetically responsive particles or microparticles, such as paramagnetic beads (e.g., such as Dynabeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some embodiments, the sample is placed in a magnetic field, and those cells having magnetically responsive or magnetizable particles attached thereto will be attracted to the magnet and separated from the unlabeled cells.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells, e.g., by using competing non-labeled antibodies, magnetizable particles or antibodies conjugated to cleavable linkers, etc.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1 (5):355-376.

### IV. METHODS OF TREATING DISEASES AND/OR SELECTING DONORS

The present invention in another aspect provides methods of treating a disease (e.g., cancer, or immune-related disease such as infection or immune cell exhaustion) in an individual (e.g., human), and methods of selecting or excluding an individual as a suitable donor of immune cells, based on any of the target genes or target proteins described herein (e.g., SPPL3 protein), or based on one or more target genes identified using any of the target gene identification methods described herein (e.g., AICD/FasL resistant genes).

An “aberration” at a gene (e.g., target gene) refers to a genetic and/or epigenetic aberration of a gene, an aberrant expression level, and/or an aberrant activity level, and/or an aberrant modification level of the gene (or gene product, such as RNA or protein) that may lead to abnormal loss of function or reduced function and/or abnormal expression (e.g., reduced or absent) of the RNA and/or protein encoded by the gene. In some embodiments, an aberration can occur at gene level, RNA level, and/or protein level. In some embodiments, a genetic aberration comprises a change to the nucleic acid (such as DNA or RNA) or protein sequence (i.e. mutation) or an aberrant epigenetic feature associated with the gene, including, but not limited to, coding, non-coding, regulatory, enhancer, silencer, promoter, intron, exon, and untranslated regions of the gene. In some embodiments, an aberration at a gene comprises a mutation of the gene, includes, but not limited to, deletion, frameshift, insertion, indel, missense mutation, nonsense mutation, point mutation, silent mutation, splice site mutation, splice variant, and translocation. In some embodiments, the mutation may be a loss or deletion of the gene. In some embodiments, the mutation is a deleterious mutation. In some embodiments, an aberration at a gene comprises aberrant (e.g., reduced or absent) expression (e.g., mRNA or protein) of a gene compared to a control level. In some embodiments, an aberration at a gene comprises aberrant (e.g., reduced or abolished) activity of a gene product (e.g., RNA or protein) compared to a control level, such as activation or inhibition of downstream targets. In some embodiments, an aberration at a gene comprises aberrant modification (e.g., increased, decreased, or mis-modification) of a gene (e.g., at DNA level or histone level) or gene product (e.g., RNA or protein) compared to a control level, such as post-translational modification (e.g., phosphorylation, ubiquitination). In some embodiments, an aberration at a gene comprises a copy number variation of the gene. In some embodiments, the copy number variation of the gene is caused by structural rearrangement of the genome, including deletions, duplications, inversion, and translocations. In some embodiments, an aberration at a gene comprises an aberrant epigenetic feature of the gene, including, but not limited to, DNA methylation, hydroxymethylation, increased or decreased histone binding, histone methylation, histone acetylation, chromatin remodeling, and the like. In some embodiments, the aberration is determined in comparison to a control or reference, such as a reference sequence (such as a nucleic acid sequence or a protein sequence), a control expression (such as RNA or protein expression) level, a control activity (such as activation or inhibition of downstream targets) level, or a control modification (e.g., post-translational modification or epigenetic modification) level.

In some embodiments, the aberrant expression level or the aberrant activity level in a gene may be below the control level (such as about any of 10%, 20%, 30%, 40%, 60%, 70%, 80%, 90% or more below the control level). In some embodiments, the aberrant modification level in a gene (e.g., modification of DNA, nucleosome, RNA, or protein) may be below the control level (such as about any of 10%, 20%, 30%, 40%, 60%, 70%, 80%, 90% or more below the control level), or above the control level (such as about any of 10%, 20%, 30%, 40%, 60%, 70%, 80%, 90% or more above the control level). In some embodiments, the aberrant modification in a gene is a mis-modification, e.g., ubiquitination instead of phosphorylation. In some embodiments, the control level (e.g. expression level or activity level or modification level) is the average or median level (e.g. expression level or activity level or modification level) of a control population. In some embodiments, the control population is a healthy population that does not have a disease (e.g., a disease to be treated), and optionally with comparable demographic characteristics (e.g. gender, age, ethnicity, etc.) as the individual being/to be treated. In some embodiments, the control level (e.g. expression level or activity level or modification level) is a level (e.g. expression level or activity level or modification level) or average level of a healthy tissue from the same individual. In some embodiments, the control level (e.g. expression level or activity level or modification level) is a level (e.g. expression level or activity level or modification level) or average level of a same cell (e.g., from the same individual) without said aberration (e.g., target gene mutation). In some embodiments, the control or reference level is the average or median expression (RNA and/or protein expression) and/or function of the one or more target proteins (e.g., SPPL3 protein) and/or other proteins in a population of individuals.

An aberration at a gene may be determined by comparing to a reference sequence, including epigenetic patterns of the reference sequence in a control sample. In some embodiments, the reference sequence is the sequence (DNA, RNA or protein sequence) corresponding to a fully functional allele of the corresponding gene, such as an allele (e.g. the prevalent allele) of the corresponding gene present in an unmodified immune cell, e.g., precursor immune cell.

An aberration at a target gene is herein also referred to as “target gene aberration,” including but not limited to target gene mutation. An aberration at a AICD resistant gene, or FasL resistant gene, or AICD/FasL resistant gene (used interchangeably herein), is herein also referred to as “AICD resistant aberration,” “FasL resistant aberration,” or “AICD/FasL resistant aberration”, including but not limited to AICD or FasL resistant mutation, which increases resistance to AICD and/or FasL-induced death in an immune cell. In some embodiments, AICD/FasL resistant gene is selected from the group consisting of SPPL3, FADD, FAS, CASP8, ARID1A, BAK1, BID, ETS1, IKZF2, and HIST1H1B.

The “status” of an aberration at a gene may refer to the presence or absence of the aberration at the gene, or the aberrant level (expression or activity or modification level) of the gene. In some embodiments, the presence of an aberration (such as a LOF mutation) in one or more AICD/FasL resistant genes (or RNAs/proteins encoded thereof) as compared to a control, indicates that i) such immune cells may have higher resistance to AICD, can induce no or less host-versus-graft (HvG) responses (e.g., attached by host T cells and/or NK cells), and/or have better in vivo persistence, thus are ideal for use in cell-based immunotherapy (e.g., CAR-T or CAR-NK); and/or ii) such individuals may be suitable for autologous or allogeneic immune cell therapy or for donating immune cells for immune cell therapy. In some embodiments, the absence of an aberration (such as a mutation) in one or more AICD/FasL resistant genes (or RNAs/proteins encoded thereof) compared to a control, indicates that i) such immune cells may have lower resistance to AICD, may induce strong HvG responses, and/or may have poor in vivo persistence, thus less ideal for use in cell-based immunotherapy; and/or ii) the individual is less suitable for autologous or allogeneic immune cell therapy, or the individual is not an ideal donor for immune cells. In some embodiments, an aberrant level (such as expression level or activity level or modification level) of one or more AICD/FasL resistant genes is correlated with i) the resistance degree to AICD and/or HvG responses, and/or in vivo persistence degree; and/or ii) the likelihood of the individual to be an ideal immune cell donor. For example, a larger deviation of the level (e.g. expression or activity or modification level) of one or more AICD/FasL resistant genes in the direction of reducing or abolishing the gene expression and/or function indicates that the immune cell has higher resistance to AICD, induces less HvG response, and/or has longer in vivo persistence; and/or ii) the individual is more likely to respond to autologous or allogeneic immune cell therapy, or is more suitable as an immune cell donor.

In some embodiments, there is provided a method of treating a disease (e.g., cancer, or immune-related disease such as infection or immune cell exhaustion) in an individual (e.g., human), comprising administering to the individual an effective amount of any of the modified immune cells described herein (e.g., SPPL3-KO CAR-T or CAR-NK cell) or pharmaceutical compositions thereof. In some embodiments, there is provided a method of treating a disease (e.g., cancer, or immune-related disease such as infection or immune cell exhaustion) in an individual (e.g., human), comprising administering to the individual an effective amount of an immune cell (or pharmaceutical compositions thereof) modified to reduce (e.g., reducing at least about any of 10%, 20%, 30%, 40%, 60%, 70%, 80%, 90%, 95% or more) or abolish expression (RNA and/or protein expression) and/or function of one or more target proteins identified herein (e.g., AICD/FasL resistant gene-encoded proteins) or described herein (e.g., SPPL3 protein), such as SPPL3, Fas, FADD, CASP8, ARID1A, BAK1, BID, ETS1, IKZF2, and/or HIST1H1B.

In some embodiments, the modified immune cell is allogeneic. In some embodiments, the modified immune cell is autologous. In some embodiments, the modified immune cell further expresses an engineered receptor (e.g., CAR, engineered TCR, or TAC). In some embodiments, the disease is associated with the expression of a target antigen (e.g., tumor antigen), and wherein the immune cell expresses or is modified to express an engineered receptor (e.g., CAR) specifically recognizing the target antigen. Use of the modified immune cells or pharmaceutical compositions thereof for treating a disease in an individual (e.g., human) is also provided. Use of the modified immune cells or pharmaceutical compositions thereof in the manufacture of a medicament for treating a disease in an individual (e.g., human) is also provided. In some embodiments, the disease is a cancer, an infection, an inflammation, an autoimmune disease, or an immune-related disease characterized by effector cell exhaustion.

In some embodiments, is provided a method of treating a cancer (e.g., CD19+ cancer) in an individual (e.g., human), comprising administering to the individual an effective amount of SPPL3^{KO}/TCR^{KO} CAR-T (e.g., SPPL3^{KO}/TCR^{KO}/anti-CD19 CAR-T) cells or pharmaceutical compositions thereof. In some embodiments, is provided a method of treating a cancer (e.g., CD19+ cancer) in an individual (e.g., human), comprising administering to the individual an effective amount of SPPL3^{KO} CAR-T (e.g., SPPL3^{KO} anti-CD19 CAR-T) cells or pharmaceutical compositions thereof. In some embodiments, the cancer is a B cell cancer, including but not limited to, acute myeloid leukemia (AML), chronic myelogenous leukemia (CML), myelodysplastic syndrome (MDS), acute B lyinphoblastic leukemia (B-ALL), diffuse large B cell lymphoma (DLBCL), follicular lymphoma, chronic lymphocytic leukemia (CLL), hairy cell leukemia (HCL), blastic plasmacytoid dendritic cell neoplasm (BPDCN), non-Hodgkin's lymphomas (NHL), Hodgkin's lymphoma, systemic mastocytosis, and Burkitt's lymphoma.

In some embodiments, there is provided a method of identifying an individual (e.g., human) as a suitable donor of an immune cell with prolonged in vivo persistence, comprising examining the expression and/or function of one or more target proteins identified herein (e.g., AICD/FasL resistant gene-encoded proteins), such as selected from the group consisting of SPPL3, FADD, FAS, CASP8, ARID1A, BAK1, BID, ETS1, IKZF2, and HIST1H1B, in the individual (e.g., in an immune cell sample obtained from the individual), wherein the identification of reduced (e.g., reducing at least about any of 10%, 20%, 30%, 40%, 60%, 70%, 80%, 90%, 95% or more) or abolished expression and/or function of the one or more target proteins compared to a reference (e.g., an immune cell sample obtained from the reference) identifies the individual as the suitable donor. In some embodiments, the reference is the average or median expression and/or function of the one or more target proteins in a population of individuals (e.g., heathy individuals, and/or patients). In some embodiments, there is provided a method of identifying an individual (e.g., human) as a suitable donor of an immune cell with prolonged in vivo persistence, comprising examining the expression and/or function of SPPL3 protein in the individual (e.g., in an immune cell sample obtained from the individual), wherein the identification of reduced or abolished expression and/or function of the SPPL3 protein compared to a reference identifies the individual as the suitable donor. In some embodiments, the reference is the average or median expression and/or function of the SPPL3 protein in a population of individuals. In some embodiments, the method further comprises examining the expression and/or function (e.g., in an immune cell sample obtained from the individual) of one or more other proteins selected from the group consisting of TCRα, TCRβ, TCRγ, TCRδ, HLA-A, HLA-B, HLA-C, HLA-E, HLA-F, HLA-G, B2M, PD-1, TIM-3, LAG-3, CTLA-4, CISH, Fas, FADD, CASP8, ARID1A, BAK1, BID, ETS1, IKZF2, HIST1H1B, B7-H6, MICA, MICB, ULBP1, ULBP2, ULBP3, ULBP4, ULBP5, ULBP6, and ligands of NKp46 (e.g., selected from the group consisting of Fas, FADD, CASP8, ARID1A, BAK1, BID, ETS1, IKZF2, and HIST1H1B), wherein the identification of reduced (e.g., reducing at least about any of 10%, 20%, 30%, 40%, 60%, 70%, 80%, 90%, 95% or more) or abolished expression and/or function of one or more of the other proteins further identifies the individual as the suitable donor. In some embodiments, examining the expression and/or function of the one or more target proteins (e.g., SPPL3 protein) and/or the one or more other proteins comprises examining the sequence of the nucleic acid (DNA or RNA) encoding the one or more target proteins and/or the one or more other proteins, wherein the identification of an aberration (e.g., a mutation such as LOF mutation) in the nucleic acid that reduces expression and/or function of the one or more target proteins and/or the one or more other proteins identifies the individual as the suitable donor. In some embodiments, identification of an aberration (e.g., a mutation such as LOF mutation) in the nucleic acid that reduces expression and/or function of the one or more target proteins identifies the individual as the suitable donor. In some embodiments, there is provided a method of identifying an individual (e.g., human) as a suitable donor of an immune cell with prolonged in vivo persistence, comprising detecting in an immune cell sample from the individual one or more AICD/FasL resistant aberrations (e.g., AICD/FasL resistant mutations such as LOF mutations) in one or a plurality of AICD/FasL resistant genes identified using any of the target gene identification methods described herein (such as selected from the group consisting of SPPL3, FADD, FAS, CASP8, ARID1A, BAK1, BID, ETS1, IKZF2, and HIST1H1B gene), wherein the presence of the one or more AICD/FasL resistant aberrations (e.g., AICD/FasL resistant mutations, such as SPPL3 LOF mutation or KO) in the immune cell sample identifies the individual as the suitable donor. In some embodiments, the method further comprises detecting in the immune cell sample an aberration (e.g., mutation such as LOF mutation) in one or more other genes selected from the group consisting of Fas, FADD, CASP8, ARID1A, BAK1, BID, ETS1, IKZF2, HIST1H1B, PD-1, TIM-3, LAG-3, CTLA-4, and CISH, wherein the presence of the aberration in the immune cell sample further identifies the individual as the suitable donor.

In some embodiments, there is provided a method of excluding an individual (e.g., human) as a suitable donor of an immune cell with prolonged in vivo persistence, comprising examining the expression and/or function of one or more target proteins identified herein (e.g., AICD/FasL resistant gene-encoded proteins), such as selected from the group consisting of SPPL3, FADD, FAS, CASP8, ARID1A, BAK1, BID, ETS1, IKZF2, and HIST1H1B, in the individual (e.g., in an immune cell sample obtained from the individual), wherein the individual is excluded as the suitable donor if no reduced (e.g., reducing at least about any of 10%, 20%, 30%, 40%, 60%, 70%, 80%, 90%, 95% or more) or abolished expression and/or function of the one or more target proteins compared to a reference (e.g., an immune cell sample obtained from the reference) is identified. In some embodiments, the reference is the average or median expression and/or function of the one or more target proteins in a population of individuals (e.g., heathy individuals, and/or patients). In some embodiments, there is provided a method of excluding an individual (e.g., human) as a suitable donor of an immune cell with prolonged in vivo persistence, comprising examining the expression and/or function of SPPL3 protein in the individual (e.g., in an immune cell sample obtained from the individual), wherein the individual is excluded as the suitable donor if no reduced or abolished expression and/or function of the SPPL3 protein compared to a reference is identified. In some embodiments, the reference is the average or median expression and/or function of the SPPL3 protein in a population of individuals. In some embodiments, examining the expression and/or function of the one or more target proteins (e.g., SPPL3 protein) comprises examining the sequence of the nucleic acid (DNA or RNA) encoding the one or more target proteins, wherein the individual is excluded as the suitable donor if no aberration (e.g., mutation such as LOF mutation) in the nucleic acid that reduces expression and/or function of the one or more target proteins is identified. In some embodiments, there is provided a method of excluding an individual (e.g., human) as a suitable donor of an immune cell with prolonged in vivo persistence, comprising detecting in an immune cell sample from the individual one or more AICD/FasL resistant aberrations (e.g., AICD/FasL resistant mutations such as LOF mutations) in one or a plurality of AICD/FasL resistant genes identified using any of the target gene identification methods described herein (such as selected from the group consisting of SPPL3, FADD, FAS, CASP8, ARID1A, BAK1, BID, ETS1, IKZF2, and HIST1H1B gene), wherein the individual is excluded if no AICD/FasL resistant aberration (e.g., AICD/FasL resistant mutation, such as SPPL3 LOF mutation or KO) is identified in the immune cell sample.

In some embodiments, the method of treating a disease, or selecting or excluding an individual as immune cell donor, further comprises detecting the one or more AICD/FasL resistant aberrations (e.g., AICD/FasL resistant mutations), or expression and/or function of one or more target proteins identified herein or described herein (e.g., SPPL3 protein), in an immune cell sample from the individual (e.g., by NGS). In some embodiments, when the detection is on gene level (e.g., examination of mutation), the sample can be from any tissue of the individual. In some embodiments, the method further comprises identifying the one or more AICD/FasL resistant genes. In some embodiments, the method further comprises detecting aberrant (e.g., reduced or absent) expression (e.g., RNA or protein) of the one or more AICD/FasL resistant genes or expression products thereof (e.g., SPPL3) compared to a control/reference level, such as by qPCR, RNA-seq, mass spectrometry, western blot, or any other RNA or protein expression level detection methods. In some embodiments, the method further comprises detecting aberrant modification at the one or more AICD/FasL resistant genes or expression products thereof (e.g., SPPL3) compared to a control/reference level, such as epigenetic modification (e.g., DNA methylation, histone methylation, histone acetylation) or post-translational modification (e.g., phosphorylation, glycosylation, ubiquitination, nitrosylation, methylation, acetylation, lipidation and proteolysis). Any known methods for detecting modification(s) on DNA, nucleosome, RNA, or protein can be used herein, such as ChIP-seq, ChIP-qPCR, DNase-seq, MNase-seq, mass spectrometry, western blot, etc. In some embodiments, the method further comprises detecting aberrant (e.g., reduced or absent) activity of expression product (e.g., RNA or protein) of the one or more AICD/FasL resistant genes (e.g., SPPL3) compared to a control/reference level. Any suitable gene/protein function/activity testing methods can be used herein, such as detecting signal transduction, activation status (e.g., phosphorylation status) of downstream pathway molecules, glycosylation status of interaction partner(s), protein-protein binding affinity and/or specificity, metabolism, cell behavior (e.g., cell proliferation, differentiation, activation, persistence, death, cell cycle), effector function (e.g., cytokine release or cytotoxicity), etc. In some embodiments, the reference is the average or median mutation frequency, expression and/or function of the corresponding gene/protein in a population of individuals (e.g., heathy individuals, and/or patients).

In some embodiments, there is provided a method of selecting an immune cell for immune cell therapy, comprising isolating and/or enriching from an initial population of immune cells (e.g., precursor immune cells, CAR-T cells, or CAR-NK cells) an immune cell with reduced (e.g., reducing at least about 10%, 20%, 30%, 40%, 50%, 60%, 70%, 80%, 90%, 95%, or more) or no expression and/or function of one or more target proteins identified herein (e.g., AICD/FasL resistant gene-encoded protein) or described herein (e.g., SPPL3, FADD, FAS, CASP8, ARID1A, BAK1, BID, ETS1, IKZF2, or HIST1H1B protein; such as SPPL3 protein). In some embodiments, there is provided a method of selecting an immune cell for immune cell therapy, comprising isolating and/or enriching from an initial population of immune cells (e.g., precursor immune cells, CAR-T cells, or CAR-NK cells) an immune cell with reduced (e.g., reducing at least about 10%, 20%, 30%, 40%, 50%, 60%, 70%, 80%, 90%, 95%, or more) or no expression and/or function of SPPL3 protein, or has a mutation (e.g., LOF mutation) in SPPL3 gene that reduces (e.g., reducing at least about 10%, 20%, 30%, 40%, 50%, 60%, 70%, 80%, 90%, 95%, or more) or abolishes its expression (RNA and/or protein) and/or function. In some embodiments, the initial population of immune cells express an engineered receptor (e.g., CAR, TAC, engineered TCR). In some embodiments, the method further comprises introducing into the isolated/enriched immune cells a nucleic acid encoding an engineered receptor (e.g., CAR, TAC, engineered TCR). In some embodiments, the method further comprises isolating and/or enriching from the immune cells (e.g., target gene/protein-modified immune cells, or initial population of immune cells) those expressing the engineered receptor. Any cell isolation/enrichment method can be used herein, such as FACS, based on reporter expressed by the modified immune cells. Also see “isolation and enrichment of immune cells (e.g., modified immune cells)” subsection above for suitable methods. In some embodiments, the immune cells are autologous. In some embodiments, the immune cells are allogeneic.

### Diseases and Conditions

Any diseases that can be treated with an immune cell-based therapy (e.g., CAR-T or CAR-NK therapy) are be treated using any of the methods and/or modified immune cells described herein. In some embodiments, the disease is selected from the group consisting of a cancer, an infection, an inflammation, an autoimmune disease, and an immune-related disease characterized by immune cell (e.g., effector cell) exhaustion.

The methods described herein are suitable for treating a variety of cancer, including both solid cancer and hematologic cancer, as well as cancers of all stages, including early stage cancer, non-metastatic cancer, primary cancer, advanced cancer, locally advanced cancer, metastatic cancer, or cancer in remission. In some embodiments, the solid or hematologic cancer can be of any of stages I, II, III, and IV, according to the American Joint Committee on Cancer (AJCC) staging groups.

In some embodiments, the cancer is a solid cancer selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, breast cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), cutaneous T-cell lymphoma (CTCL), cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers.

In some embodiments, the cancer is a hematologic cancer chosen from one or more of acute myeloid leukemia (AML), chronic lymphocytic leukemia (CLL), acute leukemia, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm (BPDCN), Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

In some embodiments, the disease is an infection or infectious disease, such as caused by a pathogen. In some embodiments, the pathogen is any of a virus, a bacterium, a fungus, and a parasite, or a fragment thereof. In some embodiments, the pathogen comprises a pathogen-associated molecule pattern (PAMP). In other embodiments, the PAMP is selected from the group consisting of a pathogen fragment, a pathogen debris, a pathogen nucleic acid, a pathogen lipoprotein, a pathogen surface glycoprotein, a pathogen membrane component, and a component released from the pathogen. In some embodiments, the component released from the pathogen comprises a toxin. In other embodiments, the toxin is selected from the group consisting of endotoxin, lipopolysaccharide (LPS), lipoteichoic acid (LTA), wall teichoic acid (WTA) and Ricin.

In some embodiments, the bacterium is selected from the group consisting of Acinetobacter baumanii, Burkholderia cepacia, Bacterioides fragilis, Chlamydia trachomatis, Citrobacter freundii, Campylobacter jejuni, Escherichia coli, Enterobacter aerogenes, Enterobacter cloacae, Haemophilus inf b, Helicobacter pylori, Klebsiella oxytoca, K. pneumonia (MDR/CRE), Legionella pneumophila, Neisseria meningitides, Neisseria gonorrhoeae, Pseudomonas aeruginosa, Salmonella typhi, paratyphi, typhimurium, Serratia marcescens, Shigella flexneri, Stenotrophomonas maltophilia, Yersinia pseudotuberculosis, Bacillus subtilis, Clostridium neoformans, C. difficile, C. perfringens, Corynebacterium spp, Enterococcus faecalis, Enterococcus faecium, vancomycin-resistant Enterococci (VRE), Listeria monocytogenes, Mycobactrium avium, M. tuberculosis, M. leprae, Nocardia farcinica, P. acnes, Staphylococcus aureus, methicillin-susceptible Staphylococcus aureus (MSSA), methicillin-resistant Staphylococcus aureus (MRSA), Staphylococcus epidermidis, Streptococcus pyogenes, Strep Group A, Strep Group B (agalactiae) and Strep Group C. In some embodiments, the bacterium is an antibiotic-resistant bacterium. In some embodiment, the bacterium is a multi-drug resistant bacterium.

In some embodiments, the fungus is selected from the group consisting of Aspergillus spp, Blastomyces, Candida albicans, glabrata, guilliermondii, krusei, parapsilosis, tropicalis Cryptococcus, Fusarium spp., Mucor spp., Saccharomyces, and Pneumocystis jirovecii (carinii).

In some embodiments, the virus is from any one of the Orthomyxoviridae, Filoviridae, Flaviviridae, Coronaviridae, and Poxviridae families. In some embodiments, the virus is selected from the group consisting of Dengue virus, Ebola virus, EBV, Hepatitis A virus, Hepatitis B virus, Hepatitis C virus, Hepatitis D virus, HIV, HSV 1, HSV 2, Cytomegalovirus (CMV), Influenza A virus (e.g., H1N1, H5N1), Influenza B virus, Influenza C virus, Marburg virus, Human respiratory syncytial virus (RSV), SARS-CoV, MERS-CoV, SARS-CoV-2, West Nile virus, Human papillomavirus (HPV), Human rhinoviruses (HRVs), and Zica virus.

In some embodiments, the parasite is selected from the group consisting of Cryptosporidium, Leishmania, Malaria, Schistosoma, Trichomonasm and Trypanosoma.

In some embodiments, the disease is an inflammatory disease. The term of “inflammatory disease,” “inflammatory disorder” or “inflammatory condition” refers to any disease marked by inflammation, which may be caused by a multitude of inciting events, including radiant, mechanical, chemical, infections, and immunological stimuli. Inflammatory conditions can be identified via clinical and pathological features, and/or expression of well-known inflammatory-related molecules secreted by tissues and/or cells. Inflammatory diseases can refer to any diseases in which inflammation conditions present. Non-limiting examples of inflammatory diseases treatable with the present method, modified immune cells, medicament or pharmaceutical composition includes, a cardiovascular disease, arthritis, asthma, psoriasis, an inflammatory bowel disease, organ transplant rejection, lupus, an autoimmune disorder, a radiation-induced injury, cancer, a burn, trauma, a rheumatic disorder, a renal disease, an allergic disease, an infectious disease, an ocular disease, a skin disease, a gastrointestinal disease, a hepatic disease, cerebral edema, sarcoidosis, thrombocytopenia, and a spinal cord injury.

In some embodiments, the inflammatory disease is an autoimmune disease. Autoimmune disease, or autoimmunity, is the failure of an organism to recognize its own constituent parts (down to the sub-molecular levels) as “self,” which results in an immune response against its own cells and tissues. Any disease that results from such an aberrant immune response is termed an autoimmune disease. Prominent examples include Coeliac disease, diabetes mellitus type 1 (IDDM), systemic lupus erythematosus (SLE), Sjögren's syndrome, multiple sclerosis (MS), Hashimoto's thyroiditis, Graves' disease, idiopathic thrombocytopenic purpura, and rheumatoid arthritis (RA). Examples of autoimmune diseases include, but are not limited to, acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcalnephritis, erythema nodosurn, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitisubiterans. Sjogren's syndrome, primary biliary cirrhosis, Hashimoto's thyroiditis, thyrotoxicosis, scleroderma, chronic active hepatitis, polymyositis/dermatomyositis, polychondritis, pamphigus vulgaris, Wegener's granulomatosis, membranous nephropathy, amyotrophic lateral sclerosis, tabes dorsalis, giant cell arteritis/polymyalgia, perniciousanemia, rapidly progressive glomerulonephritis, psoriasis, and fibrosing alveolitis. The most common treatments are corticosteroids and cytotoxic drugs, which can be very toxic.

In some embodiments, an immune-related disease is a disease caused by excessive activation and/or proliferation of various immune cells, for example, effector T cells or cytotoxic T cells and inflammatory cells, and may include, but is not limited to, autoimmune diseases; graft versus host disease; organ transplant rejection; asthma; atopy; or acute or chronic inflammatory diseases. In some embodiments, the immune-related disease can be prevented, ameliorated, or treated by interfering with intracellular signaling pathways essential for activation and/or proliferation of immune cells (e.g., T cells).

In some embodiments, an immune-related disease is associated with or characterized by an immune cell (e.g., T cell) dysfunctional disorder. In certain embodiments, immune cell dysfunctional disorder is characterized by immune cell exhaustion (e.g., T cell exhaustion or NK cell exhaustion). In certain embodiments, the immune-related disease is selected from the group consisting of unresolved acute infection, chronic infection, and tumor immunity.

As used herein, “tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage, and tumor clearance.

During resolution of an acute inflammatory response, a subset of activated effector T cells differentiate into long-lived memory cells. By contrast, in patients with chronic infections or cancer, T cells may undergo pathologic differentiation toward a state of dysfunction, which has been termed T cell exhaustion. The root cause of T cell exhaustion is persistent antigen exposure leading to continuous TCR signaling. “T cell exhaustion” refers to decrease of T cell function, which may occur as a result of an infection (e.g., a chronic infection) or a disease. T cell exhaustion is associated with increased expression of PD-1, TIM-3, and LAG-3, apoptosis, and reduced cytokine secretion. Accordingly, the terms “ameliorate T cell exhaustion,” “inhibit T cell exhaustion,” “reduce T cell exhaustion” and the like refer to a condition of restored functionality of T cells characterized by one or more of the following: decreased expression and/or level of one or more of inhibitory immune checkpoint molecules such as PD-1, TIM-3, and LAG-3; increased memory cell formation and/or maintenance of memory markers (e.g., CD62L); prevention of apoptosis; increased antigen-induced cytokine (e.g., IL-2) production and/or secretion; enhanced cytotoxicity/killing capacity; increased recognition of tumor targets with low surface antigen; enhanced proliferation in response to antigen.

Immune cell exhaustion (e.g., T cell exhaustion) can be characterized by marked changes in metabolic function, transcriptional programming, apoptosis, loss of effector function (e.g., cytokine secretion, killing capacity), and co-expression of multiple surface inhibitory receptors. Immune cell exhaustion (e.g., T cell exhaustion) can be associated with a reduced ability of the immune system to control tumor growth and to control chronic infections. In some embodiments, exhaustion of NK cells has one or more of the following characteristics: reduced or abolished IFN-7 and/or GM-CSF expression, reduced or abolished cytotoxic or cytolytic activity (e.g., reduced expression of cytolytic molecules such as granzymes, perforin, FasL, and TRAIL), and reduced or abolished expression of surface activating receptors such as NKG2D, CD16, NCRs, CD226 (DNAM-1), and 2B4. Also see Bi and Tian, (“NK Cell Exhaustion,” Front Immunol. 2017; 8: 760). In some embodiments, exhaustion of B cells is associated with reduced or loss of CD21, CD95, and/or CD27 expression. Accordingly, the terms “ameliorate immune cell exhaustion,” “inhibit immune cell exhaustion,” “reduce immune cell exhaustion” and the like refer to a condition of restored functionality of immune cells characterized by one or more of the following: decreased expression and/or level of one or more of inhibitory immune checkpoint molecules; increased memory cell formation and/or maintenance of memory markers; prevention of apoptosis; increased antigen-induced cytokine production and/or secretion; enhanced cytotoxicity/killing capacity; increased recognition of tumor targets with low surface antigen; enhanced proliferation in response to antigen; and reduced exhaustion marker(s).

In the case of cancer, the treatment methods, modified immune cells, or pharmaceutical compositions described herein can achieve one or more of the following: (i) reduce the number of cancer cells; (ii) reduce (e.g., reducing at least about 10%) tumor size; (iii) inhibit, retard, slow to some extent (e.g., at least about 10%) and preferably stop cancer cell infiltration into peripheral organs; (iv) inhibit (i.e., slow to some extent (e.g., at least about 10%) and preferably stop) tumor metastasis; (v) inhibit (e.g., inhibiting at least about 10%) tumor growth; (vi) prevent or delay occurrence and/or recurrence of tumor; (vii) relieve to some extent one or more of the symptoms associated with the cancer; (viii) prolong (e.g., prolonging at least about 10%) patient survival; and/or (ix) reduce (e.g., reducing at least about 10%) CRS.

In the case of infectious disease, such as viral infection, the treatment methods, modified immune cells, or pharmaceutical compositions described herein can achieve one or more of the following: i) reduce (e.g., reducing at least about 10%) the number of cells infected by the pathogen; ii) reduce (e.g., reducing at least about 10%) the production or release of pathogen-derived antigens; iii) inhibit (i.e., slow to some extent (e.g., at least about 10%) and preferably stop) spread of the pathogen to uninfected cells; iv) relieve to some extent one or more symptoms associated with the infection; v) prolong (e.g., prolonging at least about 10%) patient survival; and/or (vi) reduce (e.g., reducing at least about 10%) CRS.

In the case of immune-related diseases associated with excessive immune response (e.g., inflammation, autoimmune disease), the treatment methods, modified immune cells, or pharmaceutical compositions described herein can achieve one or more of the following: i) controlling, ameliorating (e.g., reducing at least about 10%), and/or preventing the infiltration of inflammatory cells (e.g., NK cells, cytotoxic T cells); ii) controlling, ameliorating (e.g., reducing at least about 10%), and/or preventing tissue and/or organ injury and/or failure; iii) controlling, ameliorating (e.g., reducing at least about 10%), and/or preventing inflammation, CRS, sepsis, systemic inflammatory response syndrome (SIRS), septic shock, and/or multiple organ dysfunction syndrome (MODS); iv) controlling, ameliorating (e.g., reducing at least about 10%), and/or preventing cell necrosis; v) reducing (e.g., reducing at least about 10%) inflammatory markers such as IL-6, IL-8, IL-10, IL1B, IL-12, IL-15, IL-17, CCL2, IL-1α, IL-2, IL-5, IL-9, CCL4, M-CSF, MCP-1, GCSF, MIP1A, CRP, TNFα, TNFβ, IFNγ, IP10, MCP1, SAA-1; and/or vi) prolong (e.g., prolonging at least about 10%) patient survival.

In the case of immune-related diseases associated with reduced/abolished immune response (e.g., immune cell exhaustion), the treatment methods, modified immune cells, or pharmaceutical compositions described herein can modulate the immune response of a subject, such as inducing, activating, promoting, increasing, enhancing, or prolonging (e.g., at least about 10%) an immune response in the subject. For example, proliferation/activation/persistence of the modified immune cells, cytokine release, and/or cytolytic or cytotoxic activity are enhanced.

In some embodiments, the treatment methods, modified immune cells, or pharmaceutical compositions described herein can i) prolong (e.g., prolonging at least about any of 10%, 20%, 30%, 40%, 50%, 60%, 70%, 80%, 90%, 95%, 1-fold, 2-fold, 5-fold, 10-fold, 20-fold, 50-fold, 100-fold, or more) in vivo persistence, ii) reduce (e.g., reducing at least about any of 10%, 20%, 30%, 40%, 50%, 60%, 70%, 80%, 90%, 95%, or 100%) AICD, or increases (e.g., increasing at least about any of 10%, 20%, 30%, 40%, 50%, 60%, 70%, 80%, 90%, 95%, 1-fold, 2-fold, 5-fold, 10-fold, 20-fold, 50-fold, 100-fold, or more) resistance to AICD, iii) reduces (e.g., reducing at least about any of 10%, 20%, 30%, 40%, 50%, 60%, 70%, 80%, 90%, 95%, or 100%) host-versus-graft (HvG) response, such as reducing at least about 10% (e.g., at least about any of 20%, 30%, 40%, 50%, 60%, 70%, 80%, 90%, 95%, or 100%) killing by an allogeneic T cell, or reducing at least about 10% (e.g., at least about any of 20%, 30%, 40%, 50%, 60%, 70%, 80%, 90%, 95%, or 100%) killing by an autologous or allogeneic NK cell, and/or iv) do not down-regulate or eliminate expression and/or function (e.g., mediating cytotoxicity) of engineered receptors (e.g., CAR), or have at most about 30% (e.g., at most about any of 25%, 20%, 10%, 5%, 3% 1%, or less) down-regulated expression and/or function of the engineered receptors, of an immune cell (e.g., CAR-T or CAR-NK cell).

Administration of the modified immune cells or pharmaceutical compositions thereof may be carried out in any convenient manner, including by injection, transfusion, implantation or transplantation. The modified immune cells or pharmaceutical compositions thereof may be administered to a patient transarterially, subcutaneously, intradermally, intratumorally, intranodally, intramedullary, intramuscularly, intravenously, or intraperitoneally. In some embodiments, the modified immune cells or pharmaceutical compositions are administered systemically. In some embodiments, the modified immune cells or pharmaceutical compositions thereof are administered to an individual by infusion, such as intravenous infusion. Infusion techniques for immunotherapy are known in the art (see, e.g., Rosenberg et al., New Eng. J. of Med. 319: 1676 (1988)). In some embodiments, the modified immune cells or pharmaceutical compositions thereof are administered to an individual by intradermal or subcutaneous injection.

In some embodiments, the modified immune cells or pharmaceutical compositions thereof are administered by intravenous injection. In some embodiments, the modified immune cells or pharmaceutical compositions thereof are injected directly into a tumor, or a lymph node. In some embodiments, the modified immune cells or pharmaceutical compositions thereof are administered locally to a site of tumor, such as directly into tumor cells, or to a tissue having tumor cells.

Dosages of modified immune cells or pharmaceutical compositions thereof of the present invention may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46. It is within the scope of the present application that different formulations will be effective for different treatments and different disorders, and that administration intended to treat a specific organ or tissue may necessitate delivery in a manner different from that to another organ or tissue.

In some embodiments, modified immune cells or pharmaceutical compositions thereof described herein are administered at a dosage of at least about any of 10⁴, 10⁵, 10⁶, 10⁷, 10⁸, or 10⁹ cells/kg of body weight of the individual. In some embodiments, modified immune cells or pharmaceutical compositions thereof are administered at a dosage of any of about 10⁴ to about 10⁵, about 10⁵ to about 10⁶, about 10⁶ to about 10⁷, about 10⁷ to about 10⁸, about 10⁸ to about 10⁹, about 10⁴ to about 10⁹, about 10⁴ to about 10⁶, about 10⁶ to about 10⁸, or about 10⁵ to about 10⁷ cells/kg of body weight of the individual.

In some embodiments, the modified immune cells or pharmaceutical compositions thereof described herein are administered for a single time. In some embodiments, the modified immune cells or pharmaceutical compositions thereof are administered for multiple times (such as any of 2, 3, 4, 5, 6, or more times). In some embodiments, the modified immune cells or pharmaceutical compositions thereof are administered once per week, once 2 weeks, once 3 weeks, once 4 weeks, once per month, once per 2 months, once per 3 months, once per 4 months, once per 5 months, once per 6 months, once per 7 months, once per 8 months, once per 9 months, or once per year. In some embodiments, the interval between administrations is about any one of 1 week to 2 weeks, 2 weeks to 1 month, 2 weeks to 2 months, 1 month to 2 months, 1 month to 3 months, 3 months to 6 months, or 6 months to a year. The optimal dosage and treatment regime for a particular patient can readily be determined by one skilled in the art of medicine by monitoring the patient for signs of disease and adjusting the treatment accordingly.

Dosages may be administered by one or more separate administrations, or by continuous infusion. In some embodiments, the modified immune cells or pharmaceutical compositions thereof are administered in split doses, such as about any one of 2, 3, 4, 5, or more doses. In some embodiments, the split doses are administered over about a week. In some embodiments, the dose is equally split. In some embodiments, the split doses are about 20%, about 30%, about 40%, or about 50% of the total dose. In some embodiments, the interval between consecutive split doses is about 1 day, 2 days, 3 days or longer. For repeated administrations over several days or longer, depending on the condition, the treatment is sustained until a desired suppression of disease symptoms occurs. However, other dosage regimens may be useful. The progress of this therapy is easily monitored by conventional techniques and assays.

### V. METHODS OF IDENTIFYING TARGET GENES IN IMMUNE CELLS WHOSE MUTATIONS INCREASE RESISTANCE TO AICD

The present application provides methods of identifying a target gene in an immune cell that modulates (e.g., increases) resistance to AICD. Any methods (e.g., immune cell library construction, sgRNA or sgRNA^{iBAR} library construction, target gene identification, or obtaining desired cell population etc.) described in US20220064633 and WO2022143783 can be used herein, the contents of each of which are incorporated herein by reference in their entireties. Target genes identified herein are particularly useful in patient selection/exclusion in disease treatments, such as cancer, infection, inflammation, immune-related diseases characterized by effector cell exhaustion, etc., or immune cell donor selection/exclusion, or adoptive cell therapy optimization/quality control. For example, individuals carrying a mutation (e.g., inactivation) in an AICD resistant gene identified herein (e.g., one or more of SPPL3, FADD, FAS, CASP8, ARID1A, BAK1, BID, ETS1, IKZF2, and HIST1H1B, such as SPPL3), and/or with reduced or absent expression (e.g., mRNA or protein) of the AICD resistant gene compared to another individual (patient or healthy individual), and/or with reduced or abolished activity of an expression product (e.g., mRNA or protein) of the AICD resistant gene compared to another individual (patient or healthy individual), are particularly suitable for autologous or allogeneic CAR-T or CAR-NK cell therapy, or donating immune cells.

In some embodiments, there is provided a method of identifying a target gene in an immune cell (e.g., T cell, B cell, or NK cell) whose mutation increases (e.g., increasing at least about any of 10%, 20%, 30%, 40%, 50%, 60%, 70%, 80%, 90%, 95%, 1-fold, 2-fold, 5-fold, 10-fold, 20-fold, 50-fold, 100-fold, or more) resistance to AICD, comprising: a) providing an immune cell library comprising a plurality of immune cells, wherein each of the plurality of immune cells has a mutation (e.g., inactivating mutation) at a hit gene (“hit gene mutation”), wherein the hit gene in at least two of the plurality of immune cells are different from each other; b) contacting the immune cell library with a Fas ligand (FasL); c) obtaining from the immune cell library an AICD-resistant immune cell population; and d) identifying the target gene based on the difference between the profiles of hit gene mutations in the AICD-resistant immune cell population and a control immune cell population. In some embodiments, the control immune cell population is a subpopulation of the immune cell library before step b). In some embodiments, the method further comprises obtaining the subpopulation of the immune cell library before step b). In some embodiments, the control immune cell population is a same immune cell library cultured under the same condition and not contacted with the FasL. In some embodiments, the method further comprises culturing a same immune cell library under the same condition and not contacting with the FasL. In some embodiments, the profiles of hit gene mutations in the AICD-resistant immune cell population and the control immune cell population are identified by next generation sequencing (NGS). In some embodiments, the method comprises comparing the sequence counts of sequences comprising the hit gene mutations obtained from the AICD-resistant immune cell population with sequence counts of sequences comprising the hit gene mutations obtained from the control immune cell population, wherein the hit genes whose corresponding hit gene mutation sequences are identified as enriched in the AICD-resistant immune cell population compared to the control immune cell population with an false discovery rate (FDR)≤0.2 (and/or with at least about 2-fold enrichment) are identified as target genes whose mutations increase resistance to AICD (“AICD resistant genes” or “FasL resistant genes”). In some embodiments, the immune cell library has at least about 100-fold (e.g., at least about any of 200-, 300-, 600-, 1000-, 2000-, 4000-, 6000-, 8000-, 10000-, 15000-, or more) coverage for each hit gene, such as about 1000-fold to about 12,000-fold coverage for each hit gene. In some embodiments, each hit gene is targeted by at least 2 (e.g., 2, 3, 4, 5, 6, or more, such as 3, or 6 to 12) different hit gene mutations (e.g., targeting different target sites of the hit gene) in the immune cell library. In some embodiments, step c) comprise using fluorescence-activated cell sorting (FACS) or centrifugation (e.g., low speed centrifugation) to obtain the AICD-resistant immune cell population. In some embodiments, step c) further comprises contacting the immune cell library with a viability indicator, such as one or more of propidium iodide (PI), DAPI, 7-AAD, and Annexin V. In some embodiments, the AICD-resistant immune cell population is Annexin V⁻ negative and DAPI⁻ negative. In some embodiments, step c) comprise using FACS to obtain Annexin V⁻ negative and DAPI⁻ negative cells from the immune cell library. In some embodiments, step b) comprises culturing the immune cell library for about 16 hours in the presence of FasL, such as at 37° C., 5% CO₂. In some embodiments, the sequence counts of sequences comprising the hit gene mutations are subject to median ratio normalization followed by mean-variance modeling. In some embodiments, the variance of each sequence comprising the hit gene mutation (e.g., inactivating mutation) is adjusted based on data consistency among the same gene. In some embodiments, the data consistency among the different hit gene mutation (e.g., inactivating mutation) sequences corresponding to the same hit gene is determined based on the direction of the fold change of each hit gene mutation sequence, wherein the variance of the hit gene mutation sequence is increased if the fold changes of the different hit gene mutation sequences are in different directions with respect to each other (e.g., increased vs. reduced, increased vs. unchanged, or reduced vs. unchanged are all considered as different directions) for the same hit gene. In some embodiments, the immune cell library comprises at least about 100 million cells.

In some embodiments, the immune cell library is generated by contacting an initial population of immune cells (e.g., T cells such as CAR-T cells, or NK cells such as CAR-NK cells) with a mutagenic agent.

In some embodiments, the immune cell library is generated by subjecting an initial population of immune cells (e.g., T cells such as CAR-T cells, or NK cells such as CAR-NK cells) to gene editing (e.g., genome-wide, or subset of genes). In some embodiments, the immune cell library is generated by contacting an initial population of immune cells with i) a gRNA (e.g., sgRNA) library comprising a plurality of gRNA constructs, wherein each gRNA construct (e.g., lentiviral vector or lentivirus) comprises or encodes a gRNA, and wherein each gRNA comprises a guide sequence that is complementary (e.g., at least about any of 50%, 60%, 70%, 80%, 90%, 95%, 96%, 97%, 98%, 99%, or 100% complementary) to a target site in a corresponding hit gene; and ii) a Cas component comprising a Cas protein or a nucleic acid encoding the Cas protein, under a condition that allows introduction of the gRNA constructs and the Cas component into the initial population of immune cells and generation of the mutations at the hit genes. In some embodiments, the gRNA (e.g., sgRNA) library and the Cas component are introduced into the initial population of immune cells simultaneously. In some embodiments, the gRNA library and the Cas component are introduced into the initial population of immune cells sequentially. In some embodiments, the initial population of immune cells comprises a Cas component (e.g., Cas9). In some embodiments, the immune cell library is generated by contacting an initial population of immune cells comprising a Cas component (e.g., Cas9) with a gRNA (e.g., sgRNA) library comprising a plurality of gRNA constructs, wherein each gRNA construct (e.g., lentiviral vector or lentivirus) comprises or encodes a gRNA, and wherein each gRNA comprises a guide sequence that is complementary (e.g., at least about any of 50%, 60%, 70%, 80%, 90%, 95%, 96%, 97%, 98%, 99%, or 100% complementary) to a target site in a corresponding hit gene, under a condition that allows introduction of the gRNA constructs into the initial population of immune cells comprising Cas (e.g., Cas9) and generation of the mutations at the hit genes. In some embodiments, the Cas component is introduced into the initial population of immune cells before the introduction of the gRNA library. In some embodiments, the immune cell library is generated by i) contacting an initial population of immune cells with a Cas component comprising a Cas protein or a nucleic acid encoding the Cas protein (e.g., lentiviral vector or lentivirus encoding Cas9, or Cas9 mRNA), under a condition that allows introduction of the Cas component into the initial population of immune cells; ii) optionally obtaining a population of immune cells comprising the Cas component (“Cas⁺ immune cell population”; such as by FACS sorting, e.g., with a marker on the Cas-encoding vector); iii) contacting the Cas⁺ immune cell population with a gRNA (e.g., sgRNA) library comprising a plurality of sgRNA constructs, wherein each gRNA construct (e.g., lentiviral vector or lentivirus) comprises or encodes an gRNA, and wherein each gRNA comprises a guide sequence that is complementary (e.g., at least about any of 50%, 60%, 70%, 80%, 90%, 95%, 96%, 97%, 98%, 99%, or 100% complementary) to a target site in a corresponding hit gene, under a condition that allows introduction of the gRNA constructs into the immune cells (e.g., Cas⁺ immune cells) and generation of the mutations at the hit genes. In some embodiments, the Cas protein is Cas9. In some embodiments, each gRNA (e.g., crRNA) comprises the guide sequence fused to a second sequence, wherein the second sequence comprises a direct repeat (DR) sequence that interacts with the Cas protein. In some embodiments, each gRNA (e.g., sgRNA) comprises the guide sequence fused to a second sequence, wherein the second sequence comprises a repeat-anti-repeat stem loop that interacts with the Cas protein (e.g., Cas9). In some embodiments, the second sequence of each gRNA (e.g., sgRNA) further comprises a stem loop 1, a stem loop 2, and/or a stem loop 3. In some embodiments, each sgRNA further comprises an iBAR sequence (“sgRNA^{iBAR}”), wherein each sgRNA^{iBAR} is operable with the Cas protein to modify (e.g., cleave or modulate expression) the hit gene. In some embodiments, each sgRNA^{iBAR} comprises in the 5′-to-3′ direction a first stem sequence and a second stem sequence, wherein the first stem sequence hybridizes with the second stem sequence to form a dsRNA region that interacts with the Cas protein, and wherein the iBAR sequence is disposed between the 3′ end of the first stem sequence and the 5′ end of the second stem sequence. In some embodiments, the Cas protein is Cas9, and the iBAR sequence of each sgRNA^{iBAR} is inserted in the loop region of the repeat-anti-repeat stem loop. In some embodiments, each guide sequence comprises about 17 to about 23 nucleotides. In some embodiments, at least about 95% (e.g., at least about any of 96%, 97%, 98%, 99%, or more), such as at least about 99%, of the gRNA (e.g., sgRNA) constructs in the gRNA library are introduced into the initial population of immune cells. In some embodiments, each hit gene within the immune cell library or the gRNA (e.g., sgRNA) library is targeted by at least about 3 (e.g., about 6 to about 12) different gRNA constructs in at least about 3 (e.g., about 6 to about 12) different target sites of the hit gene. In some embodiments, the immune cell library has at least about 500-fold (e.g., about 600-fold to about 2000-fold) coverage for each gRNA (e.g., sgRNA) or sgRNA^{iBAR}. In some embodiments, the immune cell library has at least about 1000-fold coverage for each gRNA (e.g., sgRNA). In some embodiments, the immune cell library has at least about 1500-fold coverage for each hit gene, such as about 2000-fold to about 6000-fold (e.g., 5000-fold) coverage for each hit gene. In some embodiments, the gRNA (e.g., sgRNA) library comprises at least about 2000 gRNA constructs. In some embodiments, each gRNA (e.g., sgRNA) construct in the gRNA library is a plasmid. In some embodiments, each gRNA (e.g., sgRNA) construct in the gRNA library is a viral vector (e.g., lentiviral vector). In some embodiments, the gRNA (e.g., sgRNA) library is contacted with the initial population of immune cells at an MOI of at least about 2 (e.g., 3).

In some embodiments, the immune cell library is generated by contacting an initial population of immune cells with i) an sgRNA^{iBAR} library comprising a plurality of sets of sgRNA^{iBAR} constructs, wherein each set of sgRNA^{iBAR} constructs comprise three or more (e.g., four) sgRNA^{iBAR} constructs (e.g., lentiviral vector or lentivirus) each comprising or encoding an sgRNA^{iBAR}, wherein each sgRNA^{iBAR} comprises a guide sequence and an iBAR sequence, wherein the guide sequences for the three or more (e.g., four) sgRNA^{iBAR} constructs are the same and are complementary (e.g., at least about any of 50%, 60%, 70%, 80%, 90%, 95%, 96%, 97%, 98%, 99%, or 100% complementary) to a same target site of a hit gene, wherein the iBAR sequence for each of the three or more (e.g., four) sgRNA^{iBAR} constructs is different from each other, wherein the guide sequence of each set of sgRNA^{iBAR} constructs is complementary to a different target site of a hit gene (e.g., different hit genes, or different sites within the same hit gene), and wherein each sgRNA^{iBAR} is operable with a Cas (e.g., Cas9) protein to modify the target site; and ii) a Cas (e.g., Cas9) component comprising a Cas protein or a nucleic acid encoding the Cas protein (e.g., lentiviral vector or lentivirus encoding Cas9, or Cas9 mRNA), under a condition that allows introduction of the sgRNA^{iBAR} constructs and the Cas component into the initial population of immune cells and generation of the mutations at the hit genes. In some embodiments, the initial population of immune cells comprises a Cas component (e.g., Cas9). In some embodiments, the immune cell library is generated by contacting an initial population of immune cells comprising a Cas (e.g., Cas9) component with an sgRNA^{iBAR} library comprising a plurality of sets of sgRNA^{iBAR} constructs, wherein each set of sgRNA^{iBAR} constructs comprise three or more (e.g., four) sgRNA^{iBAR} constructs (e.g., lentiviral vector or lentivirus) each comprising or encoding an sgRNA^{iBAR}, wherein each sgRNA^{iBAR} comprises a guide sequence and an iBAR sequence, wherein the guide sequences for the three or more (e.g., four) sgRNA^{iBAR} constructs are the same and are complementary (e.g., at least about any of 50%, 60%, 70%, 80%, 90%, 95%, 96%, 97%, 98%, 99%, or 100% complementary) to a same target site of a hit gene, wherein the iBAR sequence for each of the three or more (e.g., four) sgRNA^{iBAR} constructs is different from each other, wherein the guide sequence of each set of sgRNA^{iBAR} constructs is complementary to a different target site of a hit gene (e.g., different hit genes, or different sites within the same hit gene), and wherein each sgRNA^{iBAR} is operable with a Cas (e.g., Cas9) protein to modify the target site, under a condition that allows introduction of the sgRNA^{iBAR} constructs into the initial population of immune cells comprising Cas (e.g., Cas9) and generation of the mutations at the hit genes. In some embodiments, the immune cell library is generated by i) contacting an initial population of immune cells with a Cas component comprising a Cas protein or a nucleic acid encoding the Cas protein (e.g., lentiviral vector or lentivirus encoding Cas9, or Cas9 mRNA), under a condition that allows introduction of the Cas component into the initial population of immune cells; ii) optionally obtaining a population of immune cells comprising the Cas component (“Cas⁺ immune cell population”; such as by FACS sorting, e.g., with a marker on the Cas-encoding vector); iii) contacting the Cas⁺ immune cell population with an sgRNA^{iBAR} library comprising a plurality of sets of sgRNA^{iBAR} constructs, wherein each set of sgRNA^{iBAR} constructs comprise three or more (e.g., four) sgRNA^{iBAR} constructs (e.g., lentiviral vector or lentivirus) each comprising or encoding an sgRNA^{iBAR} wherein each sgRNA^{iBAR} comprises a guide sequence and an iBAR sequence, wherein the guide sequences for the three or more (e.g., four) sgRNA^{iBAR} constructs are the same and are complementary (e.g., at least about any of 50%, 60%, 70%, 80%, 90%, 95%, 96%, 97%, 98%, 99%, or 100% complementary) to a same target site of a hit gene, wherein the iBAR sequence for each of the three or more (e.g., four) sgRNA^{iBAR} constructs is different from each other, wherein the guide sequence of each set of sgRNA^{iBAR} constructs is complementary to a different target site of a hit gene (e.g., different hit genes, or different sites within the same hit gene), and wherein each sgRNA^{iBAR} is operable with a Cas (e.g., Cas9) protein to modify the target site, under a condition that allows introduction of the sgRNA^{iBAR} constructs into the immune cells (e.g., Cas⁺ immune cells) and generation of the mutations at the hit genes. In some embodiments, the immune cell library is generated by contacting an initial population of immune cells with i) an sgRNA^{iBAR} library comprising a plurality of sets of sgRNA^{iBAR} constructs, wherein each set of sgRNA^{iBAR} constructs comprise three or more (e.g., four) sgRNA^{iBAR} constructs each comprising or encoding an sgRNA^{iBAR}, wherein each sgRNA^{iBAR} comprises a guide sequence, a second sequence, and an iBAR sequence, wherein the guide sequences for the three or more (e.g., four) sgRNA^{iBAR} constructs are the same and are complementary (e.g., at least about any of 50%, 60%, 70%, 80%, 90%, 95%, 96%, 97%, 98%, 99%, or 100% complementary) to a same target site of a hit gene, wherein the iBAR sequence for each of the three or more (e.g., four) sgRNA^{iBAR} constructs is different from each other, wherein the guide sequence is fused to a second sequence, wherein the second sequence comprises a repeat-anti-repeat stem loop that interacts with a Cas (e.g., Cas9) protein, wherein the iBAR sequence is inserted in the loop region of the repeat-anti-repeat stem loop, wherein the guide sequence of each set of sgRNA^{iBAR} constructs is complementary to a different target site of a hit gene (e.g., different hit genes, or different target sites of the same hit gene), and wherein each sgRNA^{iBAR} is operable with the Cas (e.g., Cas9) protein to modify the target site; and ii) a Cas (e.g., Cas9) component comprising a Cas (e.g., Cas9) protein or a nucleic acid encoding the Cas (e.g., Cas9) protein, under a condition that allows introduction of the sgRNA^{iBAR} constructs and the Cas (e.g., Cas9) component into the initial population of immune cells and generation of the mutations at the hit genes. In some embodiments, the Cas component (e.g., Cas9) is introduced into the immune cells before the introduction of the sgRNA^{iBAR} library. In some embodiments, the sgRNA^{iBAR} library is introduced into the immune cells before the introduction of the Cas component (e.g., Cas9). In some embodiments, the Cas component (e.g., Cas9) and the sgRNA^{iBAR} library are introduced into the immune cells at the same time. In some embodiments, each iBAR sequence comprises about 1 to about 50 (such as 6) nucleotides. In some embodiments, each set of sgRNA^{iBAR} constructs comprises four sgRNA^{iBAR} constructs, and the iBAR sequence for each of the four sgRNA^{iBAR} constructs is different from each other. In some embodiments, the sgRNA^{iBAR} library comprises at least about 100 (e.g., 2000) sets of sgRNA^{iBAR} constructs. In some embodiments, the sgRNA^{iBAR} library comprises at least about 2000 sgRNA^{iBAR} constructs. In some embodiments, the iBAR sequences for at least two sgRNA^{iBAR} constructs among different sets of sgRNA^{iBAR} constructs are the same (e.g., the first set and the second set of sgRNA^{iBAR} constructs have at least 1, 2, 3, 4, or more shared iBAR sequences among the two sets of sgRNA^{iBAR} constructs). In some embodiments, the iBAR sequences for at least two sets of sgRNA^{iBAR} constructs are the same. In some embodiments, each sgRNA^{iBAR} construct in the sgRNA^{iBAR} library is a plasmid. In some embodiments, each sgRNA^{iBAR} construct in the sgRNA^{iBAR} library is a viral vector (e.g., lentiviral vector). In some embodiments, the sgRNA^{iBAR} library is contacted with the initial population of immune cells at an MOI of more than about 2 (e.g., at least about 3, 5, or 10), such as 3. In some embodiments, the sgRNA^{iBAR} library comprising a plurality of sgRNA^{iBAR} constructs comprises or encodes sgRNA^{iBAR} with guide sequences complementary to target sites of hit genes related to one or more of AICD, GvHD, HvG, immune cell proliferation, differentiation, maturation, activation, persistence, homeostasis, and effector function. In some embodiments, at least about 95% (e.g., at least about any of 96%, 97%, 98%, 99%, or more), such as at least about 99%, of the sgRNA^{iBAR} constructs in the sgRNA^{iBAR} library are introduced into the initial population of immune cells. In some embodiments, each hit gene within the immune cell library or the sgRNA^{iBAR} library is targeted by 3 different sets of sgRNA^{iBAR} constructs at 3 different target sites of the hit gene. In some embodiments, the immune cell library has at least about 500-fold coverage for each sgRNA^{iBAR}, such as about 1000- to about 1500-fold coverage for each sgRNA^{iBAR}. In some embodiments, the immune cell library has at least about 1000-fold coverage for each sgRNA^{iBAR}. In some embodiments, the immune cell library has at least about 2000-fold coverage for each set of sgRNAs^{iBAR}, such as about 4000- to about 6000-fold coverage for each set of sgRNAs^{iBAR}. In some embodiments, the immune cell library has at least about 2000-fold coverage for each hit gene, such as about 4000-fold to about 12,000-fold coverage for each hit gene.

Screening methods using sgRNA^{iBAR} libraries described herein in some embodiments can improve target identification and data reproducibility by statistical analysis and reduce FDR and. In conventional CRISPR/Cas-based screening methods using a pooled sgRNA library, a high-quality cell library expressing gRNAs are generated using a low MOI during cell library construction to ensure that each cell harbors on average less than one sgRNA or paired guide RNA (“pgRNA”). Because the sgRNA molecules in a library are randomly integrated in the transfected cells, a sufficiently low MOI ensures that each cell expresses a single sgRNA, thereby minimizing the FDR of the screen. To further reduce FDR and increase data reproducibility, in-depth coverage of gRNAs and multiple biological replicates are often necessary to obtain hit genes with high statistical significance. The conventional screen methods face difficulties when a large number of genome-wide screens are needed, when cell materials for library construction are limited, or when one conducts more challenging screens (i.e., in vivo screen) for which it is difficult to arrange the experimental replications or control the MOI. The screening methods using sgRNA^{iBAR} libraries described herein overcome the difficulties by including an iBAR sequence in each sgRNA, which enables collection of internal replicates within each sgRNA set having the same guide sequence but different iBAR sequences. Such iBAR method can reduce experimental noise. For example, an iBAR with four nucleotides for each sgRNA, as demonstrated in WO2020125762 (the content of which is incorporated herein by reference in its entirety), can provide sufficient internal replicates to evaluate data consistency among different sgRNA^{iBAR} constructs targeting the same genomic locus. The high level of consistency between the two independent experiments in WO2020125762 indicates that one experimental replicate is sufficient for CRISPR/Cas screens using the iBAR method. Because library coverage is significantly increased with a high MOI during viral transduction of host cells, the cell number in the initial cell population could be reduced more than 20-fold to reach the same library coverage, as demonstrated in the constructed genome-wide human library in WO2020125762. By the same token, workload for each genome-wide screen using sgRNA^{iBAR} can be reduced proportionally. Using sgRNAs with different iBAR sequences, one could then trace the performance of each guide sequence multiple times within the same experiment by counting both the guide sequence and the corresponding iBAR nucleotide sequences, thereby drastically reducing FDR, and increasing efficiency and liability. Transduction efficiency and library coverage could be further increased, a high viral titer is used during the viral transduction step, for example, with MOI>1 (e.g., MOI>1.5, MOI>2, MOI>2.5, MOI>3, MOI>3.5, MOI>4, MOI>4.5, MOI>5, MOI>5.5, MOI>6, MOI>6.5, MOI>7, MOI>7.5, MOI>8, MOI>8.5, MOI>9, MOI>9.5 or MOI>10; such as, MOI is about any of 1, 1.5, 2, 2.5, 3, 3.5, 4, 4.5, 5, 5.5, 6, 6.5, 7, 7.5, 8, 8.5, 9, 9.5, or 10).

In some embodiments, there is provided a method of identifying a target gene in an immune cell (e.g., T cell such as CAR-T cell) whose mutation (e.g., LOF mutation) increases resistance to AICD, comprising: a) providing an immune cell library comprising an gRNA (e.g. sgRNA) or sgRNA^{iBAR} library described herein targeting one or more hit genes; b) contacting the immune cell library with a FasL; c) obtaining from the immune cell library an AICD-resistant immune cell population; and d) identifying the target gene based on the difference between the profiles of gRNA or sgRNAs^{iBAR} or hit gene mutations in the AICD-resistant immune cell population and a control immune cell population. In some embodiments, the control immune cell population is a subpopulation of the immune cell library before step b). In some embodiments, the control immune cell population is a same immune cell library cultured under the same condition and not contacted with the FasL. In some embodiments, the method further comprises obtaining the control immune cell population before step b). In some embodiments, the method further comprises culturing a same immune cell library under the same condition and not contacting with the FasL. In some embodiments, there is provided a method of identifying a target gene in an immune cell (e.g., T cell such as CAR-T cell) whose mutation (e.g., LOF mutation) increases resistance to AICD, comprising: a1) providing an immune cell library comprising an gRNA (e.g., sgRNA) or sgRNA^{iBAR} library described herein targeting one or more hit genes; a2) optionally growing the immune cell library (e.g., at 37° C., 5% CO₂ for about 6 days); a3) obtaining a subpopulation of the immune cell library from a1) or a2) as a control immune cell population; b) contacting the immune cell library (from step a1) or a2), accordingly) with a FasL; c) obtaining from the immune cell library from step b) an AICD-resistant immune cell population; and d) identifying the target gene based on the difference between the profiles of gRNA (e.g., sgRNA) or sgRNAs^{iBAR} or hit gene mutations in the AICD-resistant immune cell population and the control immune cell population. In some embodiments, there is provided a method of identifying a target gene in an immune cell (e.g., T cell such as CAR-T cell) whose mutation (e.g., LOF mutation) increases resistance to AICD, comprising: a1) providing an immune cell library comprising an gRNA (e.g., sgRNA) or sgRNA^{iBAR} library described herein targeting one or more hit genes; a2) culturing a same immune cell library under the same condition and not subjected to step b) as a control immune cell population; b) contacting the immune cell library from step a1) with a FasL; c) obtaining from the immune cell library from step b) an AICD-resistant immune cell population; and d) identifying the target gene based on the difference between the profiles of gRNA (e.g., sgRNA) or sgRNAs^{iBAR} or hit gene mutations in the AICD-resistant immune cell population and the control immune cell population. In some embodiments, step c) comprise using FACS or centrifugation (e.g., low speed) to obtain the AICD-resistant immune cell population. In some embodiments, step c) further comprises contacting the immune cell library after step b) with a viability indicator, such as one or more of PI, DAPI, 7-AAD, and Annexin V. In some embodiments, the AICD-resistant immune cell population is Annexin V⁻ negative and DAPI⁻ negative. In some embodiments, step b) comprises culturing the immune cell library for about 16 hours in the presence of FasL. In some embodiments, there is provided a method of identifying a target gene in an immune cell (e.g., T cell such as CAR-T cell) whose mutation (e.g., LOF mutation) increases resistance to AICD, comprising: a1) providing an immune cell library comprising an gRNA (e.g., sgRNA) or sgRNA^{iBAR} library described herein targeting one or more hit genes; a2) optionally growing the immune cell library (e.g., at 37° C., 5% CO₂ for about 6 days); b) contacting the immune cell library (from a1) or a2), accordingly) with FasL; c) obtaining from the immune cell library (after step b)) an AICD-resistant immune cell population by centrifugation (e.g., low speed); and d) identifying the target gene based on the difference between the profiles of gRNA (e.g., sgRNA) or sgRNAs^{iBAR} or hit gene mutations in the AICD-resistant immune cell population and a control immune cell population. In some embodiments, the control immune cell population is obtained by culturing a same immune cell library under the same condition and not contacting with the FasL. In some embodiments, the sequence counts obtained from the AICD-resistant immune cell population are compared to corresponding sequence counts obtained from the control immune cell population to provide fold changes (e.g., actual fold changes, or derivatives of fold changes such as log 2 or log 10 fold changes). In some embodiments, the identification of the target gene is based on the difference between the profiles of gRNAs (e.g., sgRNAs) or sgRNAs^{iBAR} in the AICD-resistant immune cell population and the control immune cell population. In some embodiments, the profiles of gRNAs (e.g., sgRNAs) or sgRNAs^{iBAR} in the AICD-resistant immune cell population and the control immune cell population are identified by NGS. In some embodiments, identifying the target gene in step d) comprises: comparing the gRNAs (e.g., sgRNAs) or sgRNA^{iBAR} (or guide sequence thereof) sequence counts obtained from the AICD-resistant immune cell population with gRNA (e.g., sgRNA) or sgRNA^{iBAR} (or guide sequence thereof) sequence counts obtained from the control immune cell population, wherein: the hit genes whose corresponding gRNA (e.g., sgRNA) or sgRNA^{iBAR} guide sequences are identified as enriched in the AICD-resistant immune cell population compared to the control immune cell population with an FDR≤0.2 (and/or with at least about 2-fold enrichment) are identified as target genes whose mutations increase resistance to AICD (AICD resistant genes). Hence in some embodiments, there is provided a method of identifying a target gene in an immune cell (e.g., T cell such as CAR-T cell) whose mutation (e.g., LOF mutation) increases resistance to AICD, comprising: a1) providing an immune cell library comprising an gRNA (e.g., sgRNA^{iBAR}) library described herein targeting one or more hit genes, and optionally growing the immune cell library (e.g., at 37° C., 5% CO₂ for about 6 days); a2) culturing a same immune cell library under the same condition and not subjected to step b) as a control immune cell library; b) contacting the immune cell library (from step a1)) with FasL; c) using low speed centrifugation to obtain alive cells from the immune cell library after step b), thereby obtaining an AICD-resistant immune cell population; and d) identifying the target gene based on the difference between the profiles of gRNAs (e.g., sgRNAs^{iBAR}) in the AICD-resistant immune cell population and the control immune cell population (e.g., by NGS), wherein step d) comprises i) comparing gRNA (e.g., sgRNA^{iBAR}) sequence counts obtained from the AICD-resistant immune cell population with gRNA (e.g., sgRNA^{iBAR}) sequence counts obtained from the control immune cell population, wherein the hit genes whose corresponding gRNA (e.g., sgRNA^{iBAR}) guide sequences are identified as enriched in the AICD-resistant immune cell population compared to the control immune cell population with an FDR≤0.2 (and/or with at least about 2-fold enrichment) are identified as target genes whose mutations increase resistance to AICD (AICD resistant genes). In some embodiments, the gRNA (e.g., sgRNA) or sgRNA^{iBAR} library targets genes related to one or more of AICD, GvHD, HvG, immune cell proliferation, differentiation, maturation, activation, persistence, homeostasis, and effector function. In some embodiments, the immune cell library has about 100-fold to about 2000-fold coverage for each sgRNA^{iBAR} such as about 1000-fold coverage for each sgRNA^{iBAR}. In some embodiments, the immune cell library has at least about 400-fold coverage for each hit gene, e.g., about 1200-fold to about 12,000-fold coverage for each hit gene. In some embodiments, identifying the target gene in step d) comprises: i) identifying the gRNA (e.g., sgRNA) or sgRNA^{iBAR} sequence in the AICD-resistant immune cell population; and ii) identifying the hit gene corresponding to the guide sequence of the gRNAs (e.g., sgRNAs) or sgRNAs^{iBAR}. In some embodiments, identifying the target gene in step d) comprises: i) obtaining sgRNA^{iBAR} sequences in the AICD-resistant immune cell population; ii) ranking the corresponding guide sequences of the sgRNA^{iBAR} sequences based on sequence counts, wherein the ranking comprises adjusting the rank of each guide sequence based on data consistency among the iBAR sequences in the sgRNA^{iBAR} sequences corresponding to the guide sequence; and iii) identifying the hit gene corresponding to a guide sequence ranked above a predetermined threshold level. In some embodiments, the method is a positive screening. In some embodiments, the gRNA (e.g., sgRNA) or sgRNA^{iBAR} sequence counts are subject to median ratio normalization followed by mean-variance modeling. In some embodiments, the variance of each guide sequence is adjusted based on data consistency among the iBAR sequences in the sgRNA^{iBAR} sequences corresponding to the guide sequence. In some embodiments, the data consistency among the iBAR sequences in the sgRNA^{iBAR} sequences corresponding to each guide sequence is determined based on the direction of the fold change of each iBAR sequence, wherein the variance of the guide sequence is increased if the fold changes of the iBAR sequences are in different directions with respect to each other (e.g., increased vs. reduced, increased vs. unchanged, or reduced vs. unchanged). In some embodiments, the variance of each guide sequence is adjusted based on data consistency among the same gene. In some embodiments, the data consistency among the different guide sequences corresponding to the same hit gene is determined based on the direction of the fold change of each guide sequence, wherein the variance of the guide sequence is increased if the fold changes of the different guide sequences are in different directions with respect to each other (e.g., increased vs. reduced, increased vs. unchanged, or reduced vs. unchanged are all considered as different directions) for the same hit gene.

In some embodiments, the immune cell library is subjected to at least two (e.g., 2, 3, 4, or more) rounds of FasL treatment. In some embodiments, after obtaining the AICD-resistant immune cell population in step c), the AICD-resistant immune cell population is cultured under a condition (e.g., e.g., at 37° C., 5% CO₂) to grow to about the same or similar (e.g., at most about 10% variance) number of immune cells as the immune cell library before FasL treatment (e.g., 100 million cells), such as for about 5 to about 7 days, e.g., 6 days, or about 3 PDT. In some embodiments, after contacting the immune cell library with FasL (e.g., culturing in the present of FasL for about 16 hours), the FasL-treated immune cell library is contacted with a viability indicator, e.g., one or more of PI, DAPI, 7-AAD, and Annexin V, and immune cell apoptosis rate or survival rate is examined. For example, Annexin V⁻ negative and DAPI⁻ negative cells are alive cells or cells more resistant to AICD/FasL treatment.

In some embodiments, there is provided a method of identifying a target gene in an immune cell (e.g., T cell such as CAR-T cell) whose mutation (e.g., LOF mutation) increases resistance to AICD, comprising: a) providing an immune cell library comprising an gRNA (e.g. sgRNA) or sgRNA^{iBAR} library described herein targeting one or more hit genes; b1) contacting the immune cell library with a first FasL (“first FasL treatment step”); c1-i) obtaining from the immune cell library a first AICD-resistant immune cell population (“first obtaining step”; such as by low speed centrifugation or FACS); c1-ii) optionally culturing the first AICD-resistant immune cell population for about 6 days (“optional first recovery step”); b2) optionally contacting the first AICD-resistant immune cell population with a second FasL (“optional second FasL treatment step”); c2-i) optionally obtaining from step b2) a second AICD-resistant immune cell population (“optional second obtaining step”; such as by low speed centrifugation or FACS); c2-ii) optionally culturing the second AICD-resistant immune cell population for about 6 days (“optional second recovery step”); b3) optionally contacting the second AICD-resistant immune cell population with a third FasL (“optional third FasL treatment step”); c3-i) optionally obtaining from step b3) a final AICD-resistant immune cell population (“optional third obtaining step”; such as by low speed centrifugation or FACS); c3-ii) optionally culturing the final AICD-resistant immune cell population for about 6 days (optional third recovery step); and d) identifying the target gene based on the difference between the profiles of gRNA (e.g., sgRNA) or sgRNAs^{iBAR} or hit gene mutations in the final AICD-resistant immune cell population and a control immune cell population. In some embodiments, the first, second, and third FasL have the same concentration. In some embodiments, the first, second, and third FasL have different concentrations. In some embodiments, there is provided a method of identifying a target gene in an immune cell (e.g., T cell such as CAR-T cell) whose mutation (e.g., LOF mutation) increases resistance to AICD, comprising: a) providing an immune cell library comprising an gRNA (e.g. sgRNA) or sgRNA^{iBAR} library described herein targeting one or more hit genes; b1) contacting the immune cell library with a first FasL (“first FasL treatment step”); c1-i) centrifuging the immune cell library after step b1) to obtain a first AICD-resistant immune cell population (“first obtaining step”); c1-ii) culturing the first AICD-resistant immune cell population for about 6 days (“first recovery step”); b2) contacting the first AICD-resistant immune cell population with the FasL (“second FasL treatment step”); c2-i) centrifuging the first AICD-resistant immune cell population after step b2) to obtain a second AICD-resistant immune cell population (“second obtaining step”); c2-ii) culturing the second AICD-resistant immune cell population for about 6 days (“second recovery step”); b3) contacting the second AICD-resistant immune cell population with the FasL (“third FasL treatment step”); c3-i) centrifuging the second AICD-resistant immune cell population after step b3) to obtain a final AICD-resistant immune cell population (“third obtaining step”); c3-ii) culturing the final AICD-resistant immune cell population for about 6 days (“third recovery step”); and d) comparing sequence counts of gRNA (e.g., sgRNA) or sgRNAs^{iBAR} or sequences comprising hit gene mutations obtained from the final AICD-resistant immune cell population with sequence counts of gRNA (e.g., sgRNA) or sgRNAs^{iBAR} or sequences comprising hit gene mutations obtained from a control immune cell population, wherein the hit genes whose corresponding gRNA (e.g., sgRNA) or sgRNAs^{iBAR} guide sequences or sequences comprising the hit gene mutations are identified as enriched in the final AICD-resistant immune cell population compared to the control immune cell population with an FDR≤0.2 (and/or with at least about 2-fold enrichment) are identified as target genes whose mutations increase resistance to AICD (AICD resistant genes). In some embodiments, the control immune cell population a same immune cell library cultured under the same condition (e.g., at 37° C., 5% CO₂, same duration as the test immune cell library) and not contacted with any FasL. In some embodiments, the method further comprises culturing a same immune cell library under the same condition and not contacting with any FasL. In some embodiments, the immune cell library has about 100-fold to about 2000-fold coverage for each sgRNA^{iBAR}, such as about 1000-fold coverage for each sgRNA^{iBAR}. In some embodiments, the immune cell library has at least about 400-fold coverage for each hit gene, e.g., about 1200-fold to about 12,000-fold coverage for each hit gene. In some embodiments, the gRNA (e.g., sgRNA) or sgRNA^{iBAR} sequence counts are subject to median ratio normalization followed by mean-variance modeling. In some embodiments, the variance of each guide sequence is adjusted based on data consistency among the iBAR sequences in the sgRNA^{iBAR} sequences corresponding to the guide sequence. In some embodiments, the data consistency among the iBAR sequences in the sgRNA^{iBAR} sequences corresponding to each guide sequence is determined based on the direction of the fold change of each iBAR sequence, wherein the variance of the guide sequence is increased if the fold changes of the iBAR sequences are in different directions with respect to each other (e.g., increased vs. reduced, increased vs. unchanged, or reduced vs. unchanged). In some embodiments, the variance of each guide sequence is adjusted based on data consistency among the same gene. In some embodiments, the data consistency among the different guide sequences corresponding to the same hit gene is determined based on the direction of the fold change of each guide sequence, wherein the variance of the guide sequence is increased if the fold changes of the different guide sequences are in different directions with respect to each other (e.g., increased vs. reduced, increased vs. unchanged, or reduced vs. unchanged are all considered as different directions) for the same hit gene.

In some embodiments, any of the identification methods described herein further comprises ranking the identified target genes, wherein the target gene ranking is based on the degree of enrichment of the gRNA (e.g., sgRNA) or sgRNA^{iBAR} guide sequences or hit gene mutation sequences in the AICD-resistant immune cell population compared to the control immune cell population. In some embodiments, the gRNA (e.g., sgRNA) library is an sgRNA^{iBAR} library, and the target gene ranking is further adjusted based on data consistency among the iBAR sequences in the sgRNA^{iBAR} sequences corresponding to the guide sequence of the target gene. In some embodiments, the data consistency among the iBAR sequences in the sgRNA^{iBAR} sequences corresponding to each guide sequence is determined based on the direction of the fold change of each iBAR sequence, wherein the target gene ranking is reduced if the fold changes of the iBAR sequences are in different directions with respect to each other (e.g., increased vs. reduced, increased vs. unchanged, or reduced vs. unchanged). In some embodiments, the target gene ranking is adjusted based on data consistency of gRNA (e.g., sgRNA) or sgRNA^{iBAR} guide sequences or hit gene mutation sequences among the same gene. In some embodiments, the data consistency among the different guide sequences corresponding to the same hit gene is determined based on the direction of the fold change of each guide sequence, wherein the target gene ranking is reduced if the fold changes of the different guide sequences are in different directions with respect to each other (e.g., increased vs. reduced, increased vs. unchanged, or reduced vs. unchanged are all considered as different directions) for the same hit gene. In some embodiments, the data consistency among the different mutation sequences corresponding to the same hit gene is determined based on the direction of the fold change of each mutation sequence, wherein the target gene ranking is reduced if the fold changes of the different mutation sequences are in different directions with respect to each other (e.g., increased vs. reduced, increased vs. unchanged, or reduced vs. unchanged are all considered as different directions) for the same hit gene.

In some embodiments, any of the identification methods described herein further comprises assigning a AICD resistant score to the identified target gene, wherein target genes whose mutations increase resistance to AICD are ranked from high to low based on the fold of enrichment of the sgRNA or sgRNA^{iBAR} guide sequences or hit gene mutation sequences in the AICD-resistant immune cell population compared to the control immune cell population, and each target gene is assigned an AICD resistant score from high to low accordingly.

In some embodiments, any of the identification methods described herein further comprise validating the target gene by: a) modifying an immune cell (e.g., T cell such as CAR-T cell) by creating a mutation (e.g., inactivating mutation) in the target gene in the immune cell; b) determining its resistance to AICD (or FasL-induced apoptosis) relative to a control immune cell.

In some embodiments, the control immune cell is a same immune cell without the mutation in the target gene. In some embodiments, the control immune cell is cultured under the same condition as the immune cell comprising the mutation in the target gene. In some embodiments, the control immune cell is a same immune cell with the same mutation in the same target gene, but not contacted with FasL.

Further provided are modified immune cells obtained by inactivating one or more target genes identified by any of the methods described herein. Further provided are target genes identified by any of the methods described herein. Further provided are gRNAs or sgRNAs for targeting target genes identified by any of the methods described herein.

Guide RNA (e.g., sgRNA) Library and sgRNA^{iBAR} Library

In some embodiments, the present invention uses CRISPR/Cas guide RNAs (e.g., sgRNA) and constructs encoding the CRISPR/Cas guide RNAs to generate mutations (e.g., inactivating mutations) in one or more hit genes. In some embodiments, the mutations are generated by cleaving or base editing the hit gene (e.g., with CRISPR/Cas9). In some embodiments, the mutations are generated by modulating (e.g., repressing or reducing) the expression of the hit gene (e.g., with CRISPR/dCas fused to a repressor domain). Any gRNA (e.g., sgRNA) library or gRNA^{iBAR} library (including nucleic acids, vectors, or viruses encoding thereof, methods of making thereof) described in US20220064633 and WO2022143783 can be used herein.

In some embodiments, there is provided a gRNA (e.g., sgRNA) library comprising one or a plurality of (e.g., 1, 2, 3, 4, 5, 10, 100, 1,000, 10,000, 20,000, or more) gRNA constructs, wherein each gRNA construct (e.g., lentivirus or lentiviral vector encoding the sgRNA) comprises or encodes a gRNA, and wherein each gRNA comprises a guide sequence that is complementary (e.g., at least about any of 50%, 60%, 70%, 80%, 90%, 95%, 96%, 97%, 98%, 99%, or 100% complementary) to a target site in a corresponding hit gene. In some embodiments, the gRNA (e.g., sgRNA) library comprises a plurality of (e.g., 2, 3, 4, 5, 10, 100, 1,000, 2,000, 10,000, 20,000, or more) gRNA constructs, wherein at least two hit genes that the guide sequences are complementary to are different from each other. In some embodiments, the gRNA (e.g., sgRNA) construct comprises (or consists of) a gRNA. In some embodiments, the gRNA (e.g., sgRNA) construct encodes a gRNA. In some embodiments, the gRNA (e.g., sgRNA) construct is a plasmid that encodes the gRNA. In some embodiments, the gRNA (e.g., sgRNA) construct is a viral vector (e.g., lentiviral vector) encoding the gRNA. In some embodiments, the gRNA (e.g., sgRNA) construct is a virus (e.g., lentivirus) encoding the gRNA. In some embodiments, each gRNA (e.g., sgRNA) comprises the guide sequence fused to a second sequence, wherein the second sequence comprises a repeat-anti-repeat stem loop that interacts with a Cas protein (e.g., Cas9). In some embodiments, each gRNA (e.g., sgRNA) comprises the guide sequence fused to a second sequence, wherein the second sequence comprises a DR that interacts with a Cas protein (e.g., Cas9). In some embodiments, the second sequence of each gRNA (e.g., sgRNA) further comprises a stem loop 1, a stem loop 2, and/or a stem loop 3. In some embodiments, each guide sequence comprises about 17 to about 23 nucleotides. In some embodiments, the gRNA (e.g., sgRNA) library comprises at least about 100 gRNA constructs, such as at least about any of 200, 300, 400, 1,000, 1,600, 2,000, 4,000, 10,000, 15,000, 16,000, 19,000, 20,000, 38,000, 50,000, 100,000, 150,000, 155,000, 200,000, or more gRNA constructs. In some embodiments, the gRNA (e.g., sgRNA) library comprises about 500 to about 16,000 gRNA constructs. In some embodiments, the gRNA (e.g., sgRNA) library comprises about 2,000 to about 12,000 gRNA constructs. In some embodiments, the gRNA (e.g., sgRNA) library comprising a plurality of gRNA constructs comprises or encodes gRNAs (e.g., sgRNAs) with guide sequences complementary to target sites of every annotated gene in the genome (hereinafter also referred to as “whole-genome sgRNA library”). In some embodiments, the gRNA (e.g., sgRNA) library comprises at least two (e.g., 2, 3, 4, 5, or more, such as 3) gRNA constructs comprising or encoding gRNAs (e.g., sgRNAs) with guide sequences complementary to at least two (e.g., 2, 3, 4, 5, or more, such as 3) different target sites of the same hit gene, i.e., the gRNA library has at least two-fold coverage for that hit gene. In some embodiments, for each hit gene, the gRNA (e.g., sgRNA) library comprises at least 3 (e.g., about 6 to about 12) gRNA constructs comprising or encoding gRNAs with guide sequences complementary to at least 3 (e.g., about 6 to about 12) different target sites of the same hit gene. In some embodiments, the gRNA (e.g., sgRNA) library comprises at least two (e.g., 2, 3, 4, 5, or more, such as 3) gRNA constructs comprising or encoding gRNAs with guide sequences complementary to at least two (e.g., 2, 3, 4, 5, or more, such as 3) different target sites within the same hit gene for every annotated gene in the genome, i.e., the gRNA (e.g., sgRNA) library has at least two-fold coverage for the whole genome. In some embodiments, the gRNA (e.g., sgRNA) library further comprises one or a plurality of (e.g., 1, 2, 3, 4, 5, 10, 100, 1,000, 2,000, 10,000, or more) “negative control gRNA (e.g., sgRNA) constructs”, wherein each negative control gRNA construct (e.g., lentivirus or lentiviral vector encoding the negative control gRNA) comprises or encodes a negative control gRNA, and wherein each negative control gRNA comprises a guide sequence that is complementary to an irrelevant sequence that is not in the genome, is complementary to a control gene (e.g., known to respond the same or similar between test and control groups after gene inactivation), or is complementary to a sequence not associated with any annotated gene in the genome. In some embodiments, the gRNA (e.g., sgRNA) library further comprises negative control gRNA constructs in the amount of about 0.3% to about 30% of the number of hit gene gRNA constructs in the gRNA library. In some embodiments, the gRNA (e.g., sgRNA) library further comprises about 250 to about 4000 (e.g., about 1000) negative control gRNA constructs.

In some embodiments, the sgRNA further comprises an internal barcode (iBAR) sequence (such sgRNA is hereinafter referred to as “sgRNA^{iBAR}”). In some embodiments, the iBAR is positioned in the sgRNA such that the resulting sgRNA^{iBAR} is operable with a Cas protein (e.g., Cas9) to modify (e.g., cleave or modulate expression) the hit gene complementary to the guide sequence of the sgRNA^{iBAR}. In some embodiments, each sgRNA^{iBAR} comprises in the 5′-to-3′ direction a first stem sequence and a second stem sequence, wherein the first stem sequence hybridizes with the second stem sequence to form a double-stranded RNA (dsRNA) region that interacts with the Cas protein, and wherein the iBAR sequence is disposed between the 3′ end of the first stem sequence and the 5′ end of the second stem sequence. In some embodiments, each sgRNA^{iBAR} comprises the guide sequence fused to a second sequence, wherein the second sequence comprises a repeat-anti-repeat stem loop that interacts with the Cas protein (e.g., Cas9). In some embodiments, the second sequence of each sgRNA^{iBAR} further comprises a stem loop 1, a stem loop 2, and/or a stem loop 3. In some embodiments, the Cas protein is Cas9, and the iBAR sequence of each sgRNA^{iBAR} is inserted in the loop region of the repeat-anti-repeat stem loop. In some embodiments, each sgRNA^{iBAR} comprises from 5′-to-3′: a guide sequence, a repeat-anti-repeat stem loop with iBAR sequence inserted in the loop region, a stem loop 1, a stem loop 2, and a stem loop 3. In some embodiments, there is provided an sgRNA^{iBAR} library comprising a plurality of sets of sgRNA^{iBAR} constructs, wherein each set of sgRNA^{iBAR} constructs comprise three or more (e.g., 3, 4, 5, or more, such as 4) sgRNA^{iBAR} constructs (e.g., lentivirus or lentiviral vector encoding the sgRNAs^{iBAR}) each comprising or encoding an sgRNA^{iBAR}, wherein each sgRNA^{iBAR} comprises a guide sequence and an iBAR sequence, wherein the guide sequences for the three or more sgRNA^{iBAR} constructs are the same, wherein the iBAR sequence for each of the three or more sgRNA^{iBAR} constructs is different from each other, and wherein the guide sequence of each set of sgRNA^{iBAR} constructs is complementary (e.g., at least about any of 50%, 60%, 70%, 80%, 90%, 95%, 96%, 97%, 98%, 99%, or 100% complementary) to a different target site in a corresponding hit gene (e.g., different hit genes, or different sites within the same hit gene). In some embodiments, each set of sgRNA^{iBAR} constructs comprises four sgRNA^{iBAR} constructs, and wherein the iBAR sequence for each of the four sgRNA^{iBAR} constructs is different from each other. Hence in some embodiments, there is provided an sgRNA^{iBAR} library comprising a plurality of sets of sgRNA^{iBAR} constructs, wherein each set of sgRNA^{iBAR} constructs comprise four sgRNA^{iBAR} constructs each comprising or encoding an sgRNA^{iBAR}, wherein each sgRNA^{iBAR} comprises a guide sequence and an iBAR sequence, wherein the guide sequences for the four sgRNA^{iBAR} constructs are the same, wherein the iBAR sequence for each of the four sgRNA^{iBAR} constructs is different from each other, and wherein the guide sequence of each set of sgRNA^{iBAR} constructs is complementary (e.g., at least about any of 50%, 60%, 70%, 80%, 90%, 95%, 96%, 97%, 98%, 99%, or 100% complementary) to a different target site in a corresponding hit gene (e.g., different hit genes, or different sites within the same hit gene). In some embodiments, the sgRNA^{iBAR} library comprises at least about 100 (e.g., at least about any of 200, 400, 1,000, 1,300, 1,600, 4,000, 10,000, 15,000, 19,000, 20,000, 38,000, 50,000, 100,000, 150,000, 155,000, 200,000, or more) sets of sgRNA^{iBAR} constructs, such as about 1000 to about 65,000 (e.g., 57,342) sets of sgRNA^{iBAR} constructs. In some embodiments, the iBAR sequences for at least two sgRNA^{iBAR} constructs among different sets of sgRNA^{iBAR} constructs are the same (e.g., the first set and the second set of sgRNA^{iBAR} constructs have at least 1, 2, 3, 4, or more shared iBAR sequences among the two sets of sgRNA^{iBAR} constructs). In some embodiments, the iBAR sequences for at least two sets of sgRNA^{iBAR} constructs are the same. In some embodiments, the sgRNA^{iBAR} library comprising a plurality of sets sgRNA^{iBAR} constructs comprises or encodes sgRNAs^{iBAR} with guide sequences complementary to target sites of every annotated gene in the genome (hereinafter also referred to as “whole-genome sgRNA^{iBAR} library”). In some embodiments, the hit gene encodes a protein that is expressed within a cell or on cell surface. In some embodiments, the sgRNA^{iBAR} library comprises at least two (e.g., 2, 3, 4, 5, or more, such as 3) sets sgRNA^{iBAR} constructs comprising or encoding sgRNAs^{iBAR} with guide sequences complementary to at least two (e.g., 2, 3, 4, 5, or more, such as 3) different target sites of the same hit gene, i.e., the sgRNA^{iBAR} library has at least two-fold coverage for that hit gene. In some embodiments, for each hit gene, the sgRNA^{iBAR} library comprises 3 sets sgRNA^{iBAR} constructs comprising or encoding sgRNAs^{iBAR} with guide sequences complementary to 3 different target sites of the same hit gene. In some embodiments, the sgRNA^{iBAR} library comprises at least two (e.g., 2, 3, 4, 5, or more, such as 3) sets sgRNA^{iBAR} constructs comprising or encoding sgRNAs^{iBAR} with guide sequences complementary to at least two (e.g., 2, 3, 4, 5, or more, such as 3) different target sites within the same hit gene for every annotated gene in the genome, i.e., the sgRNA^{iBAR} library has at least two-fold coverage for the whole genome. In some embodiments, each guide sequence comprises about 17 to about 23 nucleotides. In some embodiments, each iBAR sequence comprises about 1 to about 50 (e.g., about 6) nucleotides. In some embodiments, the sgRNA^{iBAR} construct comprises (or consists of) an sgRNA^{iBAR}. In some embodiments, the sgRNA^{iBAR} construct encodes an sgRNA^{iBAR}. In some embodiments, the sgRNA^{iBAR} construct is a plasmid that encodes the sgRNA^{iBAR}. In some embodiments, the sgRNA^{iBAR} construct is a viral vector (e.g., lentiviral vector) encoding the sgRNA^{iBAR}. In some embodiments, the sgRNA^{iBAR} construct is a virus (e.g., lentivirus) encoding the sgRNA^{iBAR}. Different sgRNA^{iBAR} constructs of a set having different iBAR sequences can be used in a single gene-editing and screening experiment to provide replicate data. In some embodiments, the sgRNA^{iBAR} library further comprises one or a plurality of sets of “negative control sgRNA^{iBAR} constructs”, wherein each set of negative control sgRNA^{iBAR} constructs comprise three or more (e.g., 3, 4, 5, or more, such as 4) negative control sgRNA^{iBAR} constructs (e.g., lentivirus or lentiviral vector encoding the negative control sgRNAs^{iBAR}) each comprising or encoding a negative control sgRNA^{iBAR}, wherein each negative control sgRNA^{iBAR} comprises a guide sequence and an iBAR sequence, wherein the guide sequences for the three or more negative control sgRNA^{iBAR} constructs are the same, wherein the iBAR sequence for each of the three or more negative control sgRNA^{iBAR} constructs is different from each other, and wherein the guide sequence of each set of negative control sgRNA^{iBAR} constructs is complementary to a target site not associated with any annotated gene in the genome, is complementary to a control gene (e.g., known to respond the same or similar between test and control groups after gene inactivation), or is complementary to an irrelevant sequence that is not in the genome. In some embodiments, the sgRNA^{iBAR} library further comprises negative control sgRNA^{iBAR} constructs in the amount of about 0.3% to about 30% of the number of hit gene sgRNA^{iBAR} constructs in the sgRNA^{iBAR} library. In some embodiments, the sgRNA^{iBAR} library further comprises about 200 to about 20,000 negative control sgRNA^{iBAR} constructs (e.g., 1000) or sets of negative control sgRNA^{iBAR} constructs (e.g., 250 sets).

In some embodiments, there is provided a gRNA library (e.g., sgRNA or sgRNA^{iBAR} library) comprising one or more gRNA constructs (e.g., sgRNA or sgRNA^{iBAR} constructs), wherein each gRNA construct (e.g., lentivirus or lentiviral vector encoding the gRNA) comprises or encodes a gRNA (e.g., sgRNA or sgRNA^{iBAR}), and wherein each gRNA comprises a guide sequence that is complementary (e.g., at least about any of 50%, 60%, 70%, 80%, 90%, 95%, 96%, 97%, 98%, 99%, or 100% complementary) to a target site in a target gene selected from the group consisting of SPPL3, FADD, FAS, CASP8, ARID1A, BAK1, BID, ETS1, IKZF2, and HIST1H1B.

In some embodiments, the gRNA (e.g., sgRNA) library or sgRNA^{iBAR} library comprises at least about any of 1, 2, 3, 4, 5, 10, 20, 50, 100, 200, 400, 500, 1,000, 2,000, 3,000, 4,000, 5,000, 10,000, 15,000, 19,000, 20,000, 38,000, 39,000, 40,000, 50,000, 100,000, 150,000, 155,000, 200,000, 250,000 or more gRNA constructs or sgRNA^{iBAR} constructs. In some embodiments, the sgRNA library comprises about 1000 to about 300,000 sgRNA constructs. In some embodiments, the sgRNA^{iBAR} library comprises about 1000 to about 500,000 sgRNA^{iBAR} constructs. In some embodiments, the sgRNA^{iBAR} library comprises at least about any of 1, 2, 3, 4, 5, 10, 20, 50, 100, 200, 400, 500, 1,000, 2,000, 3,000, 5,000, 10,000, 15,000, 19,000, 20,000, 38,000, 50,000, 65,000, 100,000, 150,000, 200,000 or more sets of sgRNA^{iBAR} constructs. In some embodiments, the gRNA (e.g., sgRNA) library or the sgRNA^{iBAR} library targets at least about any of 1, 2, 3, 4, 5, 10, 20, 50, 100, 200, 500, 1,000, 2,000, 5,000, 10,000, 15,000, 19,000, 20,000, 38,000, 50,000 or more genes in a cell or an organism. In some embodiments, the organism is human. In some embodiments, the gRNA (e.g., sgRNA) library or the sgRNA^{iBAR} library is a whole-genome library for protein-coding genes and/or non-coding RNAs. In some embodiments, the gRNA (e.g., sgRNA) library or the sgRNA^{iBAR} library is a whole-genome library for every annotated gene. In some embodiments, the sgRNA library or the sgRNA^{iBAR} library targets at least about any of 0.1%, 0.5%, 1%, 2%, 3%, 4%, 5%, 6%, 7%, 8%, 9%, 10%, 20%, 30%, 40%, 50%, 60%, 70%, 80%, 90%, or 95% of the genes in a cell or an organism. In some embodiments, the sgRNA library or the sgRNA^{iBAR} library is a targeted library, which targets selected genes in a signaling pathway or associated with a cellular process, such as sensitivity to AICD, GvHD, HvG, immune checkpoint molecule(s), cell proliferation, differentiation, maturation, activation, persistence, homeostasis, effector function, cell cycle, transcriptional regulation, ubiquitination, apoptosis, immune response such as autoimmune, tumor metastasis, tumor malignant transformation, etc. In some embodiments, the gRNA (e.g., sgRNA) library or the sgRNA^{iBAR} library is used for a genome-wide screen associated with a particular modulated phenotype, such as high resistance to AICD or high persistence in vivo. In some embodiments, the gRNA (e.g., sgRNA) library or the sgRNA^{iBAR} library targets genes whose encoded mRNA and/or protein express within cells (in heathy cells or in disease cells). In some embodiments, the gRNA (e.g., sgRNA) library or the sgRNA^{iBAR} library targets genes whose encoded protein express on the cell surface. In some embodiments, the gRNA (e.g., sgRNA) library or the sgRNA^{iBAR} library is designed to target an eukaryotic genome, such as a mammalian genome. Exemplary genomes of interest include genomes of a rodent (mouse, rat, hamster, guinea pig), a domesticated animal (e.g., cow, sheep, cat, dog, horse, or rabbit), a non-human primate (e.g., monkey), fish (e.g., zebrafish), non-vertebrate (e.g., Drosophila melanogaster and Caenorhabditis elegans), and human.

The exact number of sgRNA constructs in an sgRNA library, or the exact number of sgRNA^{iBAR} constructs (or sets of sgRNA^{iBAR} constructs) in an sgRNA^{iBAR} library, can depend on whether the screen 1) targets genes or regulatory elements, 2) targets the complete genome, or subgroup of the genomic genes.

In some embodiments, the gRNA (e.g., sgRNA) library comprises one or more control gRNA constructs that do not target any genomic loci in a genome. In some embodiments, gRNA (e.g., sgRNA) constructs that do not target putative genomic genes can be included in a gRNA library as negative controls. In some embodiments, the sgRNA^{iBAR} library comprises one or more control sgRNA^{iBAR} constructs that do not target any genomic loci in a genome. In some embodiments, sgRNA^{iBAR} constructs that do not target putative genomic genes can be included in an sgRNA^{iBAR} library as negative controls.

The sgRNA constructs and libraries described herein may be prepared using any known nucleic acid synthesis and/or molecular cloning methods in the art. In some embodiments, the gRNA (e.g., sgRNA) library is synthesized by electrochemical means on arrays (e.g., CustomArray, Twist, Gen9), DNA printing (e.g., Agilent), or solid phase synthesis of individual oligos (e.g., by IDT). The sgRNA constructs can be amplified by PCR and cloned into an expression vector (e.g., a lentiviral vector). In some embodiments, the lentiviral vector further encodes one or more components of the CRISPR/Cas-based genetic editing system, such as the Cas protein, e.g., Cas9.

The present invention in some embodiments provides isolated nucleic acids encoding any of the gRNA (e.g., sgRNA) constructs, sgRNA^{iBAR} constructs, sets of sgRNA^{iBAR} constructs, gRNA (e.g., sgRNA) library, or sgRNA^{iBAR} library described herein. Also provided are vectors (e.g., non-viral vector, or viral vector such as lentiviral vector) and virus (e.g., lentivirus) comprising any of the nucleic acids encoding any of the gRNA (e.g., sgRNA) constructs, sgRNA^{iBAR} constructs, sets of sgRNA^{iBAR} constructs, gRNA (e.g., sgRNA) library, or sgRNA^{iBAR} library described herein. Also provided are gRNA or sgRNA molecules encoded by any one of the gRNA or sgRNA constructs or libraries described herein. Also provided are sgRNA^{iBAR} molecules encoded by any one of the sgRNA^{iBAR} constructs, sets, or libraries described herein. Compositions and kits comprising any one of the gRNA, sgRNA, or sgRNA^{iBAR} constructs, molecules, sets, or libraries are further provided.

In some embodiments, there is provided a modified immune cell comprising any one of the gRNA or sgRNA or sgRNA^{iBAR} constructs, molecules, sets, or libraries described herein. In some embodiments, there is provided an immune cell library wherein each immune cell comprises one or more gRNA (e.g., sgRNA) constructs from an gRNA library described herein, or one or more sgRNA^{iBAR} constructs from an sgRNA^{iBAR} library described herein. In some embodiments, the modified immune cells or the initial population of immune cells comprise or express one or more components of the CRISPR/Cas system, such as the Cas protein (e.g., Cas9) operable with the gRNA (e.g., sgRNA) or sgRNA^{iBAR} constructs.

iBAR Sequences

A set of sgRNA^{iBAR} construct comprises three or more sgRNA^{iBAR} constructs each comprising a different iBAR sequence.

The iBAR sequences may have any suitable length. In some embodiments, each iBAR sequence is about 1-50 nucleotides (“nt”) in length, such as about any one of Int-40 nt, Int-30 nt, Int-20 nt, 2 nt-20 nt, 3 nt-18 nt, 3 nt-16 nt, 3 nt-14 nt, 3 nt-12 nt, 3 nt-10 nt, 3 nt-9 nt, 3 nt-8 nt, 4 nt-8 nt, or 5 nt-7 nt. In some embodiments, each iBAR sequence is about any of 2 nt, 3 nt, 4 nt, 5 nt, 6 nt, 7 nt, or 8 nt long. The iBAR sequence in each sgRNA^{iBAR} construct can have the same or different lengths. The iBAR sequences within a set of sgRNA^{iBAR} constructs can have the same or different lengths. In some embodiments, the iBAR sequences within one set of sgRNA^{iBAR} constructs have different lengths from the iBAR sequences within another set of sgRNA^{iBAR} constructs. In some embodiments, the iBAR sequence is about 6 nt, hereinafter referred to as “iBAR₆.” In some embodiments, each iBAR sequence within the sgRNA^{iBAR} library is about 6 nt.

The iBAR sequences may have any suitable sequences. In some embodiments, the iBAR sequence is a DNA sequence made of any of A, T, C and/or G nucleotides. In some embodiments, the iBAR sequence is an RNA sequence made of any of A, U, C, and/or G nucleotides. In some embodiments, the iBAR sequence has non-conventional or modified nucleotides other than A, T/U, C, and G. In some embodiments, each iBAR sequence is 6 nucleotides long consisting of A, T, C, and G nucleotides. In some embodiments, the iBAR sequence in the encoded sgRNA^{iBAR} is 6 nucleotides long consisting of A, U, C, and G nucleotides.

The iBAR strategy with a streamlined analytic tool (MAGeCK^{iBAR}; Zhu et al., Genome Biol. 2019; 20:20) described herein can facilitate large-scale CRISPR/Cas screens for biomedical discoveries in various settings.

The iBAR sequence may be inserted (including appended) to any suitable regions in a guide RNA (e.g., sgRNA) that does not affect the efficiency of the gRNA in guiding the Cas nuclease (e.g., Cas9) to its target site. The iBAR sequence may also be inserted (including appended) to any suitable regions in a crRNA or a tracrRNA that does not affect the efficiency of the crRNA:tracrRNA duplex or crRNA in guiding the Cas nuclease (e.g., Cas9) to its target site. In some embodiments, the iBAR sequence is placed at the 5′ end, internal, and/or 3′ end of a gRNA (e.g., sgRNA, crRNA, tracrRNA). For example, an sgRNA may comprise various stem loops that interact with the Cas nuclease in a CRISPR complex, and the iBAR sequence may be embedded in the loop region of any one of the stem loops. In some embodiments, each sgRNA^{iBAR} sequence comprises in the 5′-to-3′ direction a first stem sequence and a second stem sequence, wherein the first stem sequence hybridizes with the second stem sequence to form a double-stranded RNA (dsRNA) region that interacts with the Cas protein, and wherein the iBAR sequence is disposed between the 3′ end of the first stem sequence and the 5′ end of the second stem sequence. In some embodiments, the guide RNA (e.g., sgRNA) further comprises a stem loop 1, a stem loop 2, and/or a stem loop 3, and wherein the iBAR sequence is inserted in the loop region of stem loop 1, stem loop 2, and/or stem loop 3.

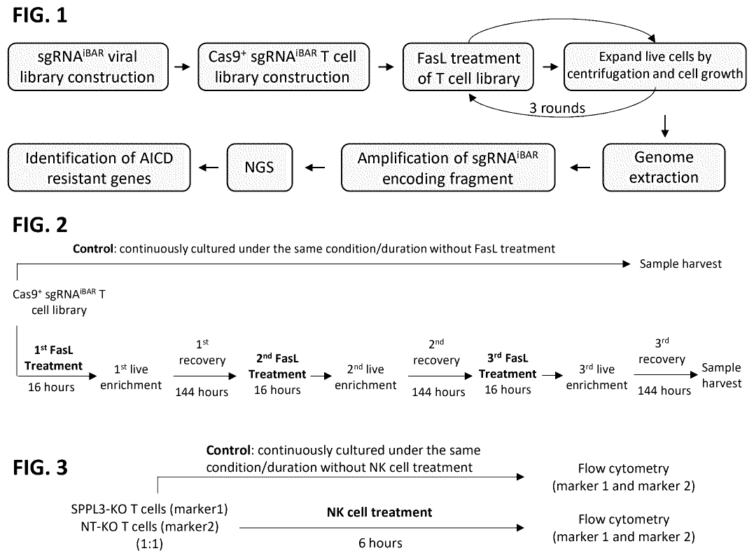

In some embodiments, the iBAR sequence is inserted in the loop region of a crRNA (e.g., loop of the direct repeat sequence) or a tracrRNA. In some embodiments, the iBAR sequence is inserted in the tetraloop, or the loop region of the repeat:anti-repeat stem loop of an sgRNA. In some embodiments, the iBAR sequence of each sgRNA^{iBAR} within the library is inserted in the loop region of the repeat-anti-repeat stem loop. The tetraloop of the Cas9 sgRNA scaffold is outside the Cas9-sgRNA ribonucleoprotein complex, which has been subject to alterations for various purposes without affecting the activity of its upstream guide sequence (Gilbert et al. Cell 159, 647-661 (2014); Zhu et al. Methods Mol Biol 1656, 175-181 (2017)). Applicant has previously demonstrated in WO2020125762 that a 6-nt-long iBAR (iBAR₆) may be embedded in the tetraloop of a typical Cas9 sgRNA scaffold without affecting the gene editing efficiency of the sgRNA or increasing off-target effects, and without sequence bias in the iBAR₆. The exemplary iBAR₆ gives rise to 4,096 barcode combinations, which provides sufficient variations for a high throughput screen (see FIG. 1A of WO2020125762).

### Guide Sequences

In some embodiments, the guide RNA (e.g., sgRNA) comprises a crRNA comprising a guide sequence (also known as a spacer sequence) capable of hybridizing to a target sequence (protospacer) in a target nucleic acid. The guide sequence hybridizes with the target sequence (e.g., a target site in a hit gene) and directs sequence-specific binding of a CRISPR complex to the target sequence. In some embodiments, the degree of complementarity between a guide sequence and its corresponding target sequence, when optimally aligned using a suitable alignment algorithm, is about or more than about any of 50%, 60%, 70%, 75%, 80%, 85%, 90%, 91%, 92%, 93%, 94%, 95%, 96%, 97%, 98%, 99%, or more (e.g., 100% complementary). A guide sequence that is “complementary” to a target site or a hit gene can be fully or partially complementary (e.g., at least about any of 50%, 60%, 70%, 80%, 90%, 95%, 96%, 97%, 98%, 99%, or 100% complementary) to the target site or the hit gene. Optimal alignment may be determined with the use of any suitable algorithm for aligning sequences, non-limiting example of which include the Smith-Waterman algorithm, the Needleman-Wimsch algorithm, algorithms based on the Burrows-Wheeler Transform. In certain embodiments, a guide sequence is about or more than about any of 10, 11, 12, 13, 14, 15, 16, 17, 18, 19, 20, 21, 22, 23, 24, 25, 26, 27, 28, 29, 30 or more nucleotides in length, such as about 17 to about 23 nucleotides. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions.

Synthetic guide sequences can be about 20 nucleotides long, but can be longer or shorter. By way of example, a guide sequence for a CRISPR/Cas9 system may consist of 20 nucleotides complementary to a target sequence (e.g., a target site in a hit gene), i.e., the guide sequence may be identical to the 20 nucleotides upstream of the PAM sequence except for the A/U difference between DNA and RNA. In some embodiments, the guide sequence comprises about 17 to about 23 nucleotides. The guide sequence of each gRNA (e.g., sgRNA) or sgRNA^{iBAR} within the library can have the same or different lengths. The guide sequences within a set of sgRNA^{iBAR} constructs can have the same or different lengths. In some embodiments, the guide sequences within one set of sgRNA^{iBAR} constructs have different lengths from the guide sequences within another set of sgRNA^{iBAR} constructs.

In some embodiments, the guide sequences within a set of sgRNA^{iBAR} constructs are the same. In some embodiments, the guide sequences within a set of sgRNA^{iBAR} constructs are the same, while the guide sequence within each set of sgRNA^{iBAR} constructs is complementary to a different target site (e.g., different hit genes, or different target sites of the same hit gene). In some embodiments, the guide sequences of at least two sets of sgRNA^{iBAR} Constructs are complementary to two different target sites of the same hit gene. In some embodiments, the guide sequences of 3 sets of sgRNA^{iBAR} constructs are complementary to 3 different target sites of the same hit gene. In some embodiments, each hit gene is targeted by at least two (e.g., 2, 3, 4 or more, such as 3) guide sequences of at least two (e.g., 2, 3, 4 or more, such as 3) sets of sgRNA^{iBAR} constructs in at least two (e.g., 2, 3, 4 or more, such as 3) different target sites. In some embodiments, the guide sequence within each set of sgRNA^{iBAR} constructs is complementary to a different hit gene in the genome.

The guide sequence in an sgRNA construct or an sgRNA^{iBAR} construct may be designed according to any known methods in the art, or any known algorithms that identify CRISPR/Cas target sites in user-defined lists with a high degree of targeting specificity in the human genome, such as Genomic Target Scan (GT-Scan) (see O'Brien et al., Bioinformatics (2014) 30:2673-2675)), DeepCRISPR, CasFinder, CHOPCHOP, CRISPRscan, etc. The guide sequence may target the coding region such as an exon or a splicing site, the 5′ untranslated region (UTR) or the 3′ untranslated region (UTR) of a gene of interest. For example, the reading frame of a gene could be disrupted by indels mediated by double-strand breaks (DSB) at a target site of a guide RNA. Alternatively, a guide RNA targeting the 5′ end of a coding sequence may be used to produce gene knockouts with high efficiency. The guide sequence may be designed and optimized according to certain sequence features for high on-target gene-editing activity and low off-target effects. For instance, the GC content of a guide sequence may be in the range of about 20% to about 70%, and sequences containing homopolymer stretches (e.g., TTTT, GGGG) may be avoided.

The guide sequence may be designed to target any genomic locus of interest (e.g., any target site of any hit gene). In some embodiments, the guide sequence targets a protein-coding gene. In some embodiments, the guide sequence targets a gene encoding an RNA, such as a small RNA (e.g., microRNA, piRNA, siRNA, snoRNA, tRNA, rRNA and snRNA), a ribosomal RNA, or a long non-coding RNA (lincRNA). In some embodiments, the guide sequence targets a non-coding region of the genome. In some embodiments, the guide sequence targets a chromosomal locus. In some embodiments, the guide sequence targets an extrachromosomal locus. In some embodiments, the guide sequence targets a mitochondrial gene. In some embodiments, the guide sequence is complementary to a target site of any annotated genes in the genome (e.g., human genome). In some embodiments, the guide sequence targets a gene whose encoded protein is expressed within a cell or on cell surface. In some embodiments, the guide sequence targets a region without any gene annotation in the genome (“non-gene region”). gRNA (e.g., sgRNA) or sgRNA^{iBAR} constructs comprising or encoding such guide sequence complementary to a non-gene region can serve as negative control.

In some embodiments, the guide sequence is designed to repress or inactivate the expression of any hit gene or target gene of interest. The hit gene or target gene may be an endogenous gene or a transgene. In some embodiments, the hit gene or target gene may be known to be associated with a particular phenotype. In some embodiments, the hit gene or target gene is a gene that has not been implicated in a particular phenotype, such as a known gene that is not known to be associated with a particular phenotype, or an unknown gene that has not been characterized. In some embodiments, the guide sequence targeted region is located on a different chromosome as the hit gene or target gene.

Other gRNA, sgRNA, or sgRNA^{iBAR} Components

In some embodiments, the gRNA, sgRNA, or sgRNA^{iBAR} comprises additional sequence element(s) that promotes formation of the CRISPR complex with the Cas protein. In some embodiments, the gRNA (e.g., sgRNA) or sgRNA^{iBAR} comprises a second sequence comprising a repeat-anti-repeat stem loop. A repeat-anti-repeat stem loop comprises a tracr mate sequence fused to a tracr sequence that is complementary to the tracr mate sequence via a loop region.

In some embodiments, the gRNA (e.g., sgRNA) comprises a crRNA comprising a direct repeat (DR) sequence capable of interacting with the Cas protein. In some embodiments, the gRNA (e.g., sgRNA) comprises a crRNA and a tracrRNA, which can be on the same RNA strand (e.g., forming an sgRNA), or on two RNA strands.

The DR sequence may be derived from the DR sequence naturally associated with the corresponding Cas protein. In some embodiments, the DR sequence is at the 5′ end of the spacer sequence. In some embodiments, the DR sequence is at the 3′ end of the spacer sequence. In some embodiments, the DR sequence comprises one or more mutations with respect to a reference (e.g., wildtype) DR sequence, such as comprising one or more mutations of a 5′ and/or 3′ extension, a 5′ and/or 3′ truncation, a nucleotide insertion, a nucleotide deletion, a nucleotide substitution, or combinations thereof, as compared with a reference DR sequence.

In some embodiments, the DR sequence contains a repeat sequence and an anti-repeat sequence. In some embodiments, a repeat sequence of the DR sequence is partially or completely substantially complementary to and hybridizes with an anti-repeat sequence of the DR sequence to form a repeat:anti-repeat duplex (also known as “stem”). In some embodiments, the repeat: anti-repeat duplex is about 6 to about 20 nucleotides in length, such as about any of 6, 8, 10, 12, 14, 16, 18, 20 nucleotides, or longer in length. In some embodiments, the repeat sequence of the DR sequence and the anti-repeat sequence of the DR sequence are linked directly or indirectly via a linker, such as a loop sequence. In the case the repeat and anti-repeat sequences are indirectly linked via a linker, the length of the repeat:anti-repeat duplex does not include the length of the linker.

In some embodiments, the repeat sequence of the DR sequence and the anti-repeat sequence of the DR sequence are linked indirectly via a loop sequence. In some embodiments, the loop sequence is about 4 to about 10 nucleotides in length, e.g., any of about 4, 5, 6, 7, 8, 9, or 10 nucleotides in length. In some embodiments, the loop sequence comprises the sequence of GAAA. In some embodiments, the DR sequence of the gRNA comprises a stem-loop. In some embodiments, the DR sequence comprises one or more stem-loops, such as about 1 to about 5 stem-loops.

Typically, in the context of an endogenous CRISPR/Cas9 system, formation of a CRISPR complex (comprising a guide sequence hybridized to a target sequence and complexed with one or more Cas proteins) results in cleavage of one or both strands in or near (e.g., within 1, 2, 3, 4, 5, 6, 7, 8, 9, 10, 20, 50, or more base pairs from) the target sequence. The tracr sequence, which may comprise or consist of all or a portion of a wild-type tracr sequence (e.g., about or more than about any of 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional. In some embodiments, the tracr sequence has at least about any of 50%, 60%, 70%, 80%, 90%, 95% or 99% of sequence complementarity along the length of the tracr mate sequence when optimally aligned. Determining optimal alignment is within the purview of one of skill in the art. For example, there are publicly and commercially available alignment algorithms and programs such as, but not limited to, ClustalW, Smith-Waterman in Matlab, Bowtie, Geneious, Biopython and SeqMan. In some embodiments, the tracr sequence is about or more than about any of 5, 6, 7, 8, 9, 10, 11, 12, 13, 14, 15, 16, 17, 18, 19, 20, 25, 30, 40, 50, or more nucleotides in length. Any one of the known tracr mate sequences and tracr sequences derived from naturally occurring CRISPR system, such as the tracr mate sequence and tracr sequence from the S. pyogenes CRISPR/Cas9 system as described in U.S. Pat. No. 8,697,359 and those described herein, may be used.

In some embodiments, the tracr sequence and tracr mate sequence are contained within a single transcript, such that hybridization between the two produces a transcript having a secondary structure, such as a stem loop (also known as a hairpin), known as the “repeat-anti-repeat stem loop.”

“Stem-loop” refers to a nucleic acid that has a secondary structure that includes regions of nucleotides known or predicted to pair and form a double-strand duplex (stem portion) that is linked by regions of unpaired, single-stranded nucleotides (loop portions). The terms “hairpin”, “hairpin loop” and “turnback” structures are also used herein to refer to stem-loops. Such structures are well known in the art, and these terms are used in accordance with their commonly known meanings in the art. As is known in the art, stem-loops do not require precise base pairing. Thus, the stem may include one or more base mismatches. Alternatively, base pairing may be exact, i.e., not including any mismatches.

In some embodiments, the loop region of the stem loop in an sgRNA construct without an iBAR sequence is four nucleotides in length, and such loop region is also referred to as the “tetraloop.” In some embodiments, the loop region has the sequence of GAAA. However, longer or shorter loop sequences may be used, or alternative sequences may be used, such as sequences including a nucleotide triplet (for example, AAA), and an additional nucleotide (for example C or G). In some embodiments, the sequence of the loop region is CAAA or AAAG. In some embodiments, the iBAR is inserted in the loop region, such as the tetraloop. For example, the iBAR sequence may be inserted before the first nucleotide, between the first nucleotide or the second nucleotide, between the second nucleotide and the third nucleotide, between the third nucleotide and the fourth nucleotide, or after the fourth nucleotide in the tetraloop. In some embodiments, the iBAR sequence replaces one or more nucleotides in the loop region.

In some embodiments, the sgRNA or sgRNA^{iBAR} comprises at least two or more stem loops, such as 2, 3, 4, or 5 stem loops. In some embodiments, the sgRNA or sgRNA^{iBAR} has at most five hairpins. In some embodiments, the sgRNA or sgRNA^{iBAR} construct further includes a transcription termination sequence, such as a polyT sequence, for example six T nucleotides.

In some embodiments, wherein the Cas protein is Cas9, each sgRNA or sgRNA^{iBAR} comprises a guide sequence fused to a second sequence comprising a repeat-anti-repeat stem loop that interacts with the Cas9. In some embodiments, the iBAR sequence is inserted in the loop region of the repeat-anti-repeat stem loop, or replaces one or more nucleotides in the loop region of the repeat-anti-repeat stem loop. In some embodiments, the second sequence of each sgRNA or sgRNA^{iBAR} further comprises a stem loop 1, stem loop 2, and/or stem loop 3. In some embodiments, the iBAR sequence is inserted in the loop region of stem loop 1, or replaces one or more nucleotides in the loop region of stem loop 1. In some embodiments, the iBAR sequence is inserted in the loop region of stem loop 2, or replaces one or more nucleotides in the loop region of stem loop 2. In some embodiments, the iBAR sequence is inserted in the loop region of stem loop 3, or replaces one or more nucleotides in the loop region of stem loop 3.

In some embodiments, each sgRNA^{iBAR} comprises in the 5′-to-3′ direction a first stem sequence and a second stem sequence, wherein the first stem sequence hybridizes with the second stem sequence to form a double-stranded RNA (dsRNA) region that interacts with the Cas protein, and wherein the iBAR sequence is disposed between the 3′ end of the first stem sequence and the 5′ end of the second stem sequence.

The invariant guide RNA hairpin sequences can be provided according to common knowledge in the art, for example, as disclosed by Nishimasu et al. (Nishimasu H, et al. Crystal structure of Cas9 in complex with guide RNA and target DNA. Cell. 2014; 156:935-949). Any invariant hairpin sequences may be used as long as they are capable of binding to a Cas nuclease once transcribed.

In some embodiments, the sgRNA comprises from 5′ to 3′: a guide sequence, a repeat-anti-repeat stem loop (e.g., with an iBAR sequence inserted in the loop region), a stem loop 1, a stem loop 2, and a stem loop 3.

### Vectors and Vehicles

In some embodiments, the gRNA (e.g., sgRNA) construct comprises one or more regulatory elements operably linked to the guide RNA sequence. In some embodiments, the sgRNA^{iBAR} construct comprises one or more regulatory elements operably linked to the guide RNA sequence and the iBAR sequence. Exemplary regulatory elements include, but are not limited to, promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences).

The gRNA (e.g., sgRNA) or sgRNA^{iBAR} constructs may be present in a vector. In some embodiments, the vector is suitable for replication and integration in eukaryotic cells, such as mammalian cells (e.g., immune cells). In some embodiments, the gRNA (e.g., sgRNA) or sgRNA^{iBAR} construct is an expression vector, such as a viral vector or a plasmid. Examples of viral vectors include, but are not limited to, adenoviral vectors, adeno-associated virus vectors, lentiviral vector, retroviral vectors, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals. It will be appreciated by those skilled in the art that the design of the expression vector can depend on such factors as the choice of the host cell to be transformed, the level of expression desired, etc. In some embodiments, the gRNA (e.g., sgRNA) or sgRNA^{iBAR} construct is a lentiviral vector. In some embodiments, the gRNA (e.g., sgRNA) or sgRNA^{iBAR} construct is a virus. In some embodiments, the gRNA (e.g., sgRNA) or sgRNA^{iBAR} construct is an adenovirus or an adeno-associated virus. In some embodiments, the gRNA (e.g., sgRNA) or sgRNA^{iBAR} construct is a lentivirus. In some embodiments, the vector further comprises a selection marker. In some embodiments, the vector further comprises one or more nucleotide sequences encoding one or more elements of the CRISPR/Cas system, such as a nucleotide sequence encoding a Cas nuclease (e.g., Cas9). In some embodiments, there is provided a vector system comprising one or more vectors encoding nucleotide sequences encoding one or more elements of the CRISPR/Cas system, and a vector comprising any one of the gRNA (e.g., sgRNA) or sgRNA^{iBAR} constructs described herein. A vector may include one or more of the following elements: an origin of replication, one or more regulatory sequences (e.g., promoters and/or enhancers) that regulate the expression of the polypeptide of interest, and/or one or more selectable marker genes (e.g., antibiotic resistance genes, or fluorescent protein-encoding genes).

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. Self-inactivating lentiviral vectors can be packaged into lentiviruses with protocols known in the art. The resulting lentiviruses can be used to transduce a mammalian cell (e.g., immune cell) using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector is a non-viral vector. In some embodiments, the vector is a transposon, such as a Sleeping Beauty transposon system, or a PiggyBac transposon system. In some embodiments, the vector is a polymer-based non-viral vector, including for example, poly (lactic-co-glycolic acid) (PLGA) and poly lactic acid (PLA), poly (ethylene imine) (PEI), and dendrimers. In some embodiments, the vector is a cationic-lipid based non-viral vector, such as cationic liposome, lipid nanoemulsion, and solid lipid nanoparticle (SLN). In some embodiments, the vector is a peptide-based gene non-viral vector, such as poly-L-lysine. Any of the known non-viral vectors suitable for gene editing can be used for introducing the sgRNA or sgRNA^{iBAR}-encoding nucleic acid to an immune cell. See, for example, Yin H. et al. Nature Rev. Genetics (2014) 15:521-555; Aronovich E L et al. “The Sleeping Beauty transposon system: a non-viral vector for gene therapy.” Hum. Mol. Genet. (2011) R1: R14-20; and Zhao S. et al. “PiggyBac transposon vectors: the tools of the human gene editing.” Transl. Lung Cancer Res. (2016) 5(1): 120-125, which are incorporated herein by reference. In some embodiments, any one or more of the nucleic acids encoding the sgRNAs or sgRNAs^{iBAR} described herein is introduced to an immune cell by a physical method, including, but not limited to electroporation, sonoporation, photoporation, magnetofection, hydroporation.

The nucleic acid can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in mammalian cells, and any of the promoters known in the art may be used in the present invention. Promoters may be roughly categorized as constitutive promoters or regulated promoters, such as inducible promoters. Exemplary constitutive promoters include, but are not limited to, cytomegalovirus immediate-early promoter (CMV IE), human elongation factors-1alpha (hEF1α), ubiquitin C promoter (UbiC), phosphoglycerokinase promoter (PGK), simian virus 40 early promoter (SV40), chicken β-Actin promoter coupled with CMV early enhancer (CAGG), a Rous Sarcoma Virus (RSV) promoter, a polyoma enhancer/herpes simplex thymidine kinase (MC1) promoter, a beta actin (β-ACT) promoter, a “myeloproliferative sarcoma virus enhancer, negative control region deleted, d1587rev primer-binding site substituted (MND)” promoter. The inducible promoter can be induced by one or more conditions, such as a physical condition, microenvironment of the immune cells (e.g., engineered immune cells), or the physiological state of the immune cells, an inducer (i.e., an inducing agent), or a combination thereof. In some embodiments, the inducing condition does not induce the expression of endogenous genes in the engineered immune cell, and/or in the subject that receives immune cell therapy. In some embodiments, the inducible promoter can be an NFAT promoter, a TETON® promoter, or an NFκB promoter.

### PAM

In certain embodiments, target sequences or hit genes as described herein are (further) selected based on optimization of one or more of target loci location, target length, target specificity, and protospacer adjacent motif (PAM) characteristics. As used herein, PAM characteristics may comprise for instance PAM sequence, PAM length, and/or PAM nucleotide (A, T, G, and/or C) contents. In certain embodiments, optimizing PAM characteristics comprises optimizing nucleotide content of a PAM. In certain embodiments, optimizing nucleotide content of PAM is selecting a PAM with a motif that maximizes abundance of a nucleotide in the one or more target loci, minimizes mutation frequency, or both. Minimizing mutation frequency can for instance be achieved by selecting PAM sequences devoid of or having low or minimal CpG. The PAM sequence is present in the DNA target sequence but not in the gRNA sequence.

PAM sequences of corresponding CRISPR/Cas systems and identification methods are well known. E.g., see D. Gleditzsch et al., RNA Biol. 2019 April; 16(4):504-517; Liu et al., Microb Cell Fact. 2020; 19(1):172; G. A. Rybnicky et al., “Spacer2PAM: A computational framework to guide experimental determination of functional CRISPR-Cas system PAM sequences,” Nucleic Acids Res. 2022 April; 50(6):3523-3534; the contents of each of which are incorporated herein by reference in their entirety.

Any DNA sequence with the correct target sequence followed by the PAM sequence can be bound by Cas9. The PAM sequence varies by the species of the bacteria from which Cas9 was derived. The most widely used Type II CRISPR system is derived from S. pyogenes and the PAM sequence is NGG located on the immediate 3′ end of the gRNA recognition sequence. The PAM sequences of Type II CRISPR systems from exemplary bacterial species include: Streptococcus pyogenes (NGG), Neisseria meningitidis (NNNNGATT), Streptococcus thermophilus (NNAGAA) and Treponema denticola (NAAAAC).

In some embodiments, the Cas protein as described herein is capable of recognizing a PAM immediately adjacent to a target sequence (e.g., at 5′ or 3′ end of the target sequence) in a target nucleic acid (e.g., hit gene). In some embodiments the PAM recognized by the Cas protein is 3 or 4 nucleotides in length. In some embodiments, engineering of the Cas protein and/or the gRNA increases the number and/or scope of the PAM sequences recognizable by the Cas protein, e.g., by about 1, 2, 3, 4, 5, 10, 50, 100, 1000, or more PAM sites. In some embodiments, engineering of the Cas protein increases the cleavage activity of the Cas protein at a particular PAM site, e.g., by about 5%, 10%, 15%, 20%, 25%, 30%, 35%, 40%, 45%, 50%, or more.

### Cas Protein

The gRNA constructs, sgRNA constructs, sgRNA^{iBAR} constructs described herein may be designed to operate with any one of the naturally-occurring or engineered CRISPR/Cas systems known in the art. In some embodiments, the gRNA construct, the sgRNA construct, or the sgRNA^{iBAR} construct is operable with a Type I CRISPR/Cas system. In some embodiments, the gRNA construct, the sgRNA construct, or the sgRNA^{iBAR} construct is operable with a Type II CRISPR/Cas system. In some embodiments, the gRNA construct, the sgRNA construct, or the sgRNA^{iBAR} construct is operable with a Type III CRISPR/Cas system. In some embodiments, the gRNA construct, the sgRNA construct, or the sgRNA^{iBAR} construct is operable with a Type IV, a Type V, or a Type VI CRISPR/Cas system. Exemplary CRISPR/Cas systems can be found in WO2013176772, WO2014065596, WO2014018423, WO2016011080, U.S. Pat. Nos. 8,697,359, 8,932,814, U.S. Ser. No. 10/113,167B2, the contents of each of which are incorporated herein by reference in their entireties for all purposes.

In certain embodiments, the gRNA (e.g., sgRNA) construct or the sgRNA^{iBAR} construct is operable with a Cas protein derived from a CRISPR/Cas type I, type II, or type III system, which has an RNA-guided polynucleotide binding and/or nuclease activity. Examples of such Cas proteins are recited in, e.g., WO2014144761 WO2014144592, WO2013176772, US20140273226, and US20140273233, which are incorporated herein by reference in their entireties.

In certain embodiments, the Cas protein is derived from a type II CRISPR-Cas system. In certain embodiments, the Cas protein is or is derived from a Cas9 protein. In certain embodiments, the Cas protein is or is derived from a bacterial Cas9 protein, including those identified in WO2014144761. In certain embodiments, the Cas protein is derived from a type VI CRISPR-Cas system. In some embodiments, the Cas protein is Cas13.

In some embodiments, the gRNA (e.g., sgRNA) construct or the sgRNA^{iBAR} construct is operable with Cas9 (also known as Csn1 and Csx12), a homolog thereof, or a modified version thereof. In some embodiments, the sgRNA construct or the sgRNA^{iBAR} construct is operable with two or more (e.g., 2, 3, 4, 5, or more) Cas proteins. In some embodiments, the gRNA (e.g., sgRNA) construct or the sgRNA^{iBAR} construct is operable with a Cas9 protein from S. pyogenes or S. pneumoniae. Cas enzymes are known in the art; for example, the amino acid sequence of S. pyogenes Cas9 protein may be found in the SwissProt database under accession number Q99ZW2.

The Cas protein (also referred herein as “Cas nuclease”) provides a desired activity, such as target binding, target nicking or cleaving activity. In certain embodiments, the desired activity is target binding. In some embodiments, the Cas protein has endonuclease activity. In certain embodiments, the desired activity is target nicking or target cleaving. In certain embodiments, the desired activity also includes a function provided by a polypeptide that is covalently fused to a Cas protein or a nuclease-deficient Cas protein. Examples of such a desired activity include a transcription regulation activity (either activation or repression), an epigenetic modification activity, or a target visualization/identification activity.

In some embodiments, the gRNA (e.g., sgRNA) construct or the sgRNA^{iBAR} construct is operable with a Cas nuclease that cleaves the target sequence, including double-strand cleavage and single-strand cleavage. In some embodiments, the gRNA (e.g., sgRNA) construct or the sgRNA^{iBAR} construct is operable with a catalytically inactive Cas (“dCas”). In some embodiments, the gRNA (e.g., sgRNA) construct or the sgRNA^{iBAR} construct is operable with a dCas of a CRISPR activation (“CRISPRa”) system, wherein the dCas is fused to a transcriptional activator. In some embodiments, the gRNA (e.g., sgRNA) construct or the sgRNA^{iBAR} construct is operable with a dCas of a CRISPR interference (CRISPRi) system. In some embodiments, the dCas is fused to a repressor domain, such as a KRAB domain. Such CRISPR/Cas systems can be used to modulate (e.g., induce, repress, increase, or reduce) gene expression. In some embodiments, the Cas protein is fusion protein comprising i) a dead Cas protein (dCas) and ii) an ABE or adenine deaminase (ADA), or a CBE or cytidine deaminase (CDA), or functional fragment thereof.

In certain embodiments, the Cas protein is a mutant of a wild type Cas protein (such as Cas9) or a fragment thereof. A Cas9 protein generally has at least two nuclease (e.g., DNase) domains. For example, a Cas9 protein can have a RuvC-like nuclease domain and an HNH-like nuclease domain. The RuvC and HNH domains work together to cut both strands in a target site to make a double-stranded break in the target polynucleotide. (Jinek et al., Science 337: 816-21). In certain embodiments, a mutant Cas9 protein is modified to contain only one functional nuclease domain (either a RuvC-like or an HNH-like nuclease domain). For example, in certain embodiments, the mutant Cas9 protein is modified such that one of the nuclease domains is deleted or mutated such that it is no longer functional (i.e., the nuclease activity is absent). In some embodiments where one of the nuclease domains is inactive, the mutant is able to introduce a nick into a double-stranded polynucleotide (such protein is termed a “nickase”) but not able to cleave the double-stranded polynucleotide. In certain embodiments, the Cas protein is modified to increase nucleic acid binding affinity and/or specificity, alter an enzymatic activity, and/or change another property of the protein. In certain embodiments, the Cas protein is truncated or modified to optimize the activity of the effector domain. In certain embodiments, both the RuvC-like nuclease domain and the HNH-like nuclease domain are modified or eliminated such that the mutant Cas9 protein is unable to nick or cleave the target polynucleotide. In certain embodiments, a Cas9 protein that lacks some or all nuclease activity relative to a wild-type counterpart, nevertheless, maintains target recognition activity to a greater or lesser extent.

In certain embodiments, the Cas protein is a fusion protein comprising a naturally-occurring Cas or a variant thereof fused to another polypeptide or an effector domain. The another polypeptide or effector domain may be, for example, a cleavage domain, a transcriptional activation domain, a transcriptional repressor domain, or an epigenetic modification domain. In certain embodiments, the fusion protein comprises a modified or mutated Cas protein in which all the nuclease domains have been inactivated or deleted. In certain embodiments, the RuvC and/or HNH domains of the Cas protein are modified or mutated such that they no longer possess nuclease activity.

In certain embodiments, the effector domain of the fusion protein is a cleavage domain obtained from any endonuclease or exonuclease with desirable properties.

In certain embodiments, the effector domain of the fusion protein is a transcriptional activation domain. In general, a transcriptional activation domain interacts with transcriptional control elements and/or transcriptional regulatory proteins (i.e., transcription factors, RNA polymerases, etc.) to increase and/or activate transcription of a gene. In certain embodiments, the transcriptional activation domain is a herpes simplex virus VP16 activation domain, VP64 (which is a tetrameric derivative of VP16), a NFκB p65 activation domain, p53 activation domains 1 and 2, a CREB (cAMP response element binding protein) activation domain, an E2A activation domain, or an NFAT (nuclear factor of activated T-cells) activation domain. In certain embodiments, the transcriptional activation domain is Gal4, Gcn4, MLL, Rtg3, Gln3, Oaf1, Pip2, Pdr1, Pdr3, Pho4, or Leu3. The transcriptional activation domain may be wild type, or modified or truncated version of the original transcriptional activation domain.

In certain embodiments, the effector domain of the fusion protein is a transcriptional repressor domain, such as inducible cAMP early repressor (ICER) domains, Kruppel-associated box A (KRAB-A) repressor domains, YY1 glycine rich repressor domains, Sp1-like repressors, E(spI) repressors, I. kappa. B repressor, or MeCP2.

In certain embodiments, the effector domain of the fusion protein is an epigenetic modification domain which alters gene expression by modifying the histone structure and/or chromosomal structure, such as a histone acetyltransferase domain, a histone deacetylase domain, a histone methyltransferase domain, a histone demethylase domain, a DNA methyltransferase domain, or a DNA demethylase domain.

In some embodiments, the effector domain of the fusion protein is an adenosine deaminase (ADA), a cytidine deaminase (CDA), or the catalytic domain of ADA or CDA to form a fusion protein capable of single base editing. In some embodiments, the deaminase or catalytic domain thereof is selected from a group consisting of an adenosine deaminase (e.g., TadA, such as, TadA8e, TadA8.17, TadA8.20, TadA9) and a catalytic domain thereof, a cytidine deaminase (e.g., APOBEC, such as, APOBEC3, for example, APOBEC3A, APOBEC3B, APOBEC3C; DddA) and a catalytic domain thereof, a bifunctional adenosine/cytidine deaminase and a catalytic domain thereof, and a functional fragment thereof, and any combination thereof. The cytidine deaminase may include a double-stranded DNA cytidine deaminase, for example, Double-stranded DNA deaminase toxin A (DddA) that catalyzes the deamination of cytidines within dsDNA.

As used herein, the term “adenosine deaminase” or “adenosine deaminase protein” refers to a protein, polypeptide, or one or more functional domains of a protein or polypeptide capable of catalyzing the conversion of adenine to a hypoxanthine by a hydrolytic deamination reaction. In some embodiments, the hypoxanthine-containing molecule is inosine (I). Adenine-containing molecules can be deoxyribonucleic acid (DNA) or ribonucleic acid (RNA).

Adenosine deaminase enzymes that can be used in conjunction with Cas proteins of the disclosure, for example the dCas proteins of the disclosure, include but are not limited to members of the family of enzymes known as RNA-acting adenosine deaminase (ADAR), members of the family of enzymes called adenosine deaminase acting on tRNA (ADAT), and other members of the adenosine deaminase domain-containing family (ADAD). In some embodiments, the adenosine deaminase is capable of targeting adenine in RNA/DNA and RNA duplexes. For example, Zheng et al. (Nucleic Acids Res. 2017, 45(6): 3369-3377) demonstrated that ADAR can perform adenosine to inosine editing reactions on RNA/DNA and RNA/RNA duplexes. In particular embodiments, adenosine deaminase has been modified to increase its ability to edit DNA in an RNA/DNA heteroduplex of an RNA duplex.

In some embodiments, the adenosine deaminase is a human ADAR, including hADAR1, hADAR2, hADAR3. In some embodiments, the adenosine deaminase is a TadA protein, such as E. coli TadA. See Kim et al., Biochemistry 45:6407-6416 (2006); Wolf et al., EMBO J. 21:3841-3851 (2002). TadA may include, but not limited to, TadA8e, TadA8.17, TadA8.20, TadA9.

As used herein, the term “cytidine deaminase” or “cytidine deaminase protein” refers to a protein, polypeptide, or one or more functional domains of a protein or polypeptide capable of catalyzing the conversion of cytosine to uracil by hydrolytic deamination. In some embodiments, the cytosine-containing molecule is cytidine (C), and the uracil-containing molecule is uridine (U). The cytosine-containing molecule may be deoxyribonucleic acid (DNA) or ribonucleic acid (RNA).

The cytidine deaminase enzymes that can be used in conjunction with the present application include, but are not limited to, members of a family of enzymes known as the Apolipoprotein B mRNA Editing Complex (APOBEC) family deaminase, activation-induced deaminase (AID), or cytidine deaminase 1 (CDA1).

In some embodiments, the cytidine deaminase is capable of targeting cytosines in single strands of DNA. In certain embodiments, the cytidine deaminase can edit on a single strand that exists outside the binding component. In some embodiments, the cytidine deaminase is capable of targeting cytosines in double strands of DNA. In some embodiments, the cytidine deaminase can edit at localized vesicles, e.g., localized vesicles formed by target editing sites but directed sequence mismatches. In certain embodiments, the cytidine deaminase may contain mutations that contribute to the focusing activity, such as those mentioned in Kim et al., Nature Biotechnology (2017) 35(4):371-377 (doi:10.1038/nbt.3803).

In some embodiments, the cytidine deaminase is human APOBEC, including hAPOBEC1 or hAPOBEC3. In some embodiments, the cytidine deaminase is a human AID. In some embodiments, the cytidine deaminase is a DddA.

In certain embodiments, the Cas protein further comprises at least one additional domain, such as a nuclear localization signal (NLS), a cell-penetrating or translocation domain, and a marker domain (e.g., a fluorescent protein marker).

The Cas protein can be introduced into immune cells as a (i) Cas protein, or (ii) mRNA encoding the Cas protein, or (iii) a linear or circular DNA encoding the protein. The Cas protein or construct encoding the Cas protein may be purified, or non-purified in a composition. Methods of introducing a protein or nucleic acid construct into a host cell are well known in the art, and are applicable to all methods described herein which requires introduction of a Cas protein or construct thereof to an immune cell. In certain embodiments, the Cas protein is delivered into an immune cell as a protein. In certain embodiments, the Cas protein is constitutively expressed from an mRNA or a DNA in a host cell (e.g., engineered immune cell such as CAR-T). In certain embodiments, the expression of Cas protein from mRNA or DNA is inducible or induced in a host cell. In certain embodiments, a Cas protein can be introduced into a host cell in Cas protein: sgRNA complex using recombinant technology known in the art. Exemplary methods of introducing a Cas protein or construct thereof have been described, e.g., in WO2014144761 WO2014144592 and WO2013176772, which are incorporated herein by reference in their entireties.

In some embodiments, the method uses a CRISPR/Cas9 system. Cas9 is a nuclease from the microbial type II CRISPR (clustered regularly interspaced short palindromic repeats) system, which has been shown to cleave DNA when paired with an sgRNA. The sgRNA directs Cas9 to complementary regions in the target genome gene, which may result in site-specific double-strand breaks (DSBs) that can be repaired in an error-prone fashion by cellular non-homologous end joining (NHEJ) machinery. Wildtype Cas9 primarily cleaves genomic sites at which the gRNA sequence is followed by a PAM sequence (-NGG). NHEJ-mediated repair of Cas9-induced DSBs induces a wide range of mutations initiated at the cleavage site which are typically small (<10 bp) insertion/deletions (indels) but can include larger (>100 bp) indels.

### Immune Cell Library

Immune cell library can be generated as described in US20220064633 or WO2022143783. In some embodiments, the immune cell library comprises a plurality of immune cells that have mutations (e.g., inactivating mutations) in at least about any of 2, 3, 4, 5, 10, 20, 50, 100, 200, 500, 1,000, 2,000, 5,000, 10,000, 20,000, 50,000, or more hit genes in a cell or organism. In some embodiments, the organism is human. In some embodiments, at least two immune cells within the immune cell library have mutations (e.g., inactivating mutation) at different target sites (e.g., different hit genes, or different sites within the same hit gene). In some embodiments, each immune cell within the immune cell library has a mutation (e.g., inactivating mutation) at a different hit gene. In some embodiments, each immune cell within the immune cell library has a mutation (e.g., inactivating mutation) at a different target site (e.g., can be within the same hit gene, or within different hit genes). In some embodiments, the immune cell library does not contain immune cells that have mutation (e.g., inactivating mutation) at the same hit gene, such as inactivating mutation at the same target site of the same hit gene, or inactivating mutations at different target sites of the same hit gene. In some embodiments, the immune cell library does not contain immune cells that have mutation (e.g., inactivating mutation) at the same target site.

In some embodiments, a plurality of (e.g., about 2, 3, 4, 5, 10, 100, 500, 1000, 2000, 5000, 10000, or more) immune cells within an immune cell library have a mutation (e.g., inactivating mutation) at the same hit gene, such immune cell library is also referred to as “having X-fold coverage for the hit gene,” wherein “X” is the number of immune cells with mutation (e.g., inactivating mutation) at the same hit gene. In some embodiments, the immune cell library described herein has at least about 2-fold, 3-fold, 4-fold, 5-fold, 10-fold, 100-fold, 200-fold, 500-fold, 1,000-fold, 2,000-fold, 5,000-fold, 10,000-fold, or more fold coverage of each hit gene, such as averagely about 600-fold to about 18000-fold, averagely about 600-fold to about 1200-fold, or averagely about 1000-fold to about 12000-fold for each hit gene. In some embodiments, the Cas9⁺ sgRNA immune cell library has averagely about 600-fold to about 1200-fold coverage for each sgRNA. In some embodiments, the Cas9⁺ sgRNA (or mutagenic agent-induced mutation) immune cell library described herein has averagely about 600-fold to about 1200-fold coverage of each hit gene. In some embodiments, the Cas9⁺ sgRNA^{iBAR} immune cell library has averagely about 100-fold to about 1,000-fold, such as about 1000-fold, coverage for each sgRNA^{iBAR}. In some embodiments, the Cas9⁺ sgRNA^{iBAR} immune cell library has averagely about 400-fold to about 4000-fold, such as about 4000-fold, coverage for each set of sgRNAs^{iBAR}. In some embodiments, the Cas9⁺ sgRNA^{iBAR} immune cell library described herein has averagely about 1200-fold to about 12,000-fold, such as about 12,000-fold, coverage of each hit gene.

### Mutations at Hit Genes

In some embodiments, all annotated genes in the genome (e.g., human genome) are selected as hit genes, the immune cell library is also referred to herein as “whole-genome immune cell library”, such as all annotated genes of the human genome. In some embodiments, the immune cell library is a targeted library, which contains mutations (e.g., inactivating mutations) at selected genes in a signaling pathway or associated with a cellular process, such as sensitivity to AICD, GvHD, HvG, immune checkpoint molecule(s), cell proliferation, differentiation, maturation, activation, persistence, homeostasis, effector function, cell cycle, transcriptional regulation, ubiquitination, apoptosis, immune response such as autoimmune, tumor metastasis, tumor malignant transformation, etc. In some embodiments, the immune cell library is used for a genome-wide screen associated with a particular modulated phenotype, such as high persistence or high resistance to AICD in vivo. In some embodiments, genes whose DNA mutation frequency are at least about 5% (e.g., at least about any of 10%, 20%, 30%, 40%, 50%, 60%, 70%, 80%, 90%, or higher) in patients with GvHD, HvG, immune-related diseases, or cancer (e.g., based on literature or databases) are selected as hit genes. In some embodiments, genes whose RNA expression levels are up-regulated or down-regulated by at least about 1.2-fold (e.g., at least about any of 1.5, 2, 3, 4, 5, 6, 7, 8, 9, 10, 50, 100 folds, or higher, such as about 2-fold) in patients with GvHD, HvG, immune-related diseases, or cancer (e.g., based on literature or databases) are selected as hit genes. In some embodiments, genes whose DNA mutation frequency are at least about 5% (e.g., at least about any of 10%, 20%, 30%, 40%, 50%, 60%, 70%, 80%, 90%, or higher) and whose RNA expression levels are up-regulated or down-regulated by more than about 2-fold (e.g., more than about any of 2.5, 3, 4, 5, 6, 7, 8, 9, 10, 50, 100 folds, or more) in patients with GvHD, HvG, immune-related diseases, or cancer (e.g., based on literature or databases) are selected as hit genes. In some embodiments, a hit gene is further selected based on that the encoded mRNA or protein expresses within a cell, or that the encoded protein expresses on the cell surface, either in heathy cells or in disease cells.

In some embodiments, the mutation at a hit gene is a pathogenic or inactivating mutation. An inactivating mutation described herein can be any mutation, such as insertion, deletion (indels), substitution, frame shift, chromosomal rearrangement, or combinations thereof, that leads to complete abolishment or elimination of a gene's expression (transcription and/or translation) and/or function. Inactivating mutations in some embodiments can completely abolish the transcription, translation, post-translation modification, association with other molecules (e.g., other molecules in a protein complex), and/or function (e.g., signal transduction or receptor activation) of a gene. In some embodiments, the mutation at a hit gene is a mutation that reduces (e.g., reduces at least about any of 5%, 10%, 20%, 30%, 40%, 50%, 60%, 70%, 80%, 90%, 95%, 96%, 97%, 98%, 99%, or more) or affects (e.g., disrupts) one or more of hit gene transcription, hit gene translation, hit gene mRNA processing, hit gene mRNA stability, hit gene mRNA function, hit gene protein function, association with other molecules (e.g., other molecules in a protein complex), and hit gene post-translation modification. The mutation (e.g., inactivating mutation) at a hit gene can be within one or more of regulatory region such as enhancer, promoter, 5′ untranslated region (UTR), 3′UTR, or the coding region such as an exon or a splicing site, of a hit gene. A hit gene described herein can be any genomic sequence, such as a protein-encoding gene, a gene encoding an RNA, such as a small RNA (e.g., microRNA, piRNA, siRNA, snoRNA, tRNA, rRNA and snRNA), a ribosomal RNA, a long non-coding RNA (lincRNA), or a mitochondrial gene. The hit gene may be known to be associated with a particular phenotype; or has not been implicated in a particular phenotype, such as a known gene that is not known to be associated with a particular phenotype, or an unknown gene that has not been characterized. In some embodiments, the hit gene is a genomic sequence that does not encode anything, or not yet known to encode anything.

Pathogenic inactivating mutations (loss-of-function) of certain genes can be determined by review of experimental evidence within the published scientific literature and review of critical regions that may be disrupted, including but not limited to frameshift, missense mutations, truncating mutations, deletions, copy number variations, nonsense mutations, and loss or deletion of the gene. Pathogenic or inactivating mutation includes but not limited to homozygous deletions, bi-allelic (double hit) mutations, splice site mutations (e.g., a 2nd or an additional splice site mutation), frameshift mutations, and nonsense mutations in coding region, missense mutations with confirmed impact.

In some embodiments, the immune cell library is generated by subjecting (e.g., contacting) an initial population of immune cells to mutagenic agents. Mutagenic agents can be classified into three categories: physical (e.g., gamma rays, ultraviolet radiations), chemical (e.g., ethyl methane sulphonate or EMS) and transposable elements (such as transposons, retrotransposons, T-DNA, retroviruses).

In some embodiments, the immune cell library is generated by subjecting an initial population of immune cells to gene editing, or RNA editing. Any known gene editing methods can be used for generating immune cell libraries described herein, such as NHEJ, HDR, Zinc-finger nucleases (ZFNs), transcription activator-like effector nucleases (TALENs), and CRISPR/Cas-based methods for gene editing or genome engineering. See, e.g., Gaj et al. (Trends Biotechnol. 2013; 31(7): 397-405). In some embodiments, the immune cell library is generated by subjecting an initial population of immune cells to gene editing via CRISPR/Cas-based methods. In some embodiments, the immune cell library is generated by subjecting an initial population of immune cells to LEAPER or CRISPR/Cas13 for RNA editing.

In some embodiments, the immune cell library is generated by contacting an initial population of immune cells with i) a gRNA (e.g., sgRNA) library or an sgRNA^{iBAR} library descried herein; and ii) a Cas component comprising a Cas protein or a nucleic acid encoding the Cas protein (e.g., Cas9), under a condition that allows introduction of the gRNA (e.g., sgRNA) constructs or sgRNA^{iBAR} constructs and the Cas component into the initial population of immune cells and generation of mutations at the hit genes.

In some embodiments, the Cas component (e.g., Cas9) is introduced into the immune cells before the introduction of the gRNA (e.g., sgRNA) library or the sgRNA^{iBAR} library. In some embodiments, the immune cells are sorted to obtain Cas⁺ immune cells before the introduction of the gRNA (e.g., sgRNA) library or the sgRNA^{iBAR} library. In some embodiments, the gRNA (e.g., sgRNA) library or the sgRNA^{iBAR} library is introduced into the immune cells before the introduction of the Cas component (e.g., Cas9). In some embodiments, the immune cells are sorted to obtain gRNA⁺ or sgRNA⁺ or sgRNA^{iBAR+} immune cells before the introduction of the Cas component (e.g., Cas9). In some embodiments, the Cas component (e.g., Cas9) and the gRNA (e.g., sgRNA) library or the sgRNA^{iBAR} library are introduced into the immune cells at the same time. In some embodiments, the immune cells are sorted to obtain Cas⁺ gRNA⁺ immune cells (Cas⁺ gRNA⁺ immune cell library) or Cas⁺ sgRNA⁺ immune cells (Cas⁺ sgRNA⁺ immune cell library) or Cas⁺ sgRNA^{iBAR+} immune cells (Cas⁺ sgRNA^{iBAR+} immune cell library).

In some embodiments, at least about 50% (such as at least about any of 60%, 70%, 80%, 90%, 95%, 96%, 97%, 98%, 99%, or more) of the gRNA (e.g., sgRNA) constructs in the gRNA (e.g., sgRNA) library, or the sgRNA^{iBAR} constructs in the sgRNA^{iBAR} library, or the sets of sgRNA^{iBAR} constructs in the sgRNA^{iBAR} library, are introduced into the initial population of immune cells, or Cas9⁺ immune cells described herein. In some embodiments, the hit gene inactivating efficiency by the gRNA (e.g., sgRNA) library or the sgRNA^{iBAR} library is at least about 80%, such as at least about any of 81%, 82%, 83%, 84%, 85%, 86%, 87%, 88%, 89%, 90%, 91%, 92%, 93%, 94%, 95%, 96%, 97%, 98%, 99%, or more, such as at least about 90%.

In some embodiments, the immune cell library comprises one or a plurality of (e.g., about 2, 3, 4, 5, 8, 10, 100, 250, 400, 500, 1,000, 2,000, 5,000, 10,000, or more) immune cells that comprise the same gRNA (e.g., sgRNA) construct or the same sgRNA^{iBAR} construct, which targets the same target site of a hit gene. Such immune cell library is also referred to as “having X-fold coverage for the gRNA/sgRNA/sgRNA^{iBAR}, or “having X-fold coverage for each gRNA/sgRNA/sgRNA^{iBAR},” wherein “X” is the number of immune cells expressing the same gRNA (e.g., sgRNA) or sgRNA^{iBAR}.

In some embodiments, the immune cell library has at least about 100-fold (e.g., at least about any of 200-, 400-, 500-, 1,000-, 5,000-, or more fold) coverage, such as at least about 500-fold coverage, for each sgRNA or mutation (e.g., mutagenic agent-induced mutation). In some embodiments, each hit gene is targeted by about 6 to about 12 different sgRNAs, or has mutations in at least 2 (e.g., about 3) different target sites. In some embodiments, the immune cell library has at least about 100-fold (e.g., at least about any of 200-, 300-, 400-, 500-, 1,000-, 5,000-, 10,000- or more fold) coverage for each hit gene, such as about 600-fold to about 1200-fold coverage for each hit gene. In some embodiments, the immune cell library has about 12,000-fold coverage for each hit gene.

In some embodiments, the immune cell library has at least about 100-fold (e.g., at least about any of 200-, 400-, 500-, 1,000-, 5,000-, or more fold) coverage for each sgRNA^{iBAR}, such as about 100-fold to about 1000-fold, or about 1000-fold coverage for each sgRNA^{iBAR}. In some embodiments, the immune cell library has at least about 400-fold (e.g., at least about any of 800-, 1000-, 2000-, 4000-, 16,000-, or more fold) coverage for each set of sgRNA^{iBAR}, such as about 400-fold to about 4000-fold, or about 4000-fold coverage for each set of sgRNAs^{iBAR}. In some embodiments, the immune cell library has at least about 100-fold (e.g., at least about any of 200-, 400-, 500-, 1,000-, 5,000-, or more fold) coverage for the sgRNA^{iBAR} library, such as about 100-fold to about 1000-fold, or about 1000-fold coverage for the sgRNA^{iBAR} library. In some embodiments, the immune cell library has at least about 400-fold (e.g., at least about any of 800-, 1000-, 2000-, 4000-, 10,000, 16,000-, or more fold) coverage for each hit gene, such as about 1200-fold to about 12,000-fold coverage for each hit gene, or about 12,000-fold coverage for each hit gene. In some embodiments, the sgRNA^{iBAR} library targets every annotated gene in the genome (i.e., the sgRNA^{iBAR} library is a whole-genome sgRNA^{iBAR} library). In some embodiments, the immune cell library has at least about 100-fold (e.g., at least about any of 400-fold, 800-fold, 1000-fold, or 1200-fold) coverage for the whole-genome sgRNA^{iBAR} library.

### Endogenous Mutation(s)

In some embodiments, the immune cells in the initial population of immune cells or in the final immune cell library (e.g., AICD-resistant immune cell population) may comprise endogenous mutation(s) not generated by the CRISPR/Cas system or mutagenic agents (e.g., EMS), such as naturally occurring mutations, or mutations in immune cells that do not meet the hit gene selection criteria (e.g., DNA mutation frequency is at least about 5%, and/or RNA expression level is up-regulated or down-regulated by more than about 2-fold in patients, and/or the encoded RNA/protein is expressed within cell or the encoded protein is expressed on the cell surface). Endogenous mutation(s) should not affect the target gene identification methods described herein, as the profiles of gRNAs (e.g., sgRNAs) or hit gene mutations in the AICD-resistant immune cell population are compared to a control immune cell population comprising the same endogenous mutation(s). In some embodiments, the endogenous mutations are mutations (e.g., LOF mutation) in one or more of: TCRα, TCRβ, TCRγ, TCRδ, HLA-A, HLA-B, HLA-C, HLA-E, HLA-F, HLA-G, B2M, PD-1, TIM-3, LAG-3, CTLA-4, CISH, SPPL3, Fas, FADD, CASP8, ARID1A, BAK1, BID, ETS1, IKZF2, HIST1H1B, B7-H6, MICA, MICB, ULBP1, ULBP2, ULBP3, ULBP4, ULBP5, ULBP6, and ligands of NKp46.

### Immune Cells

In some embodiments, the multiplicity of infection (MOI) between the viral vectors or viruses (e.g., encoding gRNA, sgRNA, or sgRNA^{iBAR}) and the host immune cells (e.g., initial population of immune cells) during the transfection is at least about 1, such as at least about any one of 1.5, 2, 2.5, 3, 3.5, 4, 4.5, 5, 5.5, 6, 6.5, 7, 7.5, 8, 8.5, 9, 9.5, 10, or higher, or about any one of 1-10, 1-3, 3-5, 5-10, 2-9, 3-8, 4-6, or 2-5. In some embodiments, the MOI between the viral vectors or viruses and the host immune cells (e.g., initial population of immune cells) during transfection is less than 1, such as less than about any of 0.8, 0.5, 0.3, or lower, such as about 0.3 to about 1. In some embodiments, the viral gRNA (e.g., sgRNA) library or the viral sgRNA^{iBAR} library is contacted with the initial population of immune cells at an MOI of at least about 2, such as at least about 3.

In some embodiments, one or more vectors driving expression of one or more elements of a CRISPR/Cas system are introduced into a host immune cell (e.g., the initial population of immune cells) simultaneously or sequentially. In some embodiments, the host immune cell (e.g., the initial population of immune cells) has been introduced a Cas nuclease (e.g., Cas9 mRNA) or is engineered to stably express CRISPR/Cas nuclease.

In some embodiments, the host immune cell (e.g., the initial population of immune cells) is an immune cell line, such as a pre-established immune cell line. The host immune cells and immune cell lines may be human immune cells or immune cell lines, or they may be non-human, mammalian immune cells or immune cell lines. In some embodiments, the host immune cell is difficult to transfect with a viral vector, such as lentiviral vector, at a low MOI (e.g., lower than 1, 0.5, or 0.3). In some embodiments, the host immune cell is difficult to edit using a CRISPR/Cas system at low MOI (e.g., lower than 1, 0.5, or 0.3). In some embodiments, the host immune cell is available at a limited quantity. In some embodiments, the immune cells (e.g., initial population of immune cells, or modified immune cells described herein) are obtained from an individual, such as a patient with cancer or immune-related disease, or a patient to be treated with immunotherapy such as CAR-T therapy. In some embodiments, the immune cells can be autologous (e.g., obtained from the same patient to be treated with cell-based immunotherapy) or allogeneic (e.g., not derived from the patient to be treated with cell-based immunotherapy, such as obtained from a healthy individual).

In some embodiments, the host immune cell, such as the initial population of immune cells, or the immune cells to be modified to comprise mutation(s) (e.g., LOF mutation) in one or more of AICD resistant genes or target genes described herein, can further express an engineered component, such as an antibody construct (e.g., immune checkpoint modulator), an engineered receptor, a cytokine construct (e.g., immunocytokine), etc. In some embodiments, the host immune cell, such as the initial population of immune cells, or the immune cells to be modified to comprise mutation(s) (e.g., LOF mutation) in one or more of AICD resistant genes or target genes described herein, comprises a CAR, such as CAR-T cells, or CAR-NK cells. In some embodiments, the engineered component (e.g., CAR) is introduced into the immune cells after introducing the gRNA (e.g., sgRNA) library, sgRNA^{iBAR} library, Cas component, hit gene mutations, target gene mutations, or AICD resistant mutations described herein. In some embodiments, the engineered component (e.g., CAR) is introduced into the immune cells before introducing the gRNA (e.g., sgRNA) library, sgRNA^{iBAR} library, Cas component, hit gene mutations, target gene mutations, or AICD resistant mutations described herein.

In some embodiments, the immune cells are derived from immune cell lines. The immune cells in some embodiments are obtained from a xenogeneic source, for example, from mouse, rat, non-human primate, and pig. In some embodiments, the immune cells are human immune cells. In some aspects, the immune cells are primary cells, such as those isolated directly from a subject and/or isolated from a subject and frozen. In some embodiments, the initial population of immune cells is homogenous. In some embodiments, the initial population of immune cells is heterogeneous, such as primary immune cells, or comprising same immune cells of mixed status (e.g., differentiation status, activation status), or mixed immune cell lines of the same immune cell type. In some embodiments, after collecting immune cells from a subject, the immune cells are sorted to obtain a subset of immune cells, e.g., using the immunomagnetic bead method. In some embodiments, immune cells are obtained from a patient directly following a treatment (e.g., chemotherapy, radiation, surgery, immunotherapy such as CAR-T therapy). It is contemplated within the context of the present invention to collect immune cells after a treatment (e.g., CAR-T, or anti-cancer drug), as host immune cells, or to test hit gene expression level change.

### Control Immune Cell Population

In some embodiments, the control immune cell population is a subpopulation of the immune cell library described herein before step b), such as obtained from the immune cell library before step b). In some embodiments, the control immune cell population and the immune cell library have been subjected to the same growth and/or treatment (e.g., viability indicator staining, FACS) conditions, but the control immune cell population is not subjected to FasL treatment. In some embodiments, the control immune cell population is a same immune cell library cultured under the same condition and not contacted with the FasL.

In some embodiments, the control immune cell population is a same initial population of immune cells without mutation at any of the hit genes, AICD resistant genes, or genes encoding any of the target proteins described herein (e.g., SPPL3). In some embodiments, the control immune cell population is a same initial population of immune cells without mutation at any gene and/or genomic region. In some embodiments, the control immune cell population is a same initial population of immune cells with mutation at one or more non-hit genes or genomic regions not associated with any hit genes (or genes encoding any of the target proteins described herein).

In some embodiments, the control immune cell population is the same as the immune cell library described herein, but without introducing either the gRNA library (e.g., sgRNA or sgRNA^{iBAR} library) or the Cas component. In some embodiments, the control immune cell population is a Cas9⁻ gRNA (e.g., sgRNA or sgRNA^{iBAR}) control immune cell library, and the immune cell library is a Cas9⁺ gRNA (e.g., sgRNA or sgRNA^{iBAR}) immune cell library.

In some embodiments, the Cas9⁻ gRNA (e.g., sgRNA or sgRNA^{iBAR}) control immune cell library is subjected to a step b) FasL treatment and a step c) of obtaining (e.g., by FACS or centrifugation) from it an AICD-resistant control immune cell population under the same condition as the Cas9⁺ gRNA (e.g., sgRNA or sgRNA^{iBAR}) immune cell library. The AICD-resistant control immune cell population do not have hit gene mutations created by CRISPR/Cas9, and the distribution of gRNAs (e.g., sgRNAs or sgRNAs^{iBAR}) in these control immune cell populations should be random; whereas AICD-resistant immune cell populations should have enrichment of certain gRNAs (e.g., sgRNAs or sgRNAs^{iBAR}) that target certain hit genes.

Contacting the Immune Cell Library with a FasL

The methods described herein comprise contacting the immune cell library or intermediate immune cell population (e.g., first or second AICD-resistant immune cell population, such as after corresponding growth recovery phase) with a FasL (hereinafter also referred to as “FasL treatment step,” “treatment step b),” or “step b)”). In some embodiments, the method comprises growing the immune cell library or intermediate immune cell population in a cell culture medium containing FasL. In some embodiments, the FasL contacting step b) is at least about 2 hours, such as at least about any of 4, 6, 8, 10, 12, 14, 16, 18, 20, 22, 24, 30, 40, 50 or longer hours. In some embodiments, the FasL contacting step b) is about 16 hours. In some embodiments, the immune cell library is subject to 2 or more (e.g., 3) rounds of FasL contacting steps, hereinafter referred to as “the first, second, third, etc. FasL treatment step,” respectively. In some embodiments, when the immune cell library is subject to 2 or more (e.g., 3) rounds of FasL contacting steps, the concentration of FasL in each round can be the same or different.

### Obtaining AICD-Resistant Immune Cell Population

The methods described herein comprise obtaining from the immune cell library described herein (e.g., immune cell library generated by mutagenic agent(s), LEAPER, Cas9⁺ gRNA (e.g., sgRNA) immune cell library, or Cas9⁺ sgRNA^{iBAR} immune cell library, the remaining immune cell library after obtaining a subpopulation as control immune cell population, or immune cell library after FasL treatment) an AICD-resistant immune cell population (hereinafter also referred to as “AICD-resistant immune cell population obtaining step,” “obtaining step c),” or “step c)”). In some embodiments, when the immune cell library is subject to 2 or more (e.g., 3) rounds of FasL contacting steps, there is one obtaining step c) after each FasL contacting step b), hereinafter referred to as “the first, second, third, etc. obtaining step,” respectively. The obtained AICD-resistant immune cell population is hereinafter referred to as “the first, second, third, etc. AICD-resistant immune cell population,” respectively; and the last AICD-resistant immune cell population is also referred to herein as the “final AICD-resistant immune cell population.” In some embodiments, step c) comprise using FACS to obtain the AICD-resistant immune cell population. Any suitable FACS protocol can be employed herein. In some embodiments, step c) comprise using fluorescent microscopy to obtain the AICD-resistant immune cell population. In some embodiments, step c) further comprises contacting the immune cell library or intermediate immune cell population with a viability indicator, such as one or more of propidium iodide (PI), DAPI, 7-AAD, and Annexin V. In some embodiments, the AICD-resistant immune cell population is Annexin V⁻ negative and DAPI⁻ negative. In some embodiments, step c) comprises low speed centrifugation to obtain the AICD-resistant immune cell population. In some embodiments, low speed centrifugation is about 100 rcf to about 500 rcf (e.g., about any of 100, 150, 200, 300, 400, or 500 rcf), such as for about 5 min to about 30 min (e.g., about any of 5, 10, 15, 20, 25, or 30 min). In some embodiments, low speed centrifugation is at about 150 rcf for about 5 minutes. In some embodiments, the obtaining step c) comprises a single step of obtaining the AICD-resistant immune cell population. In some embodiments, the AICD-resistant immune cell population obtaining step c) optionally comprises one or more additional steps, such as an optional enrichment step, or an optional recovery step.

In some embodiments, the immune cell library described herein (e.g., immune cell library generated by mutagenic agent(s), Cas9⁺ sgRNA immune cell library, Cas9⁺ sgRNA^{iBAR} immune cell library, immune cell library after FasL treatment, or the remaining immune cell library after obtaining a subpopulation as control immune cell population) is allowed to grow for a period of time or doubling time (e.g., at 37° C., 5% CO₂ for about 6 days or about 3 PDT) after the obtaining step c), and before the next round of FasL treatment step b). In some embodiments, alive cells are collected in the obtaining step c), such as using FACS to obtain Annexin V⁻ negative and DAPI⁻ negative immune cells. In some embodiments, at least about 80% (e.g., at least about any of 85%, 90%, 95%, 99%, or 100%) of the obtained AICD-resistant immune cell population from step c) are alive cells, such as obtained via low speed centrifugation. In some embodiments, an optional enrichment step (alive cell enrichment) is performed after the obtaining step c), such as FACS sorting alive cells after low speed centrifugation. In some embodiments, an optional detection step (e.g., detecting death rate or alive rate) is performed after the obtaining step c), such as using FACS or fluorescent microscopy to detect ratio or percentage of alive (e.g., Annexin V⁻ negative and DAPI⁻ negative) or dead cells in the obtained AICD-resistant immune cell population from step c).

Also see Example 1 and FIGS. 1 and 2 for exemplary methods.

### Optional Enrichment Step

The methods described herein can comprise one or more optional “enrichment steps,” if one desires to obtain a purer alive population of immune cells after the FasL treatment step b), or after obtaining the AICD-resistant immune cell population in step c), or when constructing the immune cell library (e.g., to enrich for Cas⁺ sgRNA⁺ immune cells, CAR⁺ immune cells, or Cas⁺ sgRNA⁺ CAR⁺ immune cells). In some embodiments, the enrichment step comprises sorting the immune cells to obtain purely alive immune cell population. In some embodiments, the method comprises sorting the immune cell library to obtain an alive immune cell population, a Cas⁺ gRNA⁺ (e.g., sgRNA⁺ or sgRNA^{iBAR+}) immune cell population, an alive Cas⁺ gRNA⁺ (e.g., sgRNA⁺ or sgRNA^{iBAR+}) immune cell population, a Cas⁺ gRNA⁺ (e.g., sgRNA⁺ or sgRNA^{iBAR+}) CAR⁺ immune cell population, an alive Cas⁺ gRNA⁺ (e.g., sgRNA⁺ or sgRNA^{iBAR+}) CAR⁺ immune cell population, etc. In some embodiments, such alive cell enrichment step is performed after or at the same time as, the AICD-resistant immune cell population obtaining step. For example, step c) comprises low speed centrifuging the FasL treated immune cell library to obtain an immune cell population mostly alive (mostly AICD-resistant), and optionally further comprises an enrichment step to use FACS to sort alive cells from the centrifuged mostly alive immune cell population (e.g., Annexin V⁻ negative and DAPI⁻ negative cells), thus obtaining the AICD-resistant immune cell population.

In some embodiments, the enrichment step comprises staining the immune cells (e.g., host immune cells, the initial population of immune cells, immune cells after the FasL contacting step, the AICD-resistant immune cell population, or the control immune cell population) with a cell viability marker (e.g., dye) before sorting (e.g., by FACS). Methods and reagents for assessing cell viability are well known in the art, e.g., fluorescent based or colorimetric (enzymatic) based. For example, membrane permeability-based assays such as staining with DAPI, propidium iodide (PI), 7-AAD, or amine-reactive dyes indicates dead cells; while acridine orange stains viable cells more efficiently. Carboxyfluorescein diacetate (CFDA) is a nonfluorescent, cell permeable dye that is hydrolyzed to form the fluorescent molecule carboxyfluorescein by nonspecific intracellular esterases present only in viable cells. CFDA-SE is a derivative of CFDA that is better retained upon hydrolysis, in viable cells. Tetramethylrhodamine ethyl esters (TMRE) and Tetramethylrhodamine methyl esters (TMRM) localize to mitochondria in healthy cells and to the cytoplasm in dying cells. JC-1 is a commonly used potentiometric dye. In healthy cells JC-1 localizes to the mitochondria, where it forms red fluorescent aggregates. Upon breakdown of the mitochondrial membrane potential, JC-1 diffuses throughout the cell and exists as a green fluorescent monomer. BrdU incorporation into newly synthesized DNA indicates live cells.

In some embodiments, the enrichment step comprises staining the immune cell library (e.g., after the FasL contacting step) with a cell viability marker before obtaining the AICD-resistant immune cell population. In some embodiments, the AICD-resistant immune cell population (purely or mostly alive) is further enriched by staining (e.g., immunofluorescent staining) the immune cell library (e.g., after the FasL contacting step) with a marker selected from more of Fas, HLA-A, HLA-B, HLA-C, HLA-E, B7-H6, MICA, MICB, ULBP1, ULBP2, ULBP3, ULBP4, ULBP5, ULBP6, and ligands of NKp46, and only immune cells with low expression (e.g., bottom 50%, 40%, 30%, 20%, 10%, or less) of the one or more markers are selected (e.g., by FACS) for target gene identification in step d). In some embodiments, the enrichment by cell marker is conducted before, at the same time, or after the enrichment by cell viability. In some embodiments, the control immune cell population is stained with a same cell viability marker and sorted for alive cells (or further sorted with cell surface markers for lower expression cells), before the target gene identification step, or before, at the same time, or after obtaining the AICD-resistant control immune cell population.

In some embodiments, the enrichment step comprises staining the immune cell library (e.g., after the FasL contacting step), the AICD-resistant immune cell population, and/or the control immune cell population, with the engineered component (e.g., CAR), i.e., to enrich for immune cells whose engineered component (e.g., CAR) expression is not or barely affected due to hit gene mutation(s) or during cell culture. In some embodiments, the immune cell library (e.g., after the FasL contacting step), the final AICD-resistant immune cell population, and/or the control immune cell population, are enriched for immune cells whose engineered component (e.g., CAR) expression is within at most about ±30% (e.g., at most about any of ±20%, ±10%, ±5%, or less) variation of the average of the initial population of immune cells.

Any cell sorting methods can be used herein, such as Fluorescence-activated cell sorting (FACS), Magnetic-activated cell sorting (MACS), microfluidic cell-sorting, buoyancy-activated cell sorting (BACS), etc.

### Optional Recovery Step

In some embodiments, the obtained AICD-resistant immune cell population (intermediate or final) is allowed to grow for a period of time after the obtaining step c) and before the target gene identification step d), hereinafter also referred as “the recovery step.” The immune cell population after the recovery period or step is hereinafter referred to as the “recovered immune cell library” or “recovered immune cell population.” Any suitable immune cell culture medium can be used for such recovery period. The culture medium can be changed every few hours or days (e.g., every 1, 2, 3, 4, 5, 6, or 7 days, such as about 5-7 days, or about 6 days), or after certain period or doubling time (e.g., about 2 to about 10 PDT, such as about 3 PDT). In some embodiments, the obtained AICD-resistant immune cells are passaged every 1, 2, 3, 4, 5, or more (such as 3) doubling time, to keep the same or similar (e.g., within about 10% difference) cell numbers as the initial immune cell library (before FasL treatment) before the target gene identification step d). In some embodiments, immune cells are passaged when reaching about 90% confluence. In some embodiments, the obtained AICD-resistant immune cell population (intermediate or final) is grown at 37° C., 5% CO₂ for about 6 days before the target gene identification step d).

In some embodiments, the recovery step comprises culturing the AICD-resistant immune cell population (intermediate or final) for at least about 2 hours (hrs), such as at least about any of 3 hrs, 4 hrs, 5 hrs, 6 hrs, 7 hrs, 8 hrs, 9 hrs, 10 hrs, 11 hrs, 12 hrs, 14 hrs, 16 hrs, 18 hrs, 20 hrs, 22 hrs, 24 hrs, 26 hrs, 28 hrs, 30 hrs, 32 hrs, 34 hrs, 36 hrs, 38 hrs, 40 hrs, 48 hrs, 52 hrs, 56 hrs, 60 hrs, 64 hrs, 68 hrs, 72 hrs, 78 hrs, 84 hrs, 96 hrs, 100 hrs, 120 hrs, 5 days, 6 days, 7 days, 8 days, 9 days, 10 days, 12 days, 14 days, 16 days, 18 days, 20 days, 24 days, 30 days, or longer. In some embodiments, the recovery step comprises culturing the immune cells for about 5 to about 7 days, such as 6 days. In some embodiments, the recovery step comprises culturing the immune cells at least about 25° C., such as at least about any of 26° C., 27° C., 28° C., 30° C., 35° C., 37° C., or 40° C., such as about 37° C. In some embodiments, the recovery step comprises culturing the immune cells at about 4% to about 10% CO₂, such as about 5% to about 7% CO₂, or about 5% CO₂.

The culturing condition during the recovery step should be suitable for immune cell growth/proliferation. In some embodiments, the culturing condition is (further) suitable for immune cell differentiation, maturation, and/or activation. In some embodiments, the culturing condition does not induce immune cells to a specific phenotype during expansion/growth. Such culture conditions are well known in the art. For example, in 37° C., 5% CO₂ incubator. In some embodiments, the culture medium is an immune cell (e.g., T cell) expansion medium. The type of culture media for successful culture can vary depending on the types of immune cells. In some embodiments, the culture medium is further supplemented with an agent for selectable markers, e.g., to select immune cells that do not lose transgenes (e.g., CAR, Cas and/or gRNA vector) or mutations during proliferation.

In some embodiments, an optional “harvest step” is performed after the recovery step (or after obtaining step c)) and before the target gene identification step b). In some embodiments, the immune cells after the recovery step (or after obtaining step c)) are centrifuged, and the culture medium is discarded. In some embodiments, the immune cells are adherent, and the culture medium (contains dead cells or floating cells) is discarded. In some embodiments, for adherent immune cells, after removing culture medium (contains dead cells or floating cells), remaining immune cells in the cell culture container (e.g., cell culture dish) are dissociated using trypsin and collected (e.g., transferred to a fresh container). Such obtained immune cells will be alive immune cells. In some embodiments, the immune cells are non-adherent, and after low speed centrifugation, the supernatant (contains dead cells and cell culture medium) is discarded. The immune cell harvest step in some embodiments comprises collecting the immune cells into a container (e.g., Falcon tubes, EP tubes, or centrifugation tubes) for storage or for later experiments. In some embodiments, the harvest step comprises washing the obtained immune cells, so that the immune cells are in suitable condition for storage (e.g., 4° C., −20° C., or −80° C. storage) or later experiments (e.g., FACS, cell lysis, PCR, or sequencing).

### Hit Gene Identification

The method described herein comprises identifying the hit gene in the AICD-resistant immune cell population (“hit gene identification step”). In some embodiments, the hit gene identified from the AICD-resistant immune cell population is considered as the target gene whose mutation increases resistance to AICD. Any hit gene identification methods described in US20220064633 and WO2022143783 can be used herein.

In some embodiments, the hit gene identification step comprises: i) identifying a sequence comprising the hit gene mutation (e.g., inactivating mutation) in the AICD-resistant immune cell population obtained from step c); and ii) identifying the hit gene corresponding to the sequence comprising the hit gene mutation (e.g., inactivating mutation). In some embodiments, the sequence comprising the hit gene mutation (e.g., inactivating mutation) is identified by sequencing, e.g., PCR-sequencing (e.g., Sanger sequencing), or genome-sequencing (or DNA-seq, such as next-generation sequencing or “NGS”). For example, in some embodiments, the sequences (nucleic acid fragments, PCR fragments, or whole-genome) of the AICD-resistant immune cell population are identified by sequencing, by comparing to the wild-type (or heathy individual) genomic sequence, by comparing to the genomic sequence of the control immune cell population, or by comparing to the genomic sequence of the initial population of immune cells, and sequence(s) comprising the hit gene mutation(s) (e.g., inactivating mutation(s)) can be identified and mapped to the hit gene(s). In some embodiments, the hit gene identification step further comprises isolating genomic DNA or RNA from the AICD-resistant immune cell population from step c). In some embodiments, the hit gene identification step further comprises isolating genomic DNA or RNA from the control immune cell population. In some embodiments, the hit gene identification step further comprises PCR amplification of nucleic acid sequence comprising the hit gene mutation (e.g., inactivating mutation), and/or sequences isolated from a control immune cell population.

In some embodiments, the immune cell library described herein comprises the gRNA (e.g., sgRNA) constructs or the sgRNA^{iBAR} constructs against hit genes described herein. Thus in some embodiments, the hit gene identification step comprises: i) identifying the gRNA (e.g., sgRNA) sequence or the sgRNA^{iBAR} sequence in the AICD-resistant immune cell population obtained from step c); and ii) identifying the hit gene corresponding to (targeted by) the guide sequence of the gRNA (e.g., sgRNA) or the sgRNA^{iBAR}. In some embodiments, the hit gene identification step comprises: i) identifying the gRNA (e.g., sgRNA) sequence or the sgRNA^{iBAR} sequence in the control immune cell population; and ii) identifying the hit gene corresponding to (targeted by) the guide sequence of the gRNA or the sgRNA^{iBAR}. In some embodiments, the gRNA (e.g., sgRNA) sequence or the sgRNA^{iBAR} sequence is identified by RNA sequencing (RNA-seq), e.g., RNA NGS. In some embodiments, the hit gene identification step comprises: i) identifying the nucleic acid sequence encoding the gRNA (e.g., sgRNA) or the sgRNA^{iBAR} in the AICD-resistant immune cell population obtained from step c), and/or the control immune cell population; and ii) identifying the hit gene corresponding to the guide sequence encoded by the nucleic acid sequence. In some embodiments, the nucleic acid sequence encoding the gRNA (e.g., sgRNA_ or the sgRNA^{iBAR} is identified by sequencing, e.g., PCR-sequencing (e.g., Sanger sequencing), or genome-sequencing (DNA-seq), e.g., NGS. In some embodiments, the iBAR sequences can be used for identifying the sgRNA^{iBAR} sequences or the nucleic acid sequences encoding the sgRNA^{iBAR}. In some embodiments, the hit gene identification step further comprises isolating genomic DNA or RNA from the AICD-resistant immune cell population, and/or the control immune cell population. In some embodiments, the hit gene identification step further comprises PCR amplification of nucleic acid sequence encoding the gRNA (e.g., sgRNA) or the sgRNA^{iBAR}.

Methods for DNA-seq, RNA-seq, PCR-sequencing (e.g., Sanger sequencing), DNA/RNA extraction, cDNA preparation, and data analysis are well known in the art, and can be used herein as appropriate to identify the hit gene(s) in the AICD-resistant immune cell population, and/or the control immune cell population. The sequencing data can be analyzed and aligned to the genome using any known methods in the art.

### Target Gene Identification

Any target gene identification methods described in US20220064633 and WO2022143783 can be used herein. In some embodiments, the hit gene(s) identified in the AICD-resistant immune cell population is further compared to a control (e.g., control immune cell population), and/or is further ranked and/or filtered with a predetermined threshold level. In some embodiments, gRNA (e.g., sgRNA) or sgRNA^{iBAR} sequence counts (or hit gene mutation sequence counts) obtained from the AICD-resistant immune cell population are compared with gRNA or sgRNA^{iBAR} sequence counts (or hit gene mutation sequence counts) obtained from the control immune cell population, wherein the hit genes whose corresponding gRNA or sgRNA^{iBAR} guide sequences (or hit gene mutation sequences) are identified as enriched in the AICD-resistant immune cell population compared to the control immune cell population with an FDR≤0.2 (and/or with at least about 2-fold enrichment) are identified as target genes whose mutations increase resistance to AICD (AICD resistant genes).

In some embodiments, identifying the target gene comprises: i) obtaining sequences comprising the hit gene mutations (e.g., inactivating mutations) in the AICD-resistant immune cell population obtained from step c); ii) ranking the sequences comprising the hit gene mutations (e.g., inactivating mutations) based on sequence counts; and iii) identifying the hit gene corresponding to a sequence comprising the hit gene mutation (e.g., inactivating mutation) ranked above a predetermined threshold level. In some embodiments, sequences comprising the hit gene mutations (e.g., inactivating mutations) in the AICD-resistant immune cell population are ranked from high to low based on sequence counts, or enrichment of the hit gene mutation sequences in the AICD-resistant immune cell population compared to the control immune cell population. In some embodiments, the ranking step comprises adjusting the rank of each sequence comprising the hit gene mutation (e.g., inactivating mutation) based on data consistency among all sequences comprising the hit gene mutation (e.g., inactivating mutation) corresponding to the same hit gene (or same target site of the same hit gene). For example, data inconsistency (such as different directions of fold changes relative to control) will increase variance of the sequences comprising the hit gene mutation (e.g., inactivating mutation) corresponding to the same hit gene and lower the rank of such hit gene. In some embodiments, the hit gene is identified to correspond to sequence(s) comprising the hit gene mutations (e.g., inactivating mutation(s)) that rank consistently better than expected for permuted sequences under null hypothesis based on an RRA or α-RRA algorithm. In some embodiments, the predetermined threshold level is an FDR of value “X” (e.g., 0.2), and the hit gene corresponding to a sequence comprising the hit gene mutation (e.g., inactivating mutation) with FDR≤“X” is identified as the target gene. In some embodiments, the predetermined threshold level is an enrichment of value “X”-fold (e.g., about 2-fold), and the hit gene corresponding to a sequence comprising the hit gene mutation (e.g., inactivating mutation) with enrichment≥“X”-fold is identified as the target gene. In some embodiments, the sequence comprising the hit gene mutation (e.g., inactivating mutation) is identified by sequencing, e.g., Sanger-sequencing or genome-sequencing (or DNA-seq, such as NGS).

In some embodiments, identifying the target gene comprises: i) obtaining gRNA (e.g., sgRNA) sequences or sgRNA^{iBAR} sequences in AICD-resistant immune cell population obtained from step c); ii) ranking the corresponding guide sequences of the gRNA sequences or the sgRNA^{iBAR} sequences based on sequence counts; and iii) identifying the hit gene corresponding to a guide sequence ranked above a predetermined threshold level. In some embodiments, the guide sequences of the gRNA (e.g., sgRNA) sequences or the sgRNA^{iBAR} sequences in the AICD-resistant immune cell population are ranked from high to low based on sequence counts, or enrichment of the guide sequences in the AICD-resistant immune cell population compared to the control immune cell population. In some embodiments, the ranking comprises adjusting the rank of each guide sequence of the sgRNA sequence or the sgRNA^{iBAR} sequence based on data consistency among all guide sequences corresponding to the same hit gene (or same target site of the same hit gene). For example, data inconsistency (such as different direction of fold change relative to control) will increase variance of the guide sequences corresponding to the same hit gene and lower the rank of such hit gene. In some embodiments, the hit gene is identified to correspond to guide sequence(s) that rank consistently better than expected for permuted guide sequences under null hypothesis based on an RRA or α-RRA algorithm. In some embodiments, the predetermined threshold level is an FDR of value “X” (e.g., 0.2), and the hit gene corresponding to a guide sequence with FDR≤“X” is identified as the target gene. In some embodiments, the predetermined threshold level is an enrichment of value “X”-fold (e.g., about 2-fold), and the hit gene corresponding to a guide sequence with enrichment≥“X”-fold is identified as the target gene. In some embodiments, the sgRNA sequence or the sgRNA^{iBAR} sequence is identified by RNA-seq, e.g., RNA NGS. In some embodiments, the nucleic acid sequences encoding the sgRNAs or the sgRNAs^{iBAR} are identified by genome-sequencing (DNA-seq), e.g., NGS.

In some embodiments, the immune cell library described herein comprises the sgRNA^{iBAR} constructs against hit genes described herein. In some embodiments, identifying the target gene comprises: i) obtaining sgRNA^{iBAR} sequences in the AICD-resistant immune cell population obtained from step c); ii) ranking the corresponding guide sequences of the sgRNA^{iBAR} sequences based on sequence counts, wherein the ranking comprises adjusting the rank of each guide sequence based on data consistency among the iBAR sequences in the sgRNA^{iBAR} sequences corresponding to the guide sequence; and iii) identifying the hit gene corresponding to a guide sequence ranked above a predetermined threshold level. In some embodiments, the hit gene is identified to correspond to guide sequence(s) that rank(s) consistently better than expected for permuted guide sequences under null hypothesis based on an RRA or α-RRA algorithm. In some embodiments, the predetermined threshold level is an FDR of value “X” (e.g., 0.2), and the hit gene corresponding to a guide sequence with FDR≤“X” is identified as the target gene. In some embodiments, the predetermined threshold level is at least about 2-fold enrichment.

Statistical methods may be used to determine the identity of the sequences comprising the hit gene mutations (e.g., inactivating mutations), the gRNA (e.g., sgRNA) molecules, or the sgRNA^{iBAR} molecules that are enriched in the AICD-resistant immune cell population. In some embodiments, more than one (e.g., 2, 3, or more) biological or technical replicate is conducted for an immune cell library, or a control immune cell population. In some embodiments, sequences comprising the hit gene mutations (e.g., inactivating mutations) or guide RNAs from the two or more (e.g., 2, 3, 4, or more) replicates of the test group (or control group) are combined to calculate mean and variance among replicates of the test group (or control group). Exemplary statistical methods include, but are not limited to, linear regression, generalized linear regression and hierarchical regression. In some embodiments, the sequence counts are subject to normalization methods, such as total count normalization, or median ratio normalization. In some embodiments, e.g., for positive screens, median ratio normalization is preferred. In some embodiments, for example, for sequence counts that follow a normal distribution, the sequence counts are subject to median ratio normalization followed by mean-variance modeling. In some embodiments, MAGeCK (Li, W. et al. MAGeCK enables robust identification of essential genes from genome-scale CRISPR/Cas9 knockout screens. Genome Biol 15, 554 (2014)) is used to rank sequences comprising the hit gene mutations (e.g., inactivating mutations) or guide RNA sequences, and/or to identify target genes. In some embodiments, MAGeCK^{iBAR} (Zhu et al., Genome Biol. 2019; 20:20; the content of which is incorporated herein by reference in its entirety) is used to rank sequences comprising the hit gene mutations (e.g., inactivating mutations) or guide RNA sequences, and/or to identify target genes.

In some embodiments, identifying the target gene whose mutation increases resistance to AICD is based on the difference between the profiles of gRNAs (or sgRNAs or sgRNAs^{iBAR}) or hit gene mutations in the AICD-resistant immune cell population and a control immune cell population. In some embodiments, the identification of the target gene is based on the difference between the profiles of hit gene mutations in the AICD-resistant immune cell population and the control immune cell population. In some embodiments, the identification of the target gene is based on the difference between the profiles of gRNAs (or sgRNAs or sgRNAs^{iBAR}) in the AICD-resistant immune cell population and the control immune cell population. In some embodiments, the control immune cell population is obtained from the immune cell library cultured under the same condition without subjecting to step b). In some embodiments, the profiles of gRNAs (or sgRNAs or sgRNAs^{iBAR}) or hit gene mutations in the AICD-resistant immune cell population and the control immune cell population are identified by NGS, such as DNA-seq or RNA-seq. In some embodiments, the profiles of gRNAs (or sgRNAs or sgRNAs^{iBAR}) comprise sequence counts of the gRNAs (or sgRNAs or sgRNAs^{iBAR}), or sequence counts of the corresponding guide sequences of the gRNAs (or sgRNAs or sgRNAs^{iBAR}) In some embodiments, the profiles of gRNAs (or sgRNAs or sgRNAs^{iBAR}) comprise sequence counts of the nucleic acids encoding the gRNAs (or sgRNAs or sgRNA^{iBAR}), or sequence counts of the nucleic acids encoding the guide sequences of the corresponding gRNAs (or sgRNAs or sgRNAs^{iBAR}). In some embodiments, the profiles of the hit gene mutations comprise sequence counts of the sequences comprising the hit gene mutations. In some embodiments, the methods described herein further comprise culturing a same immune cell library under the same condition without subjecting to step b).

In some embodiments, the sequence counts (e.g., the sequence counts of gRNAs or sgRNAs or sgRNAs^{iBAR} or guide sequences thereof, the sequence counts of nucleic acid sequences encoding the gRNAs or sgRNAs or sgRNAs^{iBAR} or guide sequences thereof, or sequence counts of sequences comprising the hit gene mutations) obtained from the AICD-resistant immune cell population from step c) are compared to corresponding sequence counts obtained from a control immune cell population, e.g., to provide fold changes (e.g., actual fold changes, or derivatives of fold changes such as log 2 or log 10 fold changes), for significance tests (e.g., FDR, p-value), for distribution statistics, and/or to provide gene or sequence rankings via scoring and/or deriving. In some embodiments, the control immune cell population is a same immune cell library cultured under the same condition without subjecting to step b), e.g., cultured under the same culture condition for the same amount of time as the test group from test beginning until before target gene identification step d), hereinafter also referred to as “control immune cell library.”. In some embodiments, when the immune cell library is subjected to step b) FasL treatment, the control immune cell population is discontinued for growth (e.g., by fixation).

In some embodiments, the AICD-resistant immune cell population obtained is alive immune cells. In some embodiments, identifying the target gene comprises comparing the gRNA (or sgRNA or sgRNA^{iBAR} or guide sequence thereof, or nucleic acid encoding gRNA or sgRNA or sgRNA^{iBAR} or guide sequence thereof) sequence counts obtained from the AICD-resistant immune cell population with gRNA (or sgRNA or sgRNA^{iBAR} or guide sequence thereof, or nucleic acid encoding gRNA or sgRNA or sgRNA^{iBAR} or guide sequence thereof) sequence counts obtained from the control immune cell population, wherein: i) the hit genes whose corresponding gRNA (or sgRNA or sgRNA^{iBAR}) guide sequences are identified as enriched in the AICD-resistant immune cell population compared to the control immune cell population with an FDR≤0.2 (e.g., FDR≤any of 0.15, 0.1, 0.05, 0.04, 0.03, 0.02, 0.01, 0.005, 0.001, or less) (and/or with at least about 2-fold enrichment, such as at least about any of 3-, 4-, 5-, 10-, 20-, 50-, 100-fold, or more enrichment) are identified as target genes whose mutations increase resistance to AICD. In some embodiments, the gRNA (or sgRNA or sgRNA^{iBAR} or guide sequence thereof, or nucleic acid encoding gRNA or sgRNA or sgRNA^{iBAR} or guide sequence thereof) sequence counts are subject to median ratio normalization followed by mean-variance modeling. In some embodiments, identifying the target gene comprises comparing the hit gene mutation sequence counts obtained from the AICD-resistant immune cell population with hit gene mutation sequence counts obtained from the control immune cell population, wherein: i) the hit genes whose corresponding hit gene mutation sequences are identified as enriched in the AICD-resistant immune cell population compared to the control immune cell population with an FDR≤0.2 (e.g., FDR≤any of 0.15, 0.1, 0.05, 0.04, 0.03, 0.02, 0.01, 0.005, 0.001, or less) (and/or with at least about 2-fold enrichment, such as at least about any of 3-, 4-, 5-, 10-, 20-, 50-, 100-fold, or more enrichment) are identified as target genes whose mutations increase resistance to AICD. In some embodiments, the hit gene mutation sequence counts are subject to median ratio normalization followed by mean-variance modeling.

In some embodiments, the sgRNA library is an sgRNA^{iBAR} library. In some embodiments, the variance of each guide sequence is adjusted based on data consistency among the iBAR sequences in the sgRNA^{iBAR} sequences corresponding to the guide sequence. In some embodiments, the variance of each guide sequence or sequence comprising the hit gene mutation (e.g., inactivating mutation) is adjusted based on data consistency among the same gene. “Data consistency” as used herein refers to consistency of sequencing results of the same guide sequences (e.g., sequence counts, normalized sequence counts, rankings, or fold changes) corresponding to different iBAR sequences in a screening experiment; or consistency of sequencing results of different hit gene mutations such as inactivating mutations (e.g., at different target sites of the same hit gene) or different sgRNA sequences corresponding to the same gene. A true hit from a screen theoretically should have biologically relevant performance similarities, such as similar normalized sequence counts, rankings, and/or fold changes corresponding to sgRNA^{iBAR} constructs having the same guide sequence, but different iBARs; and/or similar normalized sequence counts, rankings, and/or fold changes corresponding to the same gene but different hit gene mutation sequences such as inactivating mutation sequences (e.g., at different target sites of the hit gene) or different sgRNA sequences. Also see WO2020125762 (the content of which is incorporated herein by reference in its entirety) for how mean-variance modeling can be conducted, and how the variance of each guide sequence is adjusted based on data consistency among the iBAR sequences in the sgRNA^{iBAR} sequences corresponding to the guide sequence.

In some embodiments, the data consistency among the iBAR sequences in the sgRNA^{iBAR} sequences corresponding to each guide sequence is determined based on the direction of the fold change of each iBAR sequence, wherein the variance of the guide sequence is increased if the fold changes of the iBAR sequences are in different directions (e.g., increased vs. reduced, increased vs. unchanged, or reduced vs. unchanged are all considered as different directions) with respect to each other. In some embodiments, the data consistency among the different hit gene mutation (e.g., inactivating mutation) sequences or different sgRNA sequences corresponding to the same gene is determined based on the direction of the fold change of each hit gene mutation (e.g., inactivating mutation) sequence or each sgRNA sequence, wherein the variance of the hit gene mutation (e.g., inactivating mutation) sequence or the guide sequence is increased if the fold changes of the different hit gene mutation (e.g., inactivating mutation) sequences or the different sgRNA sequences are in different directions with respect to each other. Such data inconsistency-resulted variance increase can help rule out rare but dramatically changed hit gene mutation (e.g., inactivating mutation)/sgRNA/sgRNA^{iBAR} sequences in positive screens under high MOI. For example, for the iBAR system, due to the high MOI during library construction, there can be “free riders” of false-positive sgRNAs associated with sgRNAs against true-positive hit genes. The “free rider” described herein refers to sgRNAs targeting irrelevant sequences (e.g., irrelevant hit genes) that are mis-associated with sgRNAs targeting true-positive hit genes to enter the same immune cells. In some embodiments, the variance of sgRNAs^{iBAR} is modified based on the enrichment directions of different iBARs for each guide sequence within a set of sgRNA^{iBAR} constructs. If all iBARs of one set of sgRNA^{iBAR} constructs (i.e., all iBARs corresponding to the same guide sequence) present the same direction of fold change, i.e., all greater or less than that of the control group, then the variance of the set of sgRNA^{iBAR} constructs (or the variance of the guide sequence) would be unchanged. If iBARs of one set of sgRNA^{iBAR} constructs (or iBARs corresponding to the same guide sequence) reveal inconsistent directions of fold change relative to control, then the corresponding guide sequence is penalized by increasing its variance. In some embodiments, the final adjusted variance for inconsistent sgRNAs^{iBAR} is the model-estimated variance (e.g., by mean-variance modeling) plus the experimental variance calculated from the test group(s) and the control group(s). In some embodiments, a hit gene comprises two or more (e.g., 2, 3, 4, 5, or more, such as 3) hit gene mutations (e.g., inactivating mutations), or a hit gene is targeted by two or more (e.g., 2, 3, 4, 5, or more, such as 3) different guide sequences at different target sites (e.g., two or more different sgRNAs, or two or more sets of sgRNA^{iBAR} constructs each comprising a guide sequence targeting different target sites). In some embodiments, the data consistency among the iBAR sequences in the sgRNA^{iBAR} sequences corresponding to each guide sequence and to the same hit gene is both determined based on the direction of the fold change of each iBAR sequence, wherein the variance of the guide sequence is increased if the fold changes of the corresponding iBAR sequences are in different directions with respect to each other, and the variance of the guide sequence (or the variance of the hit gene) is further increased if the two or more (e.g., 2, 3, 4, 5, or more, such as 3) different guide sequences targeting the same hit gene have fold changes in different directions with respect to each other. For example, for sgRNA A and sgRNA B targeting different target sites of the same hit gene X, if the guide sequences of both sgRNA A and sgRNA B are enriched compared to control, the variance of each guide sequence or the hit gene do not change; if the guide sequence of sgRNA A is enriched while the guide sequence of sgRNA B is depleted or unchanged compared to control, the variance of each guide sequence or the hit gene is increased.

In some embodiments, the data consistency among the iBAR sequences in the sgRNA^{iBAR} sequences corresponding to the same hit gene is determined based on the direction of the fold change of each iBAR sequence, wherein the variance of each guide sequence targeting the same hit gene is increased if the fold changes of the iBAR sequences corresponding to the same hit gene are in different directions with respect to each other, and the variance of each guide sequence targeting the same hit gene (or the variance of the hit gene) is increased. For example, if 3 sets of sgRNAs^{iBAR} (4 sgRNAs^{iBAR} in each set) target 3 different target sites of the same hit gene, if all 12 iBAR sequences are identified as enriched compared to control, the variances of all 3 guide sequences remain unchanged; if some iBAR sequences are identified as enriched while others are identified as unchanged or depleted compared to control, the variances of all 3 guide sequences are increased.

In some embodiments, the sequences comprising hit gene mutations (e.g., inactivating mutations) at different target sites of the same hit gene whose fold changes among corresponding target sites are shown in different directions, the sgRNAs or sgRNAs^{iBAR} targeting different target sites of the same hit gene whose fold changes among corresponding target sites are shown in different directions, or the sgRNAs whose fold changes among corresponding iBARs are shown in different directions, can be penalized through the increased variance leading to lower scores and rankings for certain hit genes. For example, if 3 sets of sgRNAs^{iBAR} (4 sgRNAs^{iBAR} in each set) target 3 different target sites of the same hit gene, if all 12 iBAR sequences are identified as enriched compared to control, the hit gene has low variance and hence high ranking and/or score (e.g., high ranking AICD resistance gene, with high AICD resistance score); if some iBAR sequences are identified as enriched while others are identified as unchanged or depleted compared to control, the hit gene has high variance and hence low ranking and/or score (e.g., low ranking AICD resistance gene, with low AICD resistance score).

In a set of sgRNA^{iBAR} constructs, the ranking for the guide sequence may be adjusted based on the consistency of enrichment directions of a pre-determined threshold number x of different iBAR sequences in the set, wherein x is an integer between 1 and y. For example, if at least x iBAR sequences of the sgRNA^{iBAR} set present the same direction of fold change, i.e., all greater or less than that of the control immune cell population, then the ranking (or variance) of the guide sequence is unchanged. However, if more than y-x different iBAR sequences revealed inconsistent directions of fold change, then the sgRNA^{iBAR} set would be penalized by lowering its ranking, e.g., by increasing its variance. In some embodiments, the ranking for the sequences containing the hit gene mutations (e.g., inactivating mutations) or the guide sequences may be adjusted (or further adjusted) based on the consistency of enrichment directions of a pre-determined threshold number x of different hit gene mutations (e.g., inactivating mutations) or different guide sequences corresponding to the same hit gene, wherein x is an integer between 1 and y. For example, if at least x hit gene mutations (e.g., inactivating mutations) or x guide sequences corresponding to the same hit gene present the same direction of fold change, i.e., all greater or less than that of the control immune cell population, then the ranking (or variance) is unchanged. However, if more than y-x different hit gene mutations (e.g., inactivating mutations) or more than y-x different guide sequences revealed inconsistent directions of fold change, then the sequences comprising the hit gene mutations (e.g., inactivating mutations) or the guide sequences would be penalized by lowering their ranking, e.g., by increasing their variance.

In some embodiments, the P-value of each sequence comprising a hit gene mutation (e.g., inactivating mutation), or the P-value of each guide sequence of gRNA or sgRNA or sgRNA^{iBAR}, is calculated using the mean and variance (e.g., experimental variance, model-estimated variance, or modified variance based on data inconsistency) of the test group compared to those of the control group.

Robust Rank Aggregation (RRA; Kolde R et al. Bioinformatics. 2012; 28:573-580) or modified RRA (e.g., α-RRA in MAGeCK; Li W et al. Genome Biol. 2014; 15:554) is one of available tools for statistics and ranking in the art, which can detect genes that are ranked consistently better than expected under null hypothesis of uncorrelated inputs and assign a significance score for each gene, and combine ranking lists into a single ranking. A skilled person in the art can understand that other tools can also be used for this statistics and ranking. In some embodiments, RRA or α-RRA is employed to calculate the final score of each hit gene in order to obtain the ranking of hit genes based on mean and variance (e.g., modified variance) of every hit gene.

In some embodiments, sequences comprising the hit gene mutations (e.g., inactivating mutations), gRNA (e.g., sgRNA) guide sequences, or sgRNA^{iBAR} guide sequences (hit gene mutation (e.g., inactivating mutation)/gRNA guide/sgRNA^{iBAR} guide sequences) were ranked based on P-values calculated using the mean and variance (e.g., modified variance adjusted for data inconsistency) from the negative binomial (NB) distribution model, which is used to estimate probability of every hit gene mutation (e.g., inactivating mutation)/gRNA guide/sgRNA^{iBAR} guide sequence across biological/experimental replicates and treatment vs. control groups, then RRA or α-RRA algorithm is applied to identify positively selected hit genes corresponding to the top ranking (e.g., top a % such as top 5%) hit gene mutation (e.g., inactivating mutation)/gRNA guide/sgRNA^{iBAR} guide sequence. A lower RRA score corresponded to a stronger enrichment of the hit genes. In some embodiments, the P-values of such top-ranking hit gene mutation (e.g., inactivating mutation)/gRNA guide/sgRNA^{iBAR} guide sequence lower than a threshold (e.g., P-value<0.25) are selected, and the corresponding hit genes are identified as the target gene. In some embodiments, the FDRs of such top ranking hit gene mutation (e.g., inactivating mutation)/gRNA guide/sgRNA^{iBAR} guide sequence lower than a threshold (e.g., FDR≤0.2) are selected, and the corresponding hit genes are identified as the target gene. In some embodiments, when multiple hit gene mutation (e.g., inactivating mutation)/gRNA guide/sgRNA^{iBAR} guide sequences are designed for the same hit gene, only the top hit gene mutation (e.g., inactivating mutation)/gRNA guide/sgRNA^{iBAR} guide sequences of one gene is considered in the RRA or α-RRA calculation. In some embodiments, all hit gene mutation (e.g., inactivating mutation)/gRNA guide/sgRNA^{iBAR} guide sequences are ranked and compared by RRA or α-RRA among test (e.g., AICD-resistant immune cell population) and control groups according to their relative ranking in each group and the different distributions of the groups. All AICD-resistant immune cell population covered hit genes are ranked by comparing the skew in beta distribution of the hit gene mutation (e.g., inactivating mutation)/gRNA guide/sgRNA^{iBAR} guide sequences to the uniform null hypothesis model, and hit genes whose corresponding hit gene mutation (e.g., inactivating mutation)/gRNA guide/sgRNA^{iBAR} guide sequence rankings are consistently higher than expected with statistical significance (P-value) by permutation test and/or acceptable FDR by the Benjamini-Hochberg Procedure, are prioritized in RRA or α-RRA (lower RRA score). In some embodiments, hit genes are ranked based on ranking scores of corresponding hit gene mutation (e.g., inactivating mutation)/gRNA guide/sgRNA^{iBAR} guide sequence obtained by median ratio normalization followed by mean-variance modeling. In some embodiments, hit genes are further ranked by RRA or α-RRA taking into consideration of multiple hit gene mutation (e.g., inactivating mutation)/gRNA guide/sgRNA^{iBAR} guide sequences for the same hit gene.

Any target identification methods known in the art can be used herein. For example, the Empirical Bayesian method (identifies target by likelihood) or algorithm based therefrom (see Morgens, D. W. et al. (2016) Nat Biotechnol 34, 634-636), RIGER (Luo, J. et al. (2009). Cell 137, 835-848), STARS (Doench, J. G., et al. (2016) Nat Biotechnol 34, 184-191), Negative Binomial model-based and α-RRA algorithm such as MAGeCK (Li, W. et al. (2014) Genome Biol 15, 554), CRISPRBetaBinomial (CB²) (Jeong, H. H. et al. (2019). Genome Res 29, 999-1008).

In some embodiments, the target gene identification is a positive screening, i.e., by identifying hit gene mutation (e.g., inactivating mutation) sequences or guide sequences that are enriched in the AICD-resistant immune cell population. Hit gene mutation (e.g., inactivating mutation) sequences or guide sequences that are enriched in the AICD-resistant immune cell population rank high based on sequence counts or fold changes. In some embodiments, the enrichment in the AICD-resistant immune cell population is relative to the total sequence counts obtained from the AICD-resistant immune cell population. In some embodiments, the enrichment is relative to the corresponding sequence counts in a control immune cell population or control immune cell library, such as a control immune cell population obtained from a same immune cell library not subjected to step b). In some embodiments, the enrichment is calculated based on RRA or α-RRA algorithm.

In some embodiments, the method further comprises ranking the identified target genes, wherein the target gene ranking is based on the degree of enrichment (e.g., fold of enrichment, enrichment FDR) of the gRNA or sgRNA or sgRNA^{iBAR} guide sequences or hit gene mutations in the AICD-resistant immune cell population compared to the control immune cell population.

In some embodiments, the target gene ranking is based on the degree of enrichment of the gRNA (e.g., sgRNA) guide sequences in the AICD-resistant immune cell population compared to the control immune cell population. In some embodiments, the target gene ranking is further adjusted based on data consistency among all sequences comprising the hit gene mutation (e.g., inactivating mutation) corresponding to the same target gene. In some embodiments, the sgRNA library is an sgRNA^{iBAR} library, and the target gene ranking is further adjusting based on data consistency among the iBAR sequences in the sgRNA^{iBAR} sequences corresponding to the guide sequence of the target gene, and/or based on data consistency among all guide sequences corresponding to (e.g., same or different target sites) of the same target gene. In some embodiments, RRA or α-RRA algorithm is used for ranking the identified target genes. In some embodiments, the ranking of the identified target genes is i) based on data consistency among all sequences comprising the hit gene mutation (e.g., inactivating mutation) corresponding to the same target gene; or ii) based on data consistency among the iBAR sequences in the sgRNA^{iBAR} sequences corresponding to the guide sequence of the target gene; and/or iii) based on data consistency among all guide sequences of sgRNAs or sgRNAs^{iBAR} corresponding to (e.g., same or different target sites) of the same target gene; wherein the identified target genes are ranked from high to low based on the degree of data consistency from high to low. In some embodiments, the method further comprises assigning an AICD resistance score to the identified target gene. In some embodiments, target genes whose mutations increase resistance to AICD are ranked from high to low based on the enrichment (e.g., fold of enrichment, or enrichment FDR—the smaller the FDR, the higher the ranking) and/or based on the degree of data consistency (the higher the degree of data consistency, the higher the ranking) of the gRNAs or sgRNAs or sgRNAs^{iBAR} guide sequences (or hit gene mutation sequences) in the AICD-resistant immune cell population compared to the control immune cell population; optionally, each target gene is assigned an AICD resistance score from high to low accordingly.

In some embodiments, the method further comprising validating the identified target gene by: a) modifying an immune cell by creating a mutation (e.g., inactivating mutation) in the target gene in the immune cell, or reducing or abolish expression and/or function of the target gene or encoded product (RNA or protein); and b) determining the resistance to AICD of the modified immune cell, such as by comparing to a same immune cell not containing such mutation, or a same immune cell not treated with FasL. In some embodiments, the method comprises comparing the modified immune cell to a same immune cell not containing such mutation for one or more of the properties: AICD, GvHD, HvG, immune cell proliferation, differentiation, maturation, activation, persistence, homeostasis, and effector function (e.g., cytokine release, or cytotoxic killing of target cells). Any suitable assays known in the art (e.g., biomarkers of immune cell status and/or activity) and described herein can be used to determine such one or more immune cell properties. The mutation (e.g., inactivating mutation) in the target gene can be generated by any methods known in the art and described herein, such as by mutagenic agent, or NHEJ-, HDR-, TALEN-, ZFN-, or CRISPR/Cas-mediated gene editing (e.g., using Cas, sgRNA against the target gene), or LEAPER (for RNA editing). In some embodiments, the immune cell before creating a mutation (e.g., inactivating mutation) in the target gene contains an endogenous mutation, such as an endogenous mutation frequently occurs in immune cells of patients with cancer or immune-related diseases. In some embodiments, the immune cell before creating a mutation (e.g., inactivating mutation) in the target gene comprises an engineered component (e.g., CAR). In some embodiments, the method further comprises examining whether (and/or how much) the mutation in the target gene affects the expression and/or function of the engineered component (e.g., CAR).

### VI. KITS AND ARTICLES OF MANUFACTURE

The present application further provides kits and articles of manufacture for use in any embodiment of the methods of identifying a target gene in an immune cell described herein, such as using the gRNA (e.g., sgRNA libraries) or sgRNA^{iBAR} libraries described herein. Also provided are kits and articles of manufacture for generating modified immune cells with increased resistance to AICD, or with no or reduced expression and/or function of one or more target proteins (e.g., SPPL3) or other proteins (e.g., TCR) described herein, such as target proteins selected from SPPL3, FADD, FAS, CASP8, ARID1A, BAK1, BID, ETS1, IKZF2, and HIST1H1B.

In some embodiments, there is provided a kit for identifying a target gene in an immune cell whose mutation increases resistance to AICD, comprising any of the sgRNA libraries or sgRNA^{iBAR} libraries described herein. In some embodiments, the kit further comprises a Cas protein or a nucleic acid encoding the Cas protein (e.g., Cas9). In some embodiments, the kit further comprises one or more positive and/or negative control sets of sgRNA^{iBAR} constructs, or one or more positive and/or negative control of sgRNA constructs. In some embodiments, the kit further comprises the initial population of immune cells (e.g., CAR-T cells), or immune cells comprising the Cas component. In some embodiments, the kit further comprises data analysis software. In some embodiments, the kit comprises instructions for carrying out any one of the methods described herein.

In some embodiments, there is provided a kit for generating any of the modified immune cells described herein, such as those with no or reduced expression and/or function of one or more target proteins (e.g., SPPL3) or other proteins (e.g., TCR) described herein, or those with increased resistance to AICD, or carrying an aberration (e.g., LOF mutation) in any of the target genes identified herein. In some embodiments, the kit comprises an antisense RNA, an siRNA, or an shRNA (or a nucleic acid encoding thereof) specifically recognizing an RNA encoding any of the target proteins (e.g., SPPL3) or other proteins (e.g., TCR) described herein, or proteins encoded by any of the target genes identified herein. In some embodiments, the kit comprises a gRNA (e.g., sgRNA) or nucleic acid encoding thereof, wherein the gRNA specifically recognizes a nucleic acid sequence encoding any of the target proteins (e.g., SPPL3) or other proteins (e.g., TCR) described herein, or a nucleic acid sequence of any of the target genes identified herein. In some embodiments, the kit further comprises a Cas protein or a nucleic acid encoding the Cas protein. In some embodiments, the kit further comprises a therapeutic agent. In some embodiments, the kit comprises instructions for carrying out any one of the methods described herein.

In some embodiments, there is provided a kit for treating a disease (e.g., cancer, or immune-related disease such as infection or immune cell exhaustion) in an individual, comprising modified immune cells described herein, or agents (e.g., gRNAs, Cas) for generating aberrations in one or more of the AICD resistance genes described herein, or agents (e.g., antisense RNA, an siRNA, or an shRNA) for generating immune cells with no or reduced expression and/or function of any of the target proteins (e.g., SPPL3) or other proteins (e.g., TCR) described herein. In some embodiments, the kit further comprises additional agent suitable for treating the disease.

The kit may contain additional components, such as containers, reagents, culturing media, primers, buffers, enzymes, and the like to facilitate execution of any one of the methods described herein. In some embodiments, the kit comprises reagents, buffers and vectors for introducing the gRNA (e.g., sgRNA), gRNA (e.g., sgRNA) library, or sgRNA^{iBAR} library and the Cas protein or nucleic acid encoding the Cas protein to the immune cell. In some embodiments, the kit comprises primers, reagents and enzymes (e.g., polymerase) for preparing a sequencing library of sequences comprising hit gene mutations (e.g., inactivating mutations), gRNA (e.g., sgRNA) sequences, or sgRNA^{iBAR} sequences extracted from the AICD-resistant immune cell population or control immune cell population.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition (e.g., any modified immune cells described herein), and may have a sterile access port. Package insert refers to instructions customarily included in commercial packages that contain information about the instructions and/or warnings concerning the use of such products. It may further include other materials desirable from a commercial and user standpoint, including other buffers, diluents, filters.

### EXEMPLARY EMBODIMENTS

Embodiment 1. An immune cell, wherein the immune cell is modified to have no or reduced expression and/or function of one or more target proteins selected from the group consisting of: Signal Peptide Peptidase Like 3 (SPPL3), FADD, FAS, CASP8, ARID1A, BAK1, BID, ETS1, IKZF2, and HIST1H1B.

Embodiment 2. The immune cell of embodiment 1, wherein the immune cell is modified to have no or reduced expression and/or function of SPPL3 protein.

Embodiment 3. The immune cell of embodiment 2, wherein the immune cell has at least about 10% less activation-induced cell death (AICD) compared to a reference immune cell not having the modification to reduce or eliminate expression and/or function of the SPPL3 protein.

Embodiment 4. The immune cell of embodiment 2 or 3, wherein the expression of the SPPL3 protein is reduced or inhibited by an antisense RNA, an siRNA, or an shRNA specifically recognizing an RNA encoding the SPPL3 protein.

Embodiment 5. The immune cell of embodiment 2 or 3, wherein the immune cell is modified to express a dominant negative SPPL3 protein variant or dominant negative fragment thereof.

Embodiment 6. The immune cell of embodiment 2 or 3, wherein the immune cell is genetically modified at the SPPL3 locus or at SPPL3 RNA.

Embodiment 7. The immune cell of embodiment 6, wherein the SPPL3 locus is modified by gene editing, or wherein the SPPL3 RNA is modified by RNA editing.

Embodiment 8. The immune cell of embodiment 7, wherein the gene editing or RNA editing is mediated by non-homologous end-joining (NHEJ), homology directed repair (HDR), zinc-finger nuclease (ZFN), transcription activator-like effector nuclease (TALEN), or CRISPR/Cas.

Embodiment 9. The immune cell of embodiment 7 or 8, wherein the gene editing comprises gene knockout (KO).

Embodiment 10. The immune cell of embodiment 7 or 8, wherein the gene editing or RNA editing comprises base editing.

Embodiment 11. The immune cell of any one of embodiments 7-10, wherein the gene editing or RNA editing is mediated by CRISPR/Cas.

Embodiment 12. The immune cell of embodiment 11, wherein the gene editing or RNA editing comprises contacting a precursor immune cell with i) a guide RNA (gRNA) construct, wherein the gRNA construct comprises or encodes a gRNA comprising a guide sequence that is complementary to a target site in the SPPL3 locus or SPPL3 RNA; and optionally ii) a Cas component comprising a Cas protein or a nucleic acid encoding the Cas protein, under a condition that allows introduction of the gRNA construct and the optional Cas component into the precursor immune cell.

Embodiment 13. The immune cell of embodiment 12, wherein the precursor immune cell expresses a Cas protein.

Embodiment 14. The immune cell of embodiment 12 or 13, wherein the Cas protein has endonuclease activity.

Embodiment 15. The immune cell of embodiment 12 or 13, wherein the Cas protein is fusion protein comprising i) a dead Cas protein (dCas) and ii) an adenine deaminase (ADA) or a cytidine deaminase (CDA) or functional fragment thereof.

Embodiment 16. The immune cell of any one of embodiments 12-15, wherein the Cas protein is Cas9.

Embodiment 17. The immune cell of any one of embodiments 1-16, wherein the immune cell has or is further modified to have no or reduced expression and/or function of one or more other proteins selected from the group consisting of TCRα, TCRβ, TCRγ, TCRδ, HLA-A, HLA-B, HLA-C, HLA-E, HLA-F, HLA-G, B2M, PD-1, TIM-3, LAG-3, CTLA-4, CISH, Fas, FADD, CASP8, ARID1A, BAK1, BID, ETS1, IKZF2, HIST1H1B, B7-H6, MICA, MICB, ULBP1, ULBP2, ULBP3, ULBP4, ULBP5, ULBP6, and ligands of NKp46.

Embodiment 18. The immune cell of any one of embodiments 1-17, wherein the immune cell expresses or is further modified to express an engineered receptor.

Embodiment 19. The immune cell of embodiment 18, wherein the engineered receptor is a chimeric antigen receptor (CAR), an engineered TCR, or a T cell antigen coupler (TAC).

Embodiment 20. The immune cell of embodiment 18 or 19, wherein the engineered receptor is a CAR comprising:
i) an extracellular antigen binding domain specifically recognizing a target antigen;
ii) a transmembrane domain; and
iii) an intracellular signaling domain.

Embodiment 21. The immune cell of any one of embodiments 18-20, wherein the modification to reduce or eliminate expression and/or function of the one or more target proteins:
i) does not down-regulate or eliminate expression and/or function of the engineered receptor; or
ii) down-regulates expression and/or function of the engineered receptor by at most about 30%.

Embodiment 22. The immune cell of any one of embodiments 1-21, wherein the immune cell is a T cell, a B cell, or a natural killer (NK) cell.

Embodiment 23. The immune cell of embodiment 22, wherein the immune cell is a T cell.

Embodiment 24. The immune cell of embodiment 23, wherein T cell is selected from the group consisting of a helper CD4⁺ T cell, a cytotoxic CD8⁺ T cell, a memory T cell, a regulatory CD4⁺ T cell, a natural killer T (NKT) cell, a mucosal associated invariant T (MAIT) cell, a double negative T (DNT) cell, and a γδ T cell.

Embodiment 25. The immune cell of embodiment 23 or 24, wherein the modification to reduce or eliminate expression and/or function of the one or more target proteins:
i) reduces at least about 10% cell surface expression of one or more of Fas, B7-H6, HLA-A, HLA-B, HLA-C, HLA-E, B7-H6, MICA, MICB, ULBP1, ULBP2, ULBP3, ULBP4, ULBP5, ULBP6, and ligands of NKp46;
ii) reduces at least about 10% killing by an allogeneic T cell; and/or
iii) reduces at least about 10% killing by an autologous or allogeneic NK cell.

Embodiment 26. The immune cell of any one of embodiments 1-25, wherein the immune cell has at least about 10% longer in vivo persistence compared to a reference immune cell not having the modification to reduce or eliminate expression and/or function of the one or more target proteins.

Embodiment 27. The immune cell of any one of embodiments 1-26, which is autologous.

Embodiment 28. The immune cell of any one of embodiments 1-26, which is allogeneic.

Embodiment 29. A pharmaceutical composition comprising the immune cell of any one of embodiments 1-28, and optionally a pharmaceutically acceptable excipient.

Embodiment 30. A method of treating a disease in an individual, comprising administering to the individual an effective amount of the immune cell of any one of embodiments 1-28, or the pharmaceutical composition of embodiment 29.

Embodiment 31. The method of embodiment 30, wherein the disease is associated with the expression of a target antigen, and wherein the immune cell expresses an engineered receptor specifically recognizing the target antigen.

Embodiment 32. The method of embodiment 31, wherein the engineered receptor is a CAR.

Embodiment 33. The method of any one of embodiments 30-32, wherein the disease is a cancer, an infection, an inflammation, an autoimmune disease, or an immune-related disease characterized by effector cell exhaustion.

Embodiment 34. A method of identifying an individual as a suitable donor of an immune cell with prolonged in vivo persistence, comprising examining the expression and/or function of one or more target proteins selected from the group consisting of SPPL3, FADD, FAS, CASP8, ARID1A, BAK1, BID, ETS1, IKZF2, and HIST1H1B in the individual, wherein the identification of reduced or abolished expression and/or function of the one or more target proteins compared to a reference identifies the individual as the suitable donor.

Embodiment 35. The method of embodiment 34, comprising examining the expression and/or function of SPPL3 protein in the individual, wherein the identification of reduced or abolished expression and/or function of the SPPL3 protein compared to a reference identifies the individual as the suitable donor.

Embodiment 36. The method of embodiment 34 or 35, further comprising examining the expression and/or function of one or more other proteins selected from the group consisting of TCRα, TCRβ, TCRγ, TCRδ, HLA-A, HLA-B, HLA-C, HLA-E, HLA-F, HLA-G, B2M, PD-1, TIM-3, LAG-3, CTLA-4, CISH, Fas, FADD, CASP8, ARID1A, BAK1, BID, ETS1, IKZF2, HIST1H1B, B7-H6, MICA, MICB, ULBP1, ULBP2, ULBP3, ULBP4, ULBP5, ULBP6, and ligands of NKp46, wherein the identification of reduced or abolished expression and/or function of one or more of the other proteins further identifies the individual as the suitable donor.

Embodiment 37. A method of excluding an individual as a suitable donor of an immune cell with prolonged in vivo persistence, comprising examining the expression and/or function of one or more target proteins selected from the group consisting of SPPL3, FADD, FAS, CASP8, ARID1A, BAK1, BID, ETS1, IKZF2, and HIST1H1B in the individual, wherein the individual is excluded as the suitable donor if no reduced or abolished expression and/or function of the one or more target proteins compared to a reference is identified.

Embodiment 38. The method of embodiment 37, comprising examining the expression and/or function of SPPL3 protein in the individual, wherein the individual is excluded as the suitable donor if no reduced or abolished expression and/or function of the SPPL3 protein compared to a reference is identified.

Embodiment 39. The method of any one of embodiments 34, 36, and 37, wherein the reference is the average expression and/or function of the one or more target proteins in a population of individuals.

Embodiment 40. The method of any one of embodiments 34-39, wherein the reference is the average expression and/or function of the SPPL3 protein in a population of individuals.

Embodiment 41. The method of any one of embodiments 34, 36, 37, and 39, wherein examining the expression and/or function of the one or more target proteins and/or the one or more other proteins comprises examining the sequence of the nucleic acid encoding the one or more target proteins and/or the one or more other proteins, wherein the identification of a mutation in the nucleic acid that reduces expression and/or function of the one or more target proteins and/or the one or more other proteins identifies the individual as the suitable donor.

Embodiment 42. The method of any one of embodiments 34-41, wherein examining the expression and/or function of the SPPL3 protein and/or the one or more other proteins comprises examining the sequence of the nucleic acid encoding the SPPL3 protein and/or the one or more other proteins, wherein the identification of a mutation in the nucleic acid that reduces expression and/or function of the SPPL3 protein and/or the one or more other proteins identifies the individual as the suitable donor.

Embodiment 43. A method of i) prolonging in vivo persistence, ii) reducing AICD, and/or iii) reducing host-versus-graft (HvG) response of an immune cell, comprising modifying the immune cell to reduce or eliminate expression and/or function of one or more target proteins selected from the group consisting of: SPPL3, FADD, FAS, CASP8, ARID1A, BAK1, BID, ETS1, IKZF2, and HIST1H1B.

Embodiment 44. The method of embodiment 43, wherein the method comprises modifying the immune cell to reduce or eliminate expression and/or function of SPPL3 protein.

Embodiment 45. The method of embodiment 44, wherein the expression of the SPPL3 protein is reduced or inhibited by an antisense RNA, an siRNA, or an shRNA specifically recognizing an RNA encoding the SPPL3 protein.

Embodiment 46. The method of embodiment 44, wherein the immune cell is modified to express a dominant negative SPPL3 protein variant or dominant negative fragment thereof.

Embodiment 47. The method of embodiment 44, wherein the immune cell is genetically modified at the SPPL3 locus or at SPPL3 RNA.

Embodiment 48. The method of embodiment 47, wherein the SPPL3 locus is modified by gene editing, or wherein the SPPL3 RNA is modified by RNA editing.

Embodiment 49. The method of embodiment 48, wherein the gene editing or RNA editing is mediated by NHEJ, HDR, ZFN, TALEN, or CRISPR/Cas.

Embodiment 50. The method of embodiment 48 or 49, wherein the gene editing comprises gene KO.

Embodiment 51. The method of embodiment 48 or 49, wherein the gene editing or RNA editing comprises base editing.

Embodiment 52. The method of embodiment any one of embodiments 48-51, wherein the gene editing or RNA editing is mediated by CRISPR/Cas.

Embodiment 53. The method of embodiment 52, comprising contacting a precursor immune cell with i) a gRNA construct, wherein the gRNA construct comprises or encodes a gRNA comprising a guide sequence that is complementary to a target site in the SPPL3 locus or SPPL3 RNA; and optionally ii) a Cas component comprising a Cas protein or a nucleic acid encoding the Cas protein, under a condition that allows introduction of the gRNA construct and the optional Cas component into the precursor immune cell.

Embodiment 54. The method of embodiment 53, wherein the precursor immune cell expresses a Cas protein.

Embodiment 55. The method of embodiment 53 or 54, wherein the Cas protein has endonuclease activity.

Embodiment 56. The method of embodiment 53 or 54, wherein the Cas protein is fusion protein comprising i) a dCas and ii) an ADA or a CDA or functional fragment thereof.

Embodiment 57. The method of any one of embodiments 53-56, wherein the Cas protein is Cas9.

Embodiment 58. The method of any one of embodiments 43-57, further comprising modifying the immune cell to reduce or eliminate expression and/or function of one or more other proteins selected from the group consisting of TCRα, TCRβ, TCRγ, TCRδ, HLA-A, HLA-B, HLA-C, HLA-E, HLA-F, HLA-G, B2M, PD-1, TIM-3, LAG-3, CTLA-4, CISH, Fas, FADD, CASP8, ARID1A, BAK1, BID, ETS1, IKZF2, HIST1H1B, B7-H6, MICA, MICB, ULBP1, ULBP2, ULBP3, ULBP4, ULBP5, ULBP6, and ligands of NKp46.

Embodiment 59. The method of embodiment 58, wherein the immune cell is genetically modified at one or more loci or RNAs encoding the one or more other proteins.

Embodiment 60. The method of any one of embodiments 43-59, wherein the immune cell expresses an engineered receptor.

Embodiment 61. The method of any one of embodiments 43-59, further comprising introducing into the immune cell a nucleic acid encoding an engineered receptor.

Embodiment 62. The method of embodiment 61, wherein the nucleic acid encoding the engineered receptor, the nucleic acid encoding the gRNA against SPPL3, and/or the nucleic acid encoding the Cas protein are on different vectors.

Embodiment 63. The method of any one of embodiments 60-62, wherein the engineered receptor is a CAR, an engineered TCR, or a TAC.

Embodiment 64. The method of any one of embodiments 60-63, wherein the engineered receptor is a CAR comprising:
i) an extracellular antigen binding domain specifically recognizing a target antigen;
ii) a transmembrane domain; and
iii) an intracellular signaling domain.

Embodiment 65. The method of any one of embodiments 60-64, wherein the modification to reduce or eliminate expression and/or function of the one or more target proteins:
i) does not down-regulate or eliminate expression and/or function of the engineered receptor; or
ii) down-regulates expression and/or function of the engineered receptor by at most about 30%.

Embodiment 66. The method of any one of embodiments 43-65, wherein the immune cell is a T cell, a B cell, or an NK cell.

Embodiment 67. The method of embodiment 66, wherein the immune cell is a T cell.

Embodiment 68. The method of embodiment 67, wherein T cell is selected from the group consisting of a helper CD4+ T cell, a cytotoxic CD8+ T cell, a memory T cell, a regulatory CD4+ T cell, an NKT cell, an MAIT cell, a DNT cell, and a γδ T cell.

Embodiment 69. The method of embodiment 67 or 68, wherein the modification to reduce or eliminate expression and/or function of the one or more target proteins:
i) reduces at least about 10% cell surface expression of one or more of Fas, B7-H6, HLA-A, HLA-B, HLA-C, HLA-E, B7-H6, MICA, MICB, ULBP1, ULBP2, ULBP3, ULBP4, ULBP5, ULBP6, and ligands of NKp46;
ii) reduces at least about 10% killing by an allogeneic T cell; and/or
iii) reduces at least about 10% killing by an autologous or allogeneic NK cell.

Embodiment 70. The method of any one of embodiments 43-69, wherein the modification to reduce or eliminate expression and/or function of the one or more target proteins:
i) prolongs at least about 10% in vivo persistence of the immune cell; and/or
ii) reduces at least about 10% AICD of the immune cell, compared to a same immune cell not modified to reduce or eliminate expression and/or function of the one or more target proteins.

Embodiment 71. An immune cell obtained by the method of any one of embodiments 43-70.

Embodiment 72. The immune cell of embodiment 71, which is autologous.

Embodiment 73. The immune cell of embodiment 71, which is allogeneic.

Embodiment 74. A pharmaceutical composition comprising the immune cell of any one of embodiments 71-73, and optionally a pharmaceutically acceptable excipient.

Embodiment 75. A method of treating a disease in an individual, comprising administering to the individual an effective amount of the immune cell of any one of embodiments 71-73, or the pharmaceutical composition of embodiment 74.

Embodiment 76. The method of embodiment 75, wherein the disease is associated with the expression of a target antigen, and wherein the immune cell expresses an engineered receptor specifically recognizing the target antigen.

Embodiment 77. The method of embodiment 76, wherein the engineered receptor is a CAR.

Embodiment 78. The method of any one of embodiments 75-77, wherein the disease is a cancer, an infection, an inflammation, an autoimmune disease, or an immune-related disease characterized by effector cell exhaustion.

### EXAMPLES

The examples and exemplary embodiments below are intended to be purely exemplary of the invention and should therefore not be considered to limit the invention in any way. The following examples and detailed description are offered by way of illustration and not by way of limitation.

### Example 1. Identification of Target Genes that Modulate Immune Cell Persistence

This example provides exemplary methods for identifying target genes that modulate immune cell (e.g., CAR-T cell) persistence, such as target genes after knock-out (KO) that reduce or eliminate activation-induced cell death (AICD) due to Fas/FasL interaction, also referred to as “FasL resistant genes” or “AICD resistant genes.” Briefly, a Cas9-expressing T cell library carrying sgRNA^{iBAR} targeting each human gene was constructed for Cas9-mediated gene KO. By examining FasL-induced cell death of the Cas9⁺ sgRNA^{iBAR} T cell library constructed, genes that modulate immune cell persistence after KO can be identified. FIGS. 1, 2, and 4 show the workflow.

### 1. Construction of Human Genome-Scale sgRNA^{iBAR} Plasmid and Viral Library

Human genome-scale CRISPR sgRNA^{iBAR} library was designed and constructed similarly as described in WO2020125762 and Zhu et al. (“Guide RNAs with embedded barcodes boost CRISPR-pooled screens,” Genome Biol. 2019; 20:20), the contents of each of which are incorporated herein by reference in their entirety. Briefly, 19,114 annotated protein-coding genes were retrieved from UCSC human genome. For each gene, 3 sets of sgRNAs targeting 3 different target sites were designed using the DeepRank algorithm (see Zhu et al.), and four 6-bp iBARs (iBAR₆s) were randomly assigned to each set of sgRNAs (“sgRNAs^{iBAR}”) I.e., 3 sets of sgRNA^{iBAR} for each gene targeting 3 different target sites, each set of sgRNA^{iBAR} contained 4 sgRNAs^{iBAR}. In addition, 250 sets of control sgRNAs not targeting any human genes were designed as negative control, and four iBAR₆s were randomly assigned to each set of sgRNAs (“control sgRNA^{iBAR}”). I.e., 1 set of control sgRNA^{iBAR} for each non-target site, each set of control sgRNAs^{iBAR} contained 4 control sgRNAs^{iBAR}. The internal barcode sequence was designed to be placed in the tetra loop of the gRNA scaffold outside of the Cas9-sgRNA ribonucleoprotein complex, which did not affect the activity of its upstream guide sequence.

DNA oligonucleotides encoding the target sgRNAs^{iBAR} or control sgRNAs^{iBAR} were designed and synthesized, then PCR amplified. PCR products were purified with PCR purification kit, then cloned via Golden Gate cloning into lentiviral sgRNA^{iBAR}-expressing backbone modified in house based on pLenti-sgRNA-Lib (addgene #53121) to obtain sgRNA^{iBAR} library plasmids, which encodes 229,368 target sgRNAs^{iBAR} covering 19,114 human genes, and 1,000 control sgRNAs^{iBAR} covering 250 non-target sites. The designed CRISPR sgRNA^{iBAR} library therefore included a total of 230,368 sgRNAs^{iBAR}.

In order to ensure the abundance of sgRNAs^{iBAR} in the Jurkat T cell library (at least 1000-fold coverage for each sgRNA^{iBAR}), 10 electroporation reactions were performed using sgRNA^{iBAR} plasmids obtained above. For each electroporation reaction, 1 μL sgRNA^{iBAR} plasmids were added into a sterile 1.5 mL Eppendorf tube, 50 μL competent cells (E. coli) were further added to the tube and swirled, then electroporation was conducted. 950 μL Super Optimal Broth (SOC) medium without antibiotics was immediately added to each reaction tube, gently pipetted to mix, then incubated in a shaker at 37° C. and 225 rpm for 1 hour. The resulting bacteria were transferred to 1 L LB liquid medium supplemented with Ampicillin, cultured overnight in a shaker at 37° C. and 225 rpm. The next day, plasmid extraction was performed on the obtained bacteria using EndoFree® Plasmid Purification Kit (QIAGEN, #12391).

sgRNA^{iBAR} library lentiviruses were then obtained using standard protocol by infecting HEK293T cells. Briefly, 1×10⁷ HEK293T cells were placed in a 10 cm cell culture dish, 20 mL HEK293T complete cell culture medium (Dulbecco's Modified Eagle Medium (DMEM)+10% Fetal Bovine Serum (FBS, BioInd)+5 mg/mL penicillin+10 mg/mL streptomycin) was added, then HEK293T cells were cultured overnight in a 37° C., 5% CO₂ incubator. The next day, culture medium was discarded, 10 mL fresh serum-free medium was added to the HEK293T cells. The transfection complex was prepared using serum-free medium (4 mL, DMEM+5 mg/mL penicillin+10 mg/mL streptomycin), sgRNA^{iBAR} library plasmids obtained above (20 μg), pCMVR8.74 plasmid (20 g), and pCMV-VSV-G plasmid (2 g); after mixing, 105 μL PEI was added; after mixing, the transfection complex was let stand for 15 minutes in room temperature. The transfection complex was then added to HEK293T cells in 10 mL fresh serum-free medium, incubated in an incubator at 37° C., 5% CO₂ for 6 hours. Cell medium was then discarded. 20 mL fresh complete medium (DMEM+10% FBS+5 mg/mL penicillin+10 mg/mL streptomycin) was added to HEK293T cells, then incubated in an incubator at 37° C., 5% CO₂ for 3 days, after which the culture medium was collected and centrifuged at 1000 rpm, 4° C., 10 minutes. The supernatant containing sgRNA^{iBAR} library lentiviruses was collected and measured for viral titer, and aliquoted for future use.

### 2. Construction of Human Genome-Scale Cas9⁺ sgRNA^{iBAR} Jurkat T Cell Library

Cas9-expressing (Cas9⁺) Jurkat T cells were gift from Haopeng Wang lab, ShanghaiTech University. Cas9⁺ Jurkat T cells were cultured in “Jurkat cell complete medium” (RPMI 1640 Medium (Gibco)+10% FBS+5 mg/mL penicillin+10 mg/mL streptomycin) in 37° C., 5% CO₂ incubator.

In order to ensure the abundance of sgRNAs^{iBAR} in the Jurkat T cell library (at least 1000-fold coverage for each sgRNA^{iBAR}), sgRNA^{iBAR} library lentiviruses were added to 2×10⁷ Cas9⁺ Jurkat T cells obtained above at an MOI of 3 and gently mixed. The cell mixture was cultured in a 37° C., 5% CO₂ incubator for 24 hours for infection. 24 hours later, the culture medium was discarded, and fresh Jurkat cell complete medium (RPMI 1640 Medium (Gibco)+10% FBS+5 mg/mL penicillin+10 mg/mL streptomycin) was added. The Jurkat T cells were cultured in a 37° C., 5% CO₂ incubator. Passages of cells were conducted every three days. After two passages, “Cas9⁺ sgRNA^{iBAR} Jurkat cell library” was ready for screening.

### 3. FasL Treatment of Cas9⁺ sgRNA^{iBAR} Jurkat T Cell Library

To first determine the appropriate FasL concentration for identifying target genes, the Cas9⁺ sgRNA^{iBAR} Jurkat cell library produced above was added to a 96-well plate, 20,000 cells and 100 μl Jurkat cell complete medium per well, and cultured in a 37° C., 5% CO₂ incubator overnight. The next day, recombinant human FasL (BioLegend) was added to each well at a concentration of 0.1 ng/ml, 1 ng/ml, 10 ng/ml, or 100 ng/ml. About 16 hours post FasL addition, cell death rate was assessed using Annexin V/DAPI (eBioScience) staining and FACS. Annexin V and DAPI both stain dead cells, hence Annexin V⁻ & DAPI⁻ staining indicated alive Jurkat T cells after FasL treatment. As shown in FIG. 5, the appropriate FasL concentration for identifying target genes was 10 ng/ml.

To screen for cells resistant to AICD mediated by FasL/Fas interaction, the Cas9⁺ sgRNA^{iBAR} Jurkat T cell library produced above was added to a 150 cm² culture flask in 120 ml Jurkat cell complete medium, and cultured in a 37° C., 5% CO₂ incubator overnight. The next day, 10 ng/ml FasL (BioLegend) was added to the experiment group and allowed growth for about 16 hours. For control group, no FasL was added, Cas9⁺ sgRNA^{iBAR} Jurkat T cells were grown under same condition and duration as the experiment group. After FasL treatment for about 16 hours, Cas9⁺ sgRNA^{iBAR} Jurkat T cells in the experiment group were collected by centrifugation (alive cells in pellet). Cell survival rate post-FasL treatment was tested with Annexin V/DAPI (eBioScience) staining, with alive cells being Annexin V⁻ & DAPI⁻. The alive Cas9⁺ sgRNA^{iBAR} Jurkat T cells in the experiment group were allowed to recover for growth for about 144 hours (about 6 days), then further treated with FasL for about 16 hours, enriched for alive cells, and allowed for recovery growth for about 144 hours for another 2 rounds (i.e., total 3 rounds of FasL treatment, alive cell enrichment, and recovery growth). See FIG. 2 for exemplary screening workflow. The cell survival rate after the three rounds of FasL treatment in the experiment group was 4%, 44.8%, and 55.5%, respectively, demonstrating that cells resistant to AICD were gradually enriched.

### 4. Identification and Analysis of AICD/FasL Resistant Genes

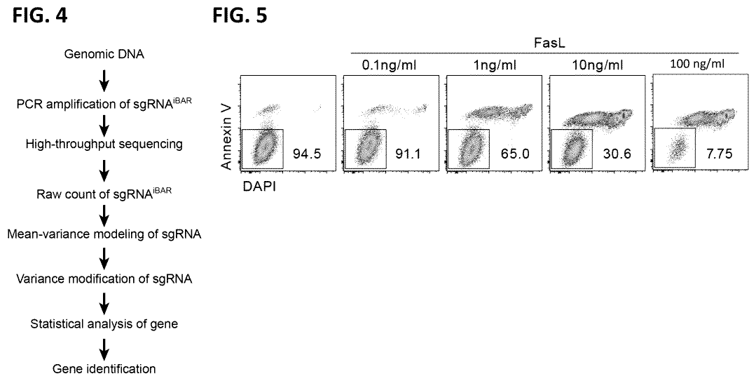

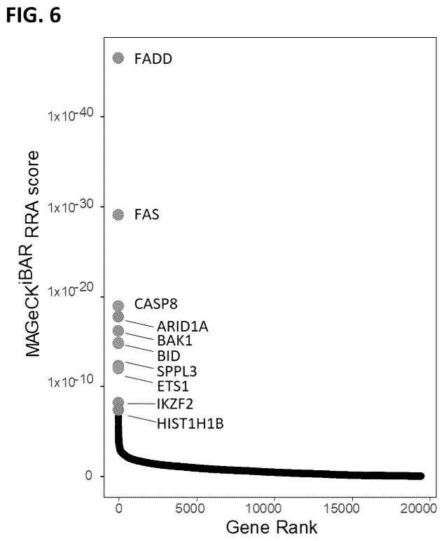

To identify target genes associated with resistance to AICD mediated by FasL, i) 1.1×10⁸ Cas9⁺ sgRNA^{iBAR} Jurkat T cells of the experiment group before FasL treatment, ii) 1.1×10⁸ Cas9⁺ sgRNA^{iBAR} Jurkat T cells of the control group before screening start, iii) 1.1×10⁸ Cas9⁺ sgRNA^{iBAR} Jurkat T cells of the experiment group after three rounds of FasL treatment and enrichment for alive cells, and iv) 5.5×10⁷ Cas9⁺ sgRNA^{iBAR} Jurkat T cells of the control group at the end of the screening, were collected. Genomic DNA was extracted from groups iii) and iv) of cells. sgRNA^{iBAR} encoding fragments were PCR amplified from the extracted genome, purified, and prepared for NGS sequencing. MAGeCK^{iBAR} algorithm was used for sequencing data analysis (see Zhu et al., “Guide RNAs with embedded barcodes boost CRISPR-pooled screens,” Genome Biol. 2019; 20:20; the content of which is incorporated herein by reference in its entirety), which contains three main parts: analysis preparation, statistical tests, and rank aggregation. Briefly, each sgRNA^{iBAR} targeted gene was scored and ranked based on the enrichment degree of each gene in the experiment group (after 3 rounds of FasL treatment and enrichment for alive cells) compared to control group (at the end of the screening), in order to determine if such gene was a candidate gene with high confidence. See FIG. 4 for target gene identification workflow. The top 10 genes whose KO led to resistance to AICD mediated by FasL (AICD/FasL resistant genes) were identified to be: FADD, FAS, CASP8, ARID1A, BAK1, BID, SPPL3, ETS1, IKZF2, HIST1H1B. See FIG. 6.

### 5. Discussion

Results obtained here demonstrate valuable targets in immune cell-based therapy (e.g., CAR-T therapy) as well as biomarkers for donor selection. For example, CAR-T cells modified to KO or reduce expression of AICD/FasL resistant genes may prolong CAR-T cell retention and/or enhance CAR-T cell activities in vivo. Aberrations (e.g., mutations such as loss-of-function (LOF) mutations) in AICD/FasL resistant genes may also serve as biomarkers that such individuals should be selected as donor of T cells (e.g., to produce allogeneic CAR-T cells). Lack of aberrations (e.g., mutations such as LOF mutations) in AICD/FasL resistant genes, on the other hand, may serve as biomarkers for not selecting such individuals for T cell donation.

### Example 2. Functional Tests of SPPL3-KO Jurkat Cells and SPPL3-KO Primary T Cells

This example demonstrates that SPPL3 KO i) provides T cells resistance to AICD mediated by FasL/Fas interaction; ii) provides T cells resistance to allogeneic T cell and/or NK cell killing; and iii) does not affect cytotoxicity mediated by CAR expressed in the T cells.

### 1. Construction of SPPL3-KO Jurkat Cells

Nucleic acid encoding sgRNA against SPPL3 locus (5′-3′: AGACAGATGCTCCAATTGGA, SEQ ID NO: 1) and nucleic acid encoding sgRNA against non-target site (“NT”; 5′-3′: AAAAAGCTTCCGCCTGATGG, SEQ ID NO: 2) were synthesized, and cloned into pLenti-sgRNA-GFP plasmid, respectively. Lentiviruses carrying such plasmids were obtained using standard protocol by infecting 293T cells, similarly as described in Example 1. Cas9⁺ Jurkat T cells were infected with lentiviruses encoding SPPL3-sgRNA or lentiviruses encoding NT-sgRNA at high MOI, similarly as described in Example 1, thus obtaining Cas9⁺ SPPL3-sgRNA+(SPPL3-KO) Jurkat T cells and Cas9⁺ NT-sgRNA+(control) Jurkat T cells, respectively.

The obtained SPPL3-KO and control Jurkat T cells were cultured for a week. A portion of the cultured cells was collected, genomic DNA was extracted, and PCR amplified at the SPPL3 locus. PCR products were sequenced. The appearance of double peaks in sequencing result indicated successful SPPL3-KO.

### 2. Construction of SPPL3-KO Primary T Cells

### Primary T Cell Isolation and Culture

After blood collection, PBMCs were separated from the donor blood samples. T cells were isolated from the PBMCs using the immunomagnetic bead method, then cultured in 37° C., 5% CO₂ incubator in X-VIVO™ 15 media containing 10% FBS, 1% GlutaMAX, and 0.1% recombinant human IL-2 (hereinafter referred to as “primary T cell complete medium”).

### Primary T Cell Activation and Expansion

3 ml Dynabeads® Human T-Activator CD3/CD28 were separated into 14 ml Falcon tubes and 2 mL PBS was added into each tube, Dynabeads® were resuspended well by pipetting, the 14 ml Falcon tubes were placed onto a magnetic rack to let stand for 1 minute, then supernatant was removed. This washing step was repeated twice. Then 5 mL primary T cell complete medium was added to each tube to resuspend washed Dynabeads® by gently pipetting.

1.2×10⁸ cultured primary T cells were transferred into T150 cell culture flasks, then 5 mL resuspended Dynabeads® were added into the primary T cells and gently mixed. The mixture was separated into 3 T150 cell flasks and cultured in 37° C., 5% CO₂ incubator for primary T cell activation and expansion.

### Primary T Cell Transduction

Primary T cells were infected with lentiviruses encoding SPPL3-sgRNA or lentiviruses encoding NT-sgRNA described above at high MOI, and gently mixed. The cell mixture was cultured overnight in a 37° C., 5% CO₂ incubator for infection. The supernatant was discarded the next day and added the fresh medium. The infected T cells were cultured overnight in a 37° C., 5% CO₂ incubator, then transferred to a 50 mL centrifugation tube, placed onto a magnetic rack, let stand for 10 minutes. The supernatant was then transferred to a new 50 mL centrifugation tube, placed onto a magnetic rack, let stand for 5 minutes, to remove as many Dynabeads® as possible. The supernatant containing T cells was transferred to a clean 50 mL centrifugation tube, centrifuged at 400 g 5 minutes, resuspended with 20 mL DPBS, washed twice, then centrifuged at 400 g 5 minutes. The supernatant was discarded, T cells were resuspended with 1.8 mL Opti-MEM® Reduced-Serum Medium and counted cell number (1.8×10⁸ T cells). These T cells were separated into nine 1.5 mL Eppendorf tubes and placed on ice. 16 μg Cas9 mRNA (synthesized by TriLink) was added into each tube, gently mixed, then cell mixture was each transferred into a 4 mm BTX electroporation cuvette for electrotransformation. T cells post-electrotransformation were transferred into a T150 cell culture flask, supplemented with primary T cell complete medium to adjust cell density to 1×10⁶ cells/mL, then cultured in a 37° C., 5% CO₂ incubator. Passages of cells were conducted every two days.

96 hours post-electrotransformation, a portion of the transfected primary T cells was collected, genomic DNA was extracted, and PCR amplified at the SPPL3 locus. PCR products were sequenced. The appearance of double peaks in sequencing result indicated successful SPPL3-KO.

### 3. Construction of CD19-KO RAJI Cells

Nucleic acid encoding sgRNA against CD19 (5′→3′: ATGAAAAGCCAGATGGCCAG, SEQ ID NO: 3) was synthesized, and cloned into pLenti-sgRNA-GFP plasmid. Lentiviruses carrying such plasmids were obtained using standard protocol by infecting 293T cells, similarly as described in Example 1.

RAJI cells were cultured in “RAJI complete medium” (RPMI 1640 Medium (Gibco)+10% FBS+5 mg/mL penicillin+10 mg/mL streptomycin) in 37° C., 5% CO₂ incubator, until sufficient cells were obtained.

Cultured RAJI cells were infected with lentiviruses encoding CD19-sgRNA at high MOI and gently mixed. The cell mixture was cultured overnight in a 37° C., 5% CO₂ incubator for infection. The supernatant containing lentiviruses was discarded the next day, and cells were added with fresh medium. The infected RAJI cells were cultured overnight in a 37° C., 5% CO₂ incubator, then transferred to nine 1.5 mL Eppendorf tubes and placed on ice. 16 μg Cas9 mRNA (synthesized by TriLink) was added into each tube, gently mixed, then cell mixture was each transferred into a 4 mm BTX electroporation cuvette for electrotransformation. RAJI cells post-electrotransformation were transferred into a T150 cell culture flask, supplemented with RAJI complete medium to adjust cell density to 1×10⁶ cells/mL, then cultured in a 37° C., 5% CO₂ incubator. Passages of cells were conducted every two days.

The obtained RAJI cells were stained with PE anti-human CD19 antibody (BioLegend), then FACS sorted for CD19-negative RAJI cells for later experiment.

### 4. Test for Resistance to FasL-Mediated AICD in SPPL3-KO Jurkat T Cells and SPPL3-KO Primary T Cells

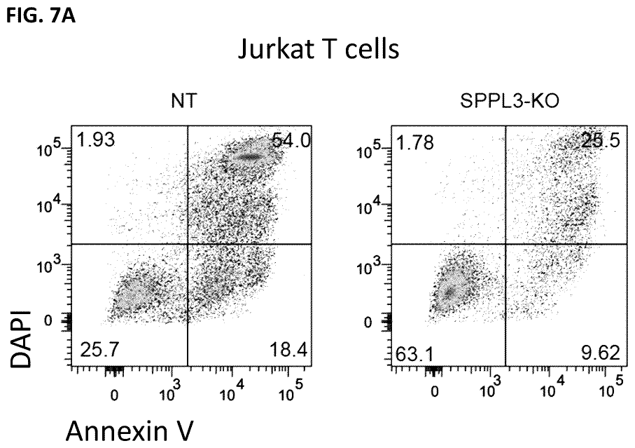

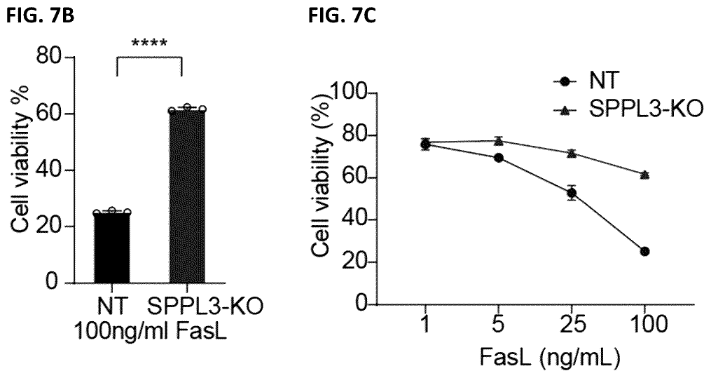

SPPL3-KO Jurkat T cells and control (NT-KO) Jurkat T cells were treated with FasL at various concentration (1 ng/mL, 5 ng/mL, 25 ng/mL, and 100 ng/mL) for about 16 hours. Cell survival rate was assayed by Annexin V/DAPI staining and FACS sorting as described in Example 1. FIGS. 7A and 7B show that 100 ng/mL FasL treatment led to 25.7% cell survival rate (Annexin V⁻ & DAPI⁻) in control (NT-KO) Jurkat T cells, which was statistically significantly lower than 63.1% cell survival rate in SPPL3-KO Jurkat T cells. FIG. 7C shows that SPPL3-KO Jurkat T cells were much more resistant to FasL-mediated AICD under various FasL concentrations compared to control Jurkat T cells. These data indicate that SPPL3-KO can reduce FasL-mediated AICD in Jurkat T cells.

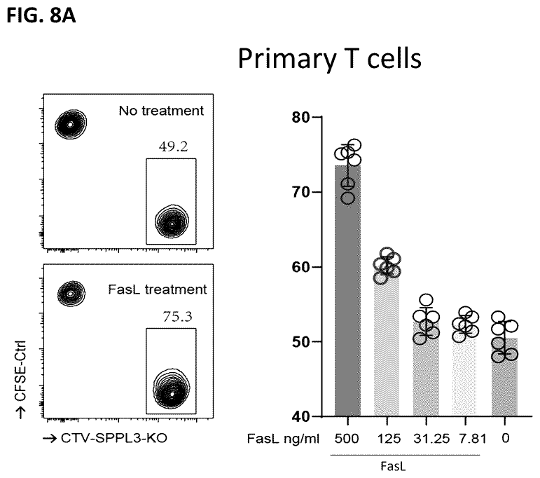

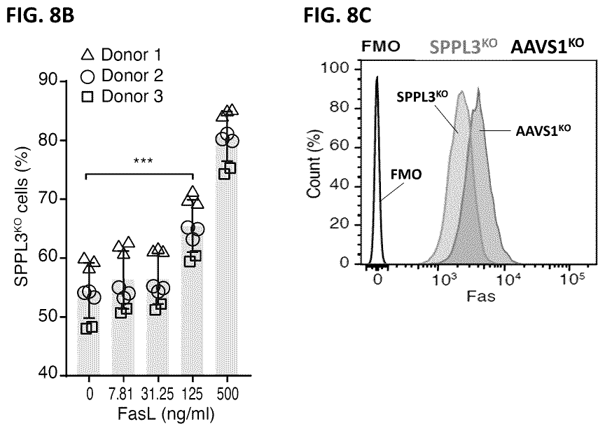

SPPL3-KO primary T cells were labeled with CellTrace™ Violet (CTV) (ThermoFisher), control (NT-KO) primary T cells were labeled with CellTrace™ carboxyfluorescein succinimidyl ester (CFSE) (ThermoFisher), and mixed at 1:1 before treated with FasL at various concentration (0 ng/mL, 7.81 ng/mL, 31.25 ng/mL, 125 ng/mL, and 500 ng/mL) for 16 hours. After 16 hours, the ratio of CTV-SPPL3-KO primary T cells vs. CFSE-NT-KO primary T cells was detected by FACS. As shown in FIG. 8A, when no FasL was added, 49.2% cells were CTV-SPPL3-KO primary T cells. The ratio of SPPL3-KO primary T cells labeled by CTV increased as the concentration of FasL increased. Under 500 ng/mL FasL treatment, 75.3% cells were CTV-SPPL3-KO primary T cells. These data indicate that SPPL3-KO can reduce FasL-mediated AICD in primary T cells.

Hence, SPPL3-KO can reduce FasL-mediated AICD, or increase resistance to FasL-mediated AICD, in both Jurkat T cells and primary T cells.

### 5. Test for Expression of Immune Cell Molecules on SPPL3-KO Jurkat T Cells and SPPL3-KO Primary T Cells

SPPL3-KO Jurkat T cells, control (NT-KO) Jurkat T cells, SPPL3-KO primary T cells, and control (NT-KO) primary T cells were separately centrifuged at 800 g for 5 minutes, after which the supernatant was discarded, and the cells were washed once in PBS. To assay their cell surface expression of HLA-A, HLA-B, HLA-C, B7-H6 (immunoligand for NK cell activating receptor NKp30), and Fas, T cells were stained with APC anti-human HLA-A,B,C Antibody (BioLegend), human B7-H6 PE-conjugated Antibody (R&D System), and PE anti-human CD95 (Fas) Antibody (Miltenyi Biotec), respectively, for 30 minutes. To assay T cell surface expression of ligands for NK cell activating receptors NKG2D and NKp46, T cells were contacted with Recombinant Human NKG2D Fc Chimera Protein (R&D System) or Recombinant Human NKp46/NCR1 Fc Chimera Protein (R&D System), respectively, for 45 minutes, followed by secondary antibody staining using Mouse Anti-Human IgG1 Hinge-PE (4E3) (Southern Biotech) for 45 minutes. The T cell surface expression levels of these molecules were measured using flow cytometry after resuspension.

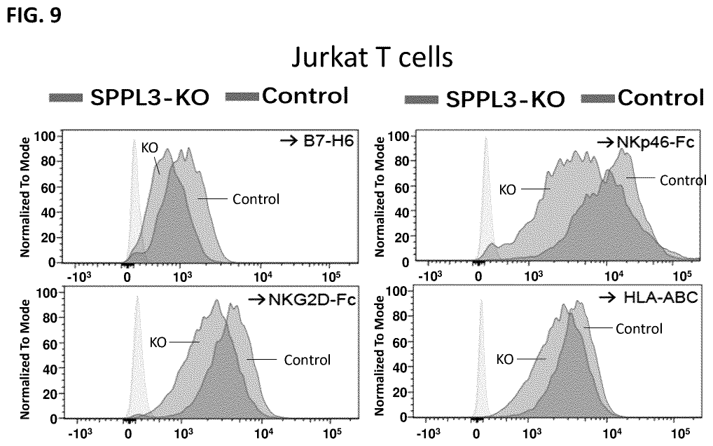

As shown in FIG. 9, compared to control (NT-KO) Jurkat T cells, SPPL3-KO Jurkat T cells had lower expression of NK cell ligands (B7-H6, NKG2D ligand, NKp46 ligand), as well as lower expression of HLA-A,B,C that can be targeted by allogeneic T cells.

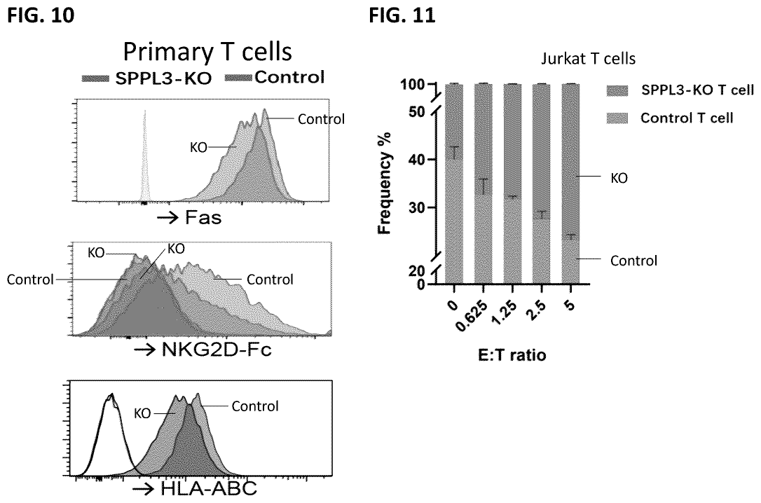

As shown in FIG. 10, compared to control (NT-KO) primary T cells, SPPL3-KO primary T cells had lower expression of Fas, NKG2D ligands, and HLA-A,B,C.

These results indicate that SPPL3-KO can i) reduce T cell surface Fas expression to reduce FasL-mediated AICD; ii) reduce T cell surface expression of NK cell activating ligands to reduce targeted cytotoxicity from NK cells; and iii) reduce T cell surface expression of HLA-A,B,C to reduce targeted cytotoxicity from allogeneic T cells.

### 6. Test for Resistance to NK Cell Killing in SPPL3-KO Jurkat T Cells and SPPL3-KO Primary T Cells

FIG. 3 shows exemplary workflow of this experiment.

SPPL3-KO Jurkat T cells were labeled with CellTrace™ Violet (CTV) (ThermoFisher). CTV-labeled SPPL3-KO Jurkat T cells and control (NT-KO) Jurkat T cells, both expressing GFP introduced by the sgRNA vector, were mixed at 1:1.

Primary natural killer (NK) cells were isolated from whole blood by negative selection using RosetteSep™ Human NK Cell Enrichment Cocktail (Stemcell), and cultured in RPMI 1640 (supplemented with 10% FBS+5 mg/mL penicillin+10 mg/mL streptomycin+100 IU/mL IL-2) at 1×10⁶ cells/mL in 37° C., 5% CO₂ incubator overnight.

Cultured NK cells (effector cells) were then added to the GFP-Jurkat cell mixture obtained above (target cells) at effector:target (E:T) ratios of 0, 0.625, 1.25, 2.5, and 5, for 6 hours. After 6 hours of NK cell treatment, the percentage of CTV-labeled SPPL3-KO Jurkat T cells vs. control (NT-KO) Jurkat T cells was examined by flow cytometry.

As shown in FIG. 11, compared to no NK cell treatment (E:T=0), the frequency of CTV-labeled SPPL3-KO Jurkat T cells increased relative to the control Jurkat T cells as the E:T ratio increased, indicating that SPPL3-KO Jurkat cells are more resistant to killing by NK cells.

To test resistance to killing by NK cells in SPPL3-KO primary T cells, SPPL3-KO primary T cells were labeled with CellTrace™ Violet (CTV) (ThermoFisher), and control (NT-KO) primary T cells were labeled with CellTrace™ CFSE (ThermoFisher), and mixed at a ratio of 1:1. Cultured NK cells from above were added to the primary T cell mixture at an E:T ratio of 0, 0.156, 0.312, 0.625, 1.25, and 2.5, for 6 hours. After 6 hours of NK cell treatment, the percentage of CTV-labeled SPPL3-KO primary T cells vs. CFSE-labeled control (NT-KO) primary T cells was examined by flow cytometry.

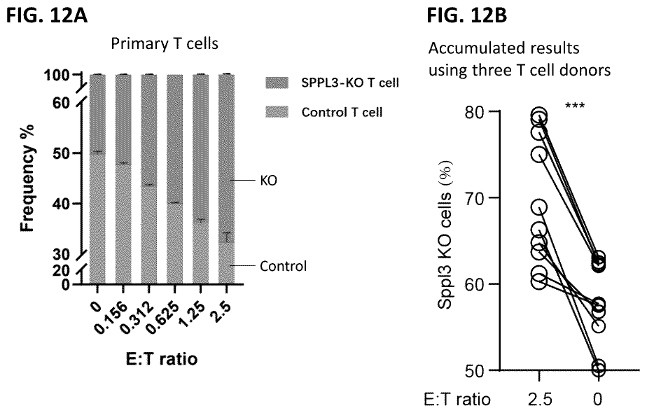

As shown in FIG. 12A, compared to no NK cell treatment (E:T=0), the frequency of CTV-labeled SPPL3-KO primary T cells increased relative to CFSE-labeled control (NT-KO) primary T cells as the E:T ratio increased, indicating that SPPL3-KO primary T cells are more resistant to killing by NK cells.

Consistent relative resistance to NK cell killing was seen when T cells from multiple T cell donors were used. Similarly, T cells from each donor (total 3 donors) were either KO for SPPL3 or mock-treated, labeled with CTV or CFSE respectively, mixed together at 1:1 ratio, then treated with NK cells. The accumulated result from 3 donors is shown in FIG. 12B: the percentage of CTV-labeled SPPL3-KO primary T cells significantly increased in the mixture of SPPL3-KO and control primary T cells as the E:T ratio increased.

To summarize, above data demonstrated that SPPL3-KO can reduce killing by NK cells.

### 7. Test for In Vitro Cytotoxicity of SPPL3-KO Anti-CD19 CAR Jurkat T Cells

Lentiviral vector encoding CTL019 (anti-CD19 CAR) was packaged into lentiviruses using standard protocol by infecting 293T cells, similarly as described in Example 1.

SPPL3-KO Jurkat T cells and control (NT-KO) Jurkat T cells were infected with CTL019 lentiviruses obtained above, and confirmed to be over 80% CAR⁺ by staining with PE-Labeled Monoclonal Anti-FMC63 scFv Antibody (ACROBiosystems; FMC63 is an IgG2a mouse monoclonal antibody specific for CD19), hereinafter referred to as “SPPL3-KO anti-CD19 CAR Jurkat T cells” and “control (NT-KO) anti-CD19 CAR Jurkat T cells,” respectively.

CD19-KO RAJI cells (expressing GFP introduced by sgRNA vector) obtained above and wildtype RAJI cells (CD19⁺) were mixed at 1:1 to make the target cell mixture. The target cell mixture was then mixed with either SPPL3-KO anti-CD19 CAR Jurkat T cells or control (NT-KO) anti-CD19 CAR Jurkat T cells (effector cells) at various E:T ratios (0 to 2). After 24 hours culture, the percentage of alive wildtype RAJI cells (CD19⁺) in the cell mixture was assayed using PE anti-human CD19 Antibody (BioLegend) via flow cytometry.

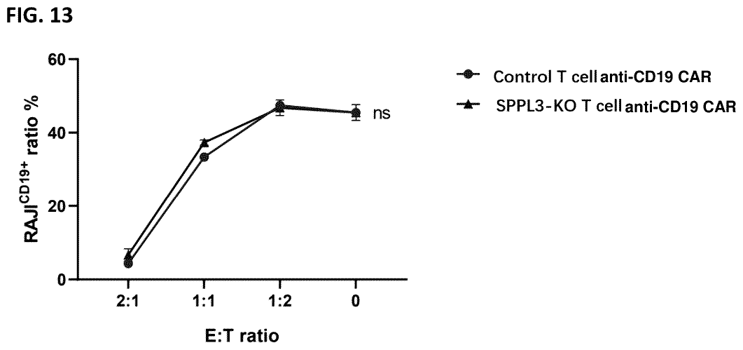

As shown in FIG. 13, SPPL3-KO anti-CD19 CAR Jurkat T cells and control (NT-KO) anti-CD19 CAR Jurkat T cells had similar killing efficiency against CD19⁺ RAJI cells, and that higher killing efficiency (lower CD19⁺ RAJI percentage) was consistently associated with higher E:T ratios compared to the group with no Jurkat T cells (E:T=0). These results indicate that SPPL3-KO does not affect cytotoxicity of CAR-T cells against target cells.

### 8. Test for Stimulation of Allogeneic T Cells by SPPL3-KO Primary T Cells

SPPL3-KO primary T cells and control (NT-KO) primary T cells were separately irradiated at a dose of 25 Gray (Gy) to produce an SPPL3-KO “stimulator” T cell population and a control “stimulator” T cell population, respectively. Primary PBMCs (“responder”) were isolated from whole blood by ficoll centrifugation (Stemcell) and labeled with CellTrace™ CFSE (ThermoFisher).

CFSE-labeled PBMCs were added to the irradiated SPPL3-KO stimulator T cell population or control stimulator T cell population, respectively, at a responder:stimulator ratio of 5:1 for 5 days. As a negative control, a population of CFSE-labeled PBMCs were cultured alone under similar condition. Cells were cultured in X-VIVO™ 15 media (Lonza) (supplemented with 10% FBS+5 mg/mL penicillin+10 mg/mL streptomycin+100 IU/mL IL-2) at 1×10⁶ cells/mL in 37° C., 5% CO₂ incubator.

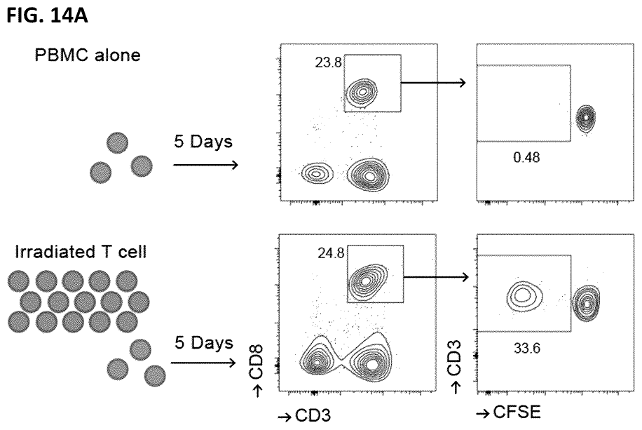

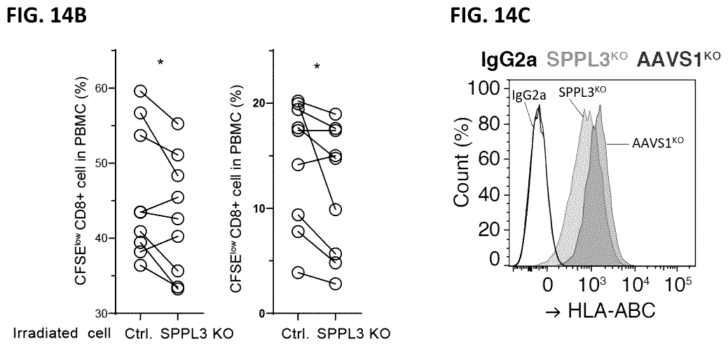

After 5 days of culture, CFSE-low CD8+ T cells were analyzed by FACS among all viable T cells (CD3+ and CFSE+). As shown in FIG. 14A, PBMCs cultured alone (negative control) had no proliferated CFSE-low CD8+ T cells (upper panel; the percentage of CFSE-low cells was 0.48); while PBMCs cultured together with irradiated primary T cells stimulated CD8+T proliferation (lower panel; the percentage of CFSE-low cells was 33.6).

FIG. 14B shows plots of percentage of CFSE-low CD8+ PBMC cells (among all viable CD3+ CFSE+ PBMC cells) co-cultured with either irradiated control primary T cells or irradiated SPPL3-KO primary T cells. 9 allogeneic responses were plotted in each plot. The two plots were based on primary T cells from two different donors. As shown in FIG. 14B, SPPL3-KO primary T cells induced statistically significantly less proliferation of allogeneic T cells (CFSE-low CD8+) compared to the control primary T cells. These data are consistent with T cells with HLA-A/B/C downregulation which trigger less allogeneic response. These data demonstrate that SPPL3 KO could mask the T cells from being recognized (e.g., by allogeneic T cells), thus effectively modulating allogeneic lymphocyte responses, and reducing HvG responses.

### Example 3. SPPL3 Ablation Shields T Cells from Allogeneic NK/T Cell Killing and Activation-Induced Cell Death (AICD)

This example demonstrates that SPPL3 ablation shields T cells from allogeneic NK/T Cell killing and activation-induced cell death (AICD).

### 1. Removal of SPPL3 Mitigated Activation-Induced T-Cell Death

SPPL3^{KO} primary T cells were generated and their resistance to FasL-induced cell death using a competitive killing assay was evaluated. To facilitate the design of universal T cells, the TCR was simultaneously ablated in the following experiments. SPPL3^{KO} T cells were labeled with Cell Trace Violet (CTV), and control AAVS1 (Adeno-Associated Virus Integration Site 1) knockout (AAVS1^{KO}) T cells were labeled with carboxyfluorescein succinimidyl ester (CFSE), and were then mixed at a 1:1 ratio for subsequent FasL treatments (FIG. 8A). The relative enrichment of CTV-positive cells following FasL treatment were assessed and compared the results to the untreated group. SPPL3^{KO} primary T cells exhibited greater resistance to FasL-induced cell death compared to AAVS1^{KO} T cells across multiple sgRNAs targeting SPPL3 and cells from different donors (FIGS. 8A and 8B). Mechanistically, a diminished binding of anti-Fas monoclonal antibody to SPPL3^{KO} T cells was detected compared to control cells, suggesting impaired FasL binding on SPPL3^{KO} T cells (FIG. 8C).

### 2. SPPL3 Ablation in Primary T Cells Mitigated the Allogeneic T Cell Killing

To test whether SPPL3^{KO} T cells may have reduced interactions with allogeneic T cells, the effects of SPPL3 ablation in T cells on allogeneic immune responses were examined using a one-way mixed lymphocyte reaction (MLR). In the MLRs, irradiated AAVS1^{KO} (control) or SPPL3^{KO} T cells were mixed with CFSE-labeled peripheral blood mononuclear cells (PBMC) from allogeneic donors (FIG. 14A). AAVS1^{KO} T cells or SPPL3^{KO} T cells had their TCR expression disrupted and were purified as CD3-negative cells prior to the cultures. These T cells could be distinguished as CD3-CD5⁺ in the MLRs. T cells from PBMCs did not proliferate in the absence of allogeneic T cells. Therefore, the stimulatory effects of irradiated T cells on allogeneic T cells were quantified by measuring the percentage of CFSE-negative T cells from PBMCs (FIG. 14A). Indeed, SPPL3^{KO} T cells had a diminished capacity to stimulate the proliferation of allogeneic T cells compared to AAVS1^{KO} T cells (FIG. 14B). A diminished binding of anti-HLA-ABC monoclonal antibody to SPPL3^{KO} T cells was confirmed (FIG. 14C), which suggested that SPPL3^{KO} T cells could evade recognition by allogeneic T cells through the TCR-HLA axis.

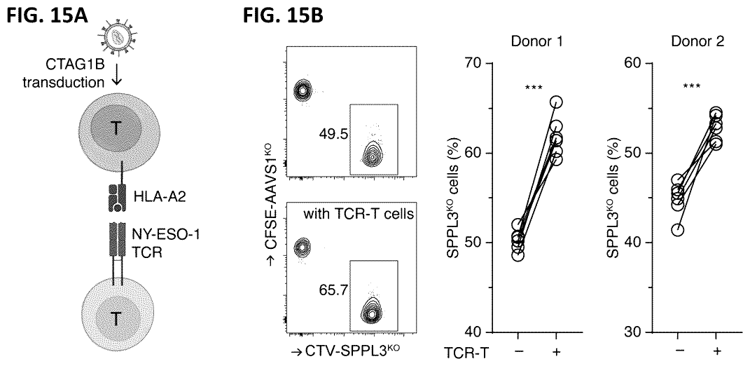

To directly assess the role of SPPL3 in TCR-mediated killing, New York esophageal squamous cell carcinoma 1 (NY-ESO-1) specific TCR T cells and transduced HLA-A2 positive T cells with CTAG1B (encoding NY-ESO-1) as targets for the TCR T cells were used (FIG. 15A). CTAG1B+/AAVS1^{KO} and CTAG1B+/SPPL3^{KO} T cells were barcoded, mixed, and co-cultured with NY-ESO-1 specific TCR T cells. As shown in FIG. 15B, SPPL3^{KO} T cells exhibited resistance to allogeneic TCR T-cell killing. Thus, the ablation of SPPL3 in T cells led to reduced HLA-ABC detection, diminished allogeneic immune stimulations, and resistance to TCR T cells killing.

### 3. SPPL3 Ablation in Primary T Cells Mitigated the Allogeneic NK Cell Killing

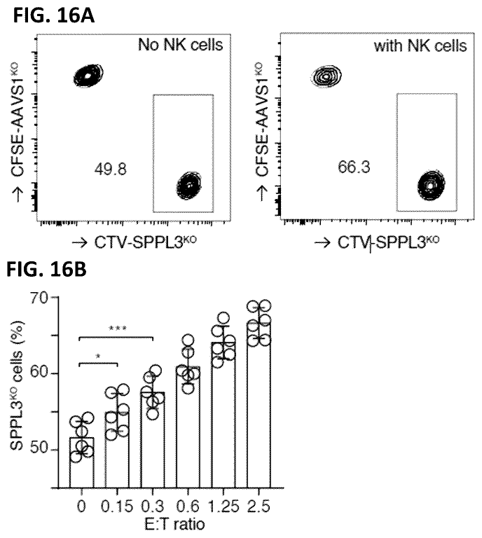

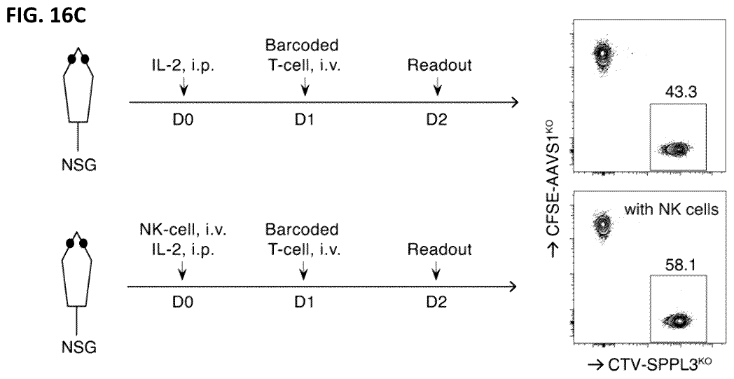

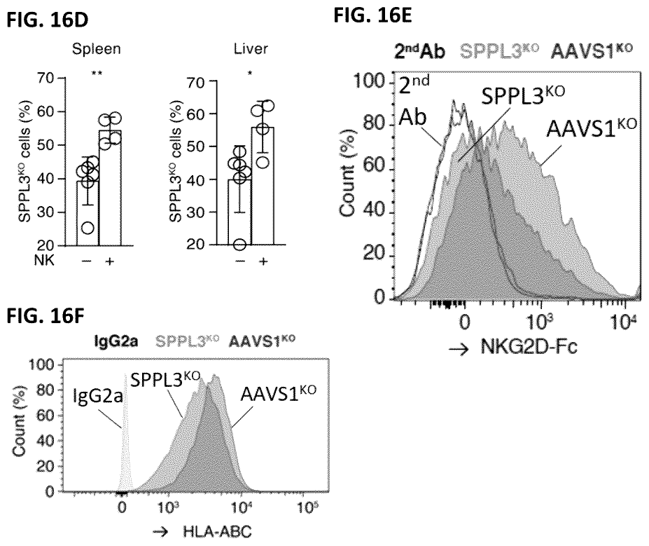

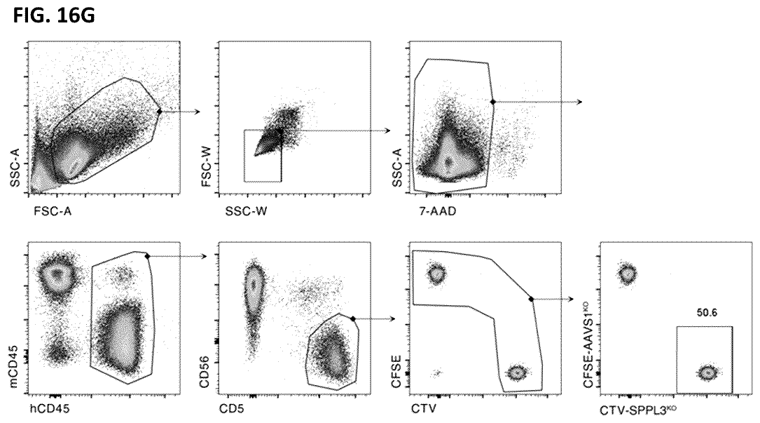

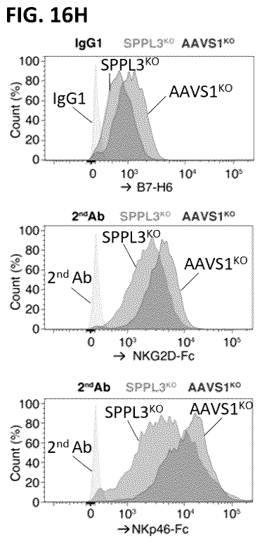

To simulate a scenario of “HLA mismatch” transplantation, NK cells killing screens using B2M-negative T cells were performed. In this setting, KIR2DL3⁻/KIR2DL1⁺/HLAC2⁺ NK populations from recipients can eliminate HLAC2⁻ allogeneic donor cells. To rule out the possibility that B2M might contribute to this resistance, B2M-sufficient target cells in primary NK cell killing assays were used. Jurkat T cells were chosen because they are tumor cells that are susceptible to NK cell killing. Upon confirming the reduced binding of anti-HLA-ABC antibody to SPPL3^{KO} Jurkat T cells (FIG. 16F), the absence of SPPL3 in Jurkat T cells was found to confer protection against NK cell killing, suggesting that the resistance to NK cell killing is independent to B2M expression (FIG. 11).

Sodium propionate (NaP) treatment was employed to increase the susceptibility of primary T cells to NK cell killing. NaP-treated activated T cells maintained their HLA-I/E expression and became more susceptible to NK cell killing, presumably because the activation of NK cell activating receptors that override the inhibitory signals mediated by HLA-I/E. To assess this sensitization, a short-term killing assay using T cells and NK cells obtained from unmatched donors was performed (FIG. 16A). The results revealed that the elimination of SPPL3 conferred a dose-dependent protection to primary T cells against NK cell killing (FIG. 16B). A similar protective effect was observed when using B2M knockout primary T cells (FIG. 12A). As shown in FIG. 16C, IL-2 or IL-2 and NK cells were injected into mice on day 0, followed by i.v. injection of SPPL3^{KO} T cells labeled with CTV or AAVS1^{KO} T cells labeled with CFSE on day 1. When the ratios of the CTV-labeled SPPL3^{KO} T cells and CFSE-labeled AAVS1^{KO} T cells were determined at day 2, primary NK cells exhibited reduced killing of SPPL3^{KO} T cells in tissue settings (FIGS. 16C, 16D, and 16G).

The consistent role of SPPL3 in conferring resistance to NK cell killing in both primary T cells and Jurkat T cells indicated its significant involvement in NK cell recognition rather than inhibition. To investigate this further, the expression of B7-H6, a ligand for NKp30, as well as ligands for NKG2D and NKp46 on Jurkat T cells, were assessed. Mechanistically, the deletion of SPPL3 resulted in diminished binding of antibodies specific to ligands associated with all evaluated activating receptors on Jurkat T cells (FIG. 16G), as well as reduced expression of ligands of NKG2D and Nkp46 on primary T cells (FIGS. 16E and 16H). These findings suggested that the knockout of SPPL3 restricted the accessibility of multiple ligands to activated NK cells, thereby providing protection against NK cell killing.

### 4. SPPL3^{KO}/TCR^{KO}/Anti-CD19 CAR-T Cells Eliminated Tumors In Vivo

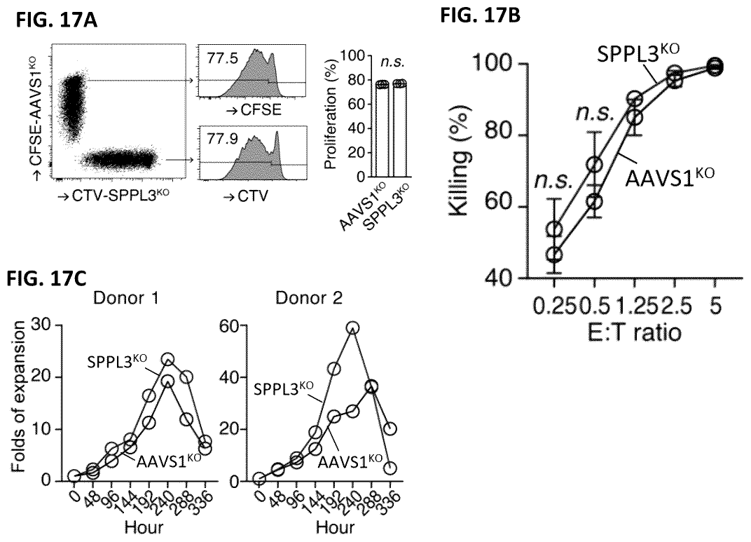

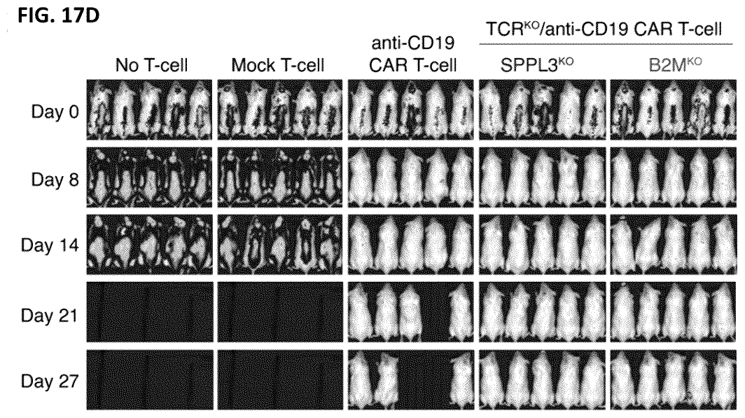

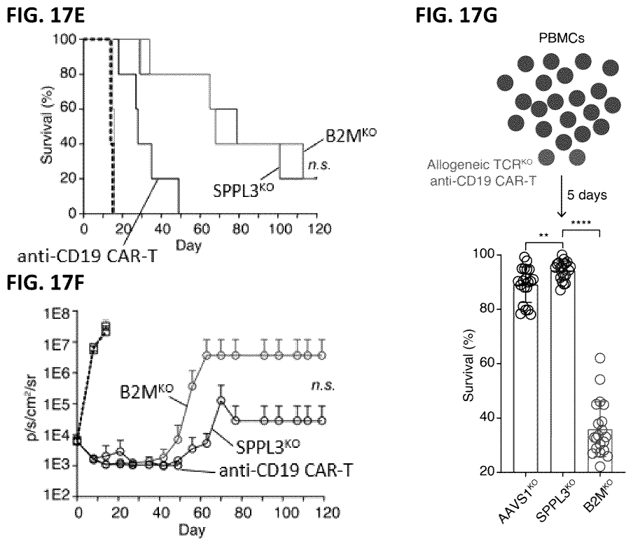

To assess the functional capabilities of SPPL3^{KO} CAR-T cells, the effector functions of CAR-T cells were evaluated. When CTV-labeled SPPL3^{KO}/TCR^{KO}/anti-CD19 CAR-T cells and CFSE-labeled AAVS1^{KO}/TCR^{KO}/anti-CD19 CAR-T cells were cultured in equal numbers and the dye dilutions were evaluated to assess proliferation, no impairment in the proliferation capacity of the SPPL3^{KO} CAR-T cells was observed (FIG. 17A). The killing capacity of SPPL3^{KO}/TCR^{KO}/anti-CD19 CAR-T cells was comparable to that of control AAVS1^{KO}/TCR^{KO}/anti-CD19 CAR-T cells (FIG. 17B). To gain further insights, the growth of SPPL3^{KO}/TCR^{KO}/anti-CD19 CAR-T cells during repetitive stimulations using CD19-positive target cells was extensively evaluated. Importantly, these cells exhibited a growth advantage over control AAVS1^{KO}/TCR^{KO}/anti-CD19 CAR-T cells (FIG. 17C). Since CAR-T cells are known to be susceptible to AICD upon repetitive antigen stimulations, the enhanced expansion observed in SPPL3^{KO}/TCR^{KO}/anti-CD19 CAR-T cells could potentially be attributed to their reduced binding to FasL.

To evaluate the in vivo antitumor activity of SPPL3^{KO}/TCR^{KO}/anti-CD19 CAR-T cells, xenograft models bearing NALM6 cells (CD19⁺ human B cell precursor leukemia cell line) were used. To determine whether SPPL3^{KO} CAR-T cells cause GvHD, wild-type anti-CD19 CAR-T cells were used as a control in the animal model. The antitumor activity of B2M^{KO}/TCR^{KO}/anti-CD19-CAR T cells, which have been extensively characterized and demonstrated with unaltered effector functions in previous studies, was benchmarked. As shown in FIG. 17D, mock T cells (negative control T cells that did not undergo any CAR engineering or gene editing) did not exhibit any anti-tumor activity compared to negative control of no human T cell treatment. In contrast, SPPL3^{KO}/TCR^{KO}/anti-CD19 CAR-T cells improved animal survival for up to 120 days, while anti-CD19 CAR-T cells without other gene editing only achieved ˜50-day survival (FIG. 17E).

Although there was no significant difference in the survival rate of mice that received SPPL3^{KO}/TCR^{KO}/anti-CD19 CAR-T cells or B2M^{KO}/TCR^{KO}/anti-CD19 CAR-T cells (FIG. 17E), as shown in FIG. 17F, SPPL3^{KO}/TCR^{KO}/anti-CD19 CAR-T cells greatly inhibited tumor growth compared to B2M^{KO}/TCR^{KO}/anti-CD19 CAR-T cells. This may be because B2M ablation resulted in the absence of HLA-I molecules on T cells, making them susceptible to NK cell targeting and killing (data not shown). Importantly, the mice that received SPPL3^{KO}/TCR^{KO}/anti-CD19 CAR-T cells did not suffer from complications of GvHD, which ultimately resulted in the death of animals, as observed in the group receiving anti-CD19 CAR-T cells (without other gene editing) from day 21 onwards (FIGS. 17D and 17E). These results suggested that cytotoxic function of SPPL3^{KO}/TCR^{KO}/anti-CD19 CAR-T cells was preserved, and even superior than traditional anti-CD19 CAR-T cells (without other gene editing).

To test whether utilizing allogeneic PBMCs, which contain multiple cell effectors and simulate an allogeneic immune environment, would provide a suitable method for evaluating the survival of these CAR-T cells, allogeneic PBMCs were mixed with anti-CD19 CAR-T cells at a ratio of 10:1, maintained them in culture for 5 days, and the survival of CAR-T cells was evaluated. B2M^{KO}/TCR^{KO}/anti-CD19 CAR-T cells displayed impaired survival compared to AAVS1^{KO}/TCR^{KO}/anti-CD19 CAR-T cells (FIG. 17G). Encouragingly, SPPL3^{KO}/TCR^{KO}/anti-CD19 CAR-T cells displayed significantly improved survival under the same allogeneic immune environment, compared to both B2M^{KO}/TCR^{KO}/anti-CD19 CAR-T cells and AAVS1^{KO}/TCR^{KO}/anti-CD19 CAR-T cells with statistical significance (FIG. 17G).

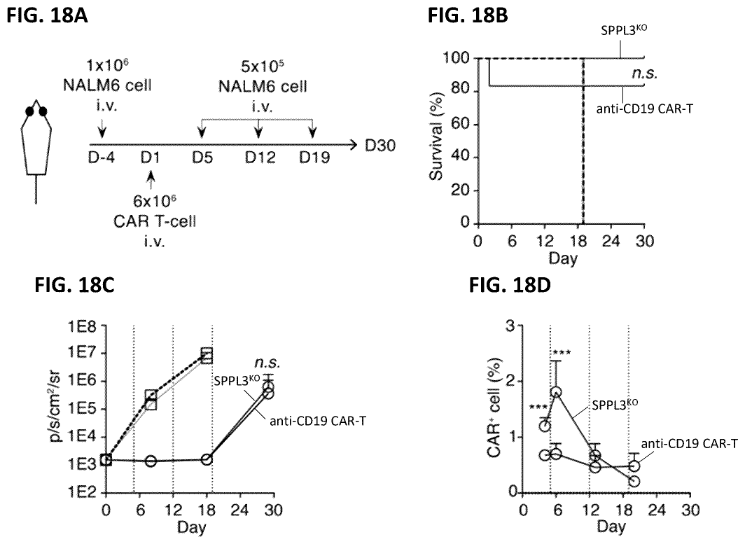

Additionally, xenograft mouse models bearing NALM6 cancer cells (CD19+ human B cell precursor leukemia cell line) were used to determine whether SPPL3^{KO}/TCR^{KO}/anti-CD19 CAR-T cells exhibited enhanced expansion in vivo upon repetitive NALM6 stimulations on days 5, 12, and 19 following administration of control anti-CD19 CAR-T cells (without other gene editing) or SPPL3^{KO}/TCR^{KO}/anti-CD19 CAR-T cells. Mice engrafted with NALM6 cells were treated with SPPL3^{KO}/TCR^{KO}/anti-CD19 CAR-T cells or control anti-CD19 CAR-T cells (without other gene editing) on day 1 of the experimental timeline and subsequently rechallenged with additional tumor cells three times (on days 5, 12, and 19) after the administration of CAR-T cells (FIG. 18A). As shown in FIG. 18B, engrafted mice administered SPPL3^{KO}/TCR^{KO}/anti-CD19 CAR-T cells showed 100% survival, compared to 80% survival of engrafted mice given control anti-CD19 CAR-T cells. The no T-cell group (dashed line) and the mock T-cell group (T cells without CAR) (solid line overlapping with dashed line) died around day 18 (FIG. 18B). Moreover, unlike the no T-cell group (dashed line) and the mock T-cell group (T cells without CAR) (solid line overlapping with dashed line), engrafted mice injected with SPPL3^{KO}/TCR^{KO}/anti-CD19 CAR-T cells or control anti-CD19 CAR-T cells showed similar tumor burden over the course of 30 days (FIG. 18C). Surprisingly, as shown in FIG. 18D, an increased number of CAR-T cells during the rechallenge experiment (dash vertical lines indicating rechallenge dates) was observed in the SPPL3^{KO}/TCR^{KO}/anti-CD19 CAR-T group compared to control anti-CD19 CAR-T group. Therefore, although SPPL3-deficient CAR T-cells killed tumor cells with comparable efficiency to control anti-CD19 CAR-T cells, not only did SPPL3^{KO}/TCR^{KO}/anti-CD19 CAR-T cells exhibit enhanced expansion upon repetitive target antigen stimulations in vivo, but 100% of engrafted mice that received SPPL3^{KO}/TCR^{KO}/anti-CD19 CAR-T cells survived. This result suggests that SPPL3 ablation positively affects the proliferation of CAR-T cells when subjected to repeated target antigen stimulation.

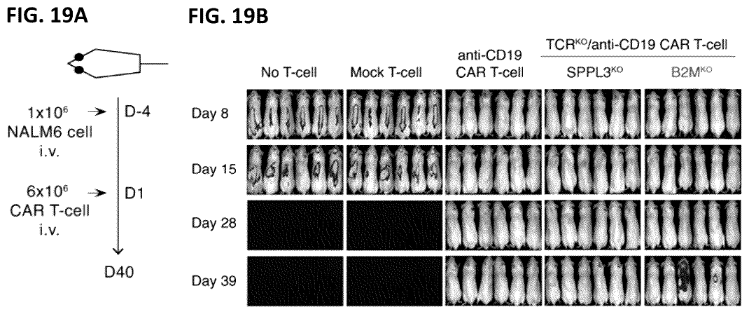

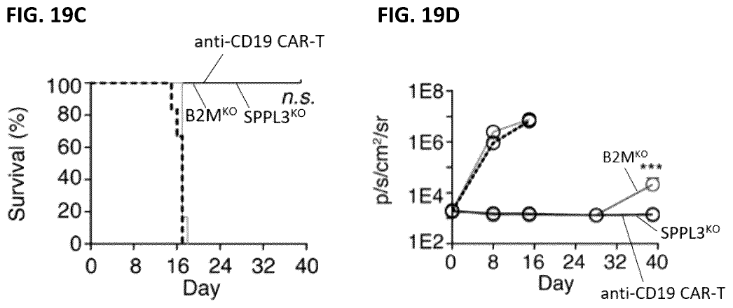

Because CAR-T cells are known to be susceptible to AICD upon repetitive target antigen stimulations and the enhanced expansion observed in SPPL3^{KO}/TCR^{KO}/anti-CD19 CAR-T cells could potentially be attributed to reduced binding to FasL, the antitumor activity of SPPL3^{KO}/TCR^{KO}/anti-CD19 CAR-T cells was further benchmarked with B2M^{KO}/TCR^{KO}/anti-CD19 CAR-T cells which was demonstrated to have unaltered effector functions. Mice were engrafted with NALM6 cancer cells four days prior to the start of the experiment, administered SPPL3^{KO}/TCR^{KO}/anti-CD19 CAR-T cells or B2M^{KO}/TCR^{KO}/anti-CD19 CAR-T cells at day 1, and observed for overall survival and tumor burden over 40 days (FIG. 19A). Mice engrafted with NALM6 (CD19+) cancer cells and subsequently administered mock T cells (T cells without CAR) or without T cell treatment did not exhibit any tumor control (FIGS. 19B-19D).

SPPL3^{KO}/TCR^{KO}/anti-CD19 CAR-T cells not only significantly inhibited tumor growth and improved animal survival, the effects were even more superior compared to control anti-CD19 CAR-T cells (without other gene editing) and B2M^{KO}/TCR^{KO}/anti-CD19 CAR-T cells (FIGS. 19B-19D). As shown in FIG. 19D, the tumor burden for engrafted mice administered SPPL3^{KO}/TCR^{KO}/anti-CD19 CAR-T cells was significantly less than that of mice given B2M^{KO}/TCR^{KO}/anti-CD19 CAR-T cells on day 40. These results demonstrate that SPPL3 ablation in anti-CD19 CAR-T cells effectively eliminates tumors in vivo. This outcome highlights the potent tumor-killing capability of SPPL3-ablated CAR-T cells, showcasing their potential as a highly effective therapeutic approach.

### 5. Materials and Methods

### Cells

Peripheral blood mononuclear cells (PBMCs) were collected from de-identified healthy donor apheresis products, isolated by Ficoll purification, and stored in liquid nitrogen prior to experimentation. Jurkat T cells were obtained from Dr. H. Wang (ShanghaiTech University) and maintained in RPMI 1640 medium (Gibco). NALM-6 cells were used as the target cells for anti-CD19 CAR-T cells. All media were supplemented with 10% fetal bovine serum (FBS, Biological Industries), penicillin, and streptomycin (Gibco). Primary T cells and NK cells were purified using a negative selection kit (STEMCELL Technologies) and expanded in X-VIVO™ 15 media (Lonza) with 100 IU/ml interleukin-2 (IL-2; Sihuan ShengWu). When applicable, primary T cells were subjected to an overnight treatment with 10 mM sodium propionate (Sigma-Aldrich) and subsequently utilized as the targets for NK cells.

### Generation of Knockout T Cells and Anti-CD19 CAR T Cells

Knockout T cells were generated using CRISPR-Cas9 technology. Anti-CD3/CD28 beads (ThermoFisher) activated T cells were electroporated with sgRNA and Cas9mRNA (synthesized by TriLink) in a 4 mm BTX electroporation cuvette. T cells post-electrotransformation were transferred into a T150 cell culture flask, supplemented with X-VIVO™ 15 media (Lonza) to adjust cell density to 1×10⁶ cells/mL. Passages of cells were conducted every two days. The KO efficiency was evaluated by a sequencing-based assay. When applicable, anti-CD3/CD28-stimulated T cells were retrovirally transduced with anti-CD19 CAR. The expression of CAR T-cells was determined 6-9 days post-transduction, and experiments were performed 6-14 days post-transduction.

### Mice

NOD/ShiLtJGpt-Prkdc^{em26Cd52}Il2rg^{em26Cd22}/Gpt mice (Strain NO. T001475) were purchased from GemPharmatech (Nanjing, China). All mice used in the study were housed and bred under specific pathogen-free conditions and handled in accordance with the guidelines of the Peking University institute of Systems Biomedicine. For NK cell killing experiments, 100,000 IU IL2 and 20 million NK cells were injected into the animals. The following day, 4 million barcoded T cells were injected, and the ratios of SPPL3^{KO} cells and AAVS1^{KO} cells were determined 24 hours later. To establish a xenograft tumor model, mice were injected with NAML6-Luc cells (1×10⁶). The mice were then randomly divided into experimental and control groups. CAR-T cells (10×10⁶) were injected into the mice via tail vein injection. The survival of the mice was monitored, and the tumor was tracked using bioluminescence imaging. The photons emitted from luciferase-expressing cells were quantified using Living Image software (PerkinElmer). A total body region of interest was drawn around each mouse, and radiance was recorded in units of photons/s/cm2/sr.

### Lentiviral Production and Infection

Lentivirus was produced by transfecting HEK293T cells with a lentiviral transfer vector, the packaging plasmid pR8.74 (#22036; Addgene), and VSVg (#158233; Addgene) using X-tremeGENE HP DNA transfection reagent (Roche). The viral supernatant was collected 72 hours after transfection, purified by passing it through a 0.45-μm filter, and added to target cells along with 8 μg/ml polybrene.

### DNA and RNA Sequences

The sequence of CTAG1B was obtained from GeneCards. The sequence of NY-ESO-TCR was obtained from a previous report. Nucleic acid encoding sgRNA targeting SPPL3 (5′-3′: AGACAGATGCTCCAATTGGA, SEQ ID NO: 1), nucleic acid encoding sgRNA targeting AAVS1 (5′-3′: GTCACCAATCCTGTCCCTAG, SEQ ID NO: 5), and nucleic acid encoding sgRNA targeting B2M (5′-3′: GAGTAGCGCGAGCACAGCTA, SEQ ID NO: 6) were synthesized by GenScript.

### Flow Cytometry Reagents and Analysis

The following antibodies, dyes, and soluble factors were obtained from BioLegend: anti-CD3e (UCHT1), anti-CD5 (UCHT2), anti-CD56 (HCD56), anti-HLA-ABC (W6/32), anti-mCD45 (30-F11), anti-hCD45 (HI30), anti-CD16/32 (2.4G2), 7-AAD, DAPI, CFSE, and recombinant human FasL. The CellTrace™ Violet Cell Proliferation Kit was from ThermoFisher, while the anti-Fas (REA738) antibody was ordered from Miltenyi Biotec. NKp46-Fc recombinant protein, NKG2D-Fc recombinant protein, and anti-B7-H6 antibody were ordered from R&D Systems. Anti-CD19 CAR antibody was obtained from ACROBiosystems. The anti-Annexin V antibody was obtained from Procell, and absolute counting beads (BioLegend) were used to calculate the absolute number of viable cells. Fluorescein labeled Concanavalin A (ConA), Lycopersicon esculentum Lectin (LEL), and Sambucus Nigra Lectin (SNA) were obtained from Vector Laboratories.

### Competitive Killing Assay

T cells were labeled with either CellTrace Violet (CTV) or carboxyfluorescein succinimidyl ester (CFSE) at a 1:1000 dilution in PBS for 5 minutes at room temperature. The dyes were quenched with media containing 10% FBS. Barcoded T cells were mixed at a 1:1 ratio and subjected to treatment with FasL, NK cells, or T cells. The ratios of cells of interest were measured by flow cytometry and compared with the results of the untreated group.

### Statistical Analysis

Paired ratios from the same donor were compared using the nonparametric Wilcoxon signed-rank test. Ratios between two groups were compared using the Mann-Whitney U test. Statistical significance was set at a two-sided P level of 0.05. The levels of significance were denoted as follows: *P<0.05; **P<0.01; ***P<0.001.

1. An immune cell, wherein the immune cell is modified to have no or reduced expression and/or function of one or more target proteins selected from the group consisting of: Signal Peptide Peptidase Like 3 (SPPL3), FADD, FAS, CASP8, ARID1A, BAK1, BID, ETS1, IKZF2, and HIST1H1B.

2. The immune cell of claim 1, wherein the immune cell is modified to have no or reduced expression and/or function of SPPL3 protein.

3. The immune cell of claim 2, wherein the immune cell has at least about 10% less activation-induced cell death (AICD) compared to a reference immune cell not having the modification to reduce or eliminate expression and/or function of the SPPL3 protein.

4. (canceled)

5. The immune cell of claim 2, wherein the immune cell is genetically modified at the SPPL3 locus or at SPPL3 RNA, and wherein the SPPL3 locus is modified by gene editing, or wherein the SPPL3 RNA is modified by RNA editing.

6. The immune cell of claim 5, wherein the gene editing or RNA editing is mediated by CRISPR/Cas.

7. The immune cell of claim 6, wherein the gene editing or RNA editing comprises contacting a precursor immune cell with i) a guide RNA (gRNA) construct, wherein the gRNA construct comprises or encodes a gRNA comprising a guide sequence that is complementary to a target site in the SPPL3 locus or SPPL3 RNA; and optionally ii) a Cas component comprising a Cas protein or a nucleic acid encoding the Cas protein, under a condition that allows introduction of the gRNA construct and the optional Cas component into the precursor immune cell.

8. (canceled)

9. The immune cell of claim 7,
(i) wherein the Cas protein has endonuclease activity; or
(ii) wherein the Cas protein is a fusion protein comprising (a) a dead Cas protein (dCas), and (b) an adenine deaminase (ADA), a cytidine deaminase (CDA), or a functional fragment thereof.

10-12. (canceled)

13. The immune cell of claim 1, wherein the immune cell has or is further modified to have no or reduced expression and/or function of one or more other proteins selected from the group consisting of TCRα, TCRβ, TCRγ, TCRδ, HLA-A, HLA-B, HLA-C, HLA-E, HLA-F, HLA-G, B2M, PD-1, TIM-3, LAG-3, CTLA-4, CISH, Fas, FADD, CASP8, ARID1A, BAK1, BID, ETS1, IKZF2, HIST1H1B, B7-H6, MICA, MICB, ULBP1, ULBP2, ULBP3, ULBP4, ULBP5, ULBP6, and ligands of NKp46.

14. The immune cell of claim 1, wherein the immune cell expresses or is further modified to express an engineered receptor.

15. The immune cell of claim 14, wherein the engineered receptor is a chimeric antigen receptor (CAR), an engineered TCR, or a T cell antigen coupler (TAC).

16. (canceled)

17. The immune cell of claim 14, wherein the modification to reduce or eliminate expression and/or function of the one or more target proteins:
i) does not down-regulate or eliminate expression and/or function of the engineered receptor; or
ii) down-regulates expression and/or function of the engineered receptor by at most about 30%.

18. The immune cell of claim 1, wherein the immune cell is a T cell, a B cell, or a natural killer (NK) cell.

19. (canceled)

20. The immune cell of claim 1, wherein the immune cell has at least about 10% longer in vivo persistence compared to a reference immune cell not having the modification to reduce or eliminate expression and/or function of the one or more target proteins.

21. The immune cell of claim 1, which is autologous or allogeneic.

22. (canceled)

23. A method of identifying an individual as a suitable donor of an immune cell with prolonged in vivo persistence, comprising examining the expression and/or function of one or more target proteins selected from the group consisting of SPPL3, FADD, FAS, CASP8, ARID1A, BAK1, BID, ETS1, IKZF2, and HIST1H1B in the individual, wherein the identification of reduced or abolished expression and/or function of the one or more target proteins compared to a reference identifies the individual as the suitable donor.

24. A method of excluding an individual as a suitable donor of an immune cell with prolonged in vivo persistence, comprising examining the expression and/or function of one or more target proteins selected from the group consisting of SPPL3, FADD, FAS, CASP8, ARID1A, BAK1, BID, ETS1, IKZF2, and HIST1H1B in the individual, wherein the individual is excluded as the suitable donor if no reduced or abolished expression and/or function of the one or more target proteins compared to a reference is identified, and wherein the reference is the average expression and/or function of the one or more target proteins in a population of individuals.

25-26. (canceled)

27. A method of i) prolonging in vivo persistence, ii) reducing AICD, and/or iii) reducing host-versus-graft (HvG) response of an immune cell, comprising modifying the immune cell to reduce or eliminate expression and/or function of one or more target proteins selected from the group consisting of: SPPL3, FADD, FAS, CASP8, ARID1A, BAK1, BID, ETS1, IKZF2, and HIST1H1B.

28-47. (canceled)

48. An immune cell obtained by the method of claim 27.

49. A pharmaceutical composition comprising the immune cell of claim 1, and optionally a pharmaceutically acceptable excipient.

50. A method of treating a disease in an individual, comprising administering to the individual an effective amount of the immune cell of claim 1.

51-53. (canceled)
